(19) United States
(12) Patent Application Publication
(10) Pub. No.: US 2023/0381130 A1
(43) Pub. Date: Nov. 30, 2023

(54) ANTIBODY SCAFFOLD STRUCTURE

(71) Applicant: Atreca, Inc., San Carlos, CA (US)

(72) Inventors: Jessica FINN, San Carlos, CA (US); Shaun M. LIPPOW, San Carlos, CA (US)

(21) Appl. No.: 18/300,198

(22) Filed: Apr. 13, 2023

Related U.S. Application Data
(63) Continuation-in-part of application No. PCT/US2021/054983, filed on Oct. 14, 2021.
(60) Provisional application No. 63/092,293, filed on Oct. 15, 2020.

(51) Int. Cl. A61K 31/216; A61P 33/14; A61K 9/00; A61K 31/42

(52) U.S. Cl. CPC A61K 31/216; A61P 33/14; A61K 9/0014; A61K 31/42

(57) ABSTRACT

Provided herein are antibodies comprising a scaffold region and methods of using such antibodies to generate antibodies with binding specificities that differ from a parent antibody that comprises the scaffold region.

### CROSS-REFERENCE TO RELATED APPLICATIONS

This application is a Continuation in Part of PCT/US2021/054983, filed Oct. 14, 2021, which claims benefit of U.S. Provisional Application No. 63/092,293, filed Oct. 15, 2020, which is herein incorporated by reference for all purposes.

### SEQUENCE LISTING

The instant application contains a Sequence Listing which has been submitted electronically in XML format and is hereby incorporated by reference in its entirety. Said XML copy, created on Jul. 27, 2023, is named 097519-1381708_2510US_SL.xml and is 2,343,004 bytes in size.

### BRIEF SUMMARY

Provided herein is an antibody scaffold that can be used for the identification of antibodies having new binding specificities compared to a parent antibody compared to the target of the parent antibody. Thus, in one aspect, the disclosure provides an antibody that binds an RNA-protein complex, wherein the antibody comprises a heavy chain variable region comprising a HCDR1, a HCDR2, and a HCDR3, and a light chain variable region comprising a LCDR1, a LCDR2, and a LCDR3,
wherein the antibody comprises a pocket that is 13-20 A deep,
and the HCDR3 comprises
i. at least 25 amino acids
ii. a first intra-HCDR3 disulfide bond, and
iii. optionally, a second intra-HCDR3 disulfide bond.

In some embodiments, the HCDR3 comprises the amino acid sequence of X¹CCX²CX³CX⁴, wherein X¹=4 amino acid residues, X²=4-7 amino acid residues, X³=6-8 amino acid residues, and X⁴=10-14 amino acid residues (SEQ ID NO: 1728); for example, in one embodiment, the HCDR3 comprises the amino acid sequence of (I/T)(S/T)X₃(F/Y)CCX₇(G/S)X₉X₁₀CX₁₂(N/S)X₁₄(D/E)TS(F/Y)CX₂(G/N)X₂₂X₂₃X₂₄,X₂₅ (F/Y)YX₂₈X₂₉(D/N)X₃₁, wherein X₃ is A, P, or S; X₇ is H, L, Q, or R; X₉ is A, G, K, or N; X₁₀ is A, N, Q, R, or S; X₁₂ is A, L, or P; X₁₄ is H, Q, R, or S; X₂₀ is A, G, or N; X₂₂ is Q, S, or Y; X₂₃ is D, F, N, or Y; X₂₄ is A, K, N, P, or Q; X₂₅ is D, Q, R, or S; X₂₈ is F, L, W, or Y; X₂₉ is F, M, or V; and X₃₁ is I, P, or V (SEQ ID NO: 1729). In another embodiment, the HCDR3 comprises the amino acid sequence of (I/T)(S/T)X₃(F/Y)X₅CX₇(G/S)X₉X₁₀CX₁₂X₁₃X₁₄(D/E)X₁₆SX₁₈X₁₉X₂₀X₂₁X₂₂X₂₃X₂₄X₂₅(F/Y)(F/Y)X₂₈X₂₉(D/N)X₃₁, wherein X₃ is A, P, S, or T; X₅ is A, C, or S; X₇ is H, L, Q, or R; X₉ is A, G, K, or N; X₁₀ is A, N, Q, R, or S; X₁₂ is A, L, or P; X₁₃ is A, N, or S; X₁₄ is H, Q, R, or S; X₁₆ is N, Q, or T; X₁₈ is F, M, or Y; X₁₉ is C, S, or V; X₂₀ is A, G, or N; X₂₁ is A, G, or N; X₂₂ is Q, S, or Y; X₂₃ is D, F, N, S, or Y; X₂₄ is A, K, N, P, Q, or S; X₂₅ is D, K, Q, R, or S; X₂₈ is F, L, W, or Y; X₂₉ is F, M, or V; and X₃₁ is I, P, or V. In some embodiment, the HCDR3 comprises the amino acid sequence of X¹CCX²CX³CX⁴, wherein X¹=4 amino acid residues, X²=4 amino acid residues, X³=7 amino acid residues, and X⁴=12 amino acid residues (SEQ ID NO: 1731). In some embodiments, the HCDR3 comprises the amino acid sequence of X¹CGGX²CX³ wherein X¹=4 amino acid residues, X²=7 amino acid residues, and X³=12 amino acid residues (SEQ ID NO: 1732). In some embodiments, the HCDR3 comprises the amino acid sequence of X¹SCX²CX³ wherein X¹=4 amino acid residues, X²=4 amino acid residues, and X³=19 amino acid residues. In a further embodiment, the HCDR3 comprises the amino acid sequence of TSSFCCRGGSCPSHDTSYCGGQYKSYYYMDV (SEQ ID NO: 21) comprises at least 1, 2, 3, 4, 5, 6, 7, 8, 9, or 10 amino acid substitutions at positions that maintain the first disulfide bond and, optionally the second disfulfide bond.

In some embodiments, the antibody comprises a pocket comprising amino acid residues 36, 37, 38, 39, 40, 52, 55, 58, 60, 61, 105, 106, 107, 108, 109, 111.5, 111.7, 111.8, 111.9, and 114 in the V_{H} per IMGT numbering and amino acid residues 37, 38, 40, 42, 55, 56, 107, and 116 in the V_{L} per IMGT numbering; and may further comprise one or more amino acid residues selected from the group consisting of 57, 111.6, 112.9, 112.7 and 116 in the V_{H} per IMGT numbering; and/or one or more amino acid residues selected from the group consisting of 39, 52, 57, 80, 105, and 106 in the V_{L} per IMGT numbering. In some embodiments, the pocket further comprises amino acid residues 57, 111.6, 112.9, 112.7 and 116 in the V_{H} per IMGT numbering; and/or amino acid residues 39, 52, 57, 80, 105, and 106 in the V_{L} per IMGT numbering. In some embodiments, the antibody comprises at least 1, 2, 3, 4, 5, 6, 7, 8, 9, 10, 11, 12, 13, 14, 15, 16, 17, 18, 19 or 20 of the following residues in the V_{H}: A, C, H, K, M, N or R at position 36; A or V at position 37, F, H, V, W, or Y at position 38; F, M or Y at position 39; S, T, or Y at position 40, W at position 52; F or R at position 55, A or S at position 58; D, H, Q, S, or T at position 60; D, E, N, or S at position 61; I, T, or V at position 105; F, S or T at position 106; A, P, S, or T at position 107; F or Y at position 108; A, C, or S at position 109; A, C, L or P at position 111.5; H, Q, R, or S at position 111.7; D or E at position 111.8; C, N, Q, or T at position 111.9; or C, F, L, W, or Y at position 114; and/or at least 1, 2, 3, 4, 5, 6, 7, or 8 of the following residues in the V_{L}: A, H, N, S, or T at position 37; A, D, F, S, T, or Y at position 38; A, D, E, L, S, T, or Y at position 40; Y at position 42; H or Y at position 55; A, H, K, M, N, or R at position 56; F, I or W at position 107; or H, K, Q, R, V, or W at position 116. In some embodiments, the antibody comprises at least 1, 2, 3, 4, or 5 of the following residues in the V_{H} per IMGT numbering: K or Q at position 57; A, C, D, N, or S at position 111.6; S at position 112.9; A, C, S, or V at position 112.7; or D or N at position 116; and/or at least 1, 2, 3, 4, 5, or 6 following residues in the V_{L} per IMGT numbering: T or V at position 39; L at position 52; D or N at position 57; K at position 80; A or S at position 105; or A, I, S, or T at position 106.

In some embodiments, the pocket comprises at least 1, 2, 3, 4, 5, 6, 7, 8, 9, 10, 11, 12, 13, 14, 15, 16, 17, 18, 19 or 20 of the following residues in the V_{H}: K at position 36; A at position 37; W at position 38; F or M at position 39; S or T at position 40; W at position 52; R at position 55; A or S at position 58; D, S, or T at position 60; D at position 61; I or T at position 105; S or T at position 106; S at position 107; F at position 108; C at position 109; P at position 111.5; H, R, or S at position 111.7; D at position 111.8; T at position 111.9; or Y at position 114; and/or at least 1, 2, 3, 4, 5, 6, 7, or 8 of the following residues in the V_{L}: H, N, or S at position 37; D, S or Y at position 38; E, L, or S at position 40; Y at position 42; H or Y at position 55; K or R at position 56; I or W at position 107; or K, R, or W at position 116. In some embodiments, the antibody further comprises at least 1, 2, 3, 4, or 5 of the following residues in the V_{H} per IMGT numbering: K at position 57; S at position 111.6; S at position 112.9; C at position 112.7; or D at position 116; and/or at least 1, 2, 3, 4, 5, or 6 following residues in the V_{L} per IMGT numbering: V at position 39; L at position 52; N at position 57; K at position 80; A or S at position 105; or A or T at position 106.

In a further aspect, provided herein is a method of generating an antibody that binds to an RNA-protein complex comprising mutating a scaffold antibody that binds to a first RNA-protein complex to generate an antibody that binds to a second RNA-protein complex and has an antigen binding specificity that differs from the antigen binding specificity of the scaffold antibody, wherein the scaffold antibody comprises a heavy chain variable region comprising a HCDR1, a HCDR2, and a HCDR3, and a light chain variable region comprising a LCDR1, a LCDR2, and a LCDR3, and comprises a pocket that is 13-20 A deep, and the HCDR3 comprises
i. at least 25 amino acids
ii. a first intra-HCDR3 disulfide bond, and
iii. optionally, a second intra-HCDR3 disulfide bond. In some embodiments of the method, the HCDR3 comprises the amino acid sequence of X¹CCX²CX³CX⁴, wherein X¹=4 amino acid residues, X²=4-7 amino acid residues, X³=6-8 amino acid residues, and X⁴=10-14 amino acid residues (SEQ ID NO: 1728); for example, in one embodiment, the HCDR3 comprises the amino acid sequence of (I/T)(S/T)X₃(F/Y)CCX₇(G/S)X₉X₁₀CX₁₂(N/S)X₁₄(D/E)TS(F/Y)CX₂₀(G/N)X₂₂X₂₃X₂₄,X₂₅ (F/Y)YX₂₈X₂₉(D/N)X₃₁, wherein X₃ is A, P, or S; X₇ is H, L, Q, or R; X₉ is A, G, K, or N; X₁₀ is A, N, Q, R, or S; X₁₂ is A, L, or P; X₁₄ is H, Q, R, or S; X₂₀ is A, G, or N; X₂₂ is Q, S, or Y; X₂₃ is D, F, N, or Y; X₂₄ is A, K, N, P, or Q; X₂₅ is D, Q, R, or S; X₂₈ is F, L, W, or Y; X₂₉ is F, M, or V; and X₃₁ is I, P, or V (SEQ ID NO: 1729). In another embodiment, the HCDR3 comprises the amino acid sequence of (I/T)(S/T)X₃(F/Y)X₅CX₇(G/S)X₉X₁₀CX₁₂X₁₃X₁₄(D/E)X₁₆SX₁₈X₁₉X₂₀X₂₁X₂₂X₂₃X₂₄X₂₅(F/Y)(F/Y)X₂₈X₂₉(D/N)X₃₁, wherein X₃ is A, P, S, or T; X₅ is A, C, or S; X₇ is H, L, Q, or R; X₉ is A, G, K, or N; X₁₀ is A, N, Q, R, or S; X₁₂ is A, L, or P; X₁₃ is A, N, or S; X₁₄ is H, Q, R, or S; X₁₆ is N, Q, or T; X₁₈ is F, M, or Y; X₁₉ is C, S, or V; X₂₀ is A, G, or N; X₂₁ is A, G, or N; X₂₂ is Q, S, or Y; X₂₃ is D, F, N, S, or Y; X₂₄ is A, K, N, P, Q, or S; X₂₅ is D, K, Q, R, or S; X₂₈ is F, L, W, or Y; X₂₉ is F, M, or V; and X₃₁ is I, P, or V. In some embodiment, the HCDR3 comprises the amino acid sequence of X¹CCX²CX³ C X⁴, wherein X¹=4 amino acid residues, X²=4 amino acid residues, X³=7 amino acid residues, and X⁴=12 amino acid residues (SEQ ID NO: 1731). In some embodiments, the HCDR3 comprises the amino acid sequence of X¹CGGX²CX³ wherein X¹=4 amino acid residues, X²=7 amino acid residues, and X³=12 amino acid residues (SEQ ID NO: 1732). In some embodiments, the HCDR3 comprises the amino acid sequence of X¹SCX²CX³ wherein X¹=4 amino acid residues, X²=4 amino acid residues, and X³=19 amino acid residues. In a further embodiment, the HCDR3 comprises the amino acid sequence of TSSFCCRGGSCPSHDTSYCGGQYKSYYYMDV (SEQ ID NO: 21) comprises at least 1, 2, 3, 4, 5, 6, 7, 8, 9, or 10 amino acid substitutions at positions that maintain the first disulfide bond and, optionally the second disfulfide bond.

In some embodiments of the method, the antibody comprises a pocket comprising amino acid residues 36, 37, 38, 39, 40, 52, 55, 58, 60, 61, 105, 106, 107, 108, 109, 111.5, 111.7, 111.8, 111.9, and 114 in the V_{H} per IMGT numbering and amino acid residues 37, 38, 40, 42, 55, 56, 107, and 116 in the V_{L} per IMGT numbering; and may further comprise one or more amino acid residues selected from the group consisting of 57, 111.6, 112.9, 112.7 and 116 in the V_{H} per IMGT numbering; and/or one or more amino acid residues selected from the group consisting of 39, 52, 57, 80, 105, and 106 in the V_{L} per IMGT numbering. In some embodiments, the pocket further comprises amino acid residues 57, 111.6, 112.9, 112.7 and 116 in the V_{H} per IMGT numbering; and/or amino acid residues 39, 52, 57, 80, 105, and 106 in the V_{L} per IMGT numbering. In some embodiments, the antibody comprises at least 1, 2, 3, 4, 5, 6, 7, 8, 9, 10, 11, 12, 13, 14, 15, 16, 17, 18, 19 or 20 of the following residues in the V_{H}: A, C, H, K, M, N or R at position 36; A or V at position 37, F, H, V, W, or Y at position 38; F, M or Y at position 39; S, T, or Y at position 40, W at position 52; F or R at position 55, A or S at position 58; D, H, Q, S, or T at position 60; D, E, N, or S at position 61; I, T, or V at position 105; F, S or T at position 106; A, P, S, or T at position 107; F or Y at position 108; A, C, or S at position 109; A, C, L or P at position 111.5; H, Q, R, or S at position 111.7; D or E at position 111.8; C, N, Q, or T at position 111.9; or C, F, L, W, or Y at position 114; and/or at least 1, 2, 3, 4, 5, 6, 7, or 8 of the following residues in the V_{L}: A, H, N, S, or T at position 37; A, D, F, S, T, or Y at position 38; A, D, E, L, S, T, or Y at position 40; Y at position 42; H or Y at position 55; A, H, K, M, N, or R at position 56; F, I or W at position 107; or H, K, Q, R, V, or W at position 116. In some embodiments, the antibody comprises at least 1, 2, 3, 4, or 5 of the following residues in the V_{H} per IMGT numbering: K or Q at position 57; A, C, D, N, or S at position 111.6; S at position 112.9; A, C, S, or V at position 112.7; or D or N at position 116; and/or at least 1, 2, 3, 4, 5, or 6 following residues in the V_{L} per IMGT numbering: T or V at position 39; L at position 52; D or N at position 57; K at position 80; A or S at position 105; or A, I, S, or T at position 106.

In some embodiments of the method, the pocket comprises at least 1, 2, 3, 4, 5, 6, 7, 8, 9, 10, 11, 12, 13, 14, 15, 16, 17, 18, 19 or 20 of the following residues in the V_{H}: K at position 36; A at position 37; W at position 38; F or M at position 39; S or T at position 40; W at position 52; R at position 55; A or S at position 58; D, S, or T at position 60; D at position 61; I or T at position 105; S or T at position 106; S at position 107; F at position 108; C at position 109; P at position 111.5; H, R, or S at position 111.7; D at position 111.8; T at position 111.9; or Y at position 114; and/or at least 1, 2, 3, 4, 5, 6, 7, or 8 of the following residues in the V_{L}: H, N, or S at position 37; D, S or Y at position 38; E, L, or S at position 40; Y at position 42; H or Y at position 55; K or R at position 56; I or W at position 107; or K, R, or W at position 116. In some embodiments, the antibody further comprises at least 1, 2, 3, 4, or 5 of the following residues in the V_{H} per IMGT numbering: K at position 57; S at position 111.6; S at position 112.9; C at position 112.7; or D at position 116; and/or at least 1, 2, 3, 4, 5, or 6 following residues in the V_{L} per IMGT numbering: V at position 39; L at position 52; N at position 57; K at position 80; A or S at position 105; or A or T at position 106.

In some embodiments of the methods, the scaffold antibody comprises an HCDR3 as set forth in Table 1B, Table 2B, Table 3B, or Table 4B. In further embodiments, the caffold antibody comprises the six CDRs of an antibody as set forth in Table 1B, Table 2B, Table 3B, or Table 4B.

In a further aspect of the methods, the method comprises generating a plurality of mutated scaffold antibodies; screening the mutated scaffold antibodies for binding to the second RNA-protein complex; and selecting a mutated scaffold antibody that binds to the second RNA-protein complex.

In another aspect of the disclosure, provided herein is a library comprising a plurality of mutated scaffold antibodies produced by the method as described herein, e.g., in the above paragraphs.

In some embodiments, a parent antibody that can be diversified to generate antibodies having new binding specificities comprises an HCDR1, HCDR2, HCDR3, LCDR1, LCDR2, and LCDR3 of a V_{H} or a V_{L} region of an antibody designated as AIP-192482, AIP-171142, AIP-165430, AIP-189526, AIP-122563, AIP-158623, AIP-155066, AIP-166120, AIP-133645, AIP-187893, AIP-142079, AIP-160470, AIP-102396, AIP-150055, AIP-167084, AIP-185304, AIP-134770, AIP-141887, AIP-196203, AIP-128195, AIP-116579, AIP-192329, AIP-197809, AIP-142489, AIP-167726, AIP-199834, AIP-143179, AIP-195587, AIP-153462, AIP-115363, AIP-151090, AIP-168083, AIP-161082, AIP-114196, AIP-189338, AIP-183190, AIP-110143, AIP-147176, AIP-134312, AIP-128243, AIP-156172, AIP-147389, AIP-124314, AIP-185291, AIP-135247, AIP-113513, AIP-102299, AIP-179097, AIP-109343, AIP-119622, AIP-191735, AIP-157078, AIP-153475, AIP-133650, AIP-190915, AIP-167400, AIP-109729, AIP-151709, AIP-136628, AIP-101601, AIP-146871, AIP-170053, AIP-199483, AIP-162041, AIP-180675, AIP-183133, AIP-191470, AIP-151167, AIP-106633, AIP-102624, AIP-109484, AIP-126080, AIP-161571, AIP-163039, AIP-101235, AIP-182061, AIP-181246, AIP-192216, AIP-171912, AIP-172872, AIP-167833, AIP-190051, AIP-145518, AIP-167533, AIP-112580, AIP-143155, AIP-119664, AIP-190526, AIP-114403, AIP-156760, AIP-103803, AIP-195588, AIP-145722, AIP-178251, AIP-116142, AIP-183350, AIP-127108, AIP-128147, AIP-109510, AIP-104086, AIP-143132, AIP-170105, AIP-169636, AIP-152243, AIP-138776, AIP-103817, AIP-130491, AIP-188155, AIP-167246, AIP-106139, AIP-198351, AIP-159326, AIP-192275, AIP-190761, AIP-166832, AIP-148062, AIP-129145, AIP-111240, AIP-153888, AIP-130915, AIP-109048, AIP-170569, AIP-154873, AIP-159037, AIP-186826, AIP-156514, AIP-157122, AIP-173276, AIP-150485, AIP-166847, AIP-124013, AIP-126285, AIP-168605, AIP-190274, AIP-136060, AIP-180422, AIP-166722, AIP-127782, AIP-189473, AIP-192571, AIP-112328, AIP-125258, AIP-150199, AIP-125062, AIP-177193, AIP-115388, AIP-107759, AIP-170221, AIP-143369, AIP-189475, AIP-102833, AIP-157045, AIP-175775, AIP-154181, AIP-125984, AIP-160829, AIP-184744, AIP-128136, AIP-181273, AIP-153125, AIP-131972, AIP-131762, AIP-194564, AIP-169752, AIP-185680, AIP-145328, AIP-182606, AIP-107388, AIP-191692, AIP-163723, AIP-167295, AIP-125646, or AIP-114111 in Table 1A, Table 2A, or Table 3A, or a variant thereof in which at least one, two, three, four, five, or all six of the CDRs contain 1 or 2 amino acid substitutions. In some embodiments, a parent antibody that can be diversified to generate antibodies having new binding specificities comprises an HCDR1, HCDR2, HCDR3, LCDR1, LCDR2, and LCDR3 of a V_{H} or a V_{L} region of an antibody designated as AIP-192482. AIP-186044, AIP-122563, AIP-160470, AIP-145518, AIP-167533, AIP-112580, AIP-150277, AIP-136060, or AIP-154708 in Table 4A, or a variant thereof in which at least one, two, three, four, five, or all six of the CDRs contain 1 or 2 amino acid substitutions.

In some embodiments, the antibody comprises an HCDR1, HCDR2, HCDR3, LCDR1, LCDR2, and LCDR3 of an antibody set forth in Table 1B, Table 2B, Table 3B, or Table 4B.

In a further aspect, the disclosure provides an antibody that binds to tumor tissue, wherein the antibody binds to an extracellular RNA-protein complex and wherein the antibody comprises:
a heavy chain variable region comprising:
an HCDR1 comprising the sequence GFTFSKAWMS (SEQ ID NO: 1), or a variant HCDR1 in which 1, 2, 3, 4, or 5 amino acids are substituted relative to the sequence;
an HCDR2 comprising the sequence RIKSVTDGETTDYAAPVKG (SEQ ID NO: 9), or a variant HCDR2 in which 1, 2, 3, 4, or 5 amino acids are substituted relative to the sequence; and
an HCDR3 comprising the sequence of an HCDR3 set forth in Table 3B, or a variant HCDR3 in which 1, 2, 3, 4, or 5 amino acids are substituted relative to the sequence; a light chain variable region comprising:
an LCDR1 comprising the sequence SGSSSNIGSSSVS (SEQ ID NO: 48), or a variant LCDR1 in which 1, 2, 3, 4, or 5 amino acids are substituted relative to the sequence;
an LCDR2 comprising the sequence KNNQRPS (SEQ ID NO: 59), or variant LCDR2 in which 1, 2, or 3 amino acids are substituted relative to the sequence; and
an LCDR3 comprising the sequence STWDDSLSVRV (SEQ ID NO: 68), or a variant
LCDR3 in which 1, 2, 3, 4, or 5 amino acids are substituted relative to the sequence.
   In some embodiments, the antibody comprises a CDR3 sequence:
TSSFCGGPSHDTSYCGGQYKSYYYMDV (SEQ ID NO: 1734),
TSSFCCRSGGSCPSHDTSYCGGQYKSYYYMDV (SEQ ID NO: 1735),
TSSFCCRGGGSCPSHDTSYCGGQYKSYYYMDV (SEQ ID NO: 1730),
TSSFCCRGGGGSCPSHDTSYCGGQYKSYYYMDV (SEQ ID NO: 1737),
TSSFCCRGGSGGSCPSHDTSYCGGQYKSYYYMDV (SEQ ID NO: 1738),
TSSFSCRGGSCPSHDTSYGGQYKSYYYMDV (SEQ ID NO: 1739),
TSSFCCRGGSCPSHDTSYCGGGQYKSYYYMDV (SEQ ID NO: 1740),
TSSFCCRGGSCPSHDTSYCGGGSGYKSYYYMDV (SEQ ID NO: 1741),
TSSFCCRGGSCPSHDTSYCGGQKSYYYMDV (SEQ ID NO: 1742),
TSSFCCRGGSCPSHDTSYCGGQYSYYYMDV (SEQ ID NO: 1743),
TSSFCCRGGSCPSHDTSYCGQYKSYYYMDV (SEQ ID NO: 1744), or
TSSFCCRGGSCPSHDTSYCGGQYKSSYYYMDV (SEQ ID NO: 1745); or a variant thereof having 1, 2, or 3 amino acid substitutions. In some embodiments, the antibody binds to an extracellular RNA-protein complex comprising a polyadenylate binding protein family member selected from PABPC 1 (PABPC1), PABPC 3 (PABPC3), or PABPC 4 (PABPC4). In some embodiments, the antibody binds to an extracellular RNA-protein complex comprising the PABPC family member is PABPC1.

In some embodiments, the disclosure provides an antibody that binds to tumor tissue, wherein the antibody comprises:
the HCDR1 comprises the sequence GFTFSKAWMS (SEQ ID NO: 1),
the HCDR2 comprises the sequence RIKSVTDGETTDYAAPVKG (SEQ ID NO: 9),
the HCDR3 comprises the sequence TSSFCCRGGSCPSHDTSYCGQYKSYYYMDV (SEQ ID NO: 1744) or
TSSFCCRGGSCPSHDTSYCGGQYKSSYYYMDV (SEQ ID NO: 1745),
the LCDR1 comprises the sequence SGSSSNIGSSSVS (SEQ ID NO: 48),
the LCDR2 comprises the sequence KNNQRPS (SEQ ID NO: 59), and
the LCDR3 comprises the sequence STWDDSLSVRV (SEQ ID NO: 68). In some embodiments, the antibody binds to an extracellular RNA-protein complex comprising a polyadenylate binding protein family member selected from PABPC 1 (PABPC1), PABPC 3 (PABPC3), or PABPC 4 (PABPC4). In some embodiments, the antibody binds to an extracellular RNA-protein complex comprising the PABPC family member is PABPC1.

### BRIEF DESCRIPTION OF THE DRAWINGS

FIG. 1 depicts a top view of the structure of AIP-192482 and pocket.

FIG. 2 depicts a front view of the structure of AIP-192482 and pocket.

FIG. 3 depicts a side view of the structure of AIP-192482 and pocket.

FIG. 4 depicts a front view of the pocket of AIP-192482, and shows the depth of the pocket.

FIG. 5 depicts the sub-loop structures of an HCDR3.

FIGS. 6A-6B1 provide the sequences of the V_{H} region and V_{L} region of AIP-192482 with conserved pocket residues indicated. Numbering is shown using Kabat, Chothia, and IMGT numbering systems. FIGS. 6A-6B1 disclose SEQ ID NOS 95 and 141, respectively, in order of appearance.

FIGS. 7A-7B1 provide the sequence of the V_{H} region and V_{L} region of AIP-192482 with pocket residues indicated, including additional pocket residues compared to FIGS. 6A-6B1. FIGS. 7A-7B1 disclose SEQ ID NOS 95 and 141, respectively, in order of appearance.

FIGS. 8A and 8B show residues observed in 10 exemplar scaffold antibodies at conserved pocket positions of the V_{H} and V_{L} regions.

FIGS. 9A and 9B show residues observed in 10 exemplar scaffold antibodies at majority conserved pocket positions of the V_{H} and V_{L} regions. FIG. 9A discloses SEQ ID NO: 1766.

FIGS. 10A and 10B show residues observed in 256 expressed scaffold antibodies at conserved pocket positions of the V_{H} and V_{L} regions.

FIGS. 11A and 11B show residues observed in 256 expressed scaffold antibodies at majority conserved pocket positions of the V_{H} and V_{L} regions.

FIG. 12 shows tumor growth data from a screen of a pool of antibodies (4 antibodies, each obtained from a lung cancer patient) and AIP-192482 antibody identified in the screen in combination with an anti-PD-1 antibody. Panel A, PBS control; Panel B, anti-PD1 antibody only; Panel C, anti-PD1 plus antibody pool; Panel E, combination of anti-PD1 antibody and AIP-192482.

FIGS. 13A-13C provide immunohistochemical data showing binding of initial lead antibody (FIG. 13A), variant AIP-133645 (FIG. 13B), and variant AIP-160470 (FIG. 13C) to tissue from tumors arising from human lung A549 cells, human pancreas BXPC3 cells, human colon cancer Colo-205 cells, or human prostate cancer PC3 cells; and tumor arising from mouse colon, breast, lung, or kidney cancer cell lines.

FIG. 14 provides the results of an analysis of AIP-192482-target complex immunoprecipitates treated with RNase or DNase.

FIG. 15 shows the results of SDS PAGE analysis of immunoprecipitation using lysate from unlabelled A549 cells s incubated with AIP-192482-conjugated M-280 beads and then crosslinked.

FIG. 16 shows gel filtration purification of AIP-192482-antigen complex.

FIGS. 17A-17G shows variant activity in an in vitro FcR engagement assay.

FIG. 18 provides data showing binding of variants to EMT6 ex vivo cells in correlation with in vivo data.

FIG. 19 provides data showing that binding of variants to ex vivo EMT6 cells correlates with in vivo outcome. C=a control anti-EGFR antibody

### DETAILED DESCRIPTION

### Terminology

As used in herein, the singular forms “a”, “an” and “the” include plural referents unless the content clearly dictates otherwise. Thus, for example, reference to “an antibody” optionally includes a combination of two or more such molecules, and the like.

The term “about” as used herein refers to the usual error range for the respective value readily known to the skilled person in this technical field, for example ±20%, ±10%, or ±5%, are within the intended meaning of the recited value.

As used herein, the term “antibody” means an isolated or recombinant binding agent that comprises the necessary variable region sequences to specifically bind an antigenic epitope. Therefore, an “antibody” as used herein is any form of antibody of any class or subclass or fragment thereof that exhibits the desired biological activity, e.g., binding a specific target antigen. Thus, it is used in the broadest sense and specifically covers a monoclonal antibody (including full-length monoclonal antibodies), human antibodies, chimeric antibodies, nanobodies, diabodies, multispecific antibodies (e.g., bispecific antibodies), and antibody fragments including but not limited to scFv, Fab, and the like so long as they exhibit the desired biological activity.

“Antibody fragments” comprise a portion of an intact antibody, for example, the antigen-binding or variable region of the intact antibody. Examples of antibody fragments include Fab, Fab′, F(ab′)₂, and Fv fragments; diabodies; linear antibodies (e.g., Zapata et al., Protein Eng. 8(10): 1057-1062 (1995)); single-chain antibody molecules (e.g., scFv); and multispecific antibodies formed from antibody fragments. Papain digestion of antibodies produces two identical antigen-binding fragments, called “Fab” fragments, each with a single antigen-binding site, and a residual “Fc” fragment, a designation reflecting the ability to crystallize readily. Pepsin treatment yields an F(ab′)₂ fragment that has two antigen combining sites and is still capable of cross-linking antigen.

An RNA-protein complex” refers to a complex that is associated with a cell, or a plurality of cells. The complex need not be integrated into the external surface of the cells, but in some embodiments, may be associated with the outside of cells as a conglomeration or aggregate of RNA and protein molecules interacting with the cell membrane, or otherwise present outside of the cell. An extracellular RNA-protein complex may also be present internally in a cell.

As used herein, “V-region” refers to an antibody variable region domain comprising the segments of Framework 1, CDR1, Framework 2, CDR2, and Framework 3, including CDR3 and Framework 4. The heavy chain V-region, V_{H}, is a consequence of rearrangement of a V-gene (HV), a D-gene (HD), and a J-gene (HJ), in what is termed V(D)J recombination during B-cell differentiation. The light chain V-region, V_{L}, is a consequence of rearrangement of a V-gene (LV) and a J-gene (LJ).

As used herein, “complementarity-determining region (CDR)” refers to the three hypervariable regions in each chain that interrupt the four “framework” regions established by the light and heavy chain variable regions. The CDRs are the primary contributors to binding to an epitope of an antigen. The CDRs of each chain are referred to as CDR1, CDR2, and CDR3, numbered sequentially starting from the N-terminus, and are also identified by the chain in which the particular CDR is located. Thus, for example, a V_{H} CDR3 (HCDR3) is located in the variable domain of the heavy chain of the antibody in which it is found, whereas a V_{L} CDR3 (LCDR3) is the CDR3 from the variable domain of the light chain of the antibody in which it is found.

The amino acid sequences of the CDRs and framework regions can be determined using various well known definitions in the art, e.g., Kabat, Chothia, international ImMunoGeneTics database (IMGT), and AbM (see, e.g., Chothia & Lesk, 1987, Canonical structures for the hypervariable regions of immunoglobulins. J. Mol. Biol. 196, 901-917; Chothia C. et al., 1989, Conformations of immunoglobulin hypervariable regions. Nature 342, 877-883; Chothia C. et al., 1992, structural repertoire of the human VH segments J. Mol. Biol. 227, 799-817; Al-Lazikani et al., J. Mol. Biol 1997, 273(4)). Definitions of antigen combining sites are also described in the following: Ruiz et al., IMGT, the international ImMunoGeneTics database. Nucleic Acids Res., 28, 219-221 (2000); and Lefranc, M.-P. IMGT, the international ImMunoGeneTics database. Nucleic Acids Res. January 1; 29(1):207-9 (2001); MacCallum et al, Antibody-antigen interactions: Contact analysis and binding site topography, J. Mol. Biol., 262 (5), 732-745 (1996); and Martin et al, Proc. Natl Acad. Sci. USA, 86, 9268-9272 (1989); Martin, et al, Methods Enzymol., 203, 121-153, (1991); Pedersen et al, Immunomethods, 1, 126, (1992); and Rees et al, In Stenberg M. J. E. (ed.), Protein Structure Prediction. Oxford University Press, Oxford, 141-172 1996). Reference to CDRs as determined by Kabat numbering are based, for example, on Kabat et al., Sequences of Proteins of Immunological Interest, 5th Ed. Public Health Service, National Institute of Health, Bethesda, MD (1991)). Chothia CDRs are determined as defined by Chothia (see, e.g., Chothia and Lesk J. Mol. Biol. 196:901-917 (1987)).

An “Fc region” refers to the constant region of an antibody excluding the first constant region immunoglobulin domain. Thus, e.g., for human immunoglobulins, “Fc” refers to the last two constant region immunoglobulin domains of IgA, IgD, and IgG, and the last three constant region immunoglobulin domains of IgE and IgM, and the flexible hinge N-terminal to these domains. For IgA and IgM Fc may include the J chain. For IgG, Fc comprises immunoglobulin domains Cγ2 and Cγ3 and the hinge between Cγ1 and Cγ. It is understood in the art that the boundaries of the Fc region may vary, however, the human IgG heavy chain Fc region is usually defined to comprise residues C226 or P230 to its carboxyl-terminus, using the numbering according to the EU index as in Kabat et al. (1991, NIH Publication 91-3242, National Technical Information Service, Springfield, Va.). The term “Fc region” may refer to this region in isolation or this region in the context of an antibody or antibody fragment. “Fc region” includes naturally occurring allelic variants of the Fc region as well as modified Fc regions, e.g., that are modified to modulate effector function or other properties such as pharmacokinetics, stability or production properties of an antibody. Fc regions also include variants that do not exhibit alterations in biological function. For example, one or more amino acids can be deleted from the N-terminus or C-terminus of the Fc region of an immunoglobulin without substantial loss of biological function. Such variants can be selected according to general rules known in the art so as to have minimal effect on activity (see, e.g., Bowie, et al., Science 247:306-1310, 1990). For example, for IgG4 antibodies, a single amino acid substitution (S228P according to Kabat numbering; designated IgG4Pro) may be introduced to abolish the heterogeneity observed in recombinant IgG4 antibody (see, e.g., Angal, et al., Mol Immunol 30:105-108, 1993).

An “EC₅₀” as used herein in the context of an Fc receptor engagement assay, refers to the half maximal effective concentration, which is the concentration of an antibody that induces a response (signal generated in engagement assay) halfway between the baseline and maximum after a specified exposure time. Fc receptor engagement assays are further described herein in the “Variant Binding Activity” section. In some embodiments, the “fold over EC₅₀” is determined by dividing the EC₅₀ of a reference antibody by the EC₅₀ of the test antibody.

The term “equilibrium dissociation constant” abbreviated (K_{D}), refers to the dissociation rate constant (k_{d}, time⁻¹) divided by the association rate constant (kₐ, time⁻¹ M⁻¹). Equilibrium dissociation constants can be measured using any method. Thus, in some embodiments antibodies comprising a scaffold region as described in the present disclosure bind to a target, e.g., RNA-protein complex, with a K_{D} of less than about 50 nM, typically less than about 25 nM, or less than 10 nM, e.g., less than about 5 nM or than about 1 nM and often less than about 10 nM as determined by surface plasmon resonance analysis using a biosensor system such as a Biacore® system performed at 37° C. In some embodiments, an antibody comprising a scaffold region of the present disclosure binds to a target, e.g., an RNA-protein complex, with a K_{D} of less than 5×10⁻⁵ M, less than 10⁻⁵ M, less than 5×10⁻⁶ M, less than 10⁻⁶ M, less than 5×10⁻⁷ M, less than 10⁻⁷ M, less than 5×10⁻⁸ M, less than 10⁻⁸ M, less than 5×10⁻⁹ M, less than 10⁻⁹ M, less than 5×10⁻¹⁰ M, less than 10⁻¹⁰ M, less than 5×10⁻¹¹ M, less than 10⁻¹¹ M, less than 5×10⁻¹² M, less than 10⁻¹² M, less than 5×10¹³ M, less than 10⁻¹³ M, less than 5×10⁻¹⁴ M, less than 10⁻¹⁴ M, less than 5×10⁻¹⁵ M, or less than 10⁻¹⁵ M or lower as measured as a bivalent antibody. In the context of the present invention, an “improved” K_{D} refers to a lower K_{D}. In some embodiments, an antibody comprising a scaffold region as described herein binds to a target, e.g., RNA-protein complex, with a K_{D} of less than 5×10⁻⁵ M, less than 10⁻⁵ M, less than 5×10⁻⁶ M, less than 10⁻⁶ M, less than 5×10⁻⁷ M, less than 10⁻⁷ M, less than 5×10⁻⁸ M, less than 10⁻⁸M, less than 5×10⁻⁹ M, less than 10⁻⁹ M, less than 5×10⁻¹⁰ M, less than 10⁻¹⁰ M, less than 5×10⁻¹¹ M, less than 10⁻¹¹ M, less than 5×10⁻¹² M, less than 10⁻¹² M, less than 5×10⁻¹³ M, less than 10⁻¹³ M, less than 5×10⁻¹⁴ M, less than 10⁻¹⁴ M, less than 5×10⁻¹⁵ M, or less than 10⁻¹⁵ M or lower as measured as a monovalent antibody, such as a monovalent Fab. In some embodiments, the antibody binds to the target, e.g., RNA-protein complex, with a K_{D} less than 100 pM, e.g., or less than 75 pM, e.g., in the range of 1 to 100 pM, when measured by surface plasmon resonance analysis using a biosensor system such as a Biacore® system performed at 37° C. In some embodiments, the antibody has a K_{D} of greater than 100 pM, e.g., in the range of 100-1000 pM or 500-1000 pM when measured by surface plasmon resonance analysis using a biosensor system such as a Biacore® system performed at 37° C.

The terms “identical” or percent “identity,” in the context of two or more polypeptide sequences, refer to two or more sequences or subsequences that are the same or have a specified percentage of amino acid residues that are the same (e.g., at least 70%, at least 75%, at least 80%, 85%, 90%, 91%, 92%, 93%, 94%, 95%, 96%, 97%, 98%, 99%, or higher) identity over a specified region, e.g., the length of the two sequences, when compared and aligned for maximum correspondence over a comparison window or designated region. Alignment for purposes of determining percent amino acid sequence identity can be performed in various methods, including those using publicly available computer software such as BLAST, BLAST-2, ALIGN or Megalign (DNASTAR) software. Examples of algorithms that are suitable for determining percent sequence identity and sequence similarity the BLAST 2.0 algorithms, which are described in Altschul et al., Nuc. Acids Res. 25:3389-3402 (1977) and Altschul et al., J. Mol. Biol. 215:403-410 (1990). Thus, for purposes of this invention, BLAST 2.0 can be used with the default parameters to determine percent sequence identity.

The terms “corresponding to,” “determined with reference to,” or “numbered with reference to” when used in the context of the identification of a given amino acid residue in a polypeptide sequence, refers to the position of the residue of a specified reference sequence when the given amino acid sequence is maximally aligned and compared to the reference sequence. Thus, for example, an amino acid residue in a V_{H} region polypeptide “corresponds to” an amino acid in the V_{H} region of SEQ ID NO:1 when the residue aligns with the amino acid in SEQ ID NO:1 when optimally aligned to SEQ ID NO:1. The polypeptide that is aligned to the reference sequence need not be the same length as the reference sequence.

A “conservative” substitution as used herein refers to a substitution of an amino acid such that charge, polarity, hydropathy (hydrophobic, neutral, or hydrophilic), and/or size of the side group chain is maintained. Illustrative sets of amino acids that may be substituted for one another include (i) positively-charged amino acids Lys and Arg; and His at pH of about 6; (ii) negatively charged amino acids Glu and Asp; (iii) aromatic amino acids Phe, Tyr and Trp; (iv) nitrogen ring amino acids His and Trp; (v) aliphatic hydrophobic amino acids Ala, Val, Leu and Ile; (vi) hydrophobic sulfur-containing amino acids Met and Cys, which are not as hydrophobic as Val, Leu, and Ile; (vii) small polar uncharged amino acids Ser, Thr, Asp, and Asn (viii) small hydrophobic or neutral amino acids Gly, Ala, and Pro; (ix) amide-comprising amino acids Asn and Gln; and (xi) beta-branched amino acids Thr, Val, and Ile. Reference to the charge of an amino acid in this paragraph refers to the charge at pH 6-7.

The terms “nucleic acid” and “polynucleotide” are used interchangeably and as used herein refer to both sense and anti-sense strands of RNA, cDNA, genomic DNA, and synthetic forms and mixed polymers of the above. In particular embodiments, a nucleotide refers to a ribonucleotide, deoxynucleotide or a modified form of either type of nucleotide, and combinations thereof. The terms also include, but is not limited to, single- and double-stranded forms of DNA. In addition, a polynucleotide, e.g., a cDNA or mRNA, may include either or both naturally occurring and modified nucleotides linked together by naturally occurring and/or non-naturally occurring nucleotide linkages. The nucleic acid molecules may be modified chemically or biochemically or may contain non-natural or derivatized nucleotide bases, as will be readily appreciated by those of skill in the art. Such modifications include, for example, labels, methylation, substitution of one or more of the naturally occurring nucleotides with an analogue, internucleotide modifications such as uncharged linkages (e.g., methyl phosphonates, phosphotriesters, phosphoramidates, carbamates, etc.), charged linkages (e.g., phosphorothioates, phosphorodithioates, etc.), pendent moieties (e.g., polypeptides), intercalators (e.g., acridine, psoralen, etc.), chelators, alkylators, and modified linkages (e.g., alpha anomeric nucleic acids, etc.). The above term is also intended to include any topological conformation, including single-stranded, double-stranded, partially duplexed, triplex, hairpinned, circular and padlocked conformations. A reference to a nucleic acid sequence encompasses its complement unless otherwise specified. Thus, a reference to a nucleic acid molecule having a particular sequence should be understood to encompass its complementary strand, with its complementary sequence. The term also includes codon-optimized nucleic acids that encode the same polypeptide sequence.

The term “vector,” as used herein, refers to a nucleic acid molecule capable of propagating another nucleic acid to which it is linked. The term includes the vector as a self-replicating nucleic acid structure as well as the vector incorporated into the genome of a host cell into which it has been introduced. A “vector” as used here refers to a recombinant construct in which a nucleic acid sequence of interest is inserted into the vector. Certain vectors are capable of directing the expression of nucleic acids to which they are operatively linked. Such vectors are referred to herein as “expression vectors”.

A “substitution,” as used herein, denotes the replacement of one or more amino acids or nucleotides by different amino acids or nucleotides, respectively.

An “isolated” nucleic acid refers to a nucleic acid molecule that has been separated from a component of its natural environment. An isolated nucleic acid includes a nucleic acid molecule contained in cells that ordinarily contain the nucleic acid molecule, but the nucleic acid molecule is present extrachromosomally or at a chromosomal location that is different from its natural chromosomal location.

“Isolated nucleic acid encoding an antibody or fragment thereof” refers to one or more nucleic acid molecules encoding antibody heavy or light chains (or fragments thereof), including such nucleic acid molecule(s) in a single vector or separate vectors, and such nucleic acid molecule(s) present at one or more locations in a host cell.

The terms “host cell,” “host cell line,” and “host cell culture” are used interchangeably and refer to cells into which exogenous nucleic acid has been introduced, including the progeny of such cells. Thus, a host cell is a recombinant host cells and includes the primary transformed cell and progeny derived therefrom without regard to the number of passages.

A polypeptide “variant,” as the term is used herein, is a polypeptide that typically differs from one or more polypeptide sequences specifically disclosed herein in one or more substitutions, deletions, additions and/or insertions. “Diversifying” an antibody sequence refers to introducing mutations to generate variants.

Scaffold Antibody that Bind to an RNA-Protein Complex

In one aspect, provided herein are scaffold antibodies that bind to an RNA-protein complex where the antibody comprises a scaffold region that has a distinct structure that favors binding interactions with an RNA-protein structure, as further detailed below. Such antibodies can be used as a template to generate antibodies having new binding specificities. A “scaffold antibody” provided herein comprises an HCDR3 at least 25 amino acid in length having at least one, and in some embodiments two, disulfide bonds in the HCDR3 loop. A second important structural feature of a scaffold antibody provided herein is a deep pocket formed between the V_{H} and V_{L} domains as further detailed below. The antibody scaffold provides an initial template in which the sequence can be diversified and the variant sequences then evaluated in vitro for binding activity to identify antibodies that have altered binding specificity compared to the parent antibody employed as a scaffold.

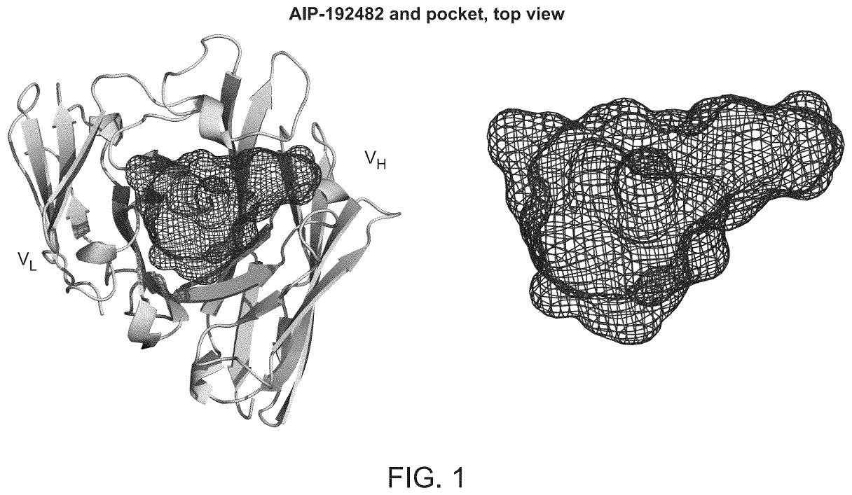

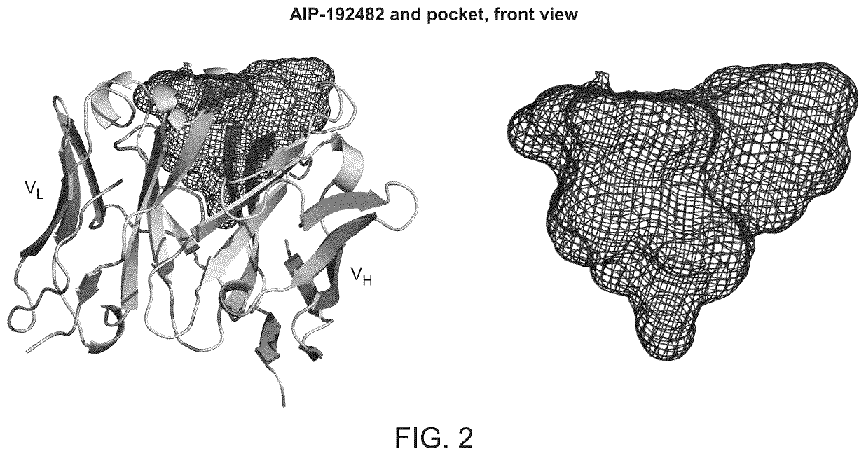

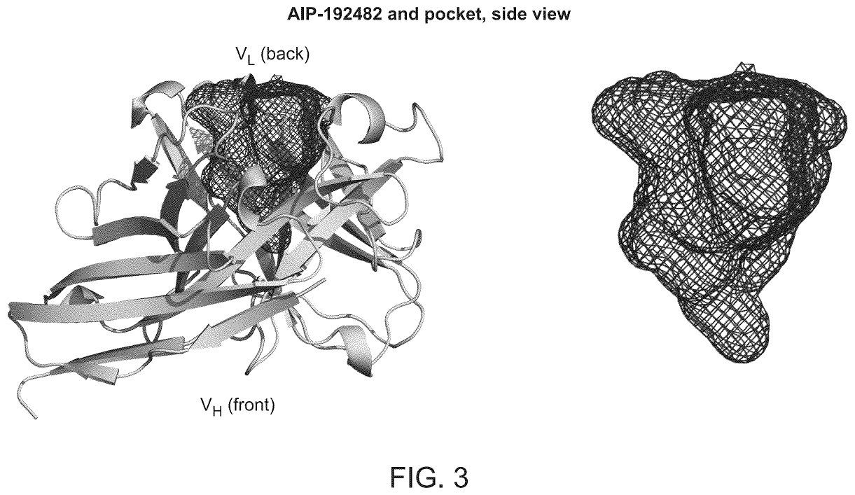

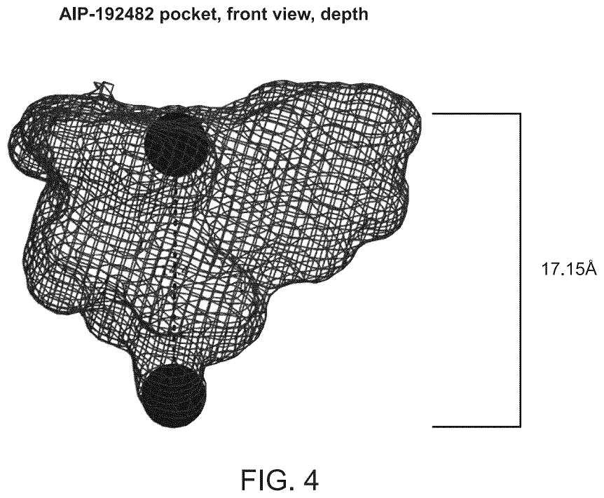

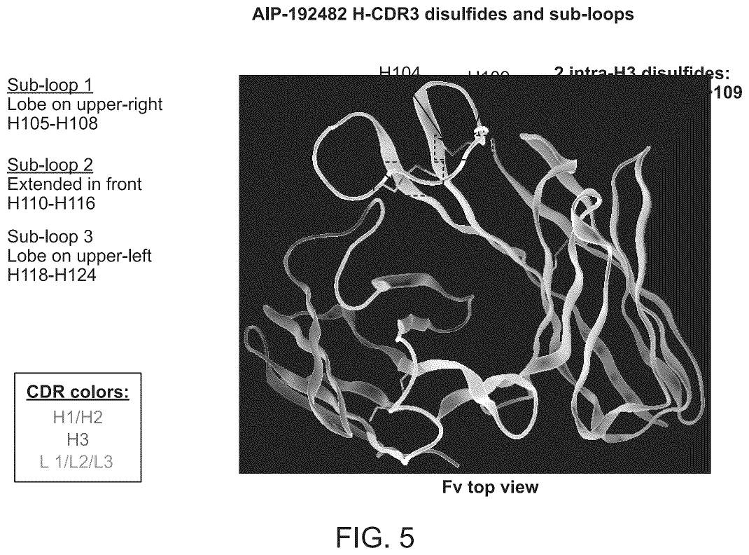

A crystal structure of an antibody (referred to as AIP-192482) comprising a scaffold region as described herein was obtained. AIP-192482 binds an extracellular RNA-protein complex comprising polyadenylated RNA and the polyadenylate-binding protein 1 (PABP-1), also referred to as PABPC-1. The sequences of the antibody V_{H} and V_{L} regions are provided in Table TA (entry no. 19) and Table 4A (entry no. 1). AIP-192482 CDR sequences are provided in Table 1B and Table 4B. This antibody has an unusually long (31-residue) HCDR3 loop. The HCDR3 loop additionally comprises two internal disfulfide bonds. The two intra-HCDR3 disulfide bonds serve to structurally divide the HCDR3 into three sub-loops: a tight, constrained loop from positions 105-108 (as numbered in the V_{H} region sequence shown in Table TA, SEQ ID NO:95); a longer, more flexible loop from positions 110-116 (also as numbered in Table 1A), including the three residues 112-114 that were not resolved in the crystal structure; and a loop from 118-124 (as numbered in Table TA). The HCDR3 loop has a structure similar to a knob that is situated so that it is tilted away from the center of the antibody, exposing a pocket. Although a class of bovine antibodies has been identified that has ultra-long HCDR3 loops that also contain disulfide bonds within the HCDR3 loops, these loops are longer (60 or more amino acids in length) than the loops of an antibody comprising a scaffold region as described herein. The additional residues in the bovine HCDR3 loops form long stalks of secondary structure that project the knob away from the surface of the Fv. In contrast, the HCDR3 loop of a scaffold antibody as described herein has a structure similar to a knob that is situated so that it is tilted away from the center of the antibody, exposing a pocket. FIGS. 1, 2, and 3 provide top, front, and side views of the Fv region and pocket of the antibody, respectively. The pocket structure is shown in FIG. 4. The sub-loop structures are depicted in FIG. 5.

In the present disclosure, a scaffold antibody comprises a pocket from about 12 Å-21 Å in depth. In some embodiments, the pocket is about 13 Å-20 Å deep. In some embodiments, the pocket regions is about 14 Å-19 Å in depth.

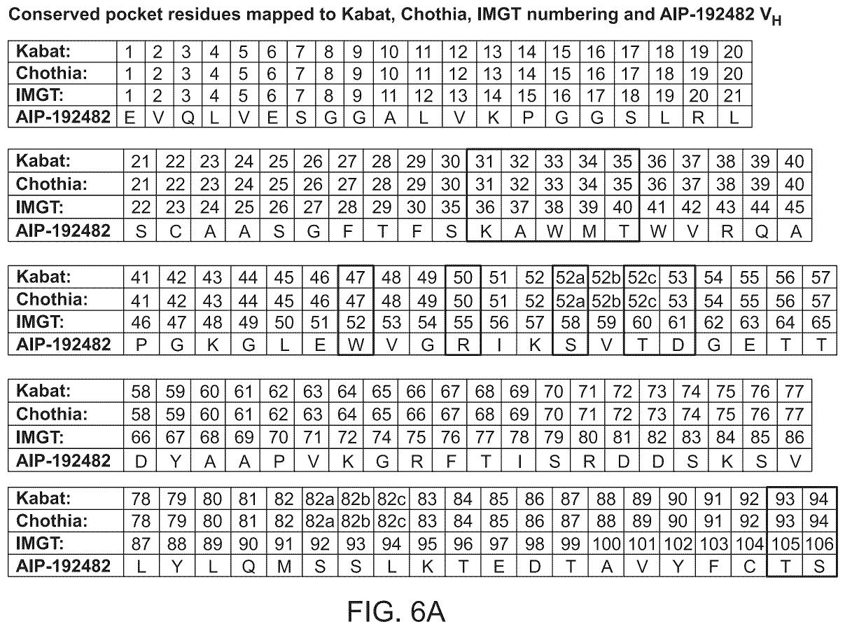

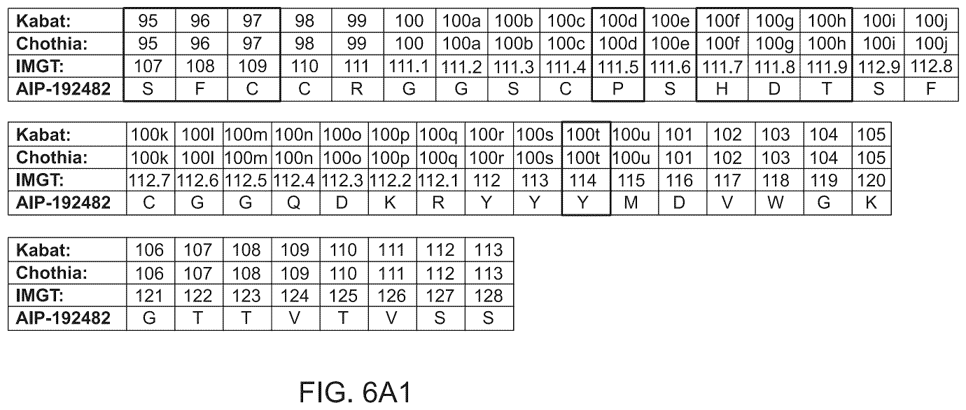

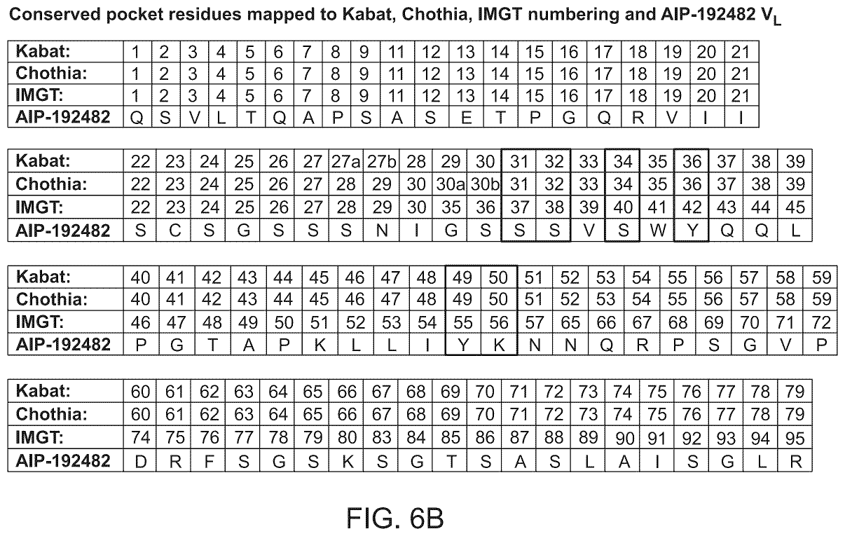

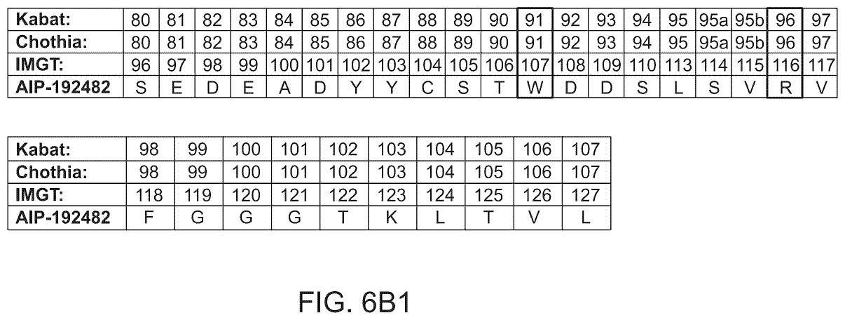

The pocket region of a scaffold antibody provided in the present disclosure comprises amino acid residues 36, 37, 38, 39, 40, 52, 55, 58, 60, 61, 105, 106, 107, 108, 109, 111.5, 111.7, 111.8, 111.9, and 114 in the V_{H} per IMGT numbering and amino acid residues 37, 38, 40, 42, 55, 56, 107, and 116 in the V_{L} per IMGT numbering. The pocket may further comprise one or more amino acid residues selected from the group consisting of 57, 111.6, 112.9, 112.7 and 116 in the V_{H} per IMGT numbering; or one or more amino acid residues selected from the group consisting of 39, 52, 57, 80, 105, and 106 in the V_{L} per IMGT numbering. In some embodiments, the antibody employed as a scaffold comprises residues 57, 111.6, 112.9, 112.7 and 116 in the V_{H} per IMGT numbering and residues 39, 52, 57, 80, 105, and 106 in the V_{L} per IMGT numbering. Pocket residues are shown in FIGS. 6A-10D.

In some embodiments, a scaffold antibody comprises at least 1, 2, 3, 4, 5, 6, 7, 8, 9, 10, 11, 12, 13, 14, 15, 16, 17, 18, 19 or 20 of the following residues in the V_{H}: A, C, H, K, M, N or R at position 36; A or V at position 37, F, H, V, W, or Y at position 38; F, M or Y at position 39; S, T, or Y at position 40, W at position 52; F or R at position 55, A or S at position 58; D, H, Q, S, or T at position 60; D, E, N, or S at position 61; I, T, or V at position 105; F, S or T at position 106; A, P, S, or T at position 107; F or Y at position 108; A, C, or S at position 109; A, C, L or P at position 111.5; H, Q, R, or S at position 111.7; D or E at position 111.8; C, N, Q, or T at position 111.9; or C, F, L, W, or Y at position 114; and/or at least 1, 2, 3, 4, 5, 6, 7, or 8 of the following residues in the V_{L}: A, H, N, S, or T at position 37; A, D, F, S, T, or Y at position 38; A, D, E, L, S, T, or Y at position 40; Y at position 42; H or Y at position 55; A, H, K, M, N, or R at position 56; F, I or W at position 107; or H, K, Q, R, V, or W at position 116. In some embodiments, the antibody comprises at least 1, 2, 3, 4, or 5 of the following residues in the V_{H} per IMGT numbering: K or Q at position 57; A, C, D, N, or S at position 111.6; S at position 112.9; A, C, S, or V at position 112.7; or D or N at position 116; and/or at least 1, 2, 3, 4, 5, or 6 following residues in the V_{L} per IMGT numbering: T or V at position 39; L at position 52; D or N at position 57; K at position 80; A or S at position 105; or A, I, S, or T at position 106. In some embodiments, the antibody comprises a substitution at one or more of the designated positions, where the substitution is a conservative substitution.

In some embodiments, the pocket comprises at least 1, 2, 3, 4, 5, 6, 7, 8, 9, 10, 11, 12, 13, 14, 15, 16, 17, 18, 19 or 20 of the following residues in the V_{H}: K at position 36; A at position 37; W at position 38; F or M at position 39; S or T at position 40; W at position 52; R at position 55; A or S at position 58; D, S, or T at position 60; D at position 61; I or T at position 105; S or T at position 106; S at position 107; F at position 108; C at position 109; P at position 111.5; H, R, or S at position 111.7; D at position 111.8; T at position 111.9; or Y at position 114; and/or at least 1, 2, 3, 4, 5, 6, 7, or 8 of the following residues in the VL: H, N, or S at position 37; D, S or Y at position 38; E, L, or S at position 40; Y at position 42; H or Y at position 55; K or R at position 56; I or W at position 107; or K, R, or W at position 116. In some embodiments, the antibody further comprises at least 1, 2, 3, 4, or 5 of the following residues in the V_{H} per IMGT numbering: K at position 57; S at position 111.6; S at position 112.9; C at position 112.7; or D at position 116; and/or at least 1, 2, 3, 4, 5, or 6 following residues in the V_{L} per IMGT numbering: V at position 39; L at position 52; N at position 57; K at position 80; A or S at position 105; or A or T at position 106.

The HCDR3 of a scaffold antibody provided herein is at least 25 amino acids in length. In some embodiments, the HCDR3 length is 25 to 37 amino acids in length. In some embodiments, the length is 27 to 36 amino acids. In some embodiments, the length ranges from 29 to 35 amino acids. In illustrative embodiments, the HCDR3 comprises the amino acid sequence of X¹CCX²CX³CX⁴, wherein X¹=4 amino acid residues, X²=4-7 amino acid residues, X³=6-8 amino acid residues, and X⁴=10-14 amino acid residues (SEQ ID NO: 1728). In other embodiments, the CDR3 comprises the amino acid sequence of X¹CCX²CX³CX⁴, wherein X¹=4 amino acid residues, X²=4 amino acid residues, X³=7 amino acid residues, and X⁴=12 amino acid residues (SEQ ID NO: 1731). In alternative embodiments, the HCDR3 comprises the amino acid sequence of X¹CGGX²CX³ wherein X¹=4 amino acid residues, X²=7 amino acid residues, and X³=12 amino acid residues (SEQ ID NO: 1732). In some embodiments, the HCDR3 comprises the amino acid sequence of X¹SCX²CX³ wherein X¹=4 amino acid residues, X²=4 amino acid residues, and X³=19 amino acid residues.

In some embodiments, the HCDR3 comprises the amino acid sequence of (I/T)(S/T)X₃(F/Y)CCX₇(G/S)X₉X₁₀CX₁₂(N/S)X₁₄(D/E)TS(F/Y)CX₂₀(G/N)X₂₂X₂₃X₂₄,X₂₅ (F/Y)YX₂₈X₂₉(D/N)X₃₁, wherein X₃ is A, P, or S; X₇ is H, L, Q, or R; X₉ is A, G, K, or N; X₁₀ is A, N, Q, R, or S; X₁₂ is A, L, or P; X₁₄ is H, Q, R, or S; X₂₀ is A, G, or N; X₂₂ is Q, S, or Y; X₂₃ is D, F, N, or Y; X₂₄ is A, K, N, P, or Q; X₂₅ is D, Q, R, or S; X₂₈ is F, L, W, or Y; X₂₉ is F, M, or V; and X₃₁ is I, P, or V (SEQ ID NO: 1729). In some embodiments, the HCDR3 comprises the amino acid sequence of (I/T)(S/T)X₃(F/Y)X₅CX₇(G/S)X₉X₁₀CX₁₂X₁₃X₁₄(D/E)X₁₆SX₁₈X₁₉X₂₀X₂₁X₂₂X₂₃X₂₄X₂₅(F/Y)(F/Y)X₂₈X₂₉(D/N)X₃₁, wherein X₃ is A, P, S, or T; X₅ is A, C, or S; X₇ is H, L, Q, or R; X₉ is A, G, K, or N; X₁₀ is A, N, Q, R, or S; X₁₂ is A, L, or P; X₁₃ is A, N, or S; X₁₄ is H, Q, R, or S; X₁₆ is N, Q, or T; X₁₈ is F, M, or Y; X₁₉ is C, S, or V; X₂₀ is A, G, or N; X₂₁ is A, G, or N; X₂₂ is Q, S, or Y; X₂₃ is D, F, N, S, or Y; X₂₄ is A, K, N, P, Q, or S; X₂₅ is D, K, Q, R, or S; X₂₈ is F, L, W, or Y; X₂₉ is F, M, or V; and X₃₁ is I, P, or V.

In some embodiments, a scaffold antibody described herein that can be employed as a parent antibody for diversification to identify new antibodies with altered binding specificity comprises a V_{H} region and a V_{L} region of an antibody identified by AIP number as set forth in any of Tables 1A, 2A, 3A, or 4A. In some embodiments, a scaffold antibody provided herein comprises a V_{H} region and a V_{L} region of an antibody identified by AIP number as set forth in any of Tables 1A, 2A, 3A, or 4A, with the proviso that the antibody is not AIP-192482. In some embodiments, the scaffold antibody comprises a V_{H} region and a V_{L} region of an antibody identified by AIP number as set forth in Table 1A. In some embodiments, the scaffold antibody comprises a V_{H} region and a V_{L} region of an antibody identified by AIP number as set forth in Table 2A. In some embodiments, the scaffold antibody comprises a V_{H} region and a V_{L} region of an antibody identified by AIP number as set forth in Table 3A. In some embodiments, the parent antibody comprises an antibody identified by AIP number as set forth in Table 4A.

In some embodiments, a scaffold antibody described herein that can be employed as a parent antibody for diversification to identify new antibodies with altered binding specificity comprises a V_{H} region and/or a V_{L} region that has 95% identity to the V_{H} region and/or V_{L} region of an antibody identified by AIP number as set forth in any of Tables 1A, 2A, 3A, and 4A. In some embodiments, the scaffold antibody comprises a V_{H} region and/or a V_{L} region that has 95% identity to the V_{H} region and/or V_{L} region of an antibody identified by AIP number as set forth in Table 1A. In some embodiments, the scaffold antibody comprises a V_{H} region and/or a V_{L} region that has 95% identity to the V_{H} region and/or V_{L} region of an antibody identified by AIP number as set forth in Table 2A. In some embodiments, the scaffold antibody comprises a V_{H} region and/or a V_{L} region that has 95% identity to the V_{H} region and/or V_{L} region of an antibody identified by AIP number as set forth in Table 3A. In some embodiments, the scaffold antibody comprises a V_{H} region and/or a V_{L} region that has 95% identity to the V_{H} region and/or V_{L} region of an antibody identified by AIP number as set forth in Table 4A.

In some embodiments, a scaffold antibody described herein that can be employed as a parent antibody for diversification to identify new antibodies with altered binding specificity compared to the parent antibody comprises at least one, two, three, four or five CDRs of an antibody identified by AIP number as set forth in any of Tables 1B, 2B, or 3B. In some embodiments, the scaffold antibody comprises six CDRs of an antibody identified by AIP number as set forth in Table 1B. In some embodiments, the scaffold antibody comprises six CDRs of an antibody identified by AIP number as set forth in Table 2B. In some embodiments, the scaffold antibody comprises six CDRs of an antibody identified by AIP number as set forth in Table 3B.

In some embodiments, a scaffold antibody described herein that can be employed as a parent antibody for diversification to identify new antibodies with altered binding specificity comprises at least one, two, three, four or five CDRs of an antibody identified by AIP number as set forth in any of Tables 1B, 2B, 3B, or 4B in which 1, 2, or 3 amino acids one or more of the CDRs are substituted with a conservative substitution.

In Tables 1A and 2A, position 140, as numbered with respect to V_{H} region sequences presented in Tables 1A and 2A, and position 110 of the V_{L} region sequences presented in Tables 1A and 2A are considered to be the last amino acids of the V_{H} and V_{L} regions, respectively, according to EU index numbering. Similarly, the last residue shown in each V_{H} and V_{L} sequence in Table 3A, although not corresponding to positions 140 and 110, respectively, of the sequences, is also considered to the last amino acids of the V_{H} and V_{L} regions. In a human IgG format (e.g., IgG1, IgG2, IgG3, or IgG4), the subsequent residue is termed the “junction codon”, and is natively encoded by the junction of the final 3′ base of the variable region gene (HJ or LJ) with the first two 5′ bases of the constant region gene (heavy or light), and exhibits amino acid variation due to variation in the final 3′ base of HJ and U. The human heavy chain junction codon can natively be Ala, Ser, Pro, or Thr, and is usually an Ala. The human kappa chain junction codon can natively be Arg or Gly, and is usually an Arg. The human lambda chain junction codon can natively be Gly, Ser, Arg, or Cys, and is usually a Ser or Gly.

CDRs as shown in Tables 1B, 2B, and 3B are defined by IMGT and Kabat. The heavy chain CDRs encompass amino acid residues from amino acid residues 26-35 (HCDR1), 50-68 (HCDR2) and 99-129 (HCDR3). The light chain CDRs encompass amino acid residues from amino acid residues 23-35 (LCDR1), 51-57 (LCDR2), and 90-100 (LCDR3). The numbering of the residues correspond to the positions in the V_{H} and V_{L} sequences in Tables 1A and 2A. The V_{H} CDRs as listed in Tables 1B, 2B, and 3B are defined as follows: HCDR1 is defined by combining Kabat and IMGT; HCDR2 is defined by Kabat; and the HCDR3 is defined by IMGT. The V_{L} CDRs as listed in Tables 1B, 2B, and 3B are defined by Kabat. As known in the art, numbering and placement of the CDRs can differ depending on the numbering system employed. It is understood that disclosure of a variable heavy and/or variable light sequence includes the disclosure of the associated CDRs, regardless of the numbering system employed.

### Diversifying a Parent Scaffold Antibody

In order to generate a library of diversified antibodies for screening, mutations are introduced into a parent scaffold antibody as described herein. Antibody vH and VL regions are expressed as disclosed herein are commonly produced using vectors and recombinant methodology well known in the art (see, e.g., Current Protocols in Molecular Biology (Ausubel, et al., John Wiley and Sons, New York, 2009 and updates through 2020).

Mutagenesis techniques are well known in the art in which nucleic acids encoding the antibody sequences are mutagenized. Mutagenesis techniques include, without limitation, directed evolution, oligonucleotide-directed mutagenesis, chemical mutagenesis, and error prone PCR techniques. For example, in some embodiments, oligonucleotide-directed mutagenesis techniques such as Kunkel mutagenesis (Kunkel et al., Methods Enzymol. 154:367-82, 1987), and variations of this protocol; or PCR site-directed mutagenesis techniques, may be employed to preserve scaffold structural elements. Other positions in a parent scaffold antibody may be mutagenized using additional mutagenesis methods, such as site-saturation mutagenesis, or cassette replacement, to enhance diversity outside of the preserved scaffold structural elements.

A library of diversified sequences can be expressed in any sort of expression system for screening that provides the ability to screen Fv regions. Such antibodies can be expressed in any suitable format, including expression as scFvs or Fabs. In some embodiments, Fv regions can be expressing the context of an IgG or other suitable format. In some embodiments, a library comprises at least 100, at least 1000, at least 10,000, at least 100,000, at least 1,000,000, or more members.

Suitable expression systems for screening antibodies for binding activity include, for example, phage display, yeast display, ribosome display, mRNA display, or mammalian cell display. Any other suitable insect, plant, or mammalian expression system can be employed to screen diversified libraries.

### Screening of Diversified Libraries

Diversified antibodies can be screened to identify antibodies that have a binding specificity that differs from the parent antibody as further described below. In some embodiments, a defined RNA-protein complex is used in the screening assay. In some embodiments, cells or tissues that comprises RNA-protein complexes that have not been characterized in detail may be employed. For examples, in some embodiments an uncharacterized RNA-protein complex associated, e.g., up-regulated, in a diseased tissue or disease state, such as cancer or autoimmune disease, may be used as the screening agent. Examples of RNA-protein binding complexes associated with cancer are described in and Pereira et al., Trend in Cancer 3:506-528, 2017; and references noted below. Examples of autoantibodies to RNA and/or protein components that can be present in RNA-protein binding complexes are described in Moinzadeh et al., Arthritis Res. & Therapy 16:R53, 2014; and Blanco et al., Clin. Exp. Immunol. 86:66-70, 1991).

In embodiments, in which the RNA-protein complex of interest has not been characterized, binding to the complex target can be evaluated by binding to tissue or cells of interest, optionally with selectivity over binding to an undesired target such as normal tissue or a non-tumor cell line. Once new antibodies are isolated that bind to the desired target, the target complex may be further evaluated to determine components of the RNA-protein complex.

In some embodiments, an RNA-protein complex may be obtained using an immunoprecipitation reaction or by proximity labeling with biotin for isolation by streptavidin. In some embodiments, recombinant RNA binding proteins may be expressed and reconstituted with RNA, e.g., poly(A+) RNA, ribosomal RNAs, or other RNAs of interest. Illustrative purification methods are provided in Jain et al., Cell 164:487-498, 2016, Hubstenberger et al., Mole. Cell 68:144-157, 2017; Ozgur et al., Cell 13:703-771, 2015; and Blokhuis et al., Acta Neuropathologica 132:175-196, 2016. See, also Urdaneta & Beckmann, Fast and unbiased purification of RNA-protein complexes after UV-cross-linking (Methods 178:72-82, 2020, available at ScienceDirect. The Example section also illustrates gel filtration purification of antibody-RNA/protein complexes.

In some embodiments, RNA-protein complexes of interest for use as a screening agent may be obtained from a source, e.g., a stress granule, by purifying a stress granule core complex or processing body complex (see, e.g., Jain et al., 2016, supra and Youn et al., Mol. Cell 69:517-532, 2018, referencing Hubstenberger et al., 2017, supra). Additional stress granule related RNA-protein complexes include stress-dependent G3BP1 complex (Markmiller et al., Cell 172, 590-604, 2018). In some embodiments, the RNA-protein complex may be associated with a disease of interest, e.g., ALS disease-associated RNA-protein complex (Blokhuis et al., 2016, supra) and Conlon & Manley, Genes and Develp. 31:1409-1528, 2017. In some embodiments, the RNA-protein complex may be from a condensate (see, e.g., Ditlev et al, J. Mol. Biol. 430:4666-4684, 2018).

In some embodiments, an RNA-protein complex of interest for use in screening may be a components of a ribonucleoprotein (RNP)-RNA condensate droplet. An RNP-RNA condensate is a large, disordered configuration of denatured RNPs that form non-specific associations with one another and RNA to form a large assemblage that phase-separates to form distinguishable droplets (see, e.g., Hyman et al., Annu Rev. Cell Dev. Biol. 30:39-58, 2014). Biomolecular condensates are two- and three dimensional compartments that concentrate specific collections of distinct molecules without an encapsulating membrane (Ditlev et al., J. Mol. Biol. 430: 4666-4684, 2018; Mullard, Nature Reviews 18:325, 2019). A “stress granule” as used herein refers to a non-membrane bound assembly or granule, typically formed in the cytoplasm, that is increased following cellular stress, such as treatment with an agent that disrupt protein synthesis. An antibody having an altered binding specificity as described herein may also bind to an RNA-protein complex that is a component of an RNP-RNA condensate droplet, a biomolecular condensate, or a stress granule.

Antibodies that bind RNA-protein complexes can be identified by repeated cycles of panning to identify antibodies that have altered binding specificities compared to the parent antibody template.

### Altered Binding Specificity

Antibodies as screened and selected in accordance with the disclosure have an altered binding specificity compared to the parent scaffold antibody used as the template for diversification. An “altered binding specificity” refers to binding activity that differs in magnitude and/or target binding compared to the parent antibody. Thus, in some embodiments, an antibody having an altered binding specificity binds to an RNA-protein complex for which the parent binding does not exhibit detectable binding under the screening conditions. In some embodiments, an antibody having an altered binding specificity does not bind to the same protein present in the RNA-protein complex, e.g., as assessed by crosslinking and/or immunoprecipitations analysis. In some embodiments, an antibody having an altered binding specificity exhibits enhanced binding to a target in binding assay compared to the parent antibody assessed in the same binding assay. In some embodiments, an antibody identified in a screen may exhibit reduced binding to the parent antibody target, but also bind an RNA-protein complex not bound by the parent. In some embodiments, an antibody identified in the screen may bind to an RNA-protein complex not bound by the parent while exhibiting similar, enhanced or decreased binding to the RNA-protein complex bound by the parent antibody. In some embodiments, an antibody identified in the screen may exhibit binding to a second RNA-protein complex as well as a third (or more) RNA-protein complex(es) in which activity differs from that of the parent scaffold antibody. In some embodiments, binding analysis may further comprise determining if treatment of an RNA-protein complex with RNase reduces binding of a candidate antibody to a target RNA-protein complex of interest. For example, in some embodiments, a candidate antibody that exhibits reduced binding e.g., at least 3-fold, at least 5-fold, at least 10-fold, at least 100-fold, or at least 1000-fold reduced binding, or no detectable binding, to an RNA-protein complex of interest following treatment with RNase, may be selected.

In some embodiments, an altered binding specificity is determined by measuring the K_{D} of a candidate antibody selected from a library of diversified sequence prepared from a parent scaffold antibody for binding to a target of interest compared to the K_{D} of the parent scaffold antibody binding to the target of interest under identical conditions. In some embodiments, a determination of altered binding specific may comprise measuring the K_{D} of the parent scaffold antibody for it target compared to that of a candidate antibody for binding to the parent scaffold antibody target. For example, a candidate antibody identified by screening may bind with a K_{D} that differs by at least 3-fold, at least 5-fold, or at least 10-fold, at least 100-fold, at least 1000-fold, or greater, for a target compared to the parent antibody. In some embodiments, a candidate antibody is selected that exhibits a K_{D} for binding to a target of interest that is decreased, i.e., binding is enhanced, compared to the scaffold antibody parent. Thus, for example, in some embodiments, an RNA-protein complex of interest may be generated in vitro by combining RNA and an RNA-binding protein and measuring the K_{D} of a candidate antibody for binding to the target by an assay such as a Biacore assay, compared to K_{D} of the parent scaffold antibody for binding to the target of interest under the same Biacore assay conditions. The K_{D} of a candidate antibody for binding to the target may be further evaluated after treatment of the target with RNase.

In some embodiments, binding activity to a target of interest, such as tumor tissue, may be assessed by measuring the ability of a candidate antibody to compete for binding to the target tissue in an in vitro competitive binding assay where the ability of the candidate antibody to inhibit binding of a parent scaffold antibody to the target tissue is measure.

In some embodiments, binding activity to a target of interest, such as a tumor cell target, may be assessed by measuring the ability of a candidate antibody to compete with the parent antibody in an ex vivo EMT6 assay as detailed in the Examples section below. In some embodiments, alternative syngeneic mouse models for the growth of tumor cells can be employed in which the tumor cells are assayed ex vivo, but obtained from tumors generated in syngeneic mouse models. Such models include syngeneic mouse models for cancers such as bladder, colon, glioma, leukemia, lung, melanoma, neuroblastoma, plasmacytoma, prostate, pancreatic, lymphoma, myeloma, liver, kidney, fibrosarcoma. Thus, for example, in some embodiments, a candidate antibody is selected that exhibits a lower EC₅₀ activity in an ex vivo syngeneic mouse model competitive binding assay compared to the parent scaffold antibody.

In some embodiments, a parent scaffold antibody binds to an extracellular RNA-protein complex that comprises mRNA and further comprises an mRNA binding protein “polyadenylate-binding protein 1” or “poly(A) binding protein 1” (PABP-1) and RNA, e.g., poly(A)-containing RNA. PABP-1 associates with the 3′ poly(A) tail of mRNA and is highly conserved among eukaryotic organisms. PABP-1 is encoded by the poly(A) binding protein cytoplasmic 1 (PABPC1) gene (see, e.g., the human PABPC1 gene sequence available under NCBI Ref. Sequence NM_002568.4). For purposes of the present disclosure, “PABPC1” is used interchangeably with “PABP-1” in referring to the polypeptide. Human PABPC1 polypeptide sequence information is available under UniProtKB accesson number P11940. Two isoforms produced by alternative splicing have been described. Isoform 1 is considered the canonical sequence and is 636 amino acids in length (see, e.g., NCBI Ref. Sequence NP_002559.2). Isoform 2 differs from the canonical sequence in that amino acids 447-553 are missing. A “human PABPC1 polypeptide” as used herein includes any PABPC1 polypeptide encoded by a human PABPC1 gene, which is localized to human chromosome 8q22.2-q23. The RNA-protein complex to which the parent scaffold antibody binds may comprises other RNA binding proteins and RNA other than polyadenylated RNA, for example, the complex may comprises other polyadenylate binding protein family members, such as PABPC4, PABPC3, MOV10, or UPF1. In some embodiments, a candidate antibody binds to an RNA-protein complex that comprises reduced levels of PABPC1 protein, relative to the amount of other RNA binding proteins in the complex, e.g., as measured by immunoprecipitation, compared to the RNA-protein complex bound by the parent scaffold antibody.

### Illustrative Diagnostic/Therapeutic Antibodies

In a further aspect, a tumor-binding antibody as provided herein, e.g., an antibody having CDRs of antibody AIP-131762, AIP-194564, AIP-169752, AIP-185680, AIP-145328, AIP-182606, AIP-107388, AIP-191692, AIP-163723, AIP-167295, AIP-125646, or AIP-114111 as set forth in Table 3B can be used as an agent for evaluating tumor or as a therapeutic agent, e.g., for the treatment of cancer. In some embodiments, an antibody comprising the V_{H} and V_{L} regions of antibody AIP-131762, AIP-194564, AIP-169752, AIP-185680, AIP-145328, AIP-182606, AIP-107388, AIP-191692, AIP-163723, AIP-167295, AIP-125646, or AIP-114111 set forth in Table 3 Å, or a variant thereof as described herein, is employed as a diagnostic or therapeutic agent.

In some embodiments, a diagnostic or therapeutic agent, e.g., for the treatment of cancer, comprises:
a heavy chain variable region comprising an HCDR1 comprising the sequence GFTFSKAWMS (SEQ ID NO: 1), or a variant HCDR1 in which 1, 2, 3, 4, or 5 amino acids are substituted relative to the sequence; an HCDR2 comprising the sequence RIKSVTDGETTDYAAPVKG (SEQ ID NO: 9), or a variant HCDR2 in which 1, 2, 3, 4, or 5 amino acids are substituted relative to the sequence; and an HCDR3 comprising the sequence of an HCDR3 set forth in Table 3B, or a variant HCDR3 in which 1, 2, 3, 4, or 5 amino acids are substituted relative to the sequence; and
a light chain variable region comprising: an LCDR1 comprising the sequence SGSSSNIGSSSVS (SEQ ID NO: 48), or a variant LCDR1 in which 1, 2, 3, 4, or 5 amino acids are substituted relative to the sequence; an LCDR2 comprising the sequence KNNQRPS (SEQ ID NO: 59), or variant LCDR2 in which 1, 2, or 3 amino acids are substituted relative to the sequence; and an LCDR3 comprising the sequence STWDDSLSVRV (SEQ ID NO: 68), or a variant LCDR3 in which 1, 2, 3, 4, or 5 amino acids are substituted relative to the sequence.
   In some embodiments, the antibody comprises an HCDR3 having a sequence
TSSFCGGPSHDTSYCGGQYKSYYYMDV (SEQ ID NO: 1734),
TSSFCCRSGGSCPSHDTSYCGGQYKSYYYMDV (SEQ ID NO: 1735),
TSSFCCRGGGSCPSHDTSYCGGQYKSYYYMDV (SEQ ID NO: 1730),
TSSFCCRGGGGSCPSHDTSYCGGQYKSYYYMDV (SEQ ID NO: 1737),
TSSFCCRGGSGGSCPSHDTSYCGGQYKSYYYMDV (SEQ ID NO: 1738),
TSSFSCRGGSCPSHDTSYGGQYKSYYYMDV (SEQ ID NO: 1739),
TSSFCCRGGSCPSHDTSYCGGGQYKSYYYMDV (SEQ ID NO: 1740),
TSSFCCRGGSCPSHDTSYCGGGSGYKSYYYMDV (SEQ ID NO: 1741),
TSSFCCRGGSCPSHDTSYCGGQKSYYYMDV (SEQ ID NO: 1742),
TSSFCCRGGSCPSHDTSYCGGQYSYYYMDV (SEQ ID NO: 1743),
TSSFCCRGGSCPSHDTSYCGQYKSYYYMDV (SEQ ID NO: 1744), or
TSSFCCRGGSCPSHDTSYCGGQYKSSYYYMDV (SEQ ID NO: 1745); or a variant thereof having 1, 2, or 3 amino acid substitutions.

In some embodiments, an antibody used as a therapeutic agent, e.g., for the treatment of cancer, comprises an HCDR1 comprising GFTFSKAWMS (SEQ ID NO: 1), an HCDR2 comprising RIKSVTDGETTDYAAPVKG (SEQ ID NO: 9), an HCDR3 comprising TSSFCCRGGSCPSHDTSYCGQYKSYYYMDV (SEQ ID NO: 1744) or TSSFCCRGGSCPSHDTSYCGGQYKSSYYYMDV (SEQ ID NO: 1745), an LCDR1 comprising SGSSSNIGSSSVS (SEQ ID NO: 48), an LCDR2 comprising KNNQRPS (SEQ ID NO: 59), and an LCDR3 comprising STWDDSLSVRV (SEQ ID NO: 68).

In some aspects, the disclosure additionally provides methods of identifying subjects who are candidates for treatment with a therapeutic anti-tumor binding antibody. Thus, in one embodiment, the invention provides a method of identifying a patient who has tumor cells that binds to an anti-tumor antibody of the present disclosure. In some embodiments, the tumor sample is from a primary tumor. In alternative embodiments, the tumor sample is a metastatic lesion. Binding of antibody to tumor cells through a binding interaction with an extracellular RNA-protein complex can be measured using any assay, such as immunohistochemistry or flow cytometry. In some embodiments, binding of antibody to at least 0.2%, 0.5%, or 1%, or at least 5% or 10%, or at least 20%, 30%, or 50%, of the tumor cells in a sample may be used as a selection criteria for determining a patient to be treated with an anti-tumor antibody as described herein. In other embodiments, analysis of components of the blood, e.g., circulating exosomes and/or extracellular RNA-protein complex, is used to identify a patient whose tumor cells are generating an extracellular RNA-protein complex.

### EXAMPLES

An antibody, AIP-192482, was identified that binds an RNA-protein complex target and functions as an anti-tumor agent via an unusual mechanism of tumor regression (see, WO2020/168231). Analysis of variants demonstrates that tolerance to sequence and paratope variation, forming the basis for a scaffold having the structural features identified as described in the following examples.

### Example 1. Acquiring Scaffold Antibody Structures

A three dimensional structure of an antibody is required for scaffold structure analysis. Although high-resolution experimentally determined structures are preferable, such as those acquired from X-ray crystallography, it is also possible to successfully model highly conserved variant sequences onto another experimentally determined structure of an antibody, i.e., by homology or comparative modeling. A structure of antibody AIP-192482 was obtained via X-ray crystallography. The sequence of AIP-192482 is provided in Tables 1 Å and 4 Å. Insufficient electron density was collected in the region of three consecutive amino acids in the HCDR3 loop (positions 111.7-111.9, IMGT numbering) and the backbone and side chain positions of these residues were not resolved. Computational modeling protocols are employed to repair small regions of missing residues in protein loops. We modeled the region of missing density in our structure using Chemical Computing Group's software Molecular Operating Environment (MOE).

One technique for comparative modeling utilizes the Rosetta software suite for macromolecular modeling. In one approach, RosettaScripts can be used to apply mutations to an experimentally determined starting structure, and energetically minimize the resulting structure (see, for example, website https doi.org/10.1021/acs.biochem.6b00444). The PackRotamersMover mover in RosettaScripts repacks sidechains and can be directed to substitute sidechains at described positions with another residue, mutating the sequence. The FastRelax mover performs rounds of packing and minimization to find preferred (i.e., low energy) conformations.

Using the Fv domain from the AIP-192482 structure and RosettaScripts, we modeled nine variants of this sequence. We initially modeled the sequences AIP-122563, AIP-186044, and AIP-160470 (see, Table 4A for sequences) starting with the AIP-192482 structure. Using RosettaScripts, we then performed computational protein design starting with the structure of AIP-160470 to find the optimal sequence via the PackRotamersMover mover. Optionally, the FavorNativeResidue mover can be used to provide a bonus to the starting sequence, requiring that the resulting variant sequence improves upon the initial score. From these designs we selected mutations to create six variants that each mutate one of the CDR loops, described as AIP-145518, AIP-167533, AIP-112580, AIP-150277, AIP-136060, and AIP-154708 (see, Table 4 Å for sequences), and used comparative modeling to generate structures for each as described above. All ten of these antibodies are exemplars of our scaffold structure.

In the 3-dimensional structures of our scaffold antibodies, we observed two notable features; a pair of disulfide bonds formed in the HCDR3 loop, and a deep pocket formed between the V_{H} and V_{L} domains. While disulfide bonds have been observed before in HCDR3 loops, the overall topology of the HCDR3 loop we observed in our scaffold antibodies has not previously been described. Furthermore, the pocket observed in these scaffold antibodies has not been observed in any other publicly available antibody structure to date.

### Example 2. Analysis of the Scaffold Antibody Pocket

To model and define the pocket we used the Rosetta application pocket measure, an implementation of LIGSITE [https website doi.org/10.1371/joumal.pcbi.1002951]. In our application of the algorithm, we centered the grid on heavy chain residue 101 (located in HCDR3 and facing into the pocket) and defined a stable grid of 16 Å to encompass the void formed between the VH and VL domains. We used a probe radius of 1.375 Å to model the pocket, and pocket PDBs were generated as output.

The pocket PDBs were analyzed in PyMOL to calculate pocket depth. The TP and TPB pseudoatoms in this resulting pocket PDB file were used to define the pocket volume. Pocket depth was measured between the bottom-most pseudoatom of the pocket volume and a pseudoatom located centrally at the top of the pocket volume, and center-to-center measurements were corrected by adding twice the probe radius (2.75 Å). The range of pocket depth measurements observed for our ten exemplar scaffold antibody structures (variants of AIP-192482) was 14.25 Å-19.35 Å. The range of pocket depth measurements for our ten exemplar scaffold antibody structures within 2 standard deviations of the mean was 13.70 Å-19.46 Å, and within 3 standard deviations of the mean the range was 12.25 Å-20.91 Å.

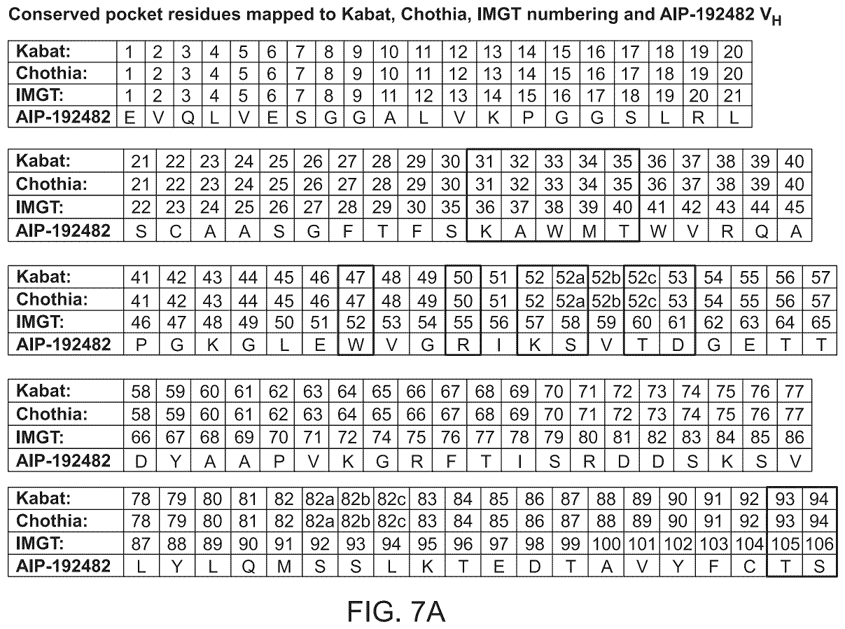

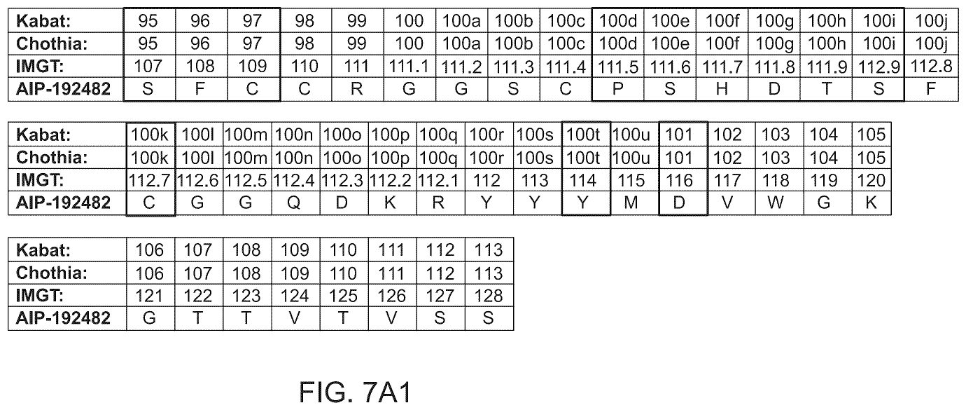

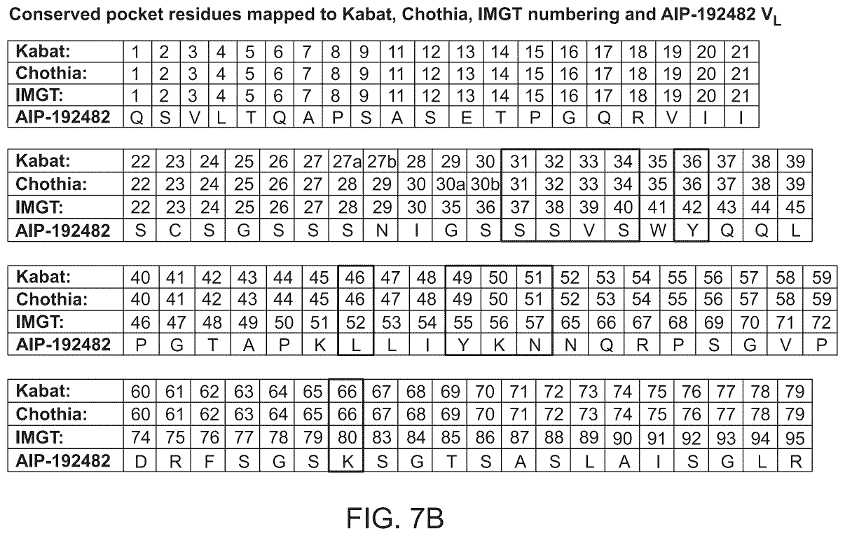

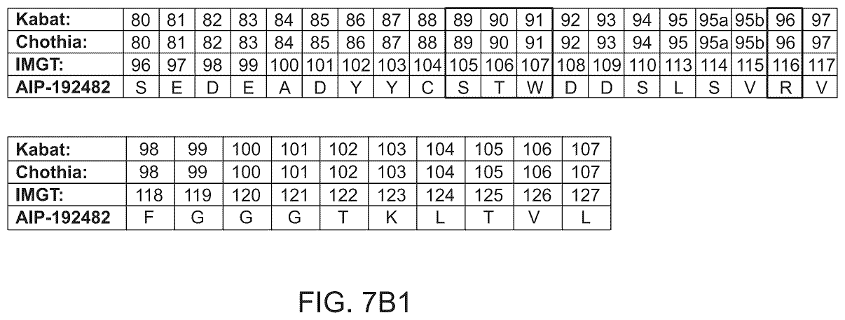

PyMOL was used to analyze the modeled pocket volumes in the context of the scaffold antibody structures, in order to determine residues of the scaffold antibodies that line the pocket. Residues with side chains within 4.0 Å of the pocket volume, measured atom center to pseudoatom center, were flagged as pocket residues. 4.0 Å approximates the longest “weak” hydrogen bond, and is used here to define possible molecule-to-molecule contacts, even if the contact is via hydrophobic atoms. The pocket residue positions conserved in our ten exemplar scaffold antibodies are described in FIGS. 6A and 6B. Pocket residue positions conserved in at least five of our ten exemplar scaffold antibodies are described in FIGS. 7A and 7B.

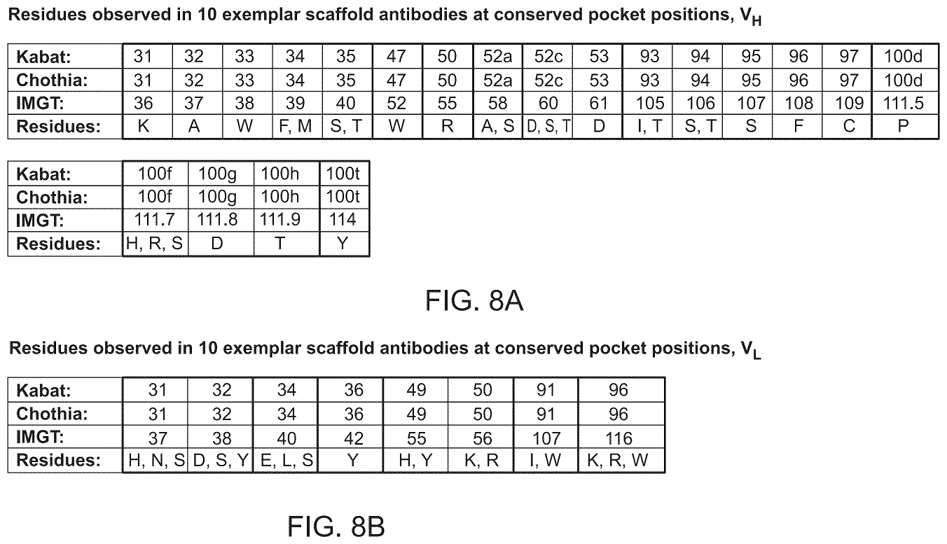

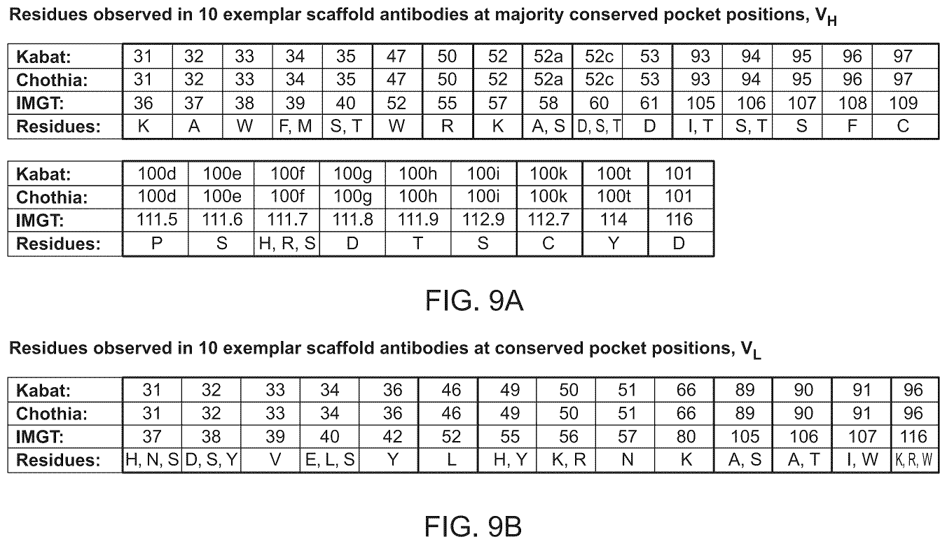

FIGS. 8A and 8B show residues observed in 10 exemplar scaffold antibodies at conserved pocket positions of the V_{H} and V_{L} regions; and FIGS. 9A and 9B show residues observed in 10 exemplar scaffold antibodies at majority conserved pocket positions of the V_{H} and V_{L} regions.

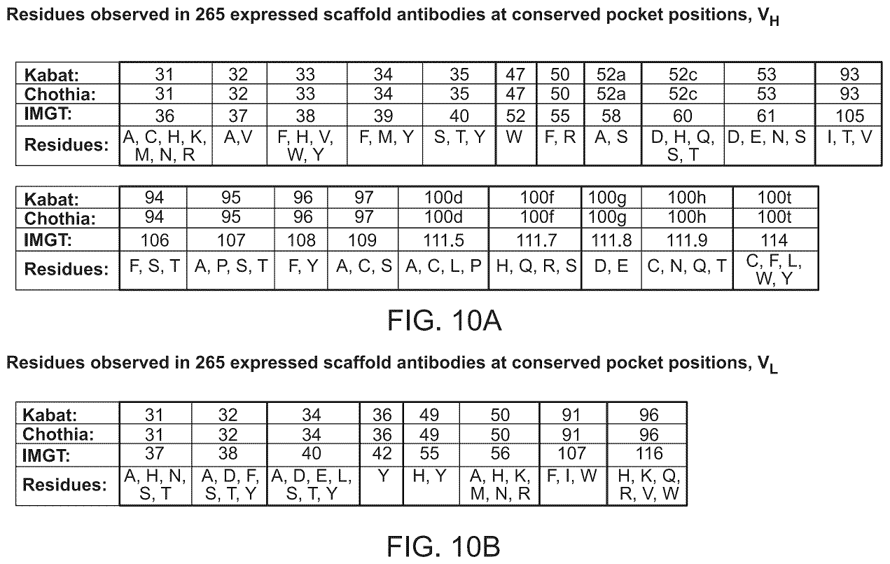

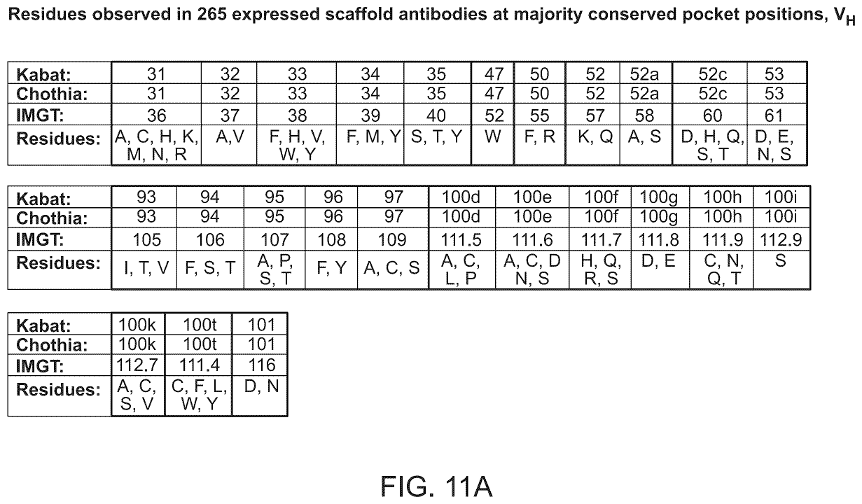

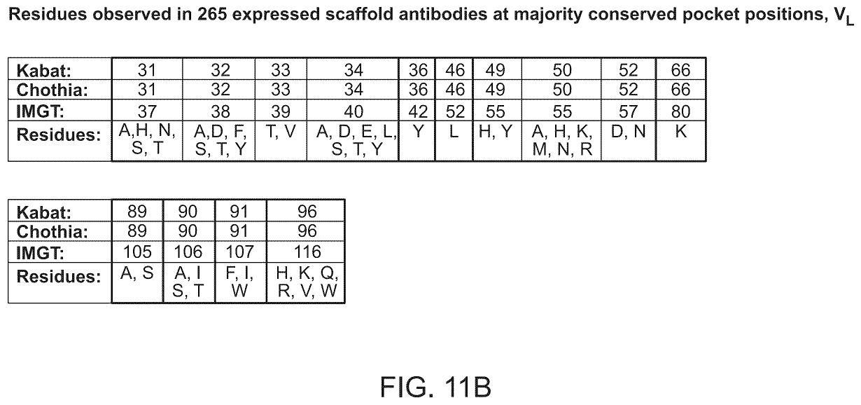

FIGS. 10A and 10B show residues observed in 256 expressed scaffold antibodies at conserved pocket positions of the V_{H} and V_{L} regions; and FIGS. 11A and 11B show residues observed in 256 expressed scaffold antibodies at majority conserved pocket positions of the V_{H} and V_{L} regions.

### Example 3. Evaluation of a AIP-192482 Anti-Tumor Effects

AIP-192482 was obtained by sequencing a plasmablast from a patient that had non-small cell lung cancer and was evaluated in an in vivo tumor model screen in combination with anti-PD1 antibody therapy.

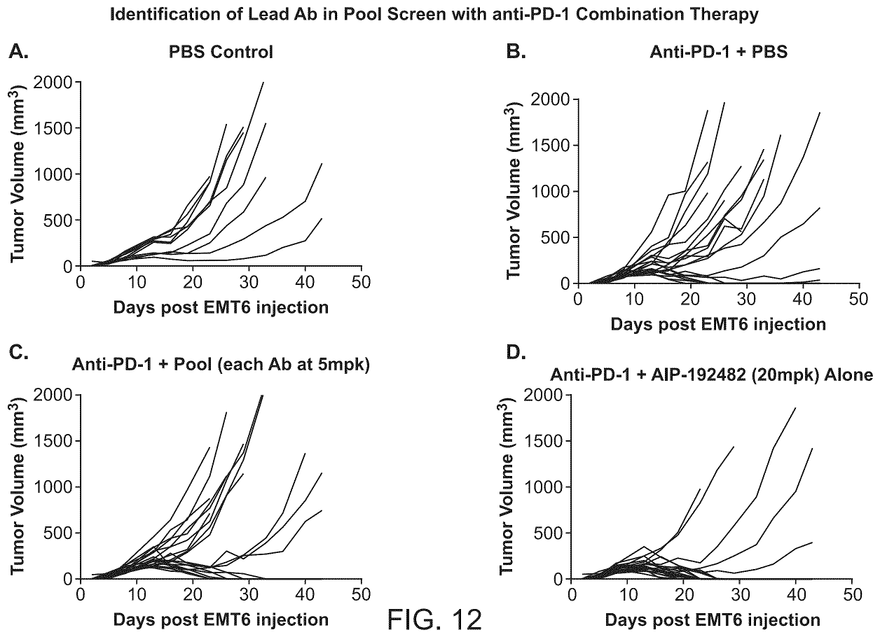

An EMT6 syngeneic ectopic breast cancer model in Balb/c mice was employed to evaluate anti-tumor activity of AIP-192482. Mice were randomized into treatment groups based on tumor volume (TV) once tumors reached an average of 75-120 mm³. Treatment with test antibodies and/or checkpoint inhibitor therapy started on the day of randomization. Unless otherwise stated, test antibodies were administered twice weekly for 3.5 weeks (TWx3.5w) by intraperitoneal (i.p.) injection. Pools of four antibodies were tested at doses of 5 mg/kg (mpk) each (20 mg/kg total antibody) by i.p. injection twice weekly for 3.5 weeks (7 doses). Animals were treated in combination with anti-mouse PD1 antibody dosed i.p. at 10 mpk, twice weekly for two weeks. AIP-192482 was also tested alone (outside of pool) at 20 mpk in combination with anti-PD1. The antibody pool containing the lead antibody showed anti-tumor activity (FIG. 12), as did the antibody tested alone in combination with anti-PD1. AIP-19248 also exhibited marked effects on survival at doses of both 5 mpk and 10 mpk (data not shown).

### Example 4. Histological Analysis of AIP-192482 and Variant Binding to Tumor Tissue

Binding of AIP-192482 and variants (further described below) AIP-160470, AIP-133645, AIP-158623, AIP-155066, AIP-136538, AIP-166120, AIP-187893, AIP-142079, AIP-184490, and AIP-104188 was evaluated in ER-positive breast tumor tissue and TAT. ER-positive breast tumor and TAT sections were stained with H&E and immunostained with the lead antibody, variant, or isotype control (IgG) at 1, 3, and 10 pg/ml. Immunoreactivities were assessed by fluorescent microscopy, and images across concentrations were captured. Positive immunoreactivity (i.e., signal above the IgG control) was detected for AIP-192482 and all of the variants (AIP-160470, AIP-133645, AIP-158623, AIP-155066, AIP-136538, AIP-166120, AIP-187893, AIP-142079, AIP-184490, and AIP-104188) with the exception of AIP-136538 (data not shown). Images were matched for similar fluorescence intensities in the ER-positive breast cancer cores, regardless of antibody concentration. With the exceptions of AIP-136538 and AIP-104188, all variants tested demonstrated fluorescent intensities within breast tumor similar to that observed with the initial lead antibody, but at 3 to 10-fold lower primary antibody concentrations. Selectivity of tumor labeling vs. TAT binding was also assessed. The ratio of tumor to adjacent tissue signal was markedly enhanced vs. lead with a subset of variants including AIP-160470 and AIP-133645.

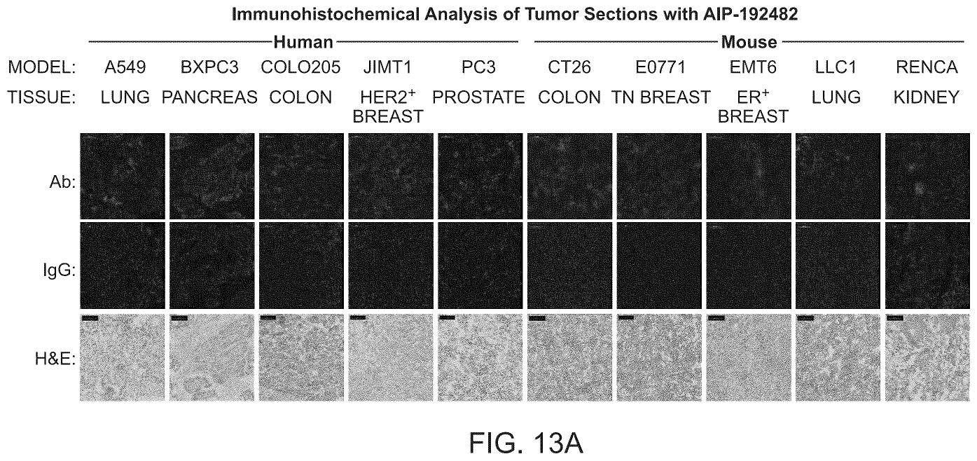

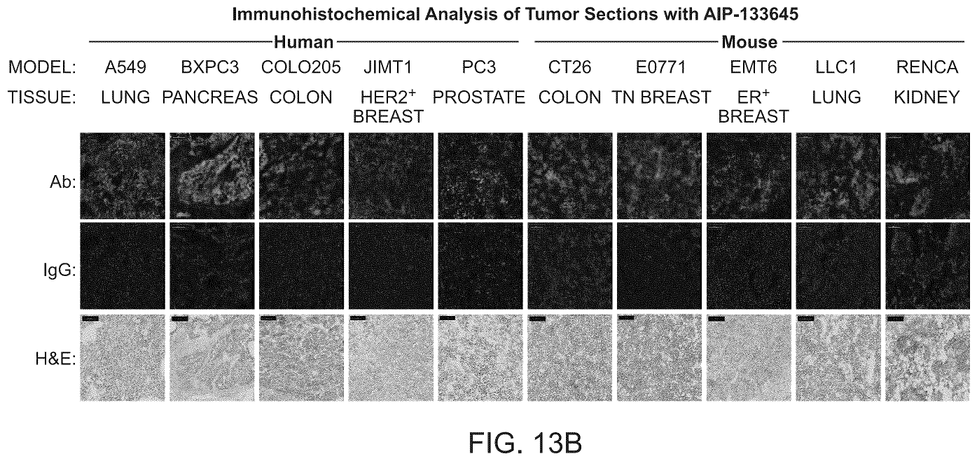

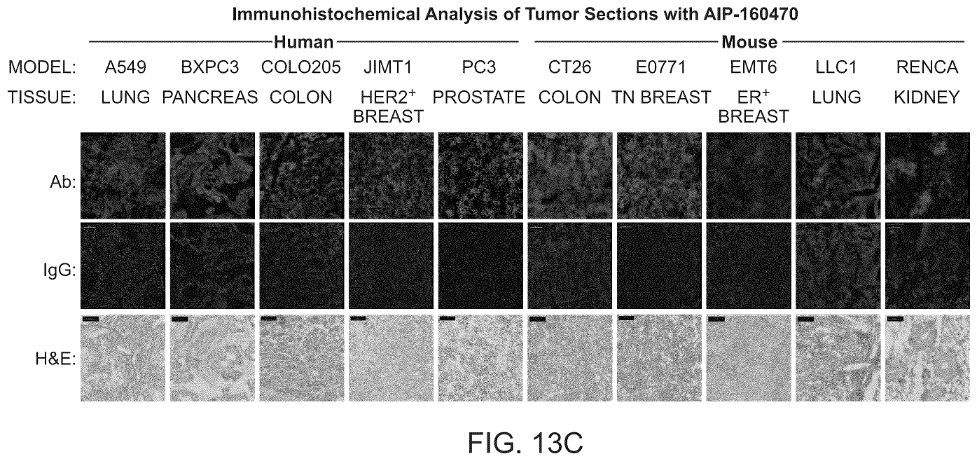

AIP-192482 and variants AIP-133645 and AIP-160470 were also evaluated for binding to tumor arising in mice inoculated with either human cancer cell lines (xenografts) or mouse cancer cell lines (syngeneic). Frozen tumors were cryosectioned onto slides. Antibodies and isotype control (IgG) were conjugated to AF647, and slides were incubated with the conjugates then counterstained with Hoechst. An adjacent section was also stained using H&E. Tumor sections stained using the variant antibodies showed enhanced signal compared to the lead for all tumors, including tumors arising from human lung A549 cells, human pancreas BXPC3 cells, human colon cancer Colo-205 cells, or human prostate cancer PC3 cells, and tumors arising from mouse colon, breast, lung, or kidney cancer cell lines (FIG. 13).

Antibody binding to EMT-6 tumors was also evaluated. Tumor samples were harvested from animals approximately 7 days post-inoculation of 10⁶ EMT-6 cells. EMT-6 binding activity was evaluated for variants AIP-157397, AIP-165430, AIP-160470, AIP-133645, AIP-158623, AIP-155066, AIP-136538, AIP-166120, AIP-187893, AIP-142079, AIP-184490, and AIP-104188 (data not shown). The background immunoreactivity was slightly higher in mouse tissue likely due to interactions with endogenous Fc receptors. While all variants exhibited binding to EMT-6 tumor cells, differential signal intensities were apparent between variants.

### Example 5. Analysis of Binding of Antibody to Target

To determine the target on tumor cells to which AIP-192482 binds, immunoprecipitation (IP) was performed with whole cell extracts. A549 cells were incubated overnight in methionine- and cysteine-free media supplemented with ³⁵S-labeled methionine and cysteine (37° C., 5% CO₂). Cells were lysed in radio-immunopreciptiation assay (RIPA) buffer (25 mM Tris·HCl pH 7.6, 150 mM NaCl, 1% NP-40, 1% sodium deoxycholate, 0.1% SDS) followed by 5 washes with buffer (1x phosphate buffer saline supplemented with 450 mM NaCl, 1 mM EDTA, 1% NP-40, 5% glycerol). IP reactions using AIP-192482 or control antibody were performed overnight using antibodies cross-linked to Dynabeads™ M-280 Tosylactivated paramagnetic beads. Beads were then collected on magnets and washed in 450 mM NaCl-containing buffer and eluted with 50 mM Tris, pH 8.0, 1 mM EDTA, and 1% sodium deoxycholate. Radiolabelled antigen bound to antibodies was eluted into 2x reducing SDS-PAGE sample buffer and resolved on 4-20% denaturing SDS-PAGE gels. Resolved proteins were visualized by phosphorimaging autoradiography. For analysis by mass spectrometry (MS), lysates were prepared using A549 cells that had not been metabolically labeled and IP reactions scaled up proportionally. Eluates were then evaluated by nanoLC-MS/MS analysis (performed by Alphalyse and MS Bioworks).

SDS-PAGE analysis identified a large number of unique proteins, ranging in size from 15.8 to 152.8 kDa. Numerous RNA-binding proteins were among the proteins identified by MS analysis. A large number of these identified proteins have been shown to be present in stress granules, see Markmiller et al., Cell, 172, 590-604, Jan. 25, 2018; and Youn et al., Molecular Cell, 69, 517-532 (2018) and Jain et al., Cell 164, 487-498 Jan. 28, 2016. Table 5 lists exemplary proteins identified in the immunoprecipitation complex pulled down by AIP-192482. These identified proteins are categorized based on whether they are also known to be in stress granules according to analyses that have been published previously: i) the biochemical isolation analysis on US-02 cells as described in Jain et al. (2016), ii) the APEX (proximity labeling) analysis performed on HEK293 cells as described in Markmiller et al. (2018), iii) the APEX (proximity labeling) analysis performed on neural progenitor cells as described in Markmiller et al. (2018), and iv) BioID (proximity labeling) analysis on HEK293 cells as described in Youn et al. (2018). Group 1 proteins (12 proteins) are among the list of proteins (20 proteins) that have been previously detected in stress granules by all four published analyses. Group 2 proteins (12 proteins) are among those list of proteins (38 proteins) that have been previously detected in stress granules by three of the four published analyses. Group 3 proteins (20 proteins) are among the list of proteins that have been previously detected in stress granules by two of the four published analyses. Group 4 proteins (63 proteins) are among the list of proteins (139) that have been previously detected in stress granules by Jain et al.

TABLE 5
Exemplary proteins in the immunoprecipitation
complex identified by MS

| Group 1 | Group 2 | Group 3 | Group 4 |
|---|---|---|---|
| TOTAL = | TOTAL = | TOTAL = | TOTAL = |
| 12/20 (60%) | 12/38 (32%) | 20/61 (33%) | 63/139 (45%) |
| ATXN2 | DHX36 | CASC3 | ACIN1 |
| FAM120A | ELAVL1 | CELF1 | ADAR |
| FUBP3 | FMR1 | DDX6 | ATXN2 |
| FXR1 | FXR2 | EDC4 | BAG2 |
| IGF2BP1 | HNRNPA1 | FUS | DDX17 |
| IGF2BP2 | HNRNPAB | GRSF1 | DDX21 |
| IGF2BP3 | PABPC1 | HNRNPA3 | DDX5 |
| PABPC4 | PCBP2 | HNRNPDL | DHX15 |
| STAU2 | PRRC2C | HNRNPH1 | DHX30 |
| SYNCRIP | PUM1 | HNRNPUL1 | DHX9 |
| UPF1 | RBMX | MOV10 | EDC4 |
| YBX3 | ZC3HAV1 | PTBP1 | EFTUD2 |
| | | PTBP3 | EIF4A3 |
| | | RBM4 | ELAVL1 |
| | | SND1 | FAM120A |
| | | SRSF9 | FBL |
| | | TNRC6A | FTSJ3 |
| | | TRIM25 | FUBP3 |
| | | TRIM56 | FUS |
| | | YBX1 | FXR1 |
| | | | HNRNPA0 |
| | | | HNRNPA1 |
| | | | HNRNPA3 |
| | | | HNRNPC |
| | | | HNRNPD |
| | | | HNRNPH1 |
| | | | HNRNPH3 |
| | | | HNRNPL |
| | | | HNRNPR |
| | | | HNRNPUL1 |
| | | | IGF2BP1 |
| | | | IGF2BP2 |
| | | | IGF2BP3 |
| | | | ILF2 |
| | | | ILF3 |
| | | | MATR3 |
| | | | MOV10 |
| | | | NOP56 |
| | | | PABPC1 |
| | | | PABPC4 |
| | | | PCBP2 |
| | | | PLEC |
| | | | PRPF8 |
| | | | PTBP1 |
| | | | RALY |
| | | | RBM14 |
| | | | RBMX |
| | | | RPL14 |
| | | | RPL4 |
| | | | RPS8 |
| | | | RRBP1 |
| | | | SF3B3 |
| | | | SNRNP200 |
| | | | SRRM2 |
| | | | SRSF10 |
| | | | STAU2 |
| | | | SYNCRIP |
| | | | TARDBP |
| | | | TRIM25 |
| | | | TRIM56 |
| | | | UPF1 |
| | | | YBX3 |
| | | | ZC3H7A |

Additional proteins identified in the MS that were present in AIP-192482 immunoprecipitates at a level of 2-fold or greater, or with a score of 2 or greater, compared to control immunoprecipitates are: ABCF1, ACIN1, ACLY, ADAR, AGO1, AGO2, AGO3, AHNAK, ATP2A2, ATXN2, BAG2, BOP1, BUB3, CAD, CASC3, CDC5L, CELF1, CLTA, CNBP, COPA, CRNKL1, DARS, DDX17, DDX18, DDX21, DDX5, DDX54, DDX6, DHX15, DHX30, DHX36, DHX57, DHX9, DICER1, DKC1, DNTTIP2, EDC4, EEF1D, EEF2, EFTUD2, EIF2AK2, EIF2S1, EIF3D, EIF3E, EIF3I, EIF4A3, EIF4G1, EIF6, ELAVL1, EPRS, FAM120A, FBL, FMR1, FTSJ3, FUBP3, FUS, FXR1, FXR2, GAR1, GEMIN4, GNL3, GRSF1, GTPBP4, HEATR1, HIST1H1B, HIST1H1C, HISTTH3A, HNRNPA0, HNRNPA1, HNRNPA2B1, HNRNPA3, HNRNPAB, HNRNPC, HNRNPD, HNRNPDL, HNRNPF, HNRNPH1, HNRNPH3, HNRNPK, HNRNPL, HNRNPM, HNRNPR, HNRNPUL1, HNRNPUL2, IGF2BP1, IGF2BP2, IGF2BP3, ILF2, ILF3, KARS, KHDRBS1, L1RE1, LARP1, MAGOHB, MAK16, MAP1B, MATR3, MBNL1, MOV10, MRTO4, MVP, MYBBP1A, MYO1B, NAT10, NCL, NHP2, NIFK, NKRF, NOL11, NOL6, NOP2, NOP56, NOP58, PABPC1, PABPC4, PCBP2, PDCD11, PES1, PGD, PLEC, PPP1CB, PRKDC, PRKRA, PRPF19, PRPF4B, PRPF8, PRRC2C, PTBP1, PTBP3, PUM1, PURA, PURB, PWP1, PWP2, RAB14, RAB2A, RACK1, RALY, RAN, RBM14, RBM34, RBM4, RBM45, RBM8A, RBMX, RCC2, RPL10A, RPL11, RPL12, RPL13A, RPL14, RPL15, RPL17, RPL18, RPL18A, RPL19, RPL21, RPL22, RPL23, RPL23A, RPL24, RPL26, RPL27, RPL29, RPL3, RPL30, RPL32, RPL34, RPL35A, RPL36, RPL4, RPL5, RPL6, RPL7, RPL7A, RPL8, RPLP0, RPLP1, RPLP2, RPS11, RPS13, RPS17, RPS23, RPS24, RPS26, RPS27A, RPS3, RPS3A, RPS5, RPS6, RPS8, RPS9, RPSA, RRBP1, RRP1, RRP9, RRS1, RSL1D1, RTCB, RUVBL2, RYDEN, SART3, SF3B3, SKIV2L2, SLC3A2, SND1, SNRNP200, SNRNP70, SNRPB, SNRPD1, SNRPD2, SNRPD3, SON, SRP68, SRP72, SRPK1, SRPK2, SRRM2, SRSF1, SRSF10, SRSF2, SRSF3, SRSF6, SRSF7, SRSF9, SSB, STAU1, STAU2, STRAP, SYNCRIP, TARBP2, TARDBP, TCOF1, TCP1, THOC2, THOC6, TNRC6A, TOP1, TRA2A, TRA2B, TRIM25, TRIM56, TTN, U2AF2, UGDH, UPF1, UTP15, UTP18, UTP4, UTP6, WDR12, WDR36, WDR43, WDR46, WDR74, WDR75, XAB2, XRCC5, XRN2, YBX1, YBX3, YTHDC2, ZC3H7A, ZC3HAV1, ZCCHC3, and ZNF326.

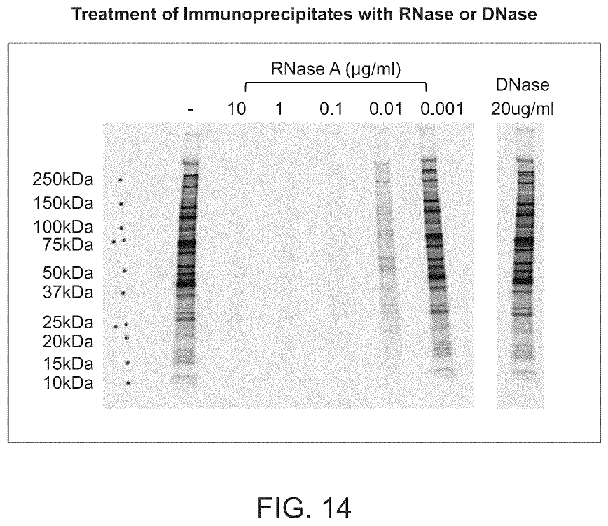

To test whether RNA is part of a complex to which AIP-192482 binds, lysates were prepared from radiolabelled A549 cells and then treated with increasing amounts of RNase A (from 0.001 to 10 μg/ml) or DNAse 1(20 μg/ml). Treated lysates were employed in IP using AIP-192482. Bound proteins were analyzed by SDS-PAGE followed by autoradiography. Results showed that treatment with RNase, but not DNase, eliminated proteins immunoprecipitated by AIP-192482 in an RNase dose-dependent fashion (FIG. 14). This effect was specific to the AIP-192482 antigen since no such effect was observed for EGFR binding to an anti-EGFR control antibody (data not shown). This result indicated that RNA contributes to AIP-192482 binding to its target.

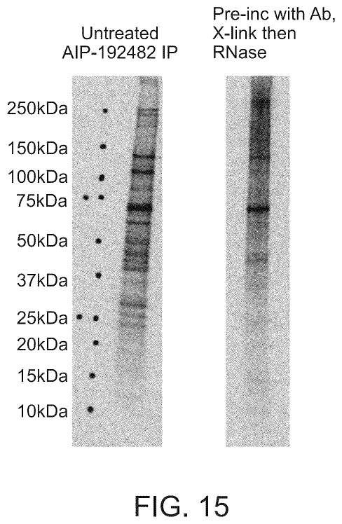

Additional MS analysis was performed to identify the composition of the target. IN one such analysis, RIPA lysate prepared from unlabelled A549 cells was incubated with AIP-192482-conjugated M-280 beads and then crosslinked using DTSSP (ThermoFisher). After quenching, lysates were treated with RNase (100 ng/ml). Beads were washed in high salt buffer and antigen eluted using 0.5 N ammonium hydroxide before performing nanoLC-MS/MS (MS Bioworks). In gel digestion was performed and proteins analyzed. One RNA binding protein, PABPC1, was present in high abundance in results from all MS runs and was enriched following RNase treatment (FIG. 15).

PABPC1 was expressed as a C-terminal FLAG fusion in 293T cells transiently transfected with a human PABPC1-FLAG expression plasmid and purified by anti-FLAG antibody chromatography. Purified PABPC1 was incubated with purified poly(A) RNA (56mer) produced via in vitro transcription of a linearized plasmid template driven by a T7 promoter. Complexed PABPC1-RNA was then incubated with monovalent AIP-192482 Fab and resulting complexes resolved on a Superose 6 gel filtration column (GE Lifesciences) at a final ratio of 15:15:1 PABPC1: AIP-192482 Fab: RNA by mass. A₂₈₀ absorbance of column eluate and SDS-PAGE analysis of column fractions (FIG. 16) indicated that AIP-192482 Fab, PABPC1 and poly(A) RNA form a high molecular size complex that elutes from the column markedly before the individual components. RNase A was also included in an AIP-192482 incubation step prior to column chromatography. A high molecular size complex was not observed for this sample.

As explained above, MS results from crosslinked, RNAse treated immunoprecipitate from A549 cells obtained using AIP-192482 showed the presence of PABPC1 in the immunoprecipitate. PABPC3/4, MOV10, UPF1, and other proteins were also identified as components present in the immunoprecipitate.

### Example 6. Confocal Imaging Showing Localization and Components of the Complexes Targeted by the Antibodies

Immunofluorescence microscopy was performed on EMT6 tumor tissues stained with antibodies. AIP-192482 was shown to co-localize with a subpopulation of G3BP-positive structures within the EMT6 tumors (data not shown). The images showed an extensive overlap between AIP-192482 signal and signal for G3BP, another marker of stress granules. The results show a presence of smaller G3BP puncta that are not positive for AIP-192482. Although a subset of these G3BP-positive structures, i.e., very small puncta, are not positive for AIP-192482 immunoreactivity, in larger aggregates, which are more heterogeneous in structure, the reactivity co-locates. This suggests that AIP-192482 associates with complexes with RNPs.

The antibodies disclosed herein were shown to target protein complexes located extracellularly via confocal microscopy. AIP-160470 reacted where CD9 reactivity was also detected. The confocal imaging showed that the AIP-160470 reactivity was clearly extracellular in nature, which corroborates the flow cytometry data as described below. Similar confocal microscopy experiments were performed with human breast carcinoma tissue, which also showed that AIP-160470 co-localized with numerous CD9-positive vesicles about 1 μm diameter that appeared extracellular.

Additional confocal microscopy imaging showed that AIP-160470 signal co-located with RNA binding proteins, including PABPC1 and MOV10, and labeling was consistent with extracellular punctae and plasma membrane in human ovarian cancer tissue.

### Example 7. Flow Cytometry Showing the Surface Localization of the Complexes Targeted by the Antibodies

EMT6 cells were treated with a chemotherapeutic agent, doxorubicin (“DOX”) or cisplatin (“CDDP”), for 16 hours. Both DOX and CDDP are known inducers of stress granules, Vilas-Boas, et al., J Neurooncol 127:253-260 (2016); and Morita et al., Biochemical and Biophysical Research Communications 417: 399-403 (2012). The treated cells were plated in a 96 well plate and stained with AIP-192482, AIP-160470, or AIP-195694 (negative control). The cells were then stained with a secondary antibody that was conjugated with Alexa 647 and analyzed on a flow cytometer. Geometric mean fluorescence corresponding to the binding of antibodies to the EMT6 cells were plotted against concentrations of the chemotherapeutic agents used in treating cells (data not shown). These data showed that these antibodies were bound to the surface of the cells, which complement the confocal data, which shows growth of EMT6 cells in vivo also induces such surface reactivity.

### Example 8. Generation of Variants of AIP-192482 and AIP-160470

Antibody variants of AIP-192482 and AIP-160470 were generated by introducing various substitutions into the CDRs, using AIP-160470 as a parental comparison sequence. The variants included individual substitutions introduced across each CDR, as well as variants in which up to four substitutions were introduced into HCDR1, up to five substitutions were introduced into HCDR2, up to thirteen substitutions were introduced into HCDR3, up to six substitutions were introduced into LCDR1, up to three substitutions were introduced into LCDR2, and up to six substitutions were introduced into LCDR3. Combinations of variant CDR1, CDR2, and CDR3 sequences were also tested. The CDR sequences of active variants are provided in Table 1B and Table 2B.

Variants of AIP-160470 comprising deletions or insertion in the HCDR3 were also generated. Sequences are provide in Table 3 Å.

Activity of antibodies in vitro was determined using an FcR engagement assay to assess activity in comparison to that of AIP-160470. Variants were tested in a FcγRIIa-H ADCP Reporter Bioassay (Promega). This assay is used to measure the potency and stability of antibodies that specifically bind and activate FcγRIIa. The assay employed Jurkat cells stably expressing human FcγRIIa-H (the high-affinity H131 variant) and NFAT-induced luciferase. Following engagement of an FcγR on Jurkat effector cells by the Fe region of a test antibody binding to a target cell, intracellular signals are generated in the Jurkat cells that result in NFAT-RE-mediated luciferase activity, which can be quantified.

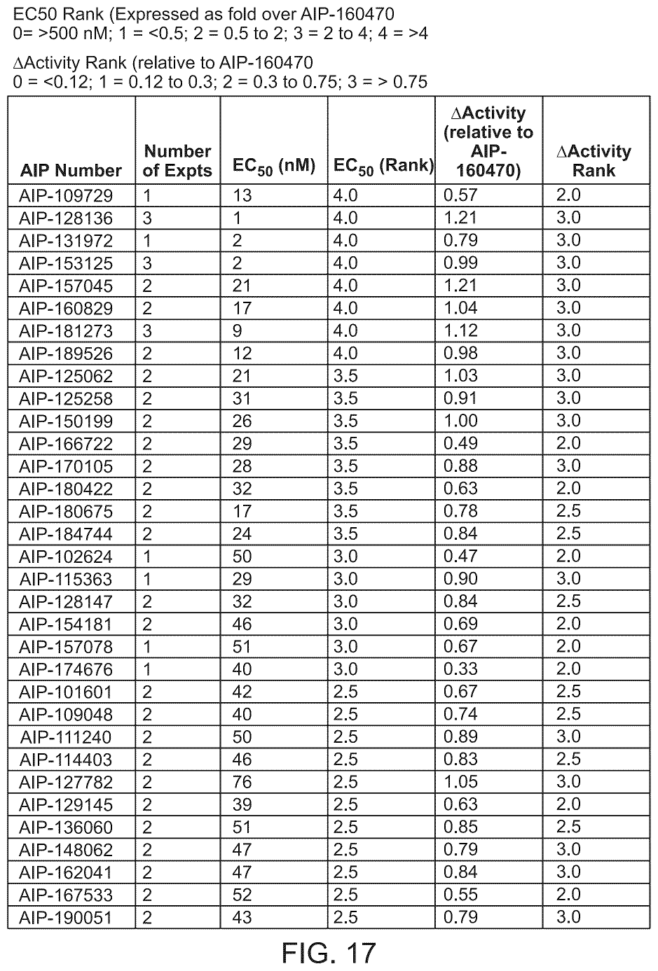

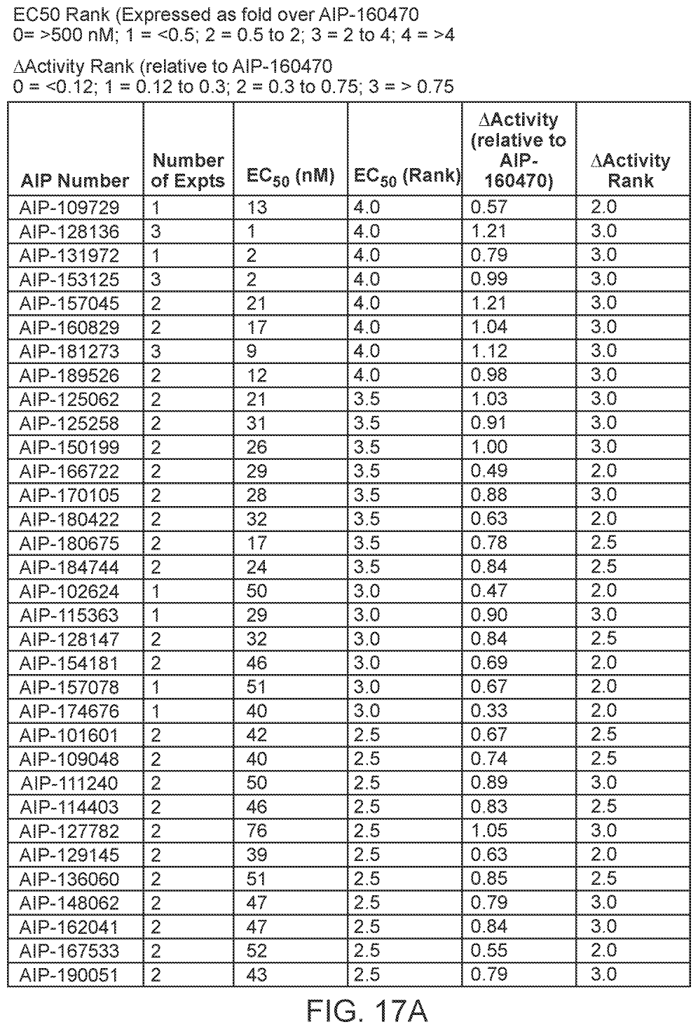

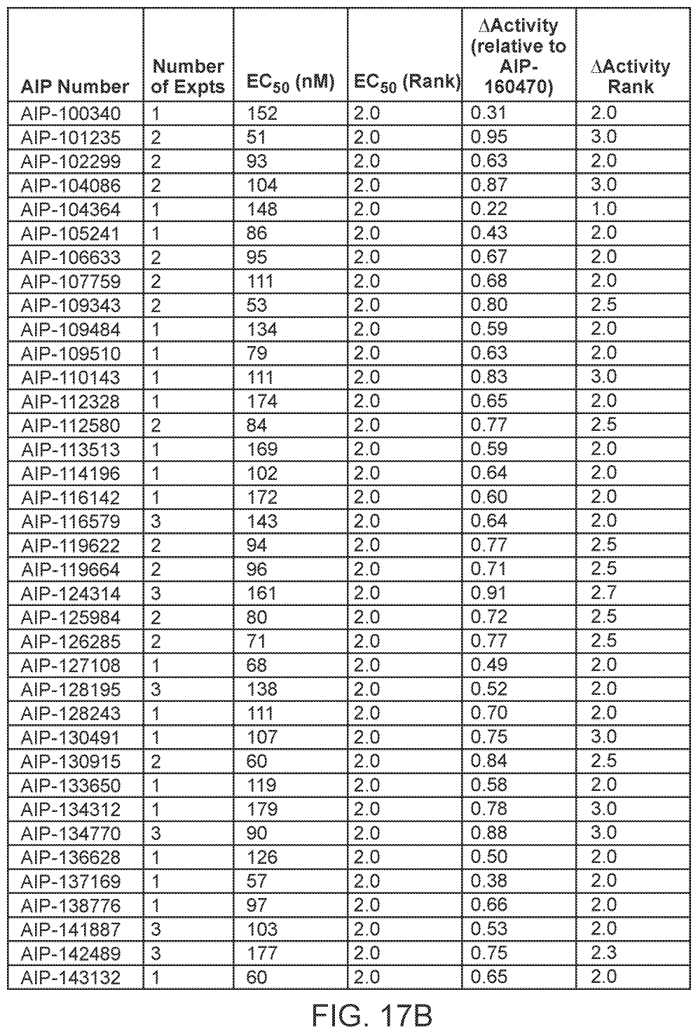

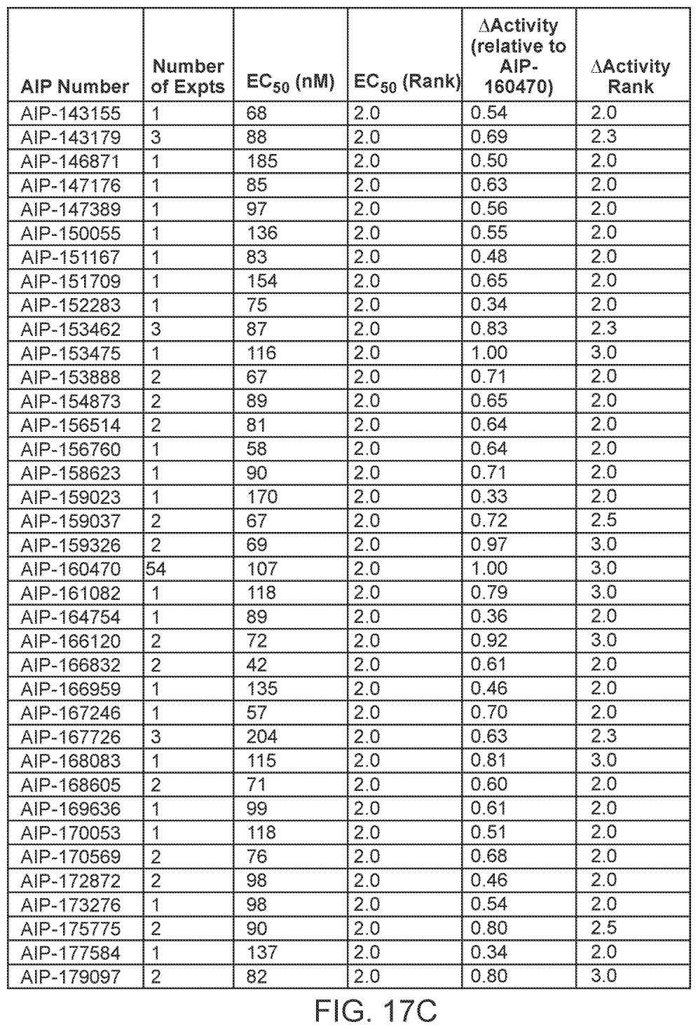

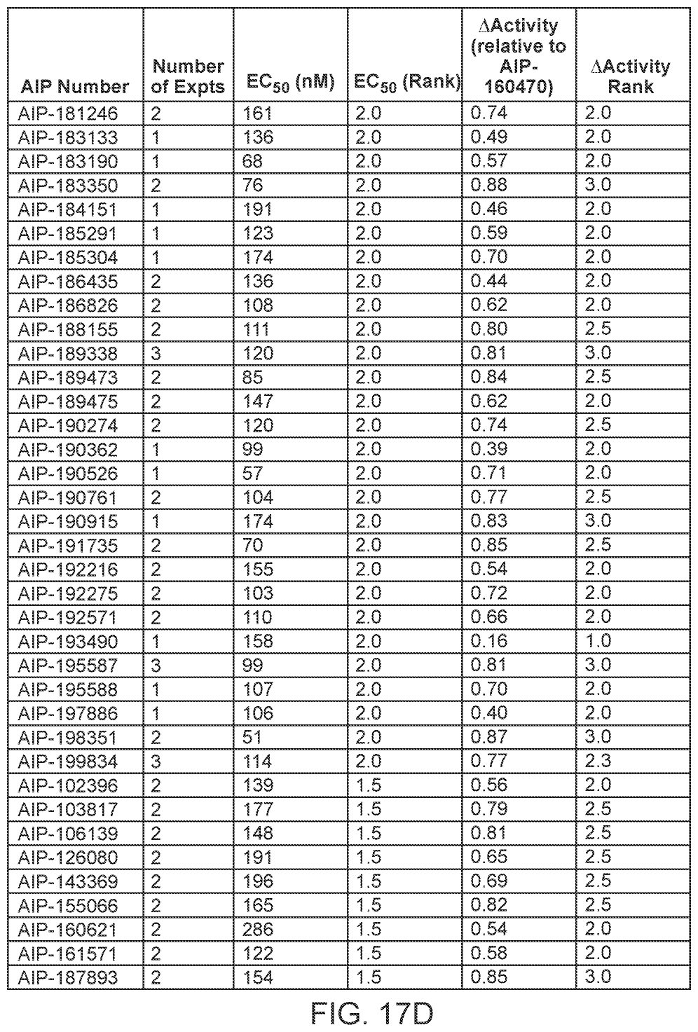

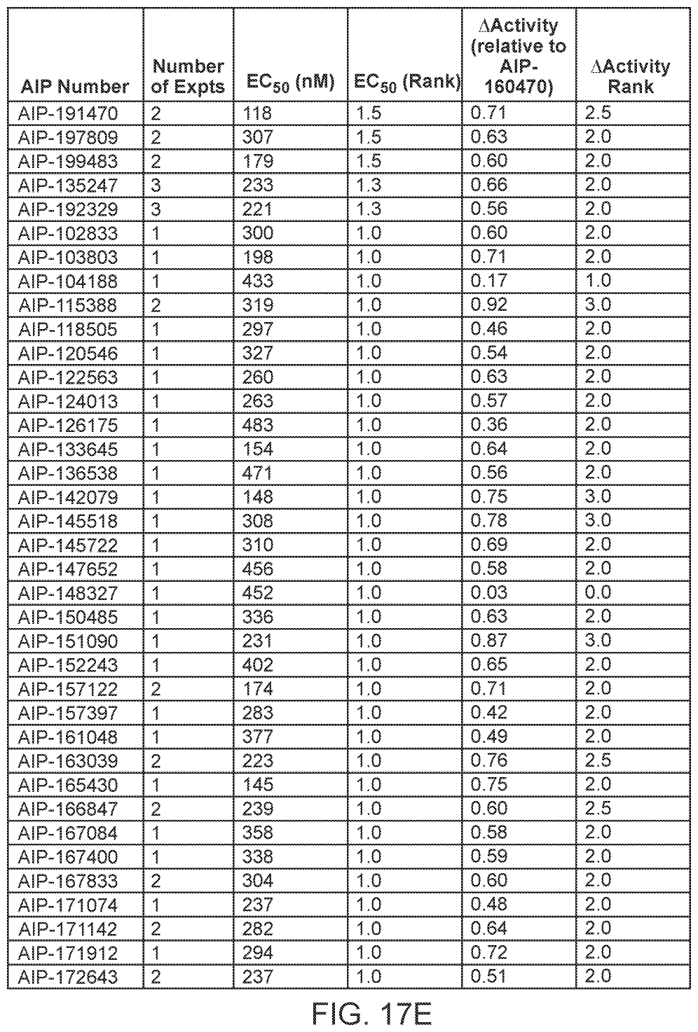

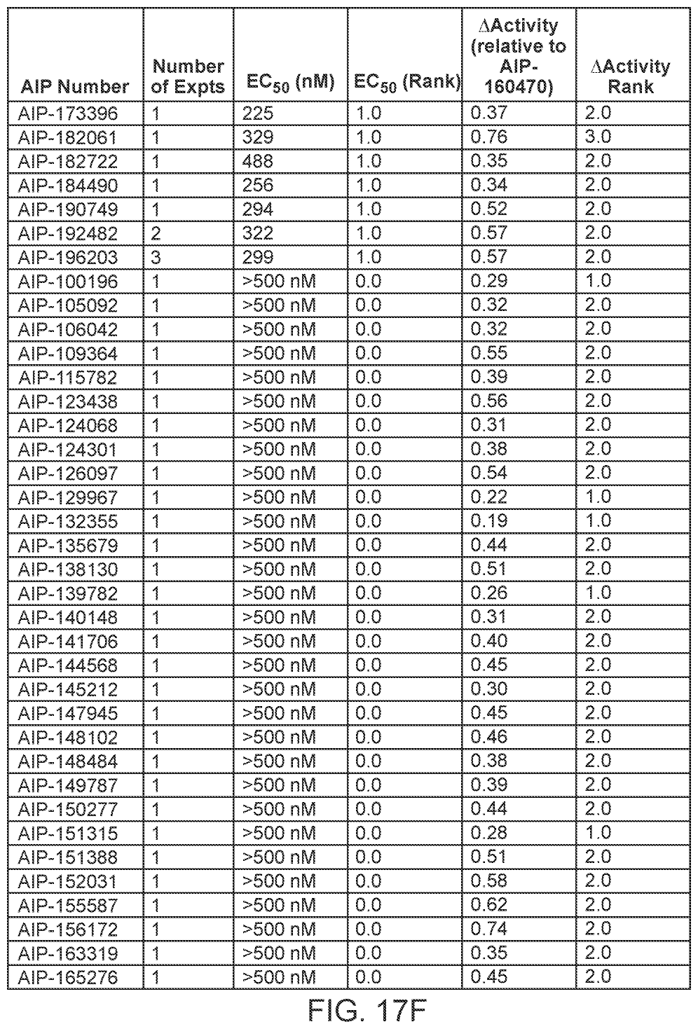

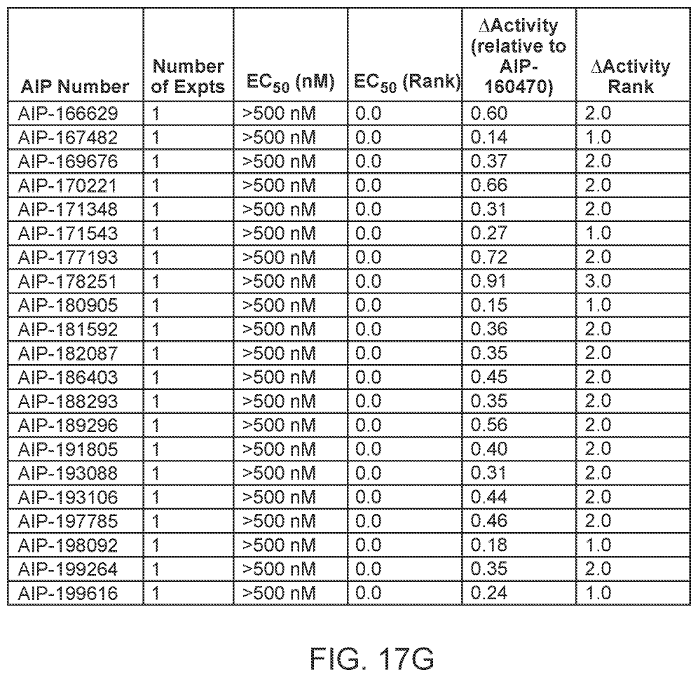

The activity of substitution variants active in the in vitro assay are shown in FIG. 17. Variants are deemed active if they exhibited an EC₅₀ of 500 nM or less; or if they have a delta activity value, relative to AIP-160470, of at least 0.5. An antibody that has a delta-value, relative to AP-160470, of zero is considered to be inactive.

The activity of variants comprising deletions or insertions in the HCDR3 of AIP-160470 were also assed in the in vitro assay. The HCDR3 sequences are provided in Table 3B. The values are shown below. The number following the AIP number reflects number of times the antibody was assayed.

| | | | Avg | Avg |
|---|---|---|---|---|
| AIP Number | EC50 | Fold | EC50 | ΔActivity |
| (N) | (nM) | ΔActivity | Rank | Rank |
| AIP-131762 (1) | >500 | 0.28 | 0.0 | 1.0 |
| AIP-194564 (2) | 472 | 0.58 | 1.0 | 2.0 |
| AIP-169752 (1) | >500 | 0.57 | 0.0 | 2.0 |
| AIP-185680 (1) | >500 | 0.29 | 0.0 | 1.0 |
| AIP-145328 (1) | >500 | 0.32 | 0.0 | 2.0 |
| AIP-182606 (1) | >500 | 0.46 | 0.0 | 2.0 |
| AIP-107388 (1) | >500 | 0.65 | 0.0 | 2.0 |
| AIP-191692 (1) | >500 | 0.39 | 0.0 | 2.0 |
| AIP-163723 (1) | >500 | 0.49 | 0.0 | 2.0 |
| AIP-16729 5(1) | >500 | 0.36 | 0.0 | 2.0 |
| AIP-125646 (2) | 446 | 0.75 | 1.0 | 2.5 |
| AIP-114111 (2) | 258 | 0.55 | 1.5 | 2.0 |

### Example 9. Ex Vivo Binding Assay Correlates with In Vivo Anti-Tumor Activity

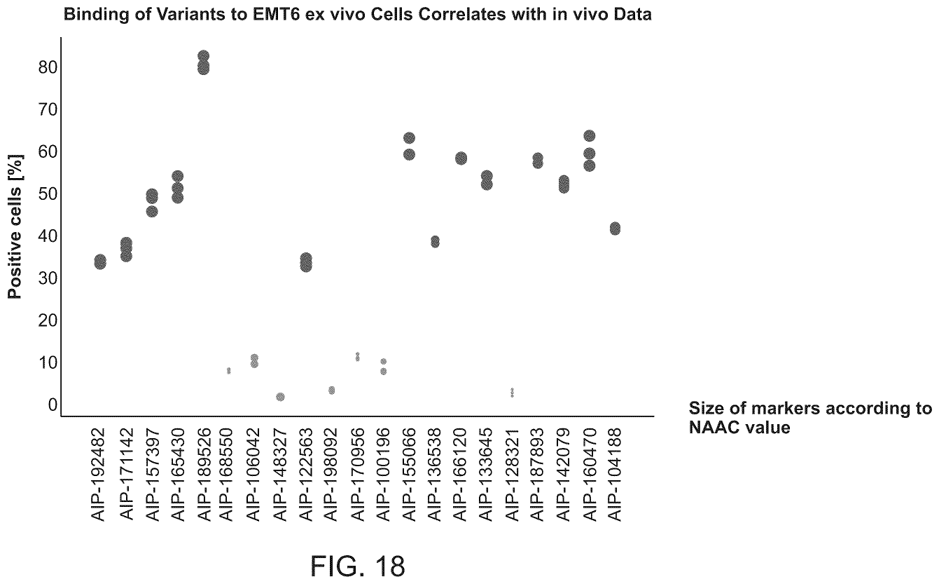

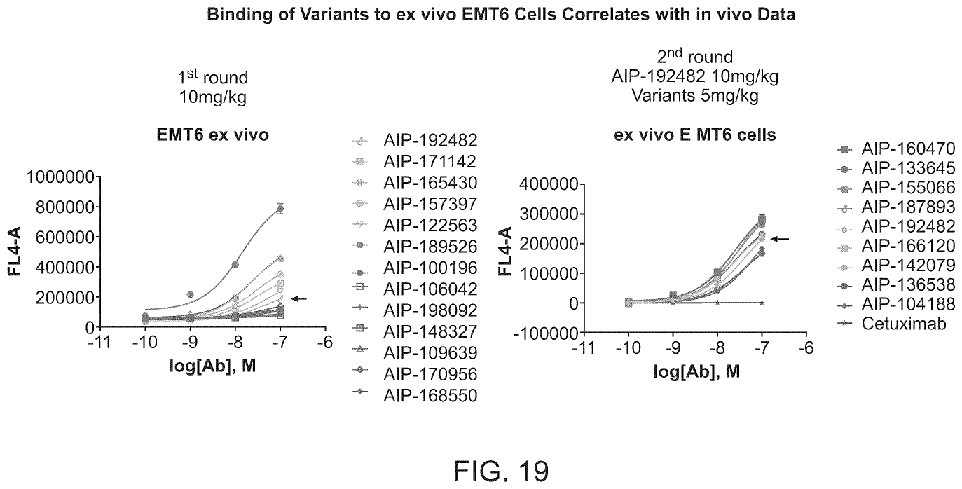

A subset of variant antibodies was evaluated to analyzed in an ex vivo binding assay to determine correlation with in vivo anti-tumor activity. Mice were injected with EMT6 tumor cells and the tumor allowed to grow to a size of about 500-600 mm³. Tumors were harvested, digested, and antibody binding to the surface of live tumor cells analyzed by flow cytometry. Binding was correlated with normalized area above the curve (NAAC) values representing tumor volume following in vivo treatment with antibody. The results showed that binding of antibodies to the ex vivo EMT6 cells largely correlated with in vivo outcome (FIGS. 18 and 19). Ex vivo flow analysis thus provides a screening assay that largely correlates with in vivo function.

Antibodies that are active in vivo are listed in Tables 1 Å and 1B. Antibodies that are active in the in vitro assay are listed in Table 2 Å and 2B; and Table 3 Å and 3B. AIP-125646 and AIP-114111 were also active in vivo.

The in vivo activity of AIP-125646 and AIP-114111 was also assessed in the EMT6 mouse model. The log-rank p-value for AIP-125646 was 2.37E-05 and that of AIP-114111 was 1.75E-04.

| | NAAC | NAAC | NAAC | NGRM | NGRM |
|---|---|---|---|---|---|
| AIP No. | n | p-value | effect size | p-value | effect size |
| AIP-125646 | 10 | 2.25E−06 | 0.3698 | 1.75E−04 | 0.2757 |
| AIP-114111 | 10 | 7.80E−05 | 0.3493 | 5.33E−06 | 0.1057 |

### Methodology for Assessing In Vivo Activity

Anti-tumor activity of antibodies was assessed using an EMT6 syngeneic mouse tumor model was used as described above (see, WO2020/168231 for details of the protocol employed) to assess the anti-tumor efficacy of mIgG2a antibodies. The procedure utilized was modified from DeFalco et al., Clin. Immunol. 187:37-45, 2018. EMT6 mouse tumor cells were propagated in culture by passaging cells every 2 to 3 days (1:10 subcultures). In brief, female 4-6-week old BALB/c mice were each inoculated in the right hind flank by subcutaneous injection with 1×10⁶ EMT6 cells in 0.2 mL Waymouth's media without supplements. The day of cell inoculation was designated as Study Day 0. An overage of >30% was included to achieve study groups with consistent and homogenous tumor volumes. Mouse tumors consistently became visible and palpable approximately three days after cell inoculation. Tumor volumes were measured 2-3 times prior to randomization. Mice were randomized on study day 7 using the ‘matched distribution’ randomization function of the StudyLog lab animal management software (version 3.1.399.23) to ensure homogenous tumor volumes. Test articles and vehicle control were prepared in formulation buffer (Dulbecco's PBS, DPBS). Starting on randomization day, test articles were dosed at 10 ml/kg based on mouse body weight via twice weekly IP injection for a total of 7 doses. All mice were dosed as scheduled or until they were removed from study based on euthanasia criteria. Tumor volumes were measured twice weekly after randomization. Tumor volumes were calculated automatically using the following equation:

Tumor Volume (mm³)=length (mm)×width² (mm)×0.5

Following test article administration, mice with tumor volumes of 0 mm³ for three consecutive measurements were considered to show a complete response (CR). Mice with consistent tumor regression that continued after closure of the dosing window were considered to show a durable response (DR).

Statistical analyses of tumor volumes were performed using the normalized area above the curve (NAAC) and the normalized growth rate metric (NGRM) developed at Atreca, Inc.

To determine the NAAC, the area between the tumor volume curve and the tumor volume endpoint of 2000 mm³ was divided by the total area possible through Study Day 35 post tumor inoculation. The total area possible is determined between the first time point at which all animals have a measurable tumor volume and Study Day 35 for tumor volumes between 0 mm³ to 2000 mm³. NAAC values are between 0 and 1. Individuals with a small area between the curve and the tumor volume endpoint have NAAC values closer to 0, and individuals with a larger area between the curve and the tumor volume endpoint have NAAC values closer to 1.

To determine the NGRM, the slope was first calculated for the log-transformed tumor volumes versus time, and then rescaled to 20 days post tumor inoculation. These slopes were then normalized to values between 0 and 1. Individuals with an increasing tumor volume over time have NGRM values closer to 0, and individuals with a stable or decreasing tumor volume over time have NGRM values closer to 1.

Statistically significant differences between the distributions for the treatment and control group were evaluated for NAAC and NGRM with a one-sided Wilcoxon rank-sum test, yielding a p-value using R version 3.4.3 (The R Foundation). Animals with final tumor volumes that did not reach 1800 mm³ and did not survive through 80% of the duration of the analysis were excluded from the analysis of NAAC and NGRM.

It is understood that the examples and embodiments described herein are for illustrative purposes only and that various modifications or changes in light thereof will be suggested to persons skilled in the art and are to be included within the spirit and purview of this application and scope of the appended claims. All publications, patents, accession numbers, and patent applications cited herein are hereby incorporated by reference for the purposes in the context of which they are cited.

TABLE 1A

| | AIP | | |
|---|---|---|---|
| | Number | V_{H} Sequence | V_{L} Sequence |
| 1 | AIP- | EVQLVESGGALVKPGGSLRLSCAASGFTFSKAWMS | QSVLTQPPSASGTPGQRVTISCSGSSSNIGSSSVSWYQQLPG |
| | 101235 | WVRQAPGKGLEWVGRIKSVTDGETTDYAAPVKGRF | TAPKLLIYKNNQRPSGVPDRFSGSKSGTSASLAISGLRSEDE |
| | | TISRDDSKSTLYLQMNSLKTEDTAVYYCTSSFCCR | ADYYCSTWDDSLSVRVFGGGTKLTVL (SEQ ID NO: 123) |
| | | SGSCPSSDTSYCNGQYKSYYYMDVWGKGTTVTVSS | |
| | | (SEQ ID NO: 77) | |
| 2 | AIP- | EVQLVESGGGLVQPGGSLRLSCAASGFTFSKAWMS | QSVLTQPPSVSAAPGQKVTISCSGSSSNIGSSSVSWYQQLPG |
| | 127782 | WVRQAPGKGLEWVGRIKSVTDGETTEYAASVKGRF | TAPKLLIYKNNQRPSGIPDRFSGSKSGTSATLGITGLQTGDE |
| | | TISRDDSKNSLYLQMNSLKTEDTAVYYCTSSFCCR | ADYYCSTWDDSLSVRVFGGGTKLTVL (SEQ ID NO: 124) |
| | | GGSCPSHDTSYCGGQYKSYYYMDVWGKGTTVTVSS | |
| | | (SEQ ID NO: 78) | |
| 3 | AIP- | EVQLVESGGALVKPGGSLRLSCAASGFTFSKAWMS | QSVLTQPPSASGTPGQRVTISCSGSSSNIGSSSVSWYQQLPG |
| | 189473 | WVRQAPGKGLEWVGRIKSVTDGETTDYAAPVKGRF | TAPKLLIYKNNQRPSGVPDRFSGSKSGTSASLAISGLRSEDE |
| | | TISRDDSKSTLYLQMNSLKTEDTAVYYCTTSFCCR | ADYYCSTWDDSLSVRVFGGGTKLTVL (SEQ ID NO: 125) |
| | | GASCPSSDTSYCAGSYKSYYFVNIWGKGTTVTVSS | |
| | | (SEQ ID NO: 79) | |
| 4 | AIP- | EVQLVESGGALVKPGGSLRLSCAASGFTFSKAWMS | QSVLTQPPSASGTPGQRVTISCSGSSSNIGSSSVSWYQQLPG |
| | 192571 | WVRQAPGKGLEWVGRIKSVTDGETTDYAAPVKGRF | TAPKLLIYKNNQRPSGVPDRFSGSKSGTSASLAISGLRSEDE |
| | | TISRDDSKSTLYLQMNSLKTEDTAVYYCTSSFCCR | ADYYCSTWDDSLSVRVFGGGTKLTVL (SEQ ID NO: 126) |
| | | GNQCPSSDTSYCGGQYPSYYYMDPWGKGTTVTVSS | |
| | | (SEQ ID NO: 80) | |
| 5 | AIP- | EVQLVESGGALVKPGGSLRLSCAASGFTFSKAWMS | QSVLTQPPSASGTPGQRVTISCSGSSSNIGSSSVSWYQQLPG |
| | 125258 | WVRQAPGKGLEWVGRIKSVTDGETTDYAAPVKGRF | TAPKLLIYKNNQRPSGVPDRFSGSKSGTSASLAISGLRSEDE |
| | | TISRDDSKSTLYLQMNSLKTEDTAVYYCISSFCCR | ADYYCSTWDDSLSVRVFGGGTKLTVL (SEQ ID NO: 127) |
| | | GKQCPSSDTSYCNGYYADYYFMDVWGKGTTVTVSS | |
| | | (SEQ ID NO: 81) | |
| 6 | AIP- | EVQLVESGGALVKPGGSLRLSCAASGFTFSKAWMS | QSVLTQPPSASGTPGQRVTISCSGSSSNIGSSSVSWYQQLPG |
| | 150199 | WVRQAPGKGLEWVGRIKSVTDGETTDYAAPVKGRF | TAPKLLIYKNNQRPSGVPDRFSGSKSGTSASLAISGLRSEDE |
| | | TISRDDSKSTLYLQMNSLKTEDTAVYYCISSFCCR | ADYYCSTWDDSLSVRVFGGGTKLTVL (SEQ ID NO: 128) |
| | | GKQCPSSDTSYCGGQFKSYYFMDVWGKGTTVTVSS | |
| | | (SEQ ID NO: 82) | |
| 7 | AIP- | EVQLVESGGALVKPGGSLRLSCAASGFTFSKAWMS | QSVLTQPPSASGTPGQRVTISCSGSSSNIGSSSVSWYQQLPG |
| | 115388 | WVRQAPGKGLEWVGRIKSVTDGETTDYAAPVKGRF | TAPKLLIYKNNQRPSGVPDRFSGSKSGTSASLAISGLRSEDE |
| | | TISRDDSKSTLYLQMNSLKTEDTAVYYCISSFCCH | ADYYCSTWDDSLSVRVFGGGTKLTVL (SEQ ID NO: 129) |
| | | SNNCPSSDTSYCNGYYKQYYFMDVWGKGTTVTVSS | |
| | | (SEQ ID NO: 83) | |
| 8 | AIP- | EVQLVESGGALVKPGGSLRLSCAASGFTYSAAWMS | QSVLTQPPSASGTPGQRVTISCSGSSTNIGSSSVTWYQQLPG |
| | 143369 | WVRQAPGKGLEWVGRIKAVHDGETTDYAAPVKGRF | TAPKLLIYKDNQRPLGVPDRFSGSKSGTSASLAISGLRSEDE |
| | | TISRDDSKSTLYLQMNSLKTEDTAVYYCTSSFCCR | ADYYCSTWDDSLSVRVFGGGTKLTVL (SEQ ID NO: 130) |
| | | GGSCPSHDTSYCGGQYKSYYYMDVWGKGTTVTVSS | |
| | | (SEQ ID NO: 84) | |
| 9 | AIP- | EVQLVESGGALVKPGGSLRLSCAASGFTFSMAWMS | QSVLTQPPSASGTPGQRVTISCSGSKSNIGSSYVSWYQQLPG |
| | 157045 | WVRQAPGKGLEWVGRIKSNTDAETTDYAAPVKGRF | TAPKLLIYMNNQRPYGVPDRFSGSKSGTSASLAISGLRSEDE |
| | | TISRDDSKSTLYLQMNSLKTEDTAVYYCTSSFCCR | ADYYCATWDDQLSVRVFGGGTKLTVL (SEQ ID NO: 131) |
| | | GGSCPSHDTSYCGGQYKSYYYMDVWGKGTTVTVSS | |
| | | (SEQ ID NO: 85) | |
| 10 | AIP- | EVQLVESGGALVKPGGSLRLSCAASGFTFSAAWMS | QSVLTQPPSASGTPGQRVTISCSGSKSNIGSSSVSWYQQLPG |
| | 175775 | WVRQAPGKGLEWVGRIKSVTDGETTDYAAPVKGRF | TAPKLLIYKDNQRPSGVPDRFSGSKSGTSASLAISGLRSEDE |
| | | TISRDDSKSTLYLQMNSLKTEDTAVYYCISSFCCR | ADYYCATWDNSLSIRVFGGGTKLTVL (SEQ ID NO: 132) |
| | | GNSCPSSDTSYCNGQYKSYYYMDVWGKGTTVTVSS | |
| | | (SEQ ID NO: 86) | |
| 11 | AIP- | EVQLVESGGALVKPGGSLRLSCAASGFTYSKAWMS | QSVLTQPPSASGTPGQRVTISCSGASSNIGSSSVSWYQQLPG |
| | 154181 | WVRQAPGKGLEWVGRIKSVQDGETTDYAAPVKGRF | TAPKLLIYKNTQRASGVPDRFSGSKSGTSASLAISGLRSEDE |
| | | TISRDDSKSTLYLQMNSLKTEDTAVYYCTSSFCCR | ADYYCSSWDDSNSVRIFGGGTKLTVL (SEQ ID NO: 133) |
| | | GGSCPSHDTSYCGGQYKSFYYMDVWGKGTTVTVSS | |
| | | (SEQ ID NO: 87) | |
| 12 | AIP- | EVQLVESGGALVKPGGSLRLSCAASGFTFAKAWMS | QSVLTQPPSASGTPGQRVTISCSGSSTNIGSSTVSWYQQLPG |
| | 125984 | WVRQAPGKGLEWVGRIKSVTDGHTTDYAAPVKGRF | TAPKLLIYKNNARPYGVPDRFSGSKSGTSASLAISGLRSEDE |
| | | TISRDDSKSTLYLQMNSLKTEDTAVYYCTSSFCCR | ADYYCATWDDSLTVRIFGGGTKLTVL (SEQ ID NO: 134) |
| | | GGSCPSHDTSYCGGQYKSYYYMDVWGKGTTVTVSS | |
| | | (SEQ ID NO: 88) | |
| 13 | AIP- | EVQLVESGGALVKPGGSLRLSCAASGFVFSKAWMS | QSVLTQPPSASGTPGQRVTISCSGASSNIGSSSVSWYQQLPG |
| | 160829 | WVRQAPGKGLEWVGRIKSVTDGGITDYAAPVKGRF | TAPKLLIYKNTQRPSGVPDRFSGSKSGTSASLAISGLRSEDE |
| | | TISRDDSKSTLYLQMNSLKTEDTAVYYCISSFCCR | ADYYCATWDDQLSVRVFGGGTKLTVL (SEQ ID NO: 135) |
| | | GNSCPSSDTSYCNGQYKSYYFMDVWGKGTTVTVSS | |
| | | (SEQ ID NO: 89) | |
| 14 | AIP- | EVQLVESGGALVKPGGSLRLSCAASGFTFSAAWMS | QSVLTQPPSASGTPGQRVTISCSGSSTNIGSSSVSWYQQLPG |
| | 184744 | WVRQAPGKGLEWVGRIKSTSDGETTDYAAPVKGRF | TAPKLLIYKDNQRPSGVPDRFSGSKSGTSASLAISGLRSEDE |
| | | TISRDDSKSTLYLQMNSLKTEDTAVYYCISSFCCR | ADYYCSSWDDSNSVRIFGGGTKLTVL (SEQ ID NO: 136) |
| | | GGSCPSRDTSYCGGQYKSYYFMDVWGKGTTVTVSS | |
| | | (SEQ ID NO: 90) | |
| 15 | AIP- | EVQLVESGGALVKPGGSLRLSCAASGFTFSKAWMS | QSVLTQPPSASGTPGQRVTISCSGSKSNIGSSSVYWYQQLPG |
| | 128136 | WVRQAPGKGLEWVGRIKSVQDGETTDYAAPVKGRF | TAPKLLIYKNNQRPYGVPDRFSGSKSGTSASLAISGLRSEDE |
| | | TISRDDSKSTLYLQMNSLKTEDTAVYYCTSSFCCR | ADYYCSTWDDALSVRVFGGGTKLTVL (SEQ ID NO: 137) |
| | | GGSCPSSDTSYCGGYYKSYYFMDVWGKGTTVTVSS | |
| | | (SEQ ID NO: 91) | |
| 16 | AIP- | EVQLVESGGALVKPGGSLRLSCAASGFTFSKAWMS | QSVLTQPPSASGTPGQRVTISCSGSSSNIGSSSVYWYQQLPG |
| | 181273 | WVRQAPGKGLEWVGRIKSVTDGETTDYAAPVKGRF | TAPKLLIYKNNQRPSGVPDRFSGSKSGTSASLAISGLRSEDE |
| | | TISRDDSKSTLYLQMNSLKTEDTAVYYCTSSFCCR | ADYYCSTWDDALSVRVFGGGTKLTVL (SEQ ID NO: 138) |
| | | GGSCPSSDTSYCGGQYKSYYFMDVWGKGTTVTVSS | |
| | | (SEQ ID NO: 92) | |
| 17 | AIP- | EVQLVESGGALVKPGGSLRLSCAASGFTFSKAWMS | QSVLTQPPSASGTPGQRVTISCSGSKSNIGSSYVSWYQQLPG |
| | 153125 | WVRQAPGKGLEWVGRIKSVQDGETTDYAAPVKGRF | TAPKLLIYKNNQRPYGVPDRFSGSKSGTSASLAISGLRSEDE |
| | | TISRDDSKSTLYLQMNSLKTEDTAVYYCTSSFCCR | ADYYCSTWDDSLSVRVFGGGTKLTVL (SEQ ID NO: 139) |
| | | GGSCPSHDTSYCGGYYKSYYYMDVWGKGTTVTVSS | |
| | | (SEQ ID NO: 93) | |
| 18 | AIP- | EVQLVESGGALVKPGGSLRLSCAASGFTFSKAWMS | QSVLTQPPSASGTPGQRVTISCSGSSSNIGSSSVSWYQQLPG |
| | 160470 | WVRQAPGKGLEWVGRIKSVTDGETTDYAAPVKGRF | TAPKLLIYKNNQRPSGVPDRFSGSKSGTSASLAISGLRSEDE |
| | | TISRDDSKSTLYLQMNSLKTEDTAVYYCTSSFCCR | ADYYCSTWDDSLSVRVFGGGTKLTVL (SEQ ID NO: 140) |
| | | GGSCPSHDTSYCGGQYKSYYYMDVWGKGTTVTVSS | |
| | | (SEQ ID NO: 94) | |
| 19 | AIP- | EVQLVESGGALVKPGGSLRLSCAASGFTFSKAWMT | QSVLTQAPSASETPGQRVIISCSGSSSNIGSSSVSWYQQLPG |
| | 192482 | WVRQAPGKGLEWVGRIKSVTDGETTDYAAPVKGRF | TAPKLLIYKNNQRPSGVPDRFSGSKSGTSASLAISGLRSEDE |
| | | TISRDDSKSVLYLQMSSLKTEDTAVYFCTSSFCCR | ADYYCSTWDDSLSVRVFGGGTKLTVL (SEQ ID NO: 141) |
| | | GGSCPSHDTSFCGGQDKRYYYMDVWGKGTTVTVSS | |
| | | (SEQ ID NO: 95) | |
| 20 | AIP- | EVQLVESGGGLVKPGGSLRLSCAASGFTFSKAWMT | QSVLTQPPSASGTPGQRVTISCSGSSSNIGSSSVSWYQQLPG |
| | 171142 | WVRQAPGKGLEWVGRIKSVTDGETTDYAAPVKGRF | TAPKLLIYKNNQRPSGVPDRFSGSKSGTSASLAISGLRSEDE |
| | | TISRDDSKNTLYLQMNSLKTEDTAVYYCTTSFCCR | ADYYCSTWDDSLSVRVFGGGTKLTVL (SEQ ID NO: 142) |
| | | GGSCPSHDTSFCGGQDKRYYYMDVWGQGTTVTVSS | |
| | | (SEQ ID NO: 96) | |
| 21 | AIP- | EVQLVESGGALVKPGGSLRLSCAASGFTFSKAWMT | QSVLTQAPSASETPGQRVIISCSGSSSNIGSSSVSWYQQLPG |
| | 157397 | WVRQAPGKGLEWVGRIKSVTDGETTDYAAPVKGRF | TAPKLLIYKNNQRPSGVPDRFSGSKSGTSASLAISGLRSEDE |
| | | TISRDDSKSVLYLQMSSLKTEDTAVYFCTSSFCCR | ADYYCSTWDDSLSVRVFGGGTKLTVL (SEQ ID NO: 143) |
| | | GGSCPSHDTSFCGGQYNRYYYMDVWGKGTTVTVSS | |
| | | (SEQ ID NO: 97) | |
| 22 | AIP- | EVQLVESGGALVKPGGSLRLSCAASGFTFSKAWMT | QSVLTQAPSASETPGQRVIISCSGSSSNIGSSSVSWYQQLPG |
| | 165430 | WVRQAPGKGLEWVGRIKSVTDGETTDYAAPVKGRF | TAPKLLIYKNNQRPSGVPDRFSGSKSGTSASLAISGLRSEDE |
| | | TISRDDSKSVLYLQMSSLKTEDTAVYFCTSSFCCR | ADYYCSTWDDSLSVRVFGGGTKLTVL (SEQ ID NO: 144) |
| | | GGSCPSHDTSFCGGQYKSYYYMDVWGKGTTVTVSS | |
| | | (SEQ ID NO: 98) | |
| 23 | AIP- | EVQLVESGGGLVKPGGSLRLSCAASGFTFSKAWMT | QSVLTQPPSASGTPGQRVTISCSGSSSNIGSSSVSWYQQLPG |
| | 189526 | WVRQAPGKGLEWVGRIKSTSDGGITDYAAPVKGRF | TAPKLLIYKNNQRPSGVPDRFSGSKSGTSASLAISGLRSEDE |
| | | TISRDDSKNTLYLQMNSLKTEDTAVYYCTTSFCCR | ADYYCSTWDDSLSVRVFGGGTKLTVL (SEQ ID NO: 145) |
| | | GGRCPSRDTSFCGGQYNSYYYMDVWGQGTTVTVSS | |
| | | (SEQ ID NO: 99) | |
| 24 | AIP- | EVQLVESGGALVKPGGSLRLSCAASGFTFSKAWMT | QSVLTQPPSASGTPGQRVTISCSGSSSNIGSSSVSWYQQLPG |
| | 122563 | WVRQAPGKGLEWVGRIKSVTDGETTDYAAPVKGRF | TAPKLLIYKNNQRPSGVPDRFSGSKSGTSASLAISGLRSEDE |
| | | TISRDDSKSTLYLQMNSLKTEDTAVYYCTSSFCCR | ADYYCSTWDDSLSVRVFGGGTKLTVL (SEQ ID NO: 146) |
| | | GGSCPSHDTSFCGGQDKRYYYMDVWGKGTTVTVSS | |
| | | (SEQ ID NO: 100) | |
| 25 | AIP- | EVQLVESGGALVKPGGSLRLSCAASGFTFSKAWMT | QSVLTQPPSASGTPGQRVTISCSGSSSNIGSSSVSWYQQLPG |
| | 158623 | WVRQAPGKGLEWVGRIKSVTDGETTDYAAPVKGRF | TAPKLLIYKNNQRPSGVPDRFSGSKSGTSASLAISGLRSEDE |
| | | TISRDDSKSTLYLQMNSLKTEDTAVYYCTSSFCCR | ADYYCSTWDDSLSVRVFGGGTKLTVL (SEQ ID NO: 147) |
| | | GGSCPSHDTSFCGGQYKSYYYMDVWGKGTTVTVSS | |
| | | (SEQ ID NO: 101) | |
| 26 | AIP- | EVQLVESGGGLVKPGGSLRLSCAASGFTFSKAWMT | QSVLTQPPSASGTPGQRVTISCSGSSSNIGSSSVSWYQQLPG |
| | 155066 | WVRQAPGKGLEWVGRIKSVTDGETTDYAAPVKGRF | TAPKLLIYKNNQRPSGVPDRFSGSKSGTSASLAISGLRSEDE |
| | | TISRDDSKNTLYLQMNSLKTEDTAVYYCTTSFCCR | ADYYCSTWDDSLSVRVFGGGTKLTVL (SEQ ID NO: 148) |
| | | GGSCPSHDTSFCGGQYKSYYYMDVWGQGTTVTVSS | |
| | | (SEQ ID NO: 102) | |
| 27 | AIP- | EVQLVESGGALVKPGGSLRLSCAASGFTFSKAWMT | QSVLTQPPSASGTPGQRVTISCSGSSSNIGSSSVSWYQQLPG |
| | 136538 | WVRQAPGKGLEWVGRIKSVTDGETTDYAAPVKGRF | TAPKLLIYKNNQRPSGVPDRFSGSKSGTSASLAISGLRSEDE |
| | | TISRDDSKSTLYLQMNSLKTEDTAVYYCTSSFCCR | ADYYCSTWDDSLSVRVFGGGTKLTVL (SEQ ID NO: 149) |
| | | GGSCPSHDTSFCGGQDSRYYYMDVWGKGTTVTVSS | |
| | | (SEQ ID NO: 103) | |
| 28 | AIP- | EVQLVESGGALVKPGGSLRLSCAASGFTFSKAWMT | QSVLTQPPSASGTPGQRVTISCSGSSSNIGSSSVSWYQQLPG |
| | 166120 | WVRQAPGKGLEWVGRIKSVTDGETTDYAAPVKGRF | TAPKLLIYKNNQRPSGVPDRFSGSKSGTSASLAISGLRSEDE |
| | | TISRDDSKSTLYLQMNSLKTEDTAVYYCTSSFCCR | ADYYCSTWDDSLSVRVFGGGTKLTVL (SEQ ID NO: 150) |
| | | GGSCPSHDTSYCGGQYKSYYYMDVWGKGTTVTVSS | |
| | | (SEQ ID NO: 104) | |
| 29 | AIP- | EVQLVESGGALVKPGGSLRLSCAASGFTFSKAWMT | QSVLTQPPSASGTPGQRVTISCSGSSSNIGSSSVSWYQQLPG |
| | 133645 | WVRQAPGKGLEWVGRIKSVTDGETTDYAAPVKGRF | TAPKLLIYKNNQRPSGVPDRFSGSKSGTSASLAISGLRSEDE |
| | | TISRDDSKSTLYLQMNSLKTEDTAVYYCTSSFCCR | ADYYCSTWDESLSVRVFGGGTKLTVL (SEQ ID NO: 151) |
| | | GGSCPSHDTSFCGGQYKSYYYMDVWGKGTTVTVSS | |
| | | (SEQ ID NO: 105) | |
| 30 | AIP- | EVQLVESGGALVKPGGSLRLSCAASGFTFSKAWMS | QSVLTQPPSASGTPGQRVTISCSGSSSNIGSSSVSWYQQLPG |
| | 187893 | WVRQAPGKGLEWVGRIKSVTDGETTDYAAPVKGRF | TAPKLLIYKNNQRPSGVPDRFSGSKSGTSASLAISGLRSEDE |
| | | TISRDDSKSTLYLQMNSLKTEDTAVYYCTSSFCCR | ADYYCSTWDDSLSVRVFGGGTKLTVL (SEQ ID NO: 152) |
| | | GGSCPSHDTSFCGGQYKSYYYMDVWGKGTTVTVSS | |
| | | (SEQ ID NO: 106) | |
| 31 | AIP- | EVQLVESGGALVKPGGSLRLSCAASGFTFSKAWMT | QSVLTQPPSASGTPGQRVTISCSGSSSNIGSSSVSWYQQLPG |
| | 142079 | WVRQAPGKGLEWVGRIKSVTDGETTDYAAPVKGRF | TAPKLLIYKNNQRPSGVPDRFSGSKSGTSASLAISGLRSEDE |
| | | TISRDDSKSTLYLQMNSLKTEDTAVYYCTSSFCCR | ADYYCSTWDDSLSVRVFGGGTKLTVL (SEQ ID NO: 153) |
| | | GGSCPSHDTSFCGGSYKSYYYMDVWGKGTTVTVSS | |
| | | (SEQ ID NO: 107) | |
| 32 | AIP- | EVQLVESGGALVKPGGSLRLSCAASGFTFSKAWMT | QSVLTQPPSASGTPGQRVTISCSGSSSNIGSSSVSWYQQLPG |
| | 104188 | WVRQAPGKGLEWVGRIKSVTDGETTDYAAPVKGRF | TAPKLLIYKNNQRPSGVPDRFSGSKSGTSASLAISGLRSEDE |
| | | TISRDDSKSTLYLQMNSLKTEDTAVYYCTSSFCCR | ADYYCSTWDESLSVRVFGGGTKLTVL (SEQ ID NO: 154) |
| | | GGSCPSHDTSYCGGQDSRYYYMDVWGKGTTVTVSS | |
| | | (SEQ ID NO: 108) | |
| 33 | AIP- | EVQLVESGGGLVKPGGSLRLSCAASGFTFSKAWMT | QSVLTQPPSASGTPGQRVTISCSGSSSNIGSSSVSWYQQLPG |
| | 106042 | WVRQAPGKGLEWVGRIKSVTEGETTDYAAPVKGRF | TAPKLLIYKNNQRPSGVPDRFSGSKSGTSASLAISGLRSEDE |
| | | TISRDDSKNTLYLQMNSLKTEDTAVYYCTTSFCCR | ADYYCSTWDESLSVRVFGGGTKLTVL (SEQ ID NO: 155) |
| | | GGSCPSHDTSFCGGQYKSYYYMDVWGQGTTVTVSS | |
| | | (SEQ ID NO: 109) | |
| 34 | AIP- | EVQLVESGGALVKPGGSLRLSCAASGFTFSKAWMT | QSVLTQPPSASGTPGQRVTISCSGSSSNIGSSSVSWYQQLPG |
| | 100196 | WVRQAPGKGLEWVGRIKSVTEGETTDYAAPVKGRF | TAPKLLIYKNNQRPSGVPDRFSGSKSGTSASLAISGLRSEDE |
| | | TISRDDSKSTLYLQMNSLKTEDTAVYYCTSSFCCR | ADYYCSTWDESLSVRVFGGGTKLTVL (SEQ ID NO: 156) |
| | | GGSCPSHDTSFCGGQYKSYYYMDVWGKGTTVTVSS | |
| | | (SEQ ID NO: 110) | |
| 35 | AIP- | EVQLVESGGALVKPGGSLRLSCAASGFTFSKAWMS | QSVLTQPPSASGTPGQRVTISCSGSSSNIGSSSVSWYQQLPG |
| | 180675 | WVRQAPGKGLEWVGRIKSVTDGETTDYAAPVKGRF | TAPKLLIYKNNQRPSGVPDRFSGSKSGTSASLAISGLRSEDE |
| | | TISRDDSKSTLYLQMNSLKTEDTAVYYCTSSFCCR | ADYYCSTWDDSLSVRVFGGGTKLTVL (SEQ ID NO: 157) |
| | | GGSCPSHDTSYCGGQYKSYYFMDVWGKGTTVTVSS | |
| | | (SEQ ID NO: 111) | |
| 36 | AIP- | EVQLVESGGALVKPGGSLRLSCAASGFTFSKAWMS | QSVLTQPPSASGTPGQRVTISCSGSSSNIGSSSVYWYQQLPG |
| | 170105 | WVRQAPGKGLEWVGRIKSVTDGETTDYAAPVKGRF | TAPKLLIYKNNQRPSGVPDRFSGSKSGTSASLAISGLRSEDE |
| | | TISRDDSKSTLYLQMNSLKTEDTAVYYCTSSFCCR | ADYYCSTWDDSLSVRVFGGGTKLTVL (SEQ ID NO: 158) |
| | | GGSCPSHDTSYCGGQYKSYYYMDVWGKGTTVTVSS | |
| | | (SEQ ID NO: 112) | |
| 37 | AIP- | EVQLVESGGALVKPGGSLRLSCAASGFTFSKAWMS | QSVLTQPPSASGTPGQRVTISCSGSSSNIGSSSVSWYQQLPG |
| | 126080 | WVRQAPGKGLEWVGRIKSVTDGETTDYAAPVKGRF | TAPKLLIYKNNQRPSGVPDRFSGSKSGTSASLAISGLRSEDE |
| | | TISRDDSKSTLYLQMNSLKTEDTAVYYCTSSFCCR | ADYYCSTWDDSLSVRVFGGGTKLTVL (SEQ ID NO: 159) |
| | | GGSCPSHDTSYCGGQNKQYYYMDVWGKGTTVTVSS | |
| | | (SEQ ID NO: 113) | |
| 38 | AIP- | EVQLVESGGALVKPGGSLRLSCAASGFTFSKAWMS | QSVLTQPPSASGTPGQRVTISCSGSSSNIGSSSVSWYQQLPG |
| | 161571 | WVRQAPGKGLEWVGRIKSVTDGETTDYAAPVKGRF | TAPKLLIYKNNQRPSGVPDRFSGSKSGTSASLAISGLRSEDE |
| | | TISRDDSKSTLYLQMNSLKTEDTAVYYCTSSFCCR | ADYYCSTWDDSLSVRVFGGGTKLTVL (SEQ ID NO: 160) |
| | | GGNCPSHETSYCGNQYKSYYYMDVWGKGTTVTVSS | |
| | | (SEQ ID NO: 114) | |
| 39 | AIP- | EVQLVESGGALVKPGGSLRLSCAASGFTFSKAWMS | QSVLTQPPSASGTPGQRVTISCSGSSSNIGSSSVSWYQQLPG |
| | 181246 | WVRQAPGKGLEWVGRIKSVTDGETTDYAAPVKGRF | TAPKLLIYKNNQRPSGVPDRFSGSKSGTSASLAISGLRSEDE |
| | | TISRDDSKSTLYLQMNSLKTEDTAVYYCTSSFCCR | ADYYCSTWDDSLSVRVFGGGTKLTVL (SEQ ID NO: 161) |
| | | GGSCPSHDTSYCGGQYKSYYYVNIWGKGTTVTVSS | |
| | | (SEQ ID NO: 115) | |
| 40 | AIP- | EVQLVESGGALVKPGGSLRLSCAASGFTFSKAWMS | QSVLTQPPSASGTPGQRVTISCSGSSSNIGSSSVSWYQQLPG |
| | 192216 | WVRQAPGKGLEWVGRIKSVTDGETTDYAAPVKGRF | TAPKLLIYKNNQRPSGVPDRFSGSKSGTSASLAISGLRSEDE |
| | | TISRDDSKSTLYLQMNSLKTEDTAVYYCTTSFCCR | ADYYCSTWDDSLSVRVFGGGTKLTVL (SEQ ID NO: 162) |
| | | GACPSHDTSYCAGSYKSYYYMDVWGKGTTVTVSS | |
| | | (SEQ ID NO: 116) | |
| 41 | AIP- | EVQLVESGGALVKPGGSLRLSCAASGFTFSKAWMS | QSVLTQPPSASGTPGQRVTISCSGSSSDIGSSSVSWYQQLPG |
| | 168605 | WVRQAPGKGLEWVGRIKSVTDGETTDYAAPVKGRF | TAPKLLIYKNNQRPSGVPDRFSGSKSGTSASLAISGLRSEDE |
| | | TISRDDSKSTLYLQMNSLKTEDTAVYYCTSSFCCR | ADYYCSTWNDALSVRVFGGGTKLTVL (SEQ ID NO: 163) |
| | | GGSCPSHDTSYCGGQYKSYYYMDVWGKGTTVTVSS | |
| | | (SEQ ID NO: 117) | |
| 42 | AIP- | EVQLVESGGALVKPGGSLRLSCAASGFTFSKAWMS | QSVLTQPPSASGTPGQRVTISCSGSSSNIGSSSVSWYQQLPG |
| | 172872 | WVRQAPGKGLEWVGRIKSVTDGEQTDYAAPVKGRF | TAPKLLIYKNTQRPSGVPDRFSGSKSGTSASLAISGLRSEDE |
| | | TISRDDSKSTLYLQMNSLKTEDTAVYYCTSSFCCR | ADYYCSTWDDSLNVRVFGGGTKLTVL (SEQ ID NO: 164) |
| | | GGSCPSHDTSYCGGQYKSYYYMDVWGKGTTVTVSS | |
| | | (SEQ ID NO: 118) | |
| 43 | AIP- | EVQLVESGGALVKPGGSLRLSCAASGFTFSKAWMT | QSVLTQPPSASGTPGQRVTISCSGSPSNIGSSSVSWYQQLPG |
| | 190051 | WVRQAPGKGLEWVGRIKSVTDGETTDYAAPVKGRF | TAPKLLIYKNNQRPSGVPDRFSGSKSGTSASLAISGLRSEDE |
| | | TISRDDSKSTLYLQMNSLKTEDTAVYYCTSSFCCR | ADYYCSTWDDALSVRVFGGGTKLTVL (SEQ ID NO: 165) |
| | | GGSCPSHDTSYCGGQYKSYYYMDVWGKGTTVTVSS | |
| | | (SEQ ID NO: 119) | |
| 44 | AIP- | EVQLVESGGALVKPGGSLRLSCAASGFTFSKAWMS | QSVLTQPPSASGTPGQRVTISCSGSSSNIGSSSVSWYQQLPG |
| | 167533 | WVRQAPGKGLEWVGRIKAADDGKQTDYAAPVKGRF | TAPKLLIYKNNQRPSGVPDRFSGSKSGTSASLAISGLRSEDE |
| | | TISRDDSKSTLYLQMNSLKTEDTAVYYCTSSFCCR | ADYYCSTWDDSLSVRVFGGGTKLTVL (SEQ ID NO: 166) |
| | | GGSCPSHDTSYCGGQYKSYYYMDVWGKGTTVTVSS | |
| | | (SEQ ID NO: 120) | |
| 45 | AIP- | EVQLVESGGALVKPGGSLRLSCAASGFTFSKAWMS | QSVLTQPPSASGTPGQRVTISCSGSSSNIGSSSVSWYQQLPG |
| | 112580 | WVRQAPGKGLEWVGRIKSVTDGETTDYAAPVKGRF | TAPKLLIYKNNQRPSGVPDRFSGSKSGTSASLAISGLRSEDE |
| | | TISRDDSKSTLYLQMNSLKTEDTAVYYCISSFCCR | ADYYCSTWDDSLSVRVFGGGTKLTVL (SEQ ID NO: 167) |
| | | GNSCPSSDTSYCNGQYKSYYYMDVWGKGTTVTVSS | |
| | | (SEQ ID NO: 121) | |
| 46 | AIP- | EVQLVESGGALVKPGGSLRLSCAASGFTFSKAWMS | QSVLTQPPSASGTPGQRVTISCSGSSSNIGSSSVSWYQQLPG |
| | 136060 | WVRQAPGKGLEWVGRIKSVTDGETTDYAAPVKGRF | TAPKLLIYKNSLRPQGVPDRFSGSKSGTSASLAISGLRSEDE |
| | | TISRDDSKSTLYLQMNSLKTEDTAVYYCTSSFCCR | ADYYCSTWDDSLSVRVFGGGTKLTVL (SEQ ID NO: 168) |
| | | GGSCPSHDTSYCGGQYKSYYYMDVWGKGTTVTVSS | |
| | | (SEQ ID NO: 122) | |
| 47 | AIP- | EVQLVESGGALVKPGGSLRLSCAASGFTFSKAWMS | QSVLTQPPSASGTPGQRVTISCSGSSSNIGSSSVSWYQQLPG |
| | 125646 | WVRQAPGKGLEWVGRIKSVTDGETTDYAAPVKGRF | TAPKLLIYKNNQRPSGVPDRFSGSKSGTSASLAISGLRSEDE |
| | | TISRDDSKSTLYLQMNSLKTEDTAVYYCTSSFCCR | ADYYCSTWDDSLSVRVFGGGTKLTVL (SEQ ID NO: 123) |
| | | GGSCPSHDTSYCGQYKSYYYMDVWGKGTTVTVSS | |
| | | (SEQ ID NO: 1746) | |

TABLE 2B

| | AIP | | | | | | |
|---|---|---|---|---|---|---|---|
| | Number | HCDR1 | HCDR2 | HCDR3 | LCDR1 | LCDR2 | LCDR3 |
| 1 | AIP- | GFTFSKAWMS | RIKSVTDGETTDYA | TSSFCCRSGSCPSS | SGSSSNIGSSSV | KNNQRPS | STWDDSLSVRV |
| | 101235 | (SEQ ID | APVKG (SEQ ID | DTSYCNGQYKSYYY | S (SEQ ID | (SEQ ID | (SEQ ID |
| | | NO: 1) | NO: 9) | MDV (SEQ ID | NO: 48) | NO: 59) | NO: 68) |
| | | | | NO: 20) | | | |
| 2 | AIP- | GFTFSKAWMS | RIKSVTDGETTEYA | TSSFCCRGGSCPSH | SGSSSNIGSSSV | KNNQRPS | STWDDSLSVRV |
| | 127782 | (SEQ ID | ASVKG (SEQ ID | DTSYCGGQYKSYYY | S (SEQ ID | (SEQ ID | (SEQ ID |
| | | NO: 1) | NO: 504) | MDV (SEQ ID | NO: 48) | NO: 59) | NO: 68) |
| | | | | NO: 21) | | | |
| 3 | AIP- | GFTFSKAWMS | RIKSVTDGETTDYA | TTSFCCRGASCPSS | SGSSSNIGSSSV | KNNQRPS | STWDDSLSVRV |
| | 189473 | (SEQ ID | APVKG (SEQ ID | DTSYCAGSYKSYYF | S (SEQ ID | (SEQ ID | (SEQ ID |
| | | NO: 1) | NO: 9) | VNI (SEQ ID | NO: 48) | NO: 59) | NO: 68) |
| | | | | NO: 22) | | | |
| 4 | AIP- | GFTFSKAWMS | RIKSVTDGETTDYA | TSSFCCRGNQCPSS | SGSSSNIGSSSV | KNNQRPS | STWDDSLSVRV |
| | 192571 | (SEQ ID | APVKG (SEQ ID | DTSYCGGQYPSYYY | S (SEQ ID | (SEQ ID | (SEQ ID |
| | | NO: 1) | NO: 9) | MDP (SEQ ID | NO: 48) | NO: 59) | NO: 68) |
| | | | | NO: 23) | | | |
| 5 | AIP- | GFTFSKAWMS | RIKSVTDGETTDYA | ISSFCCRGKQCPSS | SGSSSNIGSSSV | KNNQRPS | STWDDSLSVRV |
| | 125258 | (SEQ ID | APVKG (SEQ ID | DTSYCNGYYADYYF | S (SEQ ID | (SEQ ID | (SEQ ID |
| | | NO: 1) | NO: 9) | MDV (SEQ ID | NO: 48) | NO: 59) | NO: 68) |
| | | | | NO: 24) | | | |
| 6 | AIP- | GFTFSKAWMS | RIKSVTDGETTDYA | ISSFCCRGKQCPSS | SGSSSNIGSSSV | KNNQRPS | STWDDSLSVRV |
| | 150199 | (SEQ ID | APVKG (SEQ ID | DTSYCGGQFKSYYF | S (SEQ ID | (SEQ ID | (SEQ ID |
| | | NO: 1) | NO: 9) | MDV (SEQ ID | NO: 48) | NO: 59) | NO: 68) |
| | | | | NO: 25) | | | |
| 7 | AIP- | GFTFSKAWMS | RIKSVTDGETTDYA | ISSFCCHSNNCPSS | SGSSSNIGSSSV | KNNQRPS | STWDDSLSVRV |
| | 115388 | (SEQ ID | APVKG (SEQ ID | DTSYCNGYYKQYYF | S (SEQ ID | (SEQ ID | (SEQ ID |
| | | NO: 1) | NO: 9) | MDV (SEQ ID | NO: 48) | NO: 59) | NO: 68) |
| | | | | NO: 26) | | | |
| 8 | AIP- | GFTYSAAWMS | RIKAVHDGETTDYA | TSSFCCRGGSCPSH | SGSSTNIGSSSV | KDNQRPL | STWDDSLSVRV |
| | 143369 | (SEQ ID | APVKG (SEQ ID | DTSYCGGQYKSYYY | T (SEQ ID | (SEQ ID | (SEQ ID |
| | | NO: 2) | NO: 10) | MDV (SEQ ID | NO: 49) | NO: 60) | NO: 68) |
| | | | | NO: 21) | | | |
| 9 | AIP- | GFTFSMAWMS | RIKSNTDAETTDYA | TSSFCCRGGSCPSH | SGSKSNIGSSYV | MNNQRPY | ATWDDQLSVRV |
| | 157045 | (SEQ ID | APVKG (SEQ ID | DTSYCGGQYKSYYY | S (SEQ ID | (SEQ ID | (SEQ ID |
| | | NO: 3) | NO: 11) | MDV (SEQ ID | NO: 50) | NO: 61) | NO: 69) |
| | | | | NO: 21) | | | |
| 10 | AIP- | GFTFSAAWMS | RIKSVTDGETTDYA | ISSFCCRGNSCPSS | SGSKSNIGSSSV | KDNQRPS | ATWDNSLSIRV |
| | 175775 | (SEQ ID | APVKG (SEQ ID | DTSYCNGQYKSYYY | S (SEQ ID | (SEQ ID | (SEQ ID |
| | | NO: 4) | NO: 9) | MDV (SEQ ID | NO: 51) | NO: 62) | NO: 70) |
| | | | | NO: 27) | | | |
| 11 | AIP- | GFTYSKAWMS | RIKSVQDGETTDYA | TSSFCCRGGSCPSH | SGASSNIGSSSV | KNTQRAS | SSWDDSNSVRI |
| | 154181 | (SEQ ID | APVKG (SEQ ID | DTSYCGGQYKSFYY | S (SEQ ID | (SEQ ID | (SEQ ID |
| | | NO: 5) | NO: 12) | MDV (SEQ ID | NO: 52) | NO: 63) | NO: 71) |
| | | | | NO: 28) | | | |
| 12 | AIP- | GFTFAKAWMS | RIKSVTDGHTTDYA | TSSFCCRGGSCPSH | SGSSTNIGSSTV | KNNARPY | ATWDDSLTVRI |
| | 125984 | (SEQ ID | APVKG (SEQ ID | DTSYCGGQYKSYYY | S (SEQ ID | (SEQ ID | (SEQ ID |
| | | NO: 6) | NO: 13) | MDV (SEQ ID | NO: 53) | NO: 64) | NO: 76) |
| | | | | NO: 21) | | | |
| 13 | AIP- | GFVFSKAWMS | RIKSVTDGGITDYA | ISSFCCRGNSCPSS | SGASSNIGSSSV | KNTQRPS | ATWDDQLSVRV |
| | 160829 | (SEQ ID | APVKG | DTSYCNGQYKSYYF | S (SEQ ID | (SEQ ID | (SEQ ID |
| | | NO: 7) | (SEQ ID | MDV (SEQ ID | NO: 52) | NO: 65) | NO: 69) |
| | | | NO: 14) | NO: 29) | | | |
| 14 | AIP- | GFTFSAAWMS | RIKSTSDGETTDYA | ISSFCCRGGSCPSR | SGSSTNIGSSSV | KDNQRPS | SSWDDSNSVRI |
| | 184744 | (SEQ ID | APVKG (SEQ ID | DTSYCGGQYKSYYF | S (SEQ ID | (SEQ ID | (SEQ ID |
| | | NO: 4) | NO: 15) | MDV (SEQ ID | NO: 54) | NO: 62) | NO: 71) |
| | | | | NO: 30) | | | |
| 15 | AIP- | GFTFSKAWMS | RIKSVQDGETTDYA | TSSFCCRGGSCPSS | SGSKSNIGSSSV | KNNQRPY | STWDDALSVRV |
| | 128136 | (SEQ ID | APVKG (SEQ ID | DTSYCGGYYKSYYF | Y (SEQ ID | (SEQ ID | (SEQ ID |
| | | NO: 1) | NO: 12) | MDV (SEQ ID | NO: 55) | NO: 66) | NO: 72) |
| | | | | NO: 31) | | | |
| 16 | AIP- | GFTFSKAWMS | RIKSVTDGETTDYA | TSSFCCRGGSCPSS | SGSSSNIGSSSV | KNNQRPS | STWDDALSVRV |
| | 181273 | (SEQ ID | APVKG (SEQ ID | DTSYCGGQYKSYYF | Y (SEQ ID | (SEQ ID | (SEQ ID |
| | | NO: 1) | NO: 9) | MDV (SEQ ID | NO: 56) | NO: 59) | NO: 72) |
| | | | | NO: 32) | | | |
| 17 | AIP- | GFTFSKAWMS | RIKSVQDGETTDYA | TSSFCCRGGSCPSH | SGSKSNIGSSYV | KNNQRPY | STWDDSLSVRV |
| | 153125 | (SEQ ID | APVKG (SEQ ID | DTSYCGGYYKSYYY | S (SEQ ID | (SEQ ID | (SEQ ID |
| | | NO: 1) | NO: 12) | MDV (SEQ ID | NO: 50) | NO: 66) | NO: 68) |
| | | | | NO: 33) | | | |
| 18 | AIP- | GFTFSKAWMS | RIKSVTDGETTDYA | TSSFCCRGGSCPSH | SGSSSNIGSSSV | KNNQRPS | STWDDSLSVRV |
| | 160470 | (SEQ ID | APVKG (SEQ ID | DTSYCGGQYKSYYY | S (SEQ ID | (SEQ ID | (SEQ ID |
| | | NO: 1) | NO: 9) | MDV (SEQ ID | NO: 48) | NO: 59) | NO: 68) |
| | | | | NO: 21) | | | |
| 19 | AIP- | GFTFSKAWMT | RIKSVTDGETTDYA | TSSFCCRGGSCPSH | SGSSSNIGSSSV | KNNQRPS | STWDDSLSVRV |
| | 192482 | (SEQ ID | APVKG (SEQ ID | DTSFCGGQDKRYYY | S (SEQ ID | (SEQ ID | (SEQ ID |
| | | NO: 8) | NO: 9) | MDV (SEQ ID | NO: 48) | NO: 59) | NO: 68) |
| | | | | NO: 34) | | | |
| 20 | AIP- | GFTFSKAWMT | RIKSVTDGETTDYA | TTSFCCRGGSCPSH | SGSSSNIGSSSV | KNNQRPS | STWDDSLSVRV |
| | 171142 | (SEQ ID | APVKG (SEQ ID | DTSFCGGQDKRYYY | S (SEQ ID | (SEQ ID | (SEQ ID |
| | | NO: 8) | NO: 9) | MDV (SEQ ID | NO: 48) | NO: 59) | NO: 68) |
| | | | | NO: 35) | | | |
| 21 | AIP- | GFTFSKAWMT | RIKSVTDGETTDYA | TSSFCCRGGSCPSH | SGSSSNIGSSSV | KNNQRPS | STWDDSLSVRV |
| | 157397 | (SEQ ID | APVKG (SEQ ID | DTSFCGGQYNRYYY | S (SEQ ID | (SEQ ID | (SEQ ID |
| | | NO: 8) | NO: 9) | MDV (SEQ ID | NO: 48) | NO: 59) | NO: 68) |
| | | | | NO: 36) | | | |
| 22 | AIP- | GFTFSKAWMT | RIKSVTDGETTDYA | TSSFCCRGGSCPSH | SGSSSNIGSSSV | KNNQRPS | STWDDSLSVRV |
| | 165430 | (SEQ ID | APVKG (SEQ ID | DTSFCGGQYKSYYY | S (SEQ ID | (SEQ ID | (SEQ ID |
| | | NO: 8) | NO: 9) | MDV (SEQ ID | NO: 48) | NO: 59) | NO: 68) |
| | | | | NO: 37) | | | |
| 23 | AIP- | GFTFSKAWMT | RIKSTSDGGITDYA | TTSFCCRGGRCPSR | SGSSSNIGSSSV | KNNQRPS | STWDDSLSVRV |
| | 189526 | (SEQ ID | APVKG | DTSFCGGQYNSYYY | S (SEQ ID | (SEQ ID | (SEQ ID |
| | | NO: 8) | (SEQ ID | MDV (SEQ ID | NO: 48) | NO: 59) | NO: 68) |
| | | | NO: 16) | NO: 38) | | | |
| 24 | AIP- | GFTFSKAWMT | RIKSVTDGETTDYA | TSSFCCRGGSCPSH | SGSSSNIGSSSV | KNNQRPS | STWDDSLSVRV |
| | 122563 | (SEQ ID | APVKG (SEQ ID | DTSFCGGQDKRYYY | S (SEQ ID | (SEQ ID | (SEQ ID |
| | | NO: 8) | NO: 9) | MDV (SEQ ID | NO: 48) | NO: 59) | NO: 68) |
| | | | | NO: 34) | | | |
| 25 | AIP- | GFTFSKAWMT | RIKSVTDGETTDYA | TSSFCCRGGSCPSH | SGSSSNIGSSSV | KNNQRPS | STWDDSLSVRV |
| | 158623 | (SEQ ID | APVKG (SEQ ID | DTSFCGGQYKSYYY | S (SEQ ID | (SEQ ID | (SEQ ID |
| | | NO: 8) | NO: 9) | MDV (SEQ ID | NO: 48) | NO: 59) | NO: 68) |
| | | | | NO: 37) | | | |
| 26 | AIP- | GFTFSKAWMT | RIKSVTDGETTDYA | TTSFCCRGGSCPSH | SGSSSNIGSSSV | KNNQRPS | STWDDSLSVRV |
| | 155066 | (SEQ ID | APVKG (SEQ ID | DTSFCGGQYKSYYY | S (SEQ ID | (SEQ ID | (SEQ ID |
| | | NO: 8) | NO: 9) | MDV (SEQ ID | NO: 48) | NO: 59) | NO: 68) |
| | | | | NO: 39) | | | |
| 27 | AIP- | GFTFSKAWMT | RIKSVTDGETTDYA | TSSFCCRGGSCPSH | SGSSSNIGSSSV | KNNQRPS | STWDDSLSVRV |
| | 136538 | (SEQ ID | APVKG (SEQ ID | DTSFCGGQDSRYYY | S (SEQ ID | (SEQ ID | (SEQ ID |
| | | NO: 8) | NO: 9) | MDV (SEQ ID | NO: 48) | NO: 59) | NO: 68) |
| | | | | NO: 40) | | | |
| 28 | AIP- | GFTFSKAWMT | RIKSVTDGETTDYA | TSSFCCRGGSCPSH | SGSSSNIGSSSV | KNNQRPS | STWDDSLSVRV |
| | 166120 | (SEQ ID | APVKG (SEQ ID | DTSYCGGQYKSYYY | S (SEQ ID | (SEQ ID | (SEQ ID |
| | | NO: 8) | NO: 9) | MDV (SEQ ID | NO: 48) | NO: 59) | NO: 68) |
| | | | | NO: 21) | | | |
| 29 | AIP- | GFTFSKAWMT | RIKSVTDGETTDYA | TSSFCCRGGSCPSH | SGSSSNIGSSSV | KNNQRPS | STWDESLSVRV |
| | 133645 | (SEQ ID | APVKG (SEQ ID | DTSFCGGQYKSYYY | S (SEQ ID | (SEQ ID | (SEQ ID |
| | | NO: 8) | NO: 9) | MDV (SEQ ID | NO: 48) | NO: 59) | NO: 73) |
| | | | | NO: 37) | | | |
| 30 | AIP- | GFTFSKAWMS | RIKSVTDGETTDYA | TSSFCCRGGSCPSH | SGSSSNIGSSSV | KNNQRPS | STWDDSLSVRV |
| | 187893 | (SEQ ID | APVKG (SEQ ID | DTSFCGGQYKSYYY | S (SEQ ID | (SEQ ID | (SEQ ID |
| | | NO: 1) | NO: 9) | MDV (SEQ ID | NO: 48) | NO: 59) | NO: 68) |
| | | | | NO: 37) | | | |
| 31 | AIP- | GFTFSKAWMT | RIKSVTDGETTDYA | TSSFCCRGGSCPSH | SGSSSNIGSSSV | KNNQRPS | STWDDSLSVRV |
| | 142079 | (SEQ ID | APVKG (SEQ ID | DTSFCGGSYKSYYY | S (SEQ ID | (SEQ ID | (SEQ ID |
| | | NO: 8) | NO: 9) | MDV (SEQ ID | NO: 48) | NO: 59) | NO: 68) |
| | | | | NO: 41) | | | |
| 32 | AIP- | GFTFSKAWMT | RIKSVTDGETTDYA | TSSFCCRGGSCPSH | SGSSSNIGSSSV | KNNQRPS | STWDESLSVRV |
| | 104188 | (SEQ ID | APVKG (SEQ ID | DTSYCGGQDSRYYY | S (SEQ ID | (SEQ ID | (SEQ ID |
| | | NO: 8) | NO: 9) | MDV (SEQ ID | NO: 48) | NO: 59) | NO: 73) |
| | | | | NO: 42) | | | |
| 33 | AIP- | GFTFSKAWMT | RIKSVTEGETTDYA | TTSFCCRGGSCPSH | SGSSSNIGSSSV | KNNQRPS | STWDESLSVRV |
| | 106042 | (SEQ ID | APVKG (SEQ ID | DTSFCGGQYKSYYY | S (SEQ ID | (SEQ ID | (SEQ ID |
| | | NO: 8) | NO: 17) | MDV (SEQ ID | NO: 48) | NO: 59) | NO: 73) |
| | | | | NO: 39) | | | |
| 34 | AIP- | GFTFSKAWMT | RIKSVTEGETTDYA | TSSFCCRGGSCPSH | SGSSSNIGSSSV | KNNQRPS | STWDESLSVRV |
| | 100196 | (SEQ ID | APVKG (SEQ ID | DTSFCGGQYKSYYY | S (SEQ ID | (SEQ ID | (SEQ ID |
| | | NO: 8) | NO: 17) | MDV (SEQ ID | NO: 48) | NO: 59) | NO: 73) |
| | | | | NO: 37) | | | |
| 35 | AIP- | GFTFSKAWMS | RIKSVTDGETTDYA | TSSFCCRGGSCPSH | SGSSSNIGSSSV | KNNQRPS | STWDDSLSVRV |
| | 180675 | (SEQ ID | APVKG (SEQ ID | DTSYCGGQYKSYYF | S (SEQ ID | (SEQ ID | (SEQ ID |
| | | NO: 1) | NO: 9) | MDV (SEQ ID | NO: 48) | NO: 59) | NO: 68) |
| | | | | NO: 43) | | | |
| 36 | AIP- | GFTFSKAWMS | RIKSVTDGETTDYA | TSSFCCRGGSCPSH | SGSSSNIGSSSV | KNNQRPS | STWDDSLSVRV |
| | 170105 | (SEQ ID | APVKG (SEQ ID | DTSYCGGQYKSYYY | Y (SEQ ID | (SEQ ID | (SEQ ID |
| | | NO: 1) | NO: 9) | MDV (SEQ ID | NO: 56) | NO: 59) | NO: 68) |
| | | | | NO: 21) | | | |
| 37 | AIP- | GFTFSKAWMS | RIKSVTDGETTDYA | TSSFCCRGGSCPSH | SGSSSNIGSSSV | KNNQRPS | STWDDSLSVRV |
| | 126080 | (SEQ ID | APVKG (SEQ ID | DTSYCGGQNKQYYY | S (SEQ ID | (SEQ ID | (SEQ ID |
| | | NO: 1) | NO: 9) | MDV (SEQ ID | NO: 48) | NO: 59) | NO: 68) |
| | | | | NO: 44) | | | |
| 38 | AIP- | GFTFSKAWMS | RIKSVTDGETTDYA | TSSFCCRGGNCPSH | SGSSSNIGSSSV | KNNQRPS | STWDDSLSVRV |
| | 161571 | (SEQ ID | APVKG (SEQ ID | ETSYCGNQYKSYYY | S (SEQ ID | (SEQ ID | (SEQ ID |
| | | NO: 1) | NO: 9) | MDV (SEQ ID | NO: 48) | NO: 59) | NO: 68) |
| | | | | NO: 45) | | | |
| 39 | AIP- | GFTFSKAWMS | RIKSVTDGETTDYA | TSSFCCRGGSCPSH | SGSSSNIGSSSV | KNNQRPS | STWDDSLSVRV |
| | 181246 | (SEQ ID | APVKG (SEQ ID | DTSYCGGQYKSYYY | S (SEQ ID | (SEQ ID | (SEQ ID |
| | | NO: 1) | NO: 9) | VNI (SEQ ID | NO: 48) | NO: 59) | NO: 68) |
| | | | | NO: 46) | | | |
| 40 | AIP- | GFTFSKAWMS | RIKSVTDGETTDYA | TTSFCCRGASCPSH | SGSSSNIGSSSV | KNNQRPS | STWDDSLSVRV |
| | 192216 | (SEQ ID | APVKG (SEQ ID | DTSYCAGSYKSYYY | S (SEQ ID | (SEQ ID | (SEQ ID |
| | | NO: 1) | NO: 9) | MDV (SEQ ID | NO: 48) | NO: 59) | NO: 68) |
| | | | | NO: 47) | | | |
| 41 | AIP- | GFTFSKAWMS | RIKSVTDGETTDYA | TSSFCCRGGSCPSH | SGSSSDIGSSSV | KNNQRPS | STWNDALSVRV |
| | 168605 | (SEQ ID | APVKG (SEQ ID | DTSYCGGQYKSYYY | S (SEQ ID | (SEQ ID | (SEQ ID |
| | | NO: 1) | NO: 9) | MDV (SEQ ID | NO: 57) | NO: 59) | NO: 74) |
| | | | | NO: 21) | | | |
| 42 | AIP- | GFTFSKAWMS | RIKSVTDGEQTDYA | TSSFCCRGGSCPSH | SGSSSNIGSSSV | KNTQRPS | STWDDSLNVRV |
| | 172872 | (SEQ ID | APVKG | DTSYCGGQYKSYYY | S (SEQ ID | (SEQ ID | (SEQ ID |
| | | NO: 1) | (SEQ ID | MDV (SEQ ID | NO: 48) | NO: 65) | NO: 75) |
| | | | NO: 18) | NO: 21) | | | |
| 43 | AIP- | GFTFSKAWMT | RIKSVTDGETTDYA | TSSFCCRGGSCPSH | SGSPSNIGSSSV | KNNQRPS | STWDDALSVRV |
| | 190051 | (SEQ ID | APVKG (SEQ ID | DTSYCGGQYKSYYY | S (SEQ ID | (SEQ ID | (SEQ ID |
| | | NO: 8) | NO: 9) | MDV (SEQ ID | NO: 58) | NO: 59) | NO: 72) |
| | | | | NO: 21) | | | |
| 44 | AIP- | GFTFSKAWMS | RIKAADDGKQTDYA | TSSFCCRGGSCPSH | SGSSSNIGSSSV | KNNQRPS | STWDDSLSVRV |
| | 167533 | (SEQ ID | APVKG | DTSYCGGQYKSYYY | S (SEQ ID | (SEQ ID | (SEQ ID |
| | | NO: 1) | (SEQ ID NO: 19) | MDV (SEQ ID | NO: 48) | NO: 59) | NO: 68) |
| | | | | NO: 21) | | | |
| 45 | AIP- | GFTFSKAWMS | RIKSVTDGETTDYA | ISSFCCRGNSCPSS | SGSSSNIGSSSV | KNNQRPS | STWDDSLSVRV |
| | 112580 | (SEQ ID | APVKG (SEQ ID | DTSYCNGQYKSYYY | S (SEQ ID | (SEQ ID | (SEQ ID |
| | | NO: 1) | NO: 9) | MDV (SEQ ID | NO: 48) | NO: 59) | NO: 68) |
| | | | | NO: 27) | | | |
| 46 | AIP- | GFTFSKAWMS | RIKSVTDGETTDYA | TSSFCCRGGSCPSH | SGSSSNIGSSSV | KNSLRPQ | STWDDSLSVRV |
| | 136060 | (SEQ ID | APVKG (SEQ ID | DTSYCGGQYKSYYY | S (SEQ ID | (SEQ ID | (SEQ ID |
| | | NO: 1) | NO: 9) | MDV (SEQ ID | NO: 48) | NO: 67) | NO: 68) |
| | | | | NO: 21) | | | |
| 47 | AIP- | GFTFSKAWMS | RIKSVTDGETTDYA | TSSFCCRGGSCPSH | SGSSSNIGSSSV | KNNQRPS | STWDDSLSVRV |
| | 125646 | (SEQ ID | APVKG (SEQ ID | DTSYCGQYKSYYYM | S (SEQ ID | (SEQ ID | (SEQ ID |
| | | NO: 1) | NO: 9) | DV (SEQ ID | NO: 48) | NO: 59) | NO: 68) |
| | | | | NO: 1744) | | | |

TABLE 2A

| | AIP Number | V_{H} Sequence | V_{L} Sequence |
|---|---|---|---|
| 1 | AIP-148327 | EVQLVESGGALVKPGGSLRLSCAASGFTFSKAWMT | QSVLTQAPSASETPGQRVIISCSGSSSNIGSSSVSWYQQ |
| | | WVRQAPGKGLEWVGRIKSVTEGETTDYAAPVKGRF | LPGTAPKLLIYKNNQRPSGVPDRFSGSKSGTSASLAISG |
| | | TISRDDSKSVLYLQMSSLKTEDTAVYFCTSSFCCR | LRSEDEADYYCSTWDESLSVRVFGGGTKLTVL (SEQ |
| | | GGSCPSHDTSFCGGQDKRYYYMDVWGKGTTVTVSS | ID NO: 1527) |
| | | (SEQ ID NO: 1333) | |
| 2 | AIP-198092 | EVQLVESGGALVKPGGSLRLSCAASGFTFSKAWMT | QSVLTQPPSASGTPGQRVTISCSGSSSNIGSSSVSWYQQ |
| | | WVRQAPGKGLEWVGRIKSVTEGETTDYAAPVKGRF | LPGTAPKLLIYKNNQRPSGVPDRFSGSKSGTSASLAISG |
| | | TISRDDSKSTLYLQMNSLKTEDTAVYYCTSSFCCR | LRSEDEADYYCSTWDESLSVRVFGGGTKLTVL (SEQ |
| | | GGSCPSHDTSFCGGQDKRYYYMDVWGKGTTVTVSS | ID NO: 1528) |
| | | (SEQ ID NO: 1334) | |
| 3 | AIP-184490 | EVQLVESGGALVKPGGSLRLSCAASGFTFSKAWMT | QSVLTQPPSASGTPGQRVTISCSGSSSNIGSSSVSWYQQ |
| | | WVRQAPGKGLEWVGRIKSVTDGETTDYAAPVKGRF | LPGTAPKLLIYKNNQRPSGVPDRFSGSKSGTSASLAISG |
| | | TISRDDSKSTLYLQMNSLKTEDTAVYYCTSSFCCR | LRSEDEADYYCSTWDESLSVRVFGGGTKLTVL (SEQ |
| | | GGSCPSHDTSFCGGSYKSYYYMDVWGKGTTVTVSS | ID NO: 1529) |
| | | (SEQ ID NO: 1335) | |
| 4 | AIP-102396 | EVQLVESGGALVKPGGSLRLSCAASGFTFSKAWMS | QSVLTQPPSASGTPGQRVTISCSGSSSNIGSSSVSWYQQ |
| | | WVRQAPGKGLEWVGRIKSVTDGETTDYAAPVKGRF | LPGTAPKLLIYKNNQRPSGVPDRFSGSKSGTSASLAISG |
| | | TISRDDSKSTLYLQMNSLKTEDTAVYYCTSSFCCR | LRSEDEADYYCSTWDESLSVRVFGGGTKLTVL (SEQ |
| | | GGSCPSHDTSYCGGQYKSYYYMDVWGKGTTVTVSS | ID NO: 1530) |
| | | (SEQ ID NO: 1336) | |
| 5 | AIP-150055 | EVQLVESGGALVKPGGSLRLSCAASGYTFSKAWMS | QSVLTQPPSASGTPGQRVTISCSGSSSNIGSSSVSWYQQ |
| | | WVRQAPGKGLEWVGRIKSVTDGETTDYAAPVKGRF | LPGTAPKLLIYKNNQRPSGVPDRFSGSKSGTSASLAISG |
| | | TISRDDSKSTLYLQMNSLKTEDTAVYYCTSSFCCR | LRSEDEADYYCSTWDDSLSVRVFGGGTKLTVL (SEQ |
| | | GGSCPSHDTSYCGGQYKSYYYMDVWGKGTTVTVS | ID NO: 1531) |
| | | (SEQ ID NO: 1337) | |
| 6 | AIP-167084 | EVQLVESGGALVKPGGSLRLSCAASGFDFSKAWMS | QSVLTQPPSASGTPGQRVTISCSGSSSNIGSSSVSWYQQ |
| | | WVRQAPGKGLEWVGRIKSVTDGETTDYAAPVKGRF | LPGTAPKLLIYKNNQRPSGVPDRFSGSKSGTSASLAISG |
| | | TISRDDSKSTLYLQMNSLKTEDTAVYYCTSSFCCR | LRSEDEADYYCSTWDDSLSVRVFGGGTKLTVL (SEQ |
| | | GGSCPSHDTSYCGGQYKSYYYMDVWGKGTTVTVSS | ID NO: 1532) |
| | | (SEQ ID NO: 1338) | |
| 7 | AIP-185304 | EVQLVESGGALVKPGGSLRLSCAASGFVFSKAWMS | QSVLTQPPSASGTPGQRVTISCSGSSSNIGSSSVSWYQQ |
| | | WVRQAPGKGLEWVGRIKSVTDGETTDYAAPVKGRF | LPGTAPKLLIYKNNQRPSGVPDRFSGSKSGTSASLAISG |
| | | TISRDDSKSTLYLQMNSLKTEDTAVYYCTSSFCCR | LRSEDEADYYCSTWDDSLSVRVFGGGTKLTVL (SEQ |
| | | GGSCPSHDTSYCGGQYKSYYYMDVWGKGTTVTVSS | ID NO: 1533) |
| | | (SEQ ID NO: 1339) | |
| 8 | AIP-134770 | EVQLVESGGALVKPGGSLRLSCAASGFTYSKAWMS | QSVLTQPPSASGTPGQRVTISCSGSSSNIGSSSVSWYQQ |
| | | WVRQAPGKGLEWVGRIKSVTDGETTDYAAPVKGRF | LPGTAPKLLIYKNNQRPSGVPDRFSGSKSGTSASLAISG |
| | | TISRDDSKSTLYLQMNSLKTEDTAVYYCTSSFCCR | LRSEDEADYYCSTWDDSLSVRVFGGGTKLTVL (SEQ |
| | | GGSCPSHDTSYCGGQYKSYYYMDVWGKGTTVTVSS | ID NO: 1534) |
| | | SEQ ID NO: 1340) | |
| 9 | AIP-141887 | EVQLVESGGALVKPGGSLRLSCAASGFTFAKAWMS | QSVLTQPPSASGTPGQRVTISCSGSSSNIGSSSVSWYQQ |
| | | WVRQAPGKGLEWVGRIKSVTDGETTDYAAPVKGRF | LPGTAPKLLIYKNNQRPSGVPDRFSGSKSGTSASLAISG |
| | | TISRDDSKSTLYLQMNSLKTEDTAVYYCTSSFCCR | LRSEDEADYYCSTWDDSLSVRVFGGGTKLTVL (SEQ |
| | | GGSCPSHDTSYCGGQYKSYYYMDVWGKGTTVTVSS | ID NO: 1535) |
| | | (SEQ ID NO: 1341) | |
| 10 | AIP-196203 | EVQLVESGGALVKPGGSLRLSCAASGFTFSNAWMS | QSVLTQPPSASGTPGQRVTISCSGSSSNIGSSSVSWYQQ |
| | | WVRQAPGKGLEWVGRIKSVTDGETTDYAAPVKGRF | LPGTAPKLLIYKNNQRPSGVPDRFSGSKSGTSASLAISG |
| | | TISRDDSKSTLYLQMNSLKTEDTAVYYCTSSFCCR | LRSEDEADYYCSTWDDSLSVRVFGGGTKLTVL (SEQ |
| | | GGSCPSHDTSYCGGQYKSYYYMDVWGKGTTVTVSS | ID NO: 1536) |
| | | (SEQ ID NO: 1342) | |
| 11 | AIP-184151 | EVQLVESGGALVKPGGSLRLSCAASGFTFSRAWMS | QSVLTQPPSASGTPGQRVTISCSGSSSNIGSSSVSWYQQ |
| | | WVRQAPGKGLEWVGRIKSVTDGETTDYAAPVKGRF | LPGTAPKLLIYKNNQRPSGVPDRFSGSKSGTSASLAISG |
| | | TISRDDSKSTLYLQMNSLKTEDTAVYYCTSSFCCR | LRSEDEADYYCSTWDDSLSVRVFGGGTKLTVL (SEQ |
| | | GGSCPSHDTSYCGGQYKSYYYMDVWGKGTTVTVSS | ID NO: 1537) |
| | | (SEQ ID NO: 1343) | |
| 12 | AIP-128195 | EVQLVESGGALVKPGGSLRLSCAASGFTFSMAWMS | QSVLTQPPSASGTPGQRVTISCSGSSSNIGSSSVSWYQQ |
| | | WVRQAPGKGLEWVGRIKSVTDGETTDYAAPVKGRF | LPGTAPKLLIYKNNQRPSGVPDRFSGSKSGTSASLAISG |
| | | TISRDDSKSTLYLQMNSLKTEDTAVYYCTSSFCCR | LRSEDEADYYCSTWDDSLSVRVFGGGTKLTVL (SEQ |
| | | GGSCPSHDTSYCGGQYKSYYYMDVWGKGTTVTVSS | ID NO: 1538) |
| | | (SEQ ID NO: 1344) | |
| 13 | AIP-116579 | EVQLVESGGALVKPGGSLRLSCAASGFTFSAAWMS | QSVLTQPPSASGTPGQRVTISCSGSSSNIGSSSVSWYQQ |
| | | WVRQAPGKGLEWVGRIKSVTDGETTDYAAPVKGRF | LPGTAPKLLIYKNNQRPSGVPDRFSGSKSGTSASLAISG |
| | | TISRDDSKSTLYLQMNSLKTEDTAVYYCTSSFCCR | LRSEDEADYYCSTWDDSLSVRVFGGGTKLTVL (SEQ |
| | | GGSCPSHDTSYCGGQYKSYYYMDVWGKGTTVTVSS | ID NO: 1539) |
| | | (SEQ ID NO: 1345) | |
| 14 | AIP-192329 | EVQLVESGGALVKPGGSLRLSCAASGFTFSHAWMS | QSVLTQPPSASGTPGQRVTISCSGSSSNIGSSSVSWYQQ |
| | | WVRQAPGKGLEWVGRIKSVTDGETTDYAAPVKGRF | LPGTAPKLLIYKNNQRPSGVPDRFSGSKSGTSASLAISG |
| | | TISRDDSKSTLYLQMNSLKTEDTAVYYCTSSFCCR | LRSEDEADYYCSTWDDSLSVRVFGGGTKLTVL (SEQ |
| | | GGSCPSHDTSYCGGQYKSYYYMDVWGKGTTVTVSS | ID NO: 1540) |
| | | (SEQ ID NO: 1346) | |
| 15 | AIP-192329 | EVQLVESGGALVKPGGSLRLSCAASGFTFSHAWMS | QSVLTQPPSASGTPGQRVTISCSGSSSNIGSSSVSWYQQ |
| | | WVRQAPGKGLEWVGRIKSVTDGETTDYAAPVKGRF | LPGTAPKLLIYKNNQRPSGVPDRFSGSKSGTSASLAISG |
| | | TISRDDSKSTLYLQMNSLKTEDTAVYYCTSSFCCR | LRSEDEADYYCSTWDDSLSVRVFGGGTKLTVL (SEQ |
| | | GGSCPSHDTSYCGGQYKSYYYMDVWGKGTTVTVSS | ID NO: 1541) |
| | | (SEQ ID NO: 1347) | |
| 16 | AIP-197809 | EVQLVESGGALVKPGGSLRLSCAASGFTFSKAYMS | QSVLTQPPSASGTPGQRVTISCSGSSSNIGSSSVSWYQQ |
| | | WVRQAPGKGLEWVGRIKSVTDGETTDYAAPVKGRF | LPGTAPKLLIYKNNQRPSGVPDRFSGSKSGTSASLAISG |
| | | TISRDDSKSTLYLQMNSLKTEDTAVYYCTSSFCCR | LRSEDEADYYCSTWDDSLSVRVFGGGTKLTVL (SEQ |
| | | GGSCPSHDTSYCGGQYKSYYYMDVWGKGTTVTVSS | ID NO: 1542) |
| | | (SEQ ID NO: 1348) | |
| 17 | AIP-142489 | EVQLVESGGALVKPGGSLRLSCAASGFTFSKAWFS | QSVLTQPPSASGTPGQRVTISCSGSSSNIGSSSVSWYQQ |
| | | WVRQAPGKGLEWVGRIKSVTDGETTDYAAPVKGRF | LPGTAPKLLIYKNNQRPSGVPDRFSGSKSGTSASLAISG |
| | | TISRDDSKSTLYLQMNSLKTEDTAVYYCTSSFCCR | LRSEDEADYYCSTWDDSLSVRVFGGGTKLTVL (SEQ |
| | | GGSCPSHDTSYCGGQYKSYYYMDVWGKGTTVTVSS | ID NO: 1543) |
| | | (SEQ ID NO: 1349) | |
| 18 | AIP-148102 | EVQLVESGGALVKPGGSLRLSCAASGFTFSKAWMS | QSVLTQPPSASGTPGQRVTISCSGSSSNIGSSSVSWYQQ |
| | | WVRQAPGKGLEWVGRIQSVTDGETTDYAAPVKGRF | LPGTAPKLLIYKNNQRPSGVPDRFSGSKSGTSASLAISG |
| | | TISRDDSKSTLYLQMNSLKTEDTAVYYCTSSFCCR | LRSEDEADYYCSTWDDSLSVRVFGGGTKLTVL (SEQ |
| | | GGSCPSHDTSYCGGQYKSYYYMDVWGKGTTVTVSS | ID NO: 1544) |
| | | (SEQ ID NO: 1350) | |
| 19 | AIP-167726 | EVQLVESGGALVKPGGSLRLSCAASGFTFSKAWMS | QSVLTQPPSASGTPGQRVTISCSGSSSNIGSSSVSWYQQ |
| | | WVRQAPGKGLEWVGRIKAVTDGETTDYAAPVKGRF | LPGTAPKLLIYKNNQRPSGVPDRFSGSKSGTSASLAISG |
| | | TISRDDSKSTLYLQMNSLKTEDTAVYYCTSSFCCR | LRSEDEADYYCSTWDDSLSVRVFGGGTKLTVL (SEQ |
| | | GGSCPSHDTSYCGGQYKSYYYMDVWGKGTTVTVSS | ID NO: 1545) |
| | | (SEQ ID NO: 1351) | |
| 20 | AIP-199834 | EVQLVESGGALVKPGGSLRLSCAASGFTFSKAWMS | QSVLTQPPSASGTPGQRVTISCSGSSSNIGSSSVSWYQQ |
| | | WVRQAPGKGLEWVGRIKSATDGETTDYAAPVKGRF | LPGTAPKLLIYKNNQRPSGVPDRFSGSKSGTSASLAISG |
| | | TISRDDSKSTLYLQMNSLKTEDTAVYYCTSSFCCR | LRSEDEADYYCSTWDDSLSVRVFGGGTKLTVL (SEQ |
| | | GGSCPSHDTSYCGGQYKSYYYMDVWGKGTTVTVSS | ID NO: 1546) |
| | | (SEQ ID NO: 1352) | |
| 21 | AIP-143179 | EVQLVESGGALVKPGGSLRLSCAASGFTFSKAWMS | QSVLTQPPSASGTPGQRVTISCSGSSSNIGSSSVSWYQQ |
| | | WVRQAPGKGLEWVGRIKSNTDGETTDYAAPVKGRF | LPGTAPKLLIYKNNQRPSGVPDRFSGSKSGTSASLAISG |
| | | TISRDDSKSTLYLQMNSLKTEDTAVYYCTSSFCCR | LRSEDEADYYCSTWDDSLSVRVFGGGTKLTVL (SEQ |
| | | GGSCPSHDTSYCGGQYKSYYYMDVWGKGTTVTVSS | ID NO: 1547) |
| | | (SEQ ID NO: 1353) | |
| 22 | AIP-195587 | EVQLVESGGALVKPGGSLRLSCAASGFTFSKAWMS | QSVLTQPPSASGTPGQRVTISCSGSSSNIGSSSVSWYQQ |
| | | WVRQAPGKGLEWVGRIKSVHDGETTDYAAPVKGRF | LPGTAPKLLIYKNNQRPSGVPDRFSGSKSGTSASLAISG |
| | | TISRDDSKSTLYLQMNSLKTEDTAVYYCTSSFCCR | LRSEDEADYYCSTWDDSLSVRVFGGGTKLTVL (SEQ |
| | | GGSCPSHDTSYCGGQYKSYYYMDVWGKGTTVTVSS | ID NO: 1548) |
| | | (SEQ ID NO: 1354) | |
| 23 | AIP-153462 | EVQLVESGGALVKPGGSLRLSCAASGFTFSKAWMS | QSVLTQPPSASGTPGQRVTISCSGSSSNIGSSSVSWYQQ |
| | | WVRQAPGKGLEWVGRIKSVDDGETTDYAAPVKGRF | LPGTAPKLLIYKNNQRPSGVPDRFSGSKSGTSASLAISG |
| | | TISRDDSKSTLYLQMNSLKTEDTAVYYCTSSFCCR | LRSEDEADYYCSTWDDSLSVRVFGGGTKLTVL (SEQ |
| | | GGSCPSHDTSYCGGQYKSYYYMDVWGKGTTVTVSS | ID NO: 1549) |
| | | (SEQ ID NO: 1355) | |
| 24 | AIP-115363 | EVQLVESGGALVKPGGSLRLSCAASGFTFSKAWMS | QSVLTQPPSASGTPGQRVTISCSGSSSNIGSSSVSWYQQ |
| | | WVRQAPGKGLEWVGRIKSVQDGETTDYAAPVKGRF | LPGTAPKLLIYKNNQRPSGVPDRFSGSKSGTSASLAISG |
| | | TISRDDSKSTLYLQMNSLKTEDTAVYYCTSSFCCR | LRSEDEADYYCSTWDDSLSVRVFGGGTKLTVL (SEQ |
| | | GGSCPSHDTSYCGGQYKSYYYMDVWGKGTTVTVSS | ID NO: 1550) |
| | | (SEQ ID NO: 1356) | |
| 25 | AIP-151090 | EVQLVESGGALVKPGGSLRLSCAASGFTFSKAWMS | QSVLTQPPSASGTPGQRVTISCSGSSSNIGSSSVSWYQQ |
| | | WVRQAPGKGLEWVGRIKSVINGETTDYAAPVKGRF | LPGTAPKLLIYKNNQRPSGVPDRFSGSKSGTSASLAISG |
| | | TISRDDSKSTLYLQMNSLKTEDTAVYYCTSSFCCR | LRSEDEADYYCSTWDDSLSVRVFGGGTKLTVL (SEQ |
| | | GGSCPSHDTSYCGGQYKSYYYMDVWGKGTTVTVSS | ID NO: 1551) |
| | | (SEQ ID NO: 1357) | |
| 26 | AIP-168083 | EVQLVESGGALVKPGGSLRLSCAASGFTFSKAWMS | QSVLTQPPSASGTPGQRVTISCSGSSSNIGSSSVSWYQQ |
| | | WVRQAPGKGLEWVGRIKSVTDAETTDYAAPVKGRF | LPGTAPKLLIYKNNQRPSGVPDRFSGSKSGTSASLAISG |
| | | TISRDDSKSTLYLQMNSLKTEDTAVYYCTSSFCCR | LRSEDEADYYCSTWDDSLSVRVFGGGTKLTVL (SEQ |
| | | GGSCPSHDTSYCGGQYKSYYYMDVWGKGTTVTVSS | ID NO: 1552) |
| | | (SEQ ID NO: 1358) | |
| 27 | AIP-161082 | EVQLVESGGALVKPGGSLRLSCAASGFTFSKAWMS | QSVLTQPPSASGTPGQRVTISCSGSSSNIGSSSVSWYQQ |
| | | WVRQAPGKGLEWVGRIKSVTDGQTTDYAAPVKGRF | LPGTAPKLLIYKNNQRPSGVPDRFSGSKSGTSASLAISG |
| | | TISRDDSKSTLYLQMNSLKTEDTAVYYCTSSFCCR | LRSEDEADYYCSTWDDSLSVRVFGGGTKLTVL (SEQ |
| | | GGSCPSHDTSYCGGQYKSYYYMDVWGKGTTVTVSS | ID NO: 1553) |
| | | (SEQ ID NO: 1359) | |
| 28 | AIP-114196 | EVQLVESGGALVKPGGSLRLSCAASGFTFSKAWMS | QSVLTQPPSASGTPGQRVTISCSGSSSNIGSSSVSWYQQ |
| | | WVRQAPGKGLEWVGRIKSVTDGHTTDYAAPVKGRF | LPGTAPKLLIYKNNQRPSGVPDRFSGSKSGTSASLAISG |
| | | TISRDDSKSTLYLQMNSLKTEDTAVYYCTSSFCCR | LRSEDEADYYCSTWDDSLSVRVFGGGTKLTVL (SEQ |
| | | GGSCPSHDTSYCGGQYKSYYYMDVWGKGTTVTVSS | ID NO: 1554) |
| | | (SEQ ID NO: 1360) | |
| 29 | AIP-189338 | EVQLVESGGALVKPGGSLRLSCAASGFTFSKAWMS | QSVLTQPPSASGTPGQRVTISCSGSSSNIGSSSVSWYQQ |
| | | WVRQAPGKGLEWVGRIKSVTDGEQTDYAAPVKGRF | LPGTAPKLLIYKNNQRPSGVPDRFSGSKSGTSASLAISG |
| | | TISRDDSKSTLYLQMNSLKTEDTAVYYCTSSFCCR | LRSEDEADYYCSTWDDSLSVRVFGGGTKLTVL (SEQ |
| | | GGSCPSHDTSYCGGQYKSYYYMDVWGKGTTVTVSS | ID NO: 1555) |
| | | (SEQ ID NO: 1361) | |
| 30 | AIP-183190 | EVQLVESGGALVKPGGSLRLSCAASGFTFSKAWMS | QSVLTQPPSASGTPGQRVTISCSGSSSNIGSSSVSWYQQ |
| | | WVRQAPGKGLEWVGRIKSVTDGEATDYAAPVKGRF | LPGTAPKLLIYKNNQRPSGVPDRFSGSKSGTSASLAISG |
| | | TISRDDSKSTLYLQMNSLKTEDTAVYYCTSSFCCR | LRSEDEADYYCSTWDDSLSVRVFGGGTKLTVL (SEQ |
| | | GGSCPSHDTSYCGGQYKSYYYMDVWGKGTTVTVSS | ID NO: 1556) |
| | | (SEQ ID NO: 1362) | |
| 31 | AIP-110143 | EVQLVESGGALVKPGGSLRLSCAASGFTFSKAWMS | QSVLTQPPSASGTPGQRVTISCSGSSSNIGSSSVSWYQQ |
| | | WVRQAPGKGLEWVGRIKSVTDGETTDYASPVKGRF | LPGTAPKLLIYKNNQRPSGVPDRFSGSKSGTSASLAISG |
| | | TISRDDSKSTLYLQMNSLKTEDTAVYYCTSSFCCR | LRSEDEADYYCSTWDDSLSVRVFGGGTKLTVL (SEQ |
| | | GGSCPSHDTSYCGGQYKSYYYMDVWGKGTTVTVSS | ID NO: 1557) |
| | | (SEQ ID NO: 1363) | |
| 32 | AIP-147176 | EVQLVESGGALVKPGGSLRLSCAASGFTFSKAWMS | QSVLTQPPSASGTPGQRVTISCSGSSSNIGSSSVSWYQQ |
| | | WVRQAPGKGLEWVGRIKSVTDGETTDYAAPVQGRF | LPGTAPKLLIYKNNQRPSGVPDRFSGSKSGTSASLAISG |
| | | TISRDDSKSTLYLQMNSLKTEDTAVYYCTSSFCCR | LRSEDEADYYCSTWDDSLSVRVFGGGTKLTVL (SEQ |
| | | GGSCPSHDTSYCGGQYKSYYYMDVWGKGTTVTVSS | ID NO: 1558) |
| | | (SEQ ID NO: 1364) | |
| 33 | AIP-134312 | EVQLVESGGALVKPGGSLRLSCAASGFTFSKAWMS | QSVLTQPPSASGTPGQRVTISCSGSSSNIGSSSVSWYQQ |
| | | WVRQAPGKGLEWVGRIKSVTDGETTDYAAPVKGRF | LPGTAPKLLIYKNNQRPSGVPDRFSGSKSGTSASLAISG |
| | | TISRDDSKSTLYLQMNSLKTEDTAVYYCISSFCCR | LRSEDEADYYCSTWDDSLSVRVFGGGTKLTVL (SEQ |
| | | GGSCPSHDTSYCGGQYKSYYYMDVWGKGTTVTVSS | ID NO: 1559) |
| | | (SEQ ID NO: 1365) | |
| 34 | AIP-128243 | EVQLVESGGALVKPGGSLRLSCAASGFTFSKAWMS | QSVLTQPPSASGTPGQRVTISCSGSSSNIGSSSVSWYQQ |
| | | WVRQAPGKGLEWVGRIKSVTDGETTDYAAPVKGRF | LPGTAPKLLIYKNNQRPSGVPDRFSGSKSGTSASLAISG |
| | | TISRDDSKSTLYLQMNSLKTEDTAVYYCTTSFCCR | LRSEDEADYYCSTWDDSLSVRVFGGGTKLTVL (SEQ |
| | | GGSCPSHDTSYCGGQYKSYYYMDVWGKGTTVTVSS | ID NO: 1560) |
| | | (SEQ ID NO: 1366) | |
| 35 | AIP-156172 | EVQLVESGGALVKPGGSLRLSCAASGFTFSKAWMS | QSVLTQPPSASGTPGQRVTISCSGSSSNIGSSSVSWYQQ |
| | | WVRQAPGKGLEWVGRIKSVTDGETTDYAAPVKGRF | LPGTAPKLLIYKNNQRPSGVPDRFSGSKSGTSASLAISG |
| | | TISRDDSKSTLYLQMNSLKTEDTAVYYCTSPFCCR | LRSEDEADYYCSTWDDSLSVRVFGGGTKLTVL (SEQ |
| | | GGSCPSHDTSYCGGQYKSYYYMDVWGKGTTVTVSS | ID NO: 1561) |
| | | (SEQ ID NO: 1367) | |
| 36 | AIP-147389 | EVQLVESGGALVKPGGSLRLSCAASGFTFSKAWMS | QSVLTQPPSASGTPGQRVTISCSGSSSNIGSSSVSWYQQ |
| | | WVRQAPGKGLEWVGRIKSVTDGETTDYAAPVKGRF | LPGTAPKLLIYKNNQRPSGVPDRFSGSKSGTSASLAISG |
| | | TISRDDSKSTLYLQMNSLKTEDTAVYYCTSAFCCR | LRSEDEADYYCSTWDDSLSVRVFGGGTKLTVL (SEQ |
| | | GGSCPSHDTSYCGGQYKSYYYMDVWGKGTTVTVSS | ID NO: 1562) |
| | | (SEQ ID NO: 1368) | |
| 37 | AIP-124314 | EVQLVESGGALVKPGGSLRLSCAASGFTFSKAWMS | QSVLTQPPSASGTPGQRVTISCSGSSSNIGSSSVSWYQQ |
| | | WVRQAPGKGLEWVGRIKSVTDGETTDYAAPVKGRF | LPGTAPKLLIYKNNQRPSGVPDRFSGSKSGTSASLAISG |
| | | TISRDDSKSTLYLQMNSLKTEDTAVYYCTSSYCCR | LRSEDEADYYCSTWDDSLSVRVFGGGTKLTVL (SEQ |
| | | GGSCPSHDTSYCGGQYKSYYYMDVWGKGTTVTVSS | ID NO: 1563) |
| | | (SEQ ID NO: 1369) | |
| 38 | AIP-185291 | EVQLVESGGALVKPGGSLRLSCAASGFTFSKAWMS | QSVLTQPPSASGTPGQRVTISCSGSSSNIGSSSVSWYQQ |
| | | WVRQAPGKGLEWVGRIKSVTDGETTDYAAPVKGRF | LPGTAPKLLIYKNNQRPSGVPDRFSGSKSGTSASLAISG |
| | | TISRDDSKSTLYLQMNSLKTEDTAVYYCTSSFCCL | LRSEDEADYYCSTWDDSLSVRVFGGGTKLTVL (SEQ |
| | | GGSCPSHDTSYCGGQYKSYYYMDVWGKGTTVTVSS | ID NO: 1564) |
| | | (SEQ ID NO: 1370) | |
| 39 | AIP-135247 | EVQLVESGGALVKPGGSLRLSCAASGFTFSKAWMS | QSVLTQPPSASGTPGQRVTISCSGSSSNIGSSSVSWYQQ |
| | | WVRQAPGKGLEWVGRIKSVTDGETTDYAAPVKGRF | LPGTAPKLLIYKNNQRPSGVPDRFSGSKSGTSASLAISG |
| | | TISRDDSKSTLYLQMNSLKTEDTAVYYCTSSFCCH | LRSEDEADYYCSTWDDSLSVRVFGGGTKLTVL (SEQ |
| | | GGSCPSHDTSYCGGQYKSYYYMDVWGKGTTVTVSS | ID NO: 1565) |
| | | (SEQ ID NO: 1371) | |
| 40 | AIP-113513 | EVQLVESGGALVKPGGSLRLSCAASGFTFSKAWMS | QSVLTQPPSASGTPGQRVTISCSGSSSNIGSSSVSWYQQ |
| | | WVRQAPGKGLEWVGRIKSVTDGETTDYAAPVKGRF | LPGTAPKLLIYKNNQRPSGVPDRFSGSKSGTSASLAISG |
| | | TISRDDSKSTLYLQMNSLKTEDTAVYYCTSSFCCQ | LRSEDEADYYCSTWDDSLSVRVFGGGTKLTVL (SEQ |
| | | GGSCPSHDTSYCGGQYKSYYYMDVWGKGTTVTVSS | ID NO: 1566) |
| | | (SEQ ID NO: 1372) | |
| 41 | AIP-102299 | EVQLVESGGALVKPGGSLRLSCAASGFTFSKAWMS | QSVLTQPPSASGTPGQRVTISCSGSSSNIGSSSVSWYQQ |
| | | WVRQAPGKGLEWVGRIKSVTDGETTDYAAPVKGRF | LPGTAPKLLIYKNNQRPSGVPDRFSGSKSGTSASLAISG |
| | | TISRDDSKSTLYLQMNSLKTEDTAVYYCTSSFCCR | LRSEDEADYYCSTWDDSLSVRVFGGGTKLTVL (SEQ |
| | | SGSCPSHDTSYCGGQYKSYYYMDVWGKGTTVTVSS | ID NO: 1567) |
| | | (SEQ ID NO: 1373) | |
| 42 | AIP-179097 | EVQLVESGGALVKPGGSLRLSCAASGFTFSKAWMS | QSVLTQPPSASGTPGQRVTISCSGSSSNIGSSSVSWYQQ |
| | | WVRQAPGKGLEWVGRIKSVTDGETTDYAAPVKGRF | LPGTAPKLLIYKNNQRPSGVPDRFSGSKSGTSASLAISG |
| | | TISRDDSKSTLYLQMNSLKTEDTAVYYCTSSFCCR | LRSEDEADYYCSTWDDSLSVRVFGGGTKLTVL (SEQ |
| | | GASCPSHDTSYCGGQYKSYYYMDVWGKGTTVTVSS | ID NO: 1568) |
| | | (SEQ ID NO: 1374) | |
| 43 | AIP-109343 | EVQLVESGGALVKPGGSLRLSCAASGFTFSKAWMS | QSVLTQPPSASGTPGQRVTISCSGSSSNIGSSSVSWYQQ |
| | | WVRQAPGKGLEWVGRIKSVTDGETTDYAAPVKGRF | LPGTAPKLLIYKNNQRPSGVPDRFSGSKSGTSASLAISG |
| | | TISRDDSKSTLYLQMNSLKTEDTAVYYCTSSFCCR | LRSEDEADYYCSTWDDSLSVRVFGGGTKLTVL (SEQ |
| | | GKSCPSHDTSYCGGQYKSYYYMDVWGKGTTVTVSS | ID NO: 1569) |
| | | (SEQ ID NO: 1375) | |
| 44 | AIP-119622 | EVQLVESGGALVKPGGSLRLSCAASGFTFSKAWMS | QSVLTQPPSASGTPGQRVTISCSGSSSNIGSSSVSWYQQ |
| | | WVRQAPGKGLEWVGRIKSVTDGETTDYAAPVKGRF | LPGTAPKLLIYKNNQRPSGVPDRFSGSKSGTSASLAISG |
| | | TISRDDSKSTLYLQMNSLKTEDTAVYYCTSSFCCR | LRSEDEADYYCSTWDDSLSVRVFGGGTKLTVL (SEQ |
| | | GNSCPSHDTSYCGGQYKSYYYMDVWGKGTTVTVSS | ID NO: 1570) |
| | | (SEQ ID NO: 1376) | |
| 45 | AIP-191735 | EVQLVESGGALVKPGGSLRLSCAASGFTFSKAWMS | QSVLTQPPSASGTPGQRVTISCSGSSSNIGSSSVSWYQQ |
| | | WVRQAPGKGLEWVGRIKSVTDGETTDYAAPVKGRF | LPGTAPKLLIYKNNQRPSGVPDRFSGSKSGTSASLAISG |
| | | TISRDDSKSTLYLQMNSLKTEDTAVYYCTSSFCCR | LRSEDEADYYCSTWDDSLSVRVFGGGTKLTVL (SEQ |
| | | GGNCPSHDTSYCGGQYKSYYYMDVWGKGTTVTVSS | ID NO: 1571) |
| | | (SEQ ID NO: 1377) | |
| 46 | AIP-157078 | EVQLVESGGALVKPGGSLRLSCAASGFTFSKAWMS | QSVLTQPPSASGTPGQRVTISCSGSSSNIGSSSVSWYQQ |
| | | WVRQAPGKGLEWVGRIKSVTDGETTDYAAPVKGRF | LPGTAPKLLIYKNNQRPSGVPDRFSGSKSGTSASLAISG |
| | | TISRDDSKSTLYLQMNSLKTEDTAVYYCTSSFCCR | LRSEDEADYYCSTWDDSLSVRVFGGGTKLTVL (SEQ |
| | | GGQCPSHDTSYCGGQYKSYYYMDVWGKGTTVTVSS | ID NO: 1572) |
| | | (SEQ ID NO: 1378) | |
| 47 | AIP-153475 | EVQLVESGGALVKPGGSLRLSCAASGFTFSKAWMS | QSVLTQPPSASGTPGQRVTISCSGSSSNIGSSSVSWYQQ |
| | | WVRQAPGKGLEWVGRIKSVTDGETTDYAAPVKGRF | LPGTAPKLLIYKNNQRPSGVPDRFSGSKSGTSASLAISG |
| | | TISRDDSKSTLYLQMNSLKTEDTAVYYCTSSFCCR | LRSEDEADYYCSTWDDSLSVRVFGGGTKLTVL (SEQ |
| | | GGACPSHDTSYCGGQYKSYYYMDVWGKGTTVTVSS | ID NO: 1573) |
| | | (SEQ ID NO: 1379) | |
| 48 | AIP-133650 | EVQLVESGGALVKPGGSLRLSCAASGFTFSKAWMS | QSVLTQPPSASGTPGQRVTISCSGSSSNIGSSSVSWYQQ |
| | | WVRQAPGKGLEWVGRIKSVTDGETTDYAAPVKGRF | LPGTAPKLLIYKNNQRPSGVPDRFSGSKSGTSASLAISG |
| | | TISRDDSKSTLYLQMNSLKTEDTAVYYCTSSFCCR | LRSEDEADYYCSTWDDSLSVRVFGGGTKLTVL (SEQ |
| | | GGSCASHDTSYCGGQYKSYYYMDVWGKGTTVTVSS | ID NO: 1574) |
| | | (SEQ ID NO: 1380) | |
| 49 | AIP-190915 | EVQLVESGGALVKPGGSLRLSCAASGFTFSKAWMS | QSVLTQPPSASGTPGQRVTISCSGSSSNIGSSSVSWYQQ |
| | | WVRQAPGKGLEWVGRIKSVTDGETTDYAAPVKGRF | LPGTAPKLLIYKNNQRPSGVPDRFSGSKSGTSASLAISG |
| | | TISRDDSKSTLYLQMNSLKTEDTAVYYCTSSFCCR | LRSEDEADYYCSTWDDSLSVRVFGGGTKLTVL (SEQ |
| | | GGSCLSHDTSYCGGQYKSYYYMDVWGKGTTVTVSS | ID NO: 1575) |
| | | (SEQ ID NO: 1381) | |
| 50 | AIP-105241 | EVQLVESGGALVKPGGSLRLSCAASGFTFSKAWMS | QSVLTQPPSASGTPGQRVTISCSGSSSNIGSSSVSWYQQ |
| | | WVRQAPGKGLEWVGRIKSVTDGETTDYAAPVKGRF | LPGTAPKLLIYKNNQRPSGVPDRFSGSKSGTSASLAISG |
| | | TISRDDSKSTLYLQMNSLKTEDTAVYYCTSSFCCR | LRSEDEADYYCSTWDDSLSVRVFGGGTKLTVL (SEQ |
| | | GGSCPAHDTSYCGGQYKSYYYMDVWGKGTTVTVSS | ID NO: 1576) |
| | | (SEQ ID NO: 1382) | |
| 51 | AIP-167400 | EVQLVESGGALVKPGGSLRLSCAASGFTFSKAWMS | QSVLTQPPSASGTPGQRVTISCSGSSSNIGSSSVSWYQQ |
| | | WVRQAPGKGLEWVGRIKSVTDGETTDYAAPVKGRF | LPGTAPKLLIYKNNQRPSGVPDRFSGSKSGTSASLAISG |
| | | TISRDDSKSTLYLQMNSLKTEDTAVYYCTSSFCCR | LRSEDEADYYCSTWDDSLSVRVFGGGTKLTVL (SEQ |
| | | GGSCPNHDTSYCGGQYKSYYYMDVWGKGTTVTVSS | ID NO: 1577) |
| | | (SEQ ID NO: 1383) | |
| 52 | AIP-109729 | EVQLVESGGALVKPGGSLRLSCAASGFTFSKAWMS | QSVLTQPPSASGTPGQRVTISCSGSSSNIGSSSVSWYQQ |
| | | WVRQAPGKGLEWVGRIKSVTDGETTDYAAPVKGRF | LPGTAPKLLIYKNNQRPSGVPDRFSGSKSGTSASLAISG |
| | | TISRDDSKSTLYLQMNSLKTEDTAVYYCTSSFCCR | LRSEDEADYYCSTWDDSLSVRVFGGGTKLTVL (SEQ |
| | | GGSCPSSDTSYCGGQYKSYYYMDVWGKGTTVTVSS | ID NO: 1578) |
| | | (SEQ ID NO: 1384) | |
| 53 | AIP-151709 | EVQLVESGGALVKPGGSLRLSCAASGFTFSKAWMS | QSVLTQPPSASGTPGQRVTISCSGSSSNIGSSSVSWYQQ |
| | | WVRQAPGKGLEWVGRIKSVTDGETTDYAAPVKGRF | LPGTAPKLLIYKNNQRPSGVPDRFSGSKSGTSASLAISG |
| | | TISRDDSKSTLYLQMNSLKTEDTAVYYCTSSFCCR | LRSEDEADYYCSTWDDSLSVRVFGGGTKLTVL (SEQ |
| | | GGSCPSQDTSYCGGQYKSYYYMDVWGKGTTVTVSS | ID NO: 1579) |
| | | (SEQ ID NO: 1385) | |
| 54 | AIP-137169 | EVQLVESGGALVKPGGSLRLSCAASGFTFSKAWMS | QSVLTQPPSASGTPGQRVTISCSGSSSNIGSSSVSWYQQ |
| | | WVRQAPGKGLEWVGRIKSVTDGETTDYAAPVKGRF | LPGTAPKLLIYKNNQRPSGVPDRFSGSKSGTSASLAISG |
| | | TISRDDSKSTLYLQMNSLKTEDTAVYYCTSSFCCR | LRSEDEADYYCSTWDDSLSVRVFGGGTKLTVL (SEQ |
| | | GGSCPSHETSYCGGQYKSYYYMDVWGKGTTVTVSS | ID NO: 1580) |
| | | (SEQ ID NO: 1386) | |
| 55 | AIP-199616 | EVQLVESGGALVKPGGSLRLSCAASGFTFSKAWMS | QSVLTQPPSASGTPGQRVTISCSGSSSNIGSSSVSWYQQ |
| | | WVRQAPGKGLEWVGRIKSVTDGETTDYAAPVKGRF | LPGTAPKLLIYKNNQRPSGVPDRFSGSKSGTSASLAISG |
| | | TISRDDSKSTLYLQMNSLKTEDTAVYYCTSSFCCR | LRSEDEADYYCSTWDDSLSVRVFGGGTKLTVL (SEQ |
| | | GGSCPSHDQSYCGGQYKSYYYMDVWGKGTTVTVSS | ID NO: 1581) |
| | | (SEQ ID NO: 1387) | |
| 56 | AIP-189296 | EVQLVESGGALVKPGGSLRLSCAASGFTFSKAWMS | QSVLTQPPSASGTPGQRVTISCSGSSSNIGSSSVSWYQQ |
| | | WVRQAPGKGLEWVGRIKSVTDGETTDYAAPVKGRF | LPGTAPKLLIYKNNQRPSGVPDRFSGSKSGTSASLAISG |
| | | TISRDDSKSTLYLQMNSLKTEDTAVYYCTSSFCCR | LRSEDEADYYCSTWDDSLSVRVFGGGTKLTVL (SEQ |
| | | GGSCPSHDTSMCGGQYKSYYYMDVWGKGTTVTVSS | ID NO: 1582) |
| | | (SEQ ID NO: 1388) | |
| 57 | AIP-152283 | EVQLVESGGALVKPGGSLRLSCAASGFTFSKAWMS | QSVLTQPPSASGTPGQRVTISCSGSSSNIGSSSVSWYQQ |
| | | WVRQAPGKGLEWVGRIKSVTDGETTDYAAPVKGRF | LPGTAPKLLIYKNNQRPSGVPDRFSGSKSGTSASLAISG |
| | | TISRDDSKSTLYLQMNSLKTEDTAVYYCTSSFCCR | LRSEDEADYYCSTWDDSLSVRVFGGGTKLTVL (SEQ |
| | | GGSCPSHDTSYCAGQYKSYYYMDVWGKGTTVTVSS | ID NO: 1583) |
| | | (SEQ ID NO: 1389) | |
| 58 | AIP-136628 | EVQLVESGGALVKPGGSLRLSCAASGFTFSKAWMS | QSVLTQPPSASGTPGQRVTISCSGSSSNIGSSSVSWYQQ |
| | | WVRQAPGKGLEWVGRIKSVTDGETTDYAAPVKGRF | LPGTAPKLLIYKNNQRPSGVPDRFSGSKSGTSASLAISG |
| | | TISRDDSKSTLYLQMNSLKTEDTAVYYCTSSFCCR | LRSEDEADYYCSTWDDSLSVRVFGGGTKLTVL (SEQ |
| | | GGSCPSHDTSYCNGQYKSYYYMDVWGKGTTVTVSS | ID NO: 1584) |
| | | (SEQ ID NO: 1390) | |
| 59 | AIP-100340 | EVQLVESGGALVKPGGSLRLSCAASGFTFSKAWMS | QSVLTQPPSASGTPGQRVTISCSGSSSNIGSSSVSWYQQ |
| | | WVRQAPGKGLEWVGRIKSVTDGETTDYAAPVKGRF | LPGTAPKLLIYKNNQRPSGVPDRFSGSKSGTSASLAISG |
| | | TISRDDSKSTLYLQMNSLKTEDTAVYYCTSSFCCR | LRSEDEADYYCSTWDDSLSVRVFGGGTKLTVL (SEQ |
| | | GGSCPSHDTSYCGNQYKSYYYMDVWGKGTTVTVSS | ID NO: 1585) |
| | | (SEQ ID NO: 1391) | |
| 60 | AIP-166959 | EVQLVESGGALVKPGGSLRLSCAASGFTFSKAWMS | QSVLTQPPSASGTPGQRVTISCSGSSSNIGSSSVSWYQQ |
| | | WVRQAPGKGLEWVGRIKSVTDGETTDYAAPVKGRF | LPGTAPKLLIYKNNQRPSGVPDRFSGSKSGTSASLAISG |
| | | TISRDDSKSTLYLQMNSLKTEDTAVYYCTSSFCCR | LRSEDEADYYCSTWDDSLSVRVFGGGTKLTVL (SEQ |
| | | GGSCPSHDTSYCGAQYKSYYYMDVWGKGTTVTVSS | ID NO: 1586) |
| | | (SEQ ID NO: 1392) | |
| 61 | AIP-190362 | EVQLVESGGALVKPGGSLRLSCAASGFTFSKAWMS | QSVLTQPPSASGTPGQRVTISCSGSSSNIGSSSVSWYQQ |
| | | WVRQAPGKGLEWVGRIKSVTDGETTDYAAPVKGRF | LPGTAPKLLIYKNNQRPSGVPDRFSGSKSGTSASLAISG |
| | | TISRDDSKSTLYLQMNSLKTEDTAVYYCTSSFCCR | LRSEDEADYYCSTWDDSLSVRVFGGGTKLTVL (SEQ |
| | | GGSCPSHDTSYCGGSYKSYYYMDVWGKGTTVTVSS | ID NO: 1587) |
| | | (SEQ ID NO: 1393) | |
| 62 | AIP-101601 | EVQLVESGGALVKPGGSLRLSCAASGFTFSKAWMS | QSVLTQPPSASGTPGQRVTISCSGSSSNIGSSSVSWYQQ |
| | | WVRQAPGKGLEWVGRIKSVTDGETTDYAAPVKGRF | LPGTAPKLLIYKNNQRPSGVPDRFSGSKSGTSASLAISG |
| | | TISRDDSKSTLYLQMNSLKTEDTAVYYCTSSFCCR | LRSEDEADYYCSTWDDSLSVRVFGGGTKLTVL (SEQ |
| | | GGSCPSHDTSYCGGYYKSYYYMDVWGKGTTVTVSS | ID NO: 1588) |
| | | (SEQ ID NO: 1394) | |
| 63 | AIP-159023 | EVQLVESGGALVKPGGSLRLSCAASGFTFSKAWMS | QSVLTQPPSASGTPGQRVTISCSGSSSNIGSSSVSWYQQ |
| | | WVRQAPGKGLEWVGRIKSVTDGETTDYAAPVKGRF | LPGTAPKLLIYKNNQRPSGVPDRFSGSKSGTSASLAISG |
| | | TISRDDSKSTLYLQMNSLKTEDTAVYYCTSSFCCR | LRSEDEADYYCSTWDDSLSVRVFGGGTKLTVL (SEQ |
| | | GGSCPSHDTSYCGGQFKSYYYMDVWGKGTTVTVSS | ID NO: 1589) |
| | | (SEQ ID NO: 1395) | |
| 64 | AIP-146871 | EVQLVESGGALVKPGGSLRLSCAASGFTFSKAWMS | QSVLTQPPSASGTPGQRVTISCSGSSSNIGSSSVSWYQQ |
| | | WVRQAPGKGLEWVGRIKSVTDGETTDYAAPVKGRF | LPGTAPKLLIYKNNQRPSGVPDRFSGSKSGTSASLAISG |
| | | TISRDDSKSTLYLQMNSLKTEDTAVYYCTSSFCCR | LRSEDEADYYCSTWDDSLSVRVFGGGTKLTVL (SEQ |
| | | GGSCPSHDTSYCGGQYPSYYYMDVWGKGTTVTVSS | ID NO: 1590) |
| | | (SEQ ID NO: 1396) | |
| 65 | AIP-170053 | EVQLVESGGALVKPGGSLRLSCAASGFTFSKAWMS | QSVLTQPPSASGTPGQRVTISCSGSSSNIGSSSVSWYQQ |
| | | WVRQAPGKGLEWVGRIKSVTDGETTDYAAPVKGRF | LPGTAPKLLIYKNNQRPSGVPDRFSGSKSGTSASLAISG |
| | | TISRDDSKSTLYLQMNSLKTEDTAVYYCTSSFCCR | LRSEDEADYYCSTWDDSLSVRVFGGGTKLTVL (SEQ |
| | | GGSCPSHDTSYCGGQYASYYYMDVWGKGTTVTVSS | ID NO: 1591) |
| | | (SEQ ID NO: 1397) | |
| 66 | AIP-199483 | EVQLVESGGALVKPGGSLRLSCAASGFTFSKAWMS | QSVLTQPPSASGTPGQRVTISCSGSSSNIGSSSVSWYQQ |
| | | WVRQAPGKGLEWVGRIKSVTDGETTDYAAPVKGRF | LPGTAPKLLIYKNNQRPSGVPDRFSGSKSGTSASLAISG |
| | | TISRDDSKSTLYLQMNSLKTEDTAVYYCTSSFCCR | LRSEDEADYYCSTWDDSLSVRVFGGGTKLTVL (SEQ |
| | | GGSCPSHDTSYCGGQYQSYYYMDVWGKGTTVTVSS | ID NO: 1592) |
| | | (SEQ ID NO: 1398) | |
| 67 | AIP-161048 | EVQLVESGGALVKPGGSLRLSCAASGFTFSKAWMS | QSVLTQPPSASGTPGQRVTISCSGSSSNIGSSSVSWYQQ |
| | | WVRQAPGKGLEWVGRIKSVTDGETTDYAAPVKGRF | LPGTAPKLLIYKNNQRPSGVPDRFSGSKSGTSASLAISG |
| | | TISRDDSKSTLYLQMNSLKTEDTAVYYCTSSFCCR | LRSEDEADYYCSTWDDSLSVRVFGGGTKLTVL (SEQ |
| | | GGSCPSHDTSYCGGQYKDYYYMDVWGKGTTVTVSS | ID NO: 1593) |
| | | (SEQ ID NO: 1399) | |
| 68 | AIP-162041 | EVQLVESGGALVKPGGSLRLSCAASGFTFSKAWMS | QSVLTQPPSASGTPGQRVTISCSGSSSNIGSSSVSWYQQ |
| | | WVRQAPGKGLEWVGRIKSVTDGETTDYAAPVKGRF | LPGTAPKLLIYKNNQRPSGVPDRFSGSKSGTSASLAISG |
| | | TISRDDSKSTLYLQMNSLKTEDTAVYYCTSSFCCR | LRSEDEADYYCSTWDDSLSVRVFGGGTKLTVL (SEQ |
| | | GGSCPSHDTSYCGGQYKSFYYMDVWGKGTTVTVSS | ID NO: 1594) |
| | | (SEQ ID NO: 1400) | |
| 69 | AIP-197886 | EVQLVESGGALVKPGGSLRLSCAASGFTFSKAWMS | QSVLTQPPSASGTPGQRVTISCSGSSSNIGSSSVSWYQQ |
| | | WVRQAPGKGLEWVGRIKSVTDGETTDYAAPVKGRF | LPGTAPKLLIYKNNQRPSGVPDRFSGSKSGTSASLAISG |
| | | TISRDDSKSTLYLQMNSLKTEDTAVYYCTSSFCCR | LRSEDEADYYCSTWDDSLSVRVFGGGTKLTVL (SEQ |
| | | GGSCPSHDTSYCGGQYKSYFYMDVWGKGTTVTVSS | ID NO: 1595) |
| | | (SEQ ID NO: 1401) | |
| 70 | AIP-183133 | EVQLVESGGALVKPGGSLRLSCAASGFTFSKAWMS | QSVLTQPPSASGTPGQRVTISCSGSSSNIGSSSVSWYQQ |
| | | WVRQAPGKGLEWVGRIKSVTDGETTDYAAPVKGRF | LPGTAPKLLIYKNNQRPSGVPDRFSGSKSGTSASLAISG |
| | | TISRDDSKSTLYLQMNSLKTEDTAVYYCTSSFCCR | LRSEDEADYYCSTWDDSLSVRVFGGGTKLTVL (SEQ |
| | | GGSCPSHDTSYCGGQYKSYYWMDVWGKGTTVTVSS | ID NO: 1596) |
| | | (SEQ ID NO: 1402) | |
| 71 | AIP-191470 | EVQLVESGGALVKPGGSLRLSCAASGFTFSKAWMS | QSVLTQPPSASGTPGQRVTISCSGSSSNIGSSSVSWYQQ |
| | | WVRQAPGKGLEWVGRIKSVTDGETTDYAAPVKGRF | LPGTAPKLLIYKNNQRPSGVPDRFSGSKSGTSASLAISG |
| | | TISRDDSKSTLYLQMNSLKTEDTAVYYCTSSFCCR | LRSEDEADYYCSTWDDSLSVRVFGGGTKLTVL (SEQ |
| | | GGSCPSHDTSYCGGQYKSYYLMDVWGKGTTVTVSS | ID NO: 1597) |
| | | (SEQ ID NO: 1403) | |
| 72 | AIP-151167 | EVQLVESGGALVKPGGSLRLSCAASGFTFSKAWMS | QSVLTQPPSASGTPGQRVTISCSGSSSNIGSSSVSWYQQ |
| | | WVRQAPGKGLEWVGRIKSVTDGETTDYAAPVKGRF | LPGTAPKLLIYKNNQRPSGVPDRFSGSKSGTSASLAISG |
| | | TISRDDSKSTLYLQMNSLKTEDTAVYYCTSSFCCR | LRSEDEADYYCSTWDDSLSVRVFGGGTKLTVL (SEQ |
| | | GGSCPSHDTSYCGGQYKSYYYVDVWGKGTTVTVSS | ID NO: 1598) |
| | | (SEQ ID NO: 1404) | |
| 73 | AIP-106633 | EVQLVESGGALVKPGGSLRLSCAASGFTFSKAWMS | QSVLTQPPSASGTPGQRVTISCSGSSSNIGSSSVSWYQQ |
| | | WVRQAPGKGLEWVGRIKSVTDGETTDYAAPVKGRF | LPGTAPKLLIYKNNQRPSGVPDRFSGSKSGTSASLAISG |
| | | TISRDDSKSTLYLQMNSLKTEDTAVYYCTSSFCCR | LRSEDEADYYCSTWDDSLSVRVFGGGTKLTVL (SEQ |
| | | GGSCPSHDTSYCGGQYKSYYYFDVWGKGTTVTVSS | ID NO: 1599) |
| | | (SEQ ID NO: 1405) | |
| 74 | AIP-102624 | EVQLVESGGALVKPGGSLRLSCAASGFTFSKAWMS | QSVLTQPPSASGTPGQRVTISCSGSSSNIGSSSVSWYQQ |
| | | WVRQAPGKGLEWVGRIKSVTDGETTDYAAPVKGRF | LPGTAPKLLIYKNNQRPSGVPDRFSGSKSGTSASLAISG |
| | | TISRDDSKSTLYLQMNSLKTEDTAVYYCTSSFCCR | LRSEDEADYYCSTWDDSLSVRVFGGGTKLTVL (SEQ |
| | | GGSCPSHDTSYCGGQYKSYYYMNVWGKGTTVTVSS | ID NO: 1600) |
| | | (SEQ ID NO: 1406) | |
| 75 | AIP-109484 | EVQLVESGGALVKPGGSLRLSCAASGFTFSKAWMS | QSVLTQPPSASGTPGQRVTISCSGSSSNIGSSSVSWYQQ |
| | | WVRQAPGKGLEWVGRIKSVTDGETTDYAAPVKGRF | LPGTAPKLLIYKNNQRPSGVPDRFSGSKSGTSASLAISG |
| | | TISRDDSKSTLYLQMNSLKTEDTAVYYCTSSFCCR | LRSEDEADYYCSTWDDSLSVRVFGGGTKLTVL (SEQ |
| | | GGSCPSHDTSYCGGQYKSYYYMDIWGKGTTVTVSS | ID NO: 1601) |
| | | (SEQ ID NO: 1407) | |
| 76 | AIP-164754 | EVQLVESGGALVKPGGSLRLSCAASGFTFSKAWMS | QSVLTQPPSASGTPGQRVTISCSGSSSNIGSSSVSWYQQ |
| | | WVRQAPGKGLEWVGRIKSVTDGETTDYAAPVKGRF | LPGTAPKLLIYKNNQRPSGVPDRFSGSKSGTSASLAISG |
| | | TISRDDSKSTLYLQMNSLKTEDTAVYYCTSSFCCR | LRSEDEADYYCSTWDDSLSVRVFGGGTKLTVL (SEQ |
| | | GGSCPSHDTSYCGGQYKSYYYMDPWGKGTTVTVSS | ID NO: 1602) |
| | | (SEQ ID NO: 1408) | |
| 77 | AIP-169676 | EVQLVESGGALVKPGGSLRLSCAASGFTFSKAWMS | QSVLTQPPSASGTPGQRVTISCSGSSSNIGSSSVSWYQQ |
| | | WVRQAPGKGLEWVGRIKSVTDGETTDYAAPVKGRF | LPGTAPKLLIYKNNQRPSGVPDRFSGSKSGTSASLAISG |
| | | TISRDDSKSTLYLQMNSLKTEDTAVYYCTSSFCCR | LRSEDEADYYCSTWDDSLSVRVFGGGTKLTVL (SEQ |
| | | GGSCPSHDTSYCGGQFKDYYYMDVWGKGTTVTVSS | ID NO: 1603) |
| | | (SEQ ID NO: 1409) | |
| 78 | AIP-177584 | EVQLVESGGALVKPGGSLRLSCAASGFTFSKAWMS | QSVLTQPPSASGTPGQRVTISCSGSSSNIGSSSVSWYQQ |
| | | WVRQAPGKGLEWVGRIKSVTDGETTDYAAPVKGRF | LPGTAPKLLIYKNNQRPSGVPDRFSGSKSGTSASLAISG |
| | | TISRDDSKSTLYLQMNSLKTEDTAVYYCTSSFCCR | LRSEDEADYYCSTWDDSLSVRVFGGGTKLTVL (SEQ |
| | | GGSCPSHDTSYCGGQSKQYYYMDVWGKGTTVTVSS | ID NO: 1604) |
| | | (SEQ ID NO: 1410) | |
| 79 | AIP-174676 | EVQLVESGGALVKPGGSLRLSCAASGFTFSKAWMS | QSVLTQPPSASGTPGQRVTISCSGSSSNIGSSSVSWYQQ |
| | | WVRQAPGKGLEWVGRIKSVTDGETTDYAAPVKGRF | LPGTAPKLLIYKNNQRPSGVPDRFSGSKSGTSASLAISG |
| | | TISRDDSKSTLYLQMNSLKTEDTAVYYCTSSFCCR | LRSEDEADYYCSTWDDSLSVRVFGGGTKLTVL (SEQ |
| | | GGSCPSHDTSYCGGQSKKYYYMDVWGKGTTVTVSS | ID NO: 1605) |
| | | (SEQ ID NO: 1411) | |
| 80 | AIP-120546 | EVQLVESGGALVKPGGSLRLSCAASGFTFSKAWMS | QSVLTQPPSASGTPGQRVTISCSGSSSNIGSSSVSWYQQ |
| | | WVRQAPGKGLEWVGRIKSVTDGETTDYAAPVKGRF | LPGTAPKLLIYKNNQRPSGVPDRFSGSKSGTSASLAISG |
| | | TISRDDSKSTLYLQMNSLKTEDTAVYYCTSSFSCR | LRSEDEADYYCSTWDDSLSVRVFGGGTKLTVL (SEQ |
| | | GGSCPSHDTSYSGGQYKSYYYMDVWGKGTTVTVSS | ID NO: 1606) |
| | | (SEQ ID NO: 1412) | |
| 81 | AIP-186435 | EVQLVESGGALVKPGGSLRLSCAASGFTFSKAWMS | QSVLTQPPSASGTPGQRVTISCSGSSSNIGSSSVSWYQQ |
| | | WVRQAPGKGLEWVGRIKSVTDGETTDYAAPVKGRF | LPGTAPKLLIYKNNQRPSGVPDRFSGSKSGTSASLAISG |
| | | TISRDDSKSTLYLQMNSLKTEDTAVYYCTSSFACR | LRSEDEADYYCSTWDDSLSVRVFGGGTKLTVL (SEQ |
| | | GGSCPSHDTSYVGGQYKSYYYMDVWGKGTTVTVSS | ID NO: 1607) |
| | | (SEQ ID NO: 1413) | |
| 82 | AIP-171074 | EVQLVESGGALVKPGGSLRLSCAASGFTFSKAWMS | QSVLTQPPSASGTPGQRVTISCSGSSSNIGSSSVSWYQQ |
| | | WVRQAPGKGLEWVGRIKSVTDGETTDYAAPVKGRF | LPGTAPKLLIYKNNQRPSGVPDRFSGSKSGTSASLAISG |
| | | TISRDDSKSTLYLQMNSLKTEDTAVYYCTSSFCCR | LRSEDEADYYCSTWDDSLSVRVFGGGTKLTVL (SEQ |
| | | GNSCPAHDTSYCGGQYPSYYYMDVWGKGTTVTVSS | ID NO: 1608) |
| | | (SEQ ID NO: 1414) | |
| 83 | AIP-163039 | EVQLVESGGALVKPGGSLRLSCAASGFTFSKAWMS | QSVLTQPPSASGTPGQRVTISCSGSSSNIGSSSVSWYQQ |
| | | WVRQAPGKGLEWVGRIKSVTDGETTDYAAPVKGRF | LPGTAPKLLIYKNNQRPSGVPDRFSGSKSGTSASLAISG |
| | | TISRDDSKSTLYLQMNSLKTEDTAVYYCTSSYCCL | LRSEDEADYYCSTWDDSLSVRVFGGGTKLTVL (SEQ |
| | | GGSCPSHDTSYCGGSYKSYYYMDVWGKGTTVTVSS | ID NO: 1609) |
| | | (SEQ ID NO: 1415) | |
| 84 | AIP-147652 | EVQLVESGGALVKPGGSLRLSCAASGFTFSKAWMS | QSVLTQPPSASGTPGQRVTISCSGSSSNIGSSSVSWYQQ |
| | | WVRQAPGKGLEWVGRIKSVTDGETTDYAAPVKGRF | LPGTAPKLLIYKNNQRPSGVPDRFSGSKSGTSASLAISG |
| | | TISRDDSKSTLYLQMNSLKTEDTAVYYCTSSFCCR | LRSEDEADYYCSTWDDSLSVRVFGGGTKLTVL (SEQ |
| | | GKSCPSHDNSYCGGQYASYYYMDVWGKGTTVTVSS | ID NO: 1610) |
| | | (SEQ ID NO: 1416) | |
| 85 | AIP-182061 | EVQLVESGGALVKPGGSLRLSCAASGFTFSKAWMS | QSVLTQPPSASGTPGQRVTISCSGSSSNIGSSSVSWYQQ |
| | | WVRQAPGKGLEWVGRIKSVTDGETTDYAAPVKGRF | LPGTAPKLLIYKNNQRPSGVPDRFSGSKSGTSASLAISG |
| | | TISRDDSKSTLYLQMNSLKTEDTAVYYCTSSYCCR | LRSEDEADYYCSTWDDSLSVRVFGGGTKLTVL (SEQ |
| | | GGSCPSHDTSYCGGQFKSYYYMDPWGKGTTVTVSS | ID NO: 1611) |
| | | (SEQ ID NO: 1417) | |
| 86 | AIP-172643 | EVQLVESGGALVKPGGSLRLSCAASGFTFSKAWMS | QSVLTQPPSASGTPGQRVTISCSGSSSNIGSSSVSWYQQ |
| | | WVRQAPGKGLEWVGRIKSVTDGETTDYAAPVKGRF | LPGTAPKLLIYKNNQRPSGVPDRFSGSKSGTSASLAISG |
| | | TISRDDSKSTLYLQMNSLKTEDTAVYYCTSTFCCR | LRSEDEADYYCSTWDDSLSVRVFGGGTKLTVL (SEQ |
| | | GGSCLSHDTSYCGGQYKSYYWMDVWGKGTTVTVSS | ID NO: 1612) |
| | | (SEQ ID NO: 1418) | |
| 87 | AIP-171912 | EVQLVESGGALVKPGGSLRLSCAASGFDFSKAWMS | QSVLTQPPSASGTPGQRVTISCTGSSSNIGSSSVSWYQQ |
| | | WVRQAPGKGLEWVGRIKSVTDGQTTDYAAPVKGRF | LPGTAPKLLIYKNNQRPSGVPDRFSGSKSGTSASLAISG |
| | | TISRDDSKSTLYLQMNSLKTEDTAVYYCTSSFCCR | LRSEDEADYYCSTWDDSLSVRVFGGGTKLTVL (SEQ |
| | | GGSCPSHDTSYCGGQYKSYYYMDVWGKGTTVTVSS | ID NO: 1613) |
| | | (SEQ ID NO: 1419) | |
| 88 | AIP-167833 | EVQLVESGGALVKPGGSLRLSCAASGFTFAKAWMS | QSVLTQPPSASGTPGQRVTISCSGSSTNIGSSSVSWYQQ |
| | | WVRQAPGKGLEWVGRIKSVTDGETTDYAAPVKGRF | LPGTAPKLLIYKNNYRPSGVPDRFSGSKSGTSASLAISG |
| | | TISRDDSKSTLYLQMNSLKTEDTAVYYCTSSFCCR | LRSEDEADYYCSTWDDSLSVRVFGGGTKLTVL (SEQ |
| | | GGSCPSHDTSYCGGQYKSYYYMDVWGKGTTVTVSS | ID NO: 1614) |
| | | (SEQ ID NO: 1420) | |
| 89 | AIP-145518 | EVQLVESGGALVKPGGSLRLSCAASGFDASKAWFT | QSVLTQPPSASGTPGQRVTISCSGSSSNIGSSSVSWYQQ |
| | | WVRQAPGKGLEWVGRIKSVTDGETTDYAAPVKGRF | LPGTAPKLLIYKNNQRPSGVPDRFSGSKSGTSASLAISG |
| | | TISRDDSKSTLYLQMNSLKTEDTAVYYCTSSFCCR | LRSEDEADYYCSTWDDSLSVRVFGGGTKLTVL (SEQ |
| | | GGSCPSHDTSYCGGQYKSYYYMDVWGKGTTVTVSS | ID NO: 1615) |
| | | (SEQ ID NO: 1421) | |
| 90 | AIP-143155 | EVQLVESGGALVKPGGSLRLSCAASGFTFSKAWMS | QSVLTQPPSASGTPGQRVTISCTGSSSNIGSSSVSWYQQ |
| | | WVRQAPGKGLEWVGRIKSVTDGETTDYAAPVKGRF | LPGTAPKLLIYKNNQRPSGVPDRFSGSKSGTSASLAISG |
| | | TISRDDSKSTLYLQMNSLKTEDTAVYYCTSSFCCR | LRSEDEADYYCSTWDDSLSVRVFGGGTKLTVL (SEQ |
| | | GGSCPSHDTSYCGGQYKSYYYMDVWGKGTTVTVSS | ID NO: 1616) |
| | | (SEQ ID NO: 1422) | |
| 91 | AIP-119664 | EVQLVESGGALVKPGGSLRLSCAASGFTFSKAWMS | QSVLTQPPSASGTPGQRVTISCSGASSNIGSSSVSWYQQ |
| | | WVRQAPGKGLEWVGRIKSVTDGETTDYAAPVKGRF | LPGTAPKLLIYKNNQRPSGVPDRFSGSKSGTSASLAISG |
| | | TISRDDSKSTLYLQMNSLKTEDTAVYYCTSSFCCR | LRSEDEADYYCSTWDDSLSVRVFGGGTKLTVL (SEQ |
| | | GGSCPSHDTSYCGGQYKSYYYMDVWGKGTTVTVSS | ID NO: 1617) |
| | | (SEQ ID NO: 1423) | |
| 92 | AIP-190526 | EVQLVESGGALVKPGGSLRLSCAASGFTFSKAWMS | QSVLTQPPSASGTPGQRVTISCSGSPSNIGSSSVSWYQQ |
| | | WVRQAPGKGLEWVGRIKSVTDGETTDYAAPVKGRF | LPGTAPKLLIYKNNQRPSGVPDRFSGSKSGTSASLAISG |
| | | TISRDDSKSTLYLQMNSLKTEDTAVYYCTSSFCCR | LRSEDEADYYCSTWDDSLSVRVFGGGTKLTVL (SEQ |
| | | GGSCPSHDTSYCGGQYKSYYYMDVWGKGTTVTVSS | ID NO: 1618) |
| | | (SEQ ID NO: 1424) | |
| 93 | AIP-114403 | EVQLVESGGALVKPGGSLRLSCAASGFTFSKAWMS | QSVLTQPPSASGTPGQRVTISCSGSKSNIGSSSVSWYQQ |
| | | WVRQAPGKGLEWVGRIKSVTDGETTDYAAPVKGRF | LPGTAPKLLIYKNNQRPSGVPDRFSGSKSGTSASLAISG |
| | | TISRDDSKSTLYLQMNSLKTEDTAVYYCTSSFCCR | LRSEDEADYYCSTWDDSLSVRVFGGGTKLTVL (SEQ |
| | | GGSCPSHDTSYCGGQYKSYYYMDVWGKGTTVTVSS | ID NO: 1619) |
| | | (SEQ ID NO: 1425) | |
| 94 | AIP-156760 | EVQLVESGGALVKPGGSLRLSCAASGFTFSKAWMS | QSVLTQPPSASGTPGQRVTISCSGSSTNIGSSSVSWYQQ |
| | | WVRQAPGKGLEWVGRIKSVTDGETTDYAAPVKGRF | LPGTAPKLLIYKNNQRPSGVPDRFSGSKSGTSASLAISG |
| | | TISRDDSKSTLYLQMNSLKTEDTAVYYCTSSFCCR | LRSEDEADYYCSTWDDSLSVRVFGGGTKLTVL (SEQ |
| | | GGSCPSHDTSYCGGQYKSYYYMDVWGKGTTVTVSS | ID NO: 1620) |
| | | (SEQ ID NO: 1426) | |
| 95 | AIP-103803 | EVQLVESGGALVKPGGSLRLSCAASGFTFSKAWMS | QSVLTQPPSASGTPGQRVTISCSGSSSDIGSSSVSWYQQ |
| | | WVRQAPGKGLEWVGRIKSVTDGETTDYAAPVKGRF | LPGTAPKLLIYKNNQRPSGVPDRFSGSKSGTSASLAISG |
| | | TISRDDSKSTLYLQMNSLKTEDTAVYYCTSSFCCR | LRSEDEADYYCSTWDDSLSVRVFGGGTKLTVL (SEQ |
| | | GGSCPSHDTSYCGGQYKSYYYMDVWGKGTTVTVSS | ID NO: 1621) |
| | | (SEQ ID NO: 1427) | |
| 96 | AIP-182722 | EVQLVESGGALVKPGGSLRLSCAASGFTFSKAWMS | QSVLTQPPSASGTPGQRVTISCSGSSSNIQSSSVSWYQQ |
| | | WVRQAPGKGLEWVGRIKSVTDGETTDYAAPVKGRF | LPGTAPKLLIYKNNQRPSGVPDRFSGSKSGTSASLAISG |
| | | TISRDDSKSTLYLQMNSLKTEDTAVYYCTSSFCCR | LRSEDEADYYCSTWDDSLSVRVFGGGTKLTVL (SEQ |
| | | GGSCPSHDTSYCGGQYKSYYYMDVWGKGTTVTVSS | ID NO: 1622) |
| | | (SEQ ID NO: 1428) | |
| 97 | AIP-195588 | EVQLVESGGALVKPGGSLRLSCAASGFTFSKAWMS | QSVLTQPPSASGTPGQRVTISCSGSSSNIGHSSVSWYQQ |
| | | WVRQAPGKGLEWVGRIKSVTDGETTDYAAPVKGRF | LPGTAPKLLIYKNNQRPSGVPDRFSGSKSGTSASLAISG |
| | | TISRDDSKSTLYLQMNSLKTEDTAVYYCTSSFCCR | LRSEDEADYYCSTWDDSLSVRVFGGGTKLTVL (SEQ |
| | | GGSCPSHDTSYCGGQYKSYYYMDVWGKGTTVTVSS | ID NO: 1623) |
| | | (SEQ ID NO: 1429) | |
| 98 | AIP-145722 | EVQLVESGGALVKPGGSLRLSCAASGFTFSKAWMS | QSVLTQPPSASGTPGQRVTISCSGSSSNIGSTSVSWYQQ |
| | | WVRQAPGKGLEWVGRIKSVTDGETTDYAAPVKGRF | LPGTAPKLLIYKNNQRPSGVPDRFSGSKSGTSASLAISG |
| | | TISRDDSKSTLYLQMNSLKTEDTAVYYCTSSFCCR | LRSEDEADYYCSTWDDSLSVRVFGGGTKLTVL (SEQ |
| | | GGSCPSHDTSYCGGQYKSYYYMDVWGKGTTVTVSS | ID NO: 1624) |
| | | (SEQ ID NO: 1430) | |
| 99 | AIP-178251 | EVQLVESGGALVKPGGSLRLSCAASGFTFSKAWMS | QSVLTQPPSASGTPGQRVTISCSGSSSNIGSNSVSWYQQ |
| | | WVRQAPGKGLEWVGRIKSVTDGETTDYAAPVKGRF | LPGTAPKLLIYKNNQRPSGVPDRFSGSKSGTSASLAISG |
| | | TISRDDSKSTLYLQMNSLKTEDTAVYYCTSSFCCR | LRSEDEADYYCSTWDDSLSVRVFGGGTKLTVL (SEQ |
| | | GGSCPSHDTSYCGGQYKSYYYMDVWGKGTTVTVSS | ID NO: 1625) |
| | | (SEQ ID NO: 1431) | |
| 100 | AIP-116142 | EVQLVESGGALVKPGGSLRLSCAASGFTFSKAWMS | QSVLTQPPSASGTPGQRVTISCSGSSSNIGSASVSWYQQ |
| | | WVRQAPGKGLEWVGRIKSVTDGETTDYAAPVKGRF | LPGTAPKLLIYKNNQRPSGVPDRFSGSKSGTSASLAISG |
| | | TISRDDSKSTLYLQMNSLKTEDTAVYYCTSSFCCR | LRSEDEADYYCSTWDDSLSVRVFGGGTKLTVL (SEQ |
| | | GGSCPSHDTSYCGGQYKSYYYMDVWGKGTTVTVSS | ID NO: 1626) |
| | | (SEQ ID NO: 1432) | |
| 101 | AIP-183350 | EVQLVESGGALVKPGGSLRLSCAASGFTFSKAWMS | QSVLTQPPSASGTPGQRVTISCSGSSSNIGSSTVSWYQQ |
| | | WVRQAPGKGLEWVGRIKSVTDGETTDYAAPVKGRF | LPGTAPKLLIYKNNQRPSGVPDRFSGSKSGTSASLAISG |
| | | TISRDDSKSTLYLQMNSLKTEDTAVYYCTSSFCCR | LRSEDEADYYCSTWDDSLSVRVFGGGTKLTVL (SEQ |
| | | GGSCPSHDTSYCGGQYKSYYYMDVWGKGTTVTVSS | ID NO: 1627) |
| | | (SEQ ID NO: 1433) | |
| 102 | AIP-127108 | EVQLVESGGALVKPGGSLRLSCAASGFTFSKAWMS | QSVLTQPPSASGTPGQRVTISCSGSSSNIGSSAVSWYQQ |
| | | WVRQAPGKGLEWVGRIKSVTDGETTDYAAPVKGRF | LPGTAPKLLIYKNNQRPSGVPDRFSGSKSGTSASLAISG |
| | | TISRDDSKSTLYLQMNSLKTEDTAVYYCTSSFCCR | LRSEDEADYYCSTWDDSLSVRVFGGGTKLTVL (SEQ |
| | | GGSCPSHDTSYCGGQYKSYYYMDVWGKGTTVTVSS | ID NO: 1628) |
| | | (SEQ ID NO: 1434) | |
| 103 | AIP-128147 | EVQLVESGGALVKPGGSLRLSCAASGFTFSKAWMS | QSVLTQPPSASGTPGQRVTISCSGSSSNIGSSYVSWYQQ |
| | | WVRQAPGKGLEWVGRIKSVTDGETTDYAAPVKGRF | LPGTAPKLLIYKNNQRPSGVPDRFSGSKSGTSASLAISG |
| | | TISRDDSKSTLYLQMNSLKTEDTAVYYCTSSFCCR | LRSEDEADYYCSTWDDSLSVRVFGGGTKLTVL (SEQ |
| | | GGSCPSHDTSYCGGQYKSYYYMDVWGKGTTVTVSS | ID NO: 1629) |
| | | (SEQ ID NO: 1435) | |
| 104 | AIP-109510 | EVQLVESGGALVKPGGSLRLSCAASGFTFSKAWMS | QSVLTQPPSASGTPGQRVTISCSGSSSNIGSSSTSWYQQ |
| | | WVRQAPGKGLEWVGRIKSVTDGETTDYAAPVKGRF | LPGTAPKLLIYKNNQRPSGVPDRFSGSKSGTSASLAISG |
| | | TISRDDSKSTLYLQMNSLKTEDTAVYYCTSSFCCR | LRSEDEADYYCSTWDDSLSVRVFGGGTKLTVL (SEQ |
| | | GGSCPSHDTSYCGGQYKSYYYMDVWGKGTTVTVSS | ID NO: 1630) |
| | | (SEQ ID NO: 1436) | |
| 105 | AIP-104086 | EVQLVESGGALVKPGGSLRLSCAASGFTFSKAWMS | QSVLTQPPSASGTPGQRVTISCSGSSSNIGSSSVAWYQQ |
| | | WVRQAPGKGLEWVGRIKSVTDGETTDYAAPVKGRF | LPGTAPKLLIYKNNQRPSGVPDRFSGSKSGTSASLAISG |
| | | TISRDDSKSTLYLQMNSLKTEDTAVYYCTSSFCCR | LRSEDEADYYCSTWDDSLSVRVFGGGTKLTVL (SEQ |
| | | GGSCPSHDTSYCGGQYKSYYYMDVWGKGTTVTVS | ID NO: 1631) |
| | | (SEQ ID NO: 1437) | |
| 106 | AIP-143132 | EVQLVESGGALVKPGGSLRLSCAASGFTFSKAWMS | QSVLTQPPSASGTPGQRVTISCSGSSSNIGSSSVTWYQQ |
| | | WVRQAPGKGLEWVGRIKSVTDGETTDYAAPVKGRF | LPGTAPKLLIYKNNQRPSGVPDRFSGSKSGTSASLAISG |
| | | TISRDDSKSTLYLQMNSLKTEDTAVYYCTSSFCCR | LRSEDEADYYCSTWDDSLSVRVFGGGTKLTVL (SEQ |
| | | GGSCPSHDTSYCGGQYKSYYYMDVWGKGTTVTVSS | ID NO: 1632) |
| | | (SEQ ID NO: 1438) | |
| 107 | AIP-169636 | EVQLVESGGALVKPGGSLRLSCAASGFTFSKAWMS | QSVLTQPPSASGTPGQRVTISCSGSSSNIGSSSVSWYQQ |
| | | WVRQAPGKGLEWVGRIKSVTDGETTDYAAPVKGRF | LPGTAPKLLIYRNNQRPSGVPDRFSGSKSGTSASLAISG |
| | | TISRDDSKSTLYLQMNSLKTEDTAVYYCTSSFCCR | LRSEDEADYYCSTWDDSLSVRVFGGGTKLTVL (SEQ |
| | | GGSCPSHDTSYCGGQYKSYYYMDVWGKGTTVTVSS | ID NO: 1633) |
| | | (SEQ ID NO: 1439) | |
| 108 | AIP-152243 | EVQLVESGGALVKPGGSLRLSCAASGFTFSKAWMS | QSVLTQPPSASGTPGQRVTISCSGSSSNIGSSSVSWYQQ |
| | | WVRQAPGKGLEWVGRIKSVTDGETTDYAAPVKGRF | LPGTAPKLLIYNNNQRPSGVPDRFSGSKSGTSASLAISG |
| | | TISRDDSKSTLYLQMNSLKTEDTAVYYCTSSFCCR | LRSEDEADYYCSTWDDSLSVRVFGGGTKLTVL (SEQ |
| | | GGSCPSHDTSYCGGQYKSYYYMDVWGKGTTVTVSS | ID NO: 1634) |
| | | (SEQ ID NO: 1440) | |
| 109 | AIP-138776 | EVQLVESGGALVKPGGSLRLSCAASGFTFSKAWMS | QSVLTQPPSASGTPGQRVTISCSGSSSNIGSSSVSWYQQ |
| | | WVRQAPGKGLEWVGRIKSVTDGETTDYAAPVKGRF | LPGTAPKLLIYMNNQRPSGVPDRFSGSKSGTSASLAISG |
| | | TISRDDSKSTLYLQMNSLKTEDTAVYYCTSSFCCR | LRSEDEADYYCSTWDDSLSVRVFGGGTKLTVL (SEQ |
| | | GGSCPSHDTSYCGGQYKSYYYMDVWGKGTTVTVSS | ID NO: 1635) |
| | | (SEQ ID NO: 1441) | |
| 110 | AIP-103817 | EVQLVESGGALVKPGGSLRLSCAASGFTFSKAWMS | QSVLTQPPSASGTPGQRVTISCSGSSSNIGSSSVSWYQQ |
| | | WVRQAPGKGLEWVGRIKSVTDGETTDYAAPVKGRF | LPGTAPKLLIYANNQRPSGVPDRFSGSKSGTSASLAISG |
| | | TISRDDSKSTLYLQMNSLKTEDTAVYYCTSSFCCR | LRSEDEADYYCSTWDDSLSVRVFGGGTKLTVL (SEQ |
| | | GGSCPSHDTSYCGGQYKSYYYMDVWGKGTTVTVSS | ID NO: 1636) |
| | | (SEQ ID NO: 1442) | |
| 111 | AIP-130491 | EVQLVESGGALVKPGGSLRLSCAASGFTFSKAWMS | QSVLTQPPSASGTPGQRVTISCSGSSSNIGSSSVSWYQQ |
| | | WVRQAPGKGLEWVGRIKSVTDGETTDYAAPVKGRF | LPGTAPKLLIYHNNQRPSGVPDRFSGSKSGTSASLAISG |
| | | TISRDDSKSTLYLQMNSLKTEDTAVYYCTSSFCCR | LRSEDEADYYCSTWDDSLSVRVFGGGTKLTVL (SEQ |
| | | GGSCPSHDTSYCGGQYKSYYYMDVWGKGTTVTVSS | ID NO: 1637) |
| | | (SEQ ID NO: 1443) | |
| 112 | AIP-188155 | EVQLVESGGALVKPGGSLRLSCAASGFTFSKAWMS | QSVLTQPPSASGTPGQRVTISCSGSSSNIGSSSVSWYQQ |
| | | WVRQAPGKGLEWVGRIKSVTDGETTDYAAPVKGRF | LPGTAPKLLIYKDNQRPSGVPDRFSGSKSGTSASLAISG |
| | | TISRDDSKSTLYLQMNSLKTEDTAVYYCTSSFCCR | LRSEDEADYYCSTWDDSLSVRVFGGGTKLTVL (SEQ |
| | | GGSCPSHDTSYCGGQYKSYYYMDVWGKGTTVTVSS | ID NO: 1638) |
| | | (SEQ ID NO: 1444) | |
| 113 | AIP-167246 | EVQLVESGGALVKPGGSLRLSCAASGFTFSKAWMS | QSVLTQPPSASGTPGQRVTISCSGSSSNIGSSSVSWYQQ |
| | | WVRQAPGKGLEWVGRIKSVTDGETTDYAAPVKGRF | LPGTAPKLLIYKNTQRPSGVPDRFSGSKSGTSASLAISG |
| | | TISRDDSKSTLYLQMNSLKTEDTAVYYCTSSFCCR | LRSEDEADYYCSTWDDSLSVRVFGGGTKLTVL (SEQ |
| | | GGSCPSHDTSYCGGQYKSYYYMDVWGKGTTVTVSS | ID NO: 1639) |
| | | (SEQ ID NO: 1445) | |
| 114 | AIP-106139 | EVQLVESGGALVKPGGSLRLSCAASGFTFSKAWMS | QSVLTQPPSASGTPGQRVTISCSGSSSNIGSSSVSWYQQ |
| | | WVRQAPGKGLEWVGRIKSVTDGETTDYAAPVKGRF | LPGTAPKLLIYKNNYRPSGVPDRFSGSKSGTSASLAISG |
| | | TISRDDSKSTLYLQMNSLKTEDTAVYYCTSSFCCR | LRSEDEADYYCSTWDDSLSVRVFGGGTKLTVL (SEQ |
| | | GGSCPSHDTSYCGGQYKSYYYMDVWGKGTTVTVSS | ID NO: 1640) |
| | | (SEQ ID NO: 1446) | |
| 115 | AIP-198351 | EVQLVESGGALVKPGGSLRLSCAASGFTFSKAWMS | QSVLTQPPSASGTPGQRVTISCSGSSSNIGSSSVSWYQQ |
| | | WVRQAPGKGLEWVGRIKSVTDGETTDYAAPVKGRF | LPGTAPKLLIYKNNARPSGVPDRFSGSKSGTSASLAISG |
| | | TISRDDSKSTLYLQMNSLKTEDTAVYYCTSSFCCR | LRSEDEADYYCSTWDDSLSVRVFGGGTKLTVL (SEQ |
| | | GGSCPSHDTSYCGGQYKSYYYMDVWGKGTTVTVSS | ID NO: 1641) |
| | | (SEQ ID NO: 1447) | |
| 116 | AIP-159326 | EVQLVESGGALVKPGGSLRLSCAASGFTFSKAWMS | QSVLTQPPSASGTPGQRVTISCSGSSSNIGSSSVSWYQQ |
| | | WVRQAPGKGLEWVGRIKSVTDGETTDYAAPVKGRF | LPGTAPKLLIYKNNLRPSGVPDRFSGSKSGTSASLAISG |
| | | TISRDDSKSTLYLQMNSLKTEDTAVYYCTSSFCCR | LRSEDEADYYCSTWDDSLSVRVFGGGTKLTVL (SEQ |
| | | GGSCPSHDTSYCGGQYKSYYYMDVWGKGTTVTVSS | ID NO: 1642) |
| | | (SEQ ID NO: 1448) | |
| 117 | AIP-192275 | EVQLVESGGALVKPGGSLRLSCAASGFTFSKAWMS | QSVLTQPPSASGTPGQRVTISCSGSSSNIGSSSVSWYQQ |
| | | WVRQAPGKGLEWVGRIKSVTDGETTDYAAPVKGRF | LPGTAPKLLIYKNNQQPSGVPDRFSGSKSGTSASLAISG |
| | | TISRDDSKSTLYLQMNSLKTEDTAVYYCTSSFCCR | LRSEDEADYYCSTWDDSLSVRVFGGGTKLTVL (SEQ |
| | | GGSCPSHDTSYCGGQYKSYYYMDVWGKGTTVTVSS | ID NO: 1643) |
| | | (SEQ ID NO: 1449) | |
| 118 | AIP-190761 | EVQLVESGGALVKPGGSLRLSCAASGFTFSKAWMS | QSVLTQPPSASGTPGQRVTISCSGSSSNIGSSSVSWYQQ |
| | | WVRQAPGKGLEWVGRIKSVTDGETTDYAAPVKGRF | LPGTAPKLLIYKNNQRASGVPDRFSGSKSGTSASLAISG |
| | | TISRDDSKSTLYLQMNSLKTEDTAVYYCTSSFCCR | LRSEDEADYYCSTWDDSLSVRVFGGGTKLTVL (SEQ |
| | | GGSCPSHDTSYCGGQYKSYYYMDVWGKGTTVTVSS | ID NO: 1644) |
| | | (SEQ ID NO: 1450) | |
| 119 | AIP-166832 | EVQLVESGGALVKPGGSLRLSCAASGFTFSKAWMS | QSVLTQPPSASGTPGQRVTISCSGSSSNIGSSSVSWYQQ |
| | | WVRQAPGKGLEWVGRIKSVTDGETTDYAAPVKGRF | LPGTAPKLLIYKNNQRPYGVPDRFSGSKSGTSASLAISG |
| | | TISRDDSKSTLYLQMNSLKTEDTAVYYCTSSFCCR | LRSEDEADYYCSTWDDSLSVRVFGGGTKLTVL (SEQ |
| | | GGSCPSHDTSYCGGQYKSYYYMDVWGKGTTVTVSS | ID NO: 1645) |
| | | (SEQ ID NO: 1451) | |
| 120 | AIP-148062 | EVQLVESGGALVKPGGSLRLSCAASGFTFSKAWMS | QSVLTQPPSASGTPGQRVTISCSGSSSNIGSSSVSWYQQ |
| | | WVRQAPGKGLEWVGRIKSVTDGETTDYAAPVKGRF | LPGTAPKLLIYKNNQRPLGVPDRFSGSKSGTSASLAISG |
| | | TISRDDSKSTLYLQMNSLKTEDTAVYYCTSSFCCR | LRSEDEADYYCSTWDDSLSVRVFGGGTKLTVL (SEQ |
| | | GGSCPSHDTSYCGGQYKSYYYMDVWGKGTTVTVSS | ID NO: 1646) |
| | | (SEQ ID NO: 1452) | |
| 121 | AIP-129145 | EVQLVESGGALVKPGGSLRLSCAASGFTFSKAWMS | QSVLTQPPSASGTPGQRVTISCSGSSSNIGSSSVSWYQQ |
| | | WVRQAPGKGLEWVGRIKSVTDGETTDYAAPVKGRF | LPGTAPKLLIYKNNQRPSGVPDRFSGSKSGTSASLAISG |
| | | TISRDDSKSTLYLQMNSLKTEDTAVYYCTSSFCCR | LRSEDEADYYCATWDDSLSVRVFGGGTKLTVL (SEQ |
| | | GGSCPSHDTSYCGGQYKSYYYMDVWGKGTTVTVSS | ID NO: 1647) |
| | | (SEQ ID NO: 1453) | |
| 122 | AIP-111240 | EVQLVESGGALVKPGGSLRLSCAASGFTFSKAWMS | QSVLTQPPSASGTPGQRVTISCSGSSSNIGSSSVSWYQQ |
| | | WVRQAPGKGLEWVGRIKSVTDGETTDYAAPVKGRF | LPGTAPKLLIYKNNQRPSGVPDRFSGSKSGTSASLAISG |
| | | TISRDDSKSTLYLQMNSLKTEDTAVYYCTSSFCCR | LRSEDEADYYCSSWDDSLSVRVFGGGTKLTVL (SEQ |
| | | GGSCPSHDTSYCGGQYKSYYYMDVWGKGTTVTVSS | ID NO: 1648) |
| | | (SEQ ID NO: 1454) | |
| 123 | AIP-190749 | EVQLVESGGALVKPGGSLRLSCAASGFTFSKAWMS | QSVLTQPPSASGTPGQRVTISCSGSSSNIGSSSVSWYQQ |
| | | WVRQAPGKGLEWVGRIKSVTDGETTDYAAPVKGRF | LPGTAPKLLIYKNNQRPSGVPDRFSGSKSGTSASLAISG |
| | | TISRDDSKSTLYLQMNSLKTEDTAVYYCTSSFCCR | LRSEDEADYYCSTFDDSLSVRVFGGGTKLTVL (SEQ |
| | | GGSCPSHDTSYCGGQYKSYYYMDVWGKGTTVTVSS | ID NO: 1649) |
| | | (SEQ ID NO: 1455) | |
| 124 | AIP-153888 | EVQLVESGGALVKPGGSLRLSCAASGFTFSKAWMS | QSVLTQPPSASGTPGQRVTISCSGSSSNIGSSSVSWYQQ |
| | | WVRQAPGKGLEWVGRIKSVTDGETTDYAAPVKGRF | LPGTAPKLLIYKNNQRPSGVPDRFSGSKSGTSASLAISG |
| | | TISRDDSKSTLYLQMNSLKTEDTAVYYCTSSFCCR | LRSEDEADYYCSTWDNSLSVRVFGGGTKLTVL (SEQ |
| | | GGSCPSHDTSYCGGQYKSYYYMDVWGKGTTVTVSS | ID NO: 1650) |
| | | (SEQ ID NO: 1456) | |
| 125 | AIP-130915 | EVQLVESGGALVKPGGSLRLSCAASGFTFSKAWMS | QSVLTQPPSASGTPGQRVTISCSGSSSNIGSSSVSWYQQ |
| | | WVRQAPGKGLEWVGRIKSVTDGETTDYAAPVKGRF | LPGTAPKLLIYKNNQRPSGVPDRFSGSKSGTSASLAISG |
| | | TISRDDSKSTLYLQMNSLKTEDTAVYYCTSSFCCR | LRSEDEADYYCSTWDDQLSVRVFGGGTKLTVL (SEQ |
| | | GGSCPSHDTSYCGGQYKSYYYMDVWGKGTTVTVSS | ID NO: 1651) |
| | | (SEQ ID NO: 1457) | |
| 126 | AIP-109048 | EVQLVESGGALVKPGGSLRLSCAASGFTFSKAWMS | QSVLTQPPSASGTPGQRVTISCSGSSSNIGSSSVSWYQQ |
| | | WVRQAPGKGLEWVGRIKSVTDGETTDYAAPVKGRF | LPGTAPKLLIYKNNQRPSGVPDRFSGSKSGTSASLAISG |
| | | TISRDDSKSTLYLQMNSLKTEDTAVYYCTSSFCCR | LRSEDEADYYCSTWDDALSVRVFGGGTKLTVL (SEQ |
| | | GGSCPSHDTSYCGGQYKSYYYMDVWGKGTTVTVSS | ID NO: 1652) |
| | | (SEQ ID NO: 1458) | |
| 127 | AIP-170569 | EVQLVESGGALVKPGGSLRLSCAASGFTFSKAWMS | QSVLTQPPSASGTPGQRVTISCSGSSSNIGSSSVSWYQQ |
| | | WVRQAPGKGLEWVGRIKSVTDGETTDYAAPVKGRF | LPGTAPKLLIYKNNQRPSGVPDRFSGSKSGTSASLAISG |
| | | TISRDDSKSTLYLQMNSLKTEDTAVYYCTSSFCCR | LRSEDEADYYCSTWDDDLSVRVFGGGTKLTVL (SEQ |
| | | GGSCPSHDTSYCGGQYKSYYYMDVWGKGTTVTVSS | ID NO: 1653) |
| | | (SEQ ID NO: 1459) | |
| 128 | AIP-154873 | EVQLVESGGALVKPGGSLRLSCAASGFTFSKAWMS | QSVLTQPPSASGTPGQRVTISCSGSSSNIGSSSVSWYQQ |
| | | WVRQAPGKGLEWVGRIKSVTDGETTDYAAPVKGRF | LPGTAPKLLIYKNNQRPSGVPDRFSGSKSGTSASLAISG |
| | | TISRDDSKSTLYLQMNSLKTEDTAVYYCTSSFCCR | LRSEDEADYYCSTWDDSNSVRVFGGGTKLTVL (SEQ |
| | | GGSCPSHDTSYCGGQYKSYYYMDVWGKGTTVTVSS | ID NO: 1654) |
| | | (SEQ ID NO: 1460) | |
| 129 | AIP-159037 | EVQLVESGGALVKPGGSLRLSCAASGFTFSKAWMS | QSVLTQPPSASGTPGQRVTISCSGSSSNIGSSSVSWYQQ |
| | | WVRQAPGKGLEWVGRIKSVTDGETTDYAAPVKGRF | LPGTAPKLLIYKNNQRPSGVPDRFSGSKSGTSASLAISG |
| | | TISRDDSKSTLYLQMNSLKTEDTAVYYCTSSFCCR | LRSEDEADYYCSTWDDSSSVRVFGGGTKLTVL (SEQ |
| | | GGSCPSHDTSYCGGQYKSYYYMDVWGKGTTVTVSS | ID NO: 1655) |
| | | (SEQ ID NO: 1461) | |
| 130 | AIP-186826 | EVQLVESGGALVKPGGSLRLSCAASGFTFSKAWMS | QSVLTQPPSASGTPGQRVTISCSGSSSNIGSSSVSWYQQ |
| | | WVRQAPGKGLEWVGRIKSVTDGETTDYAAPVKGRF | LPGTAPKLLIYKNNQRPSGVPDRFSGSKSGTSASLAISG |
| | | TISRDDSKSTLYLQMNSLKTEDTAVYYCTSSFCCR | LRSEDEADYYCSTWDDSLNVRVFGGGTKLTVL (SEQ |
| | | GGSCPSHDTSYCGGQYKSYYYMDVWGKGTTVTVSS | ID NO: 1656) |
| | | (SEQ ID NO: 1462) | |
| 131 | AIP-156514 | EVQLVESGGALVKPGGSLRLSCAASGFTFSKAWMS | QSVLTQPPSASGTPGQRVTISCSGSSSNIGSSSVSWYQQ |
| | | WVRQAPGKGLEWVGRIKSVTDGETTDYAAPVKGRF | LPGTAPKLLIYKNNQRPSGVPDRFSGSKSGTSASLAISG |
| | | TISRDDSKSTLYLQMNSLKTEDTAVYYCTSSFCCR | LRSEDEADYYCSTWDDSLLVRVFGGGTKLTVL (SEQ |
| | | GGSCPSHDTSYCGGQYKSYYYMDVWGKGTTVTVSS | ID NO: 1657) |
| | | (SEQ ID NO: 1463) | |
| 132 | AIP-157122 | EVQLVESGGALVKPGGSLRLSCAASGFTFSKAWMS | QSVLTQPPSASGTPGQRVTISCSGSSSNIGSSSVSWYQQ |
| | | WVRQAPGKGLEWVGRIKSVTDGETTDYAAPVKGRF | LPGTAPKLLIYKNNQRPSGVPDRFSGSKSGTSASLAISG |
| | | TISRDDSKSTLYLQMNSLKTEDTAVYYCTSSFCCR | LRSEDEADYYCSTWDDSLTVRVFGGGTKLTVL (SEQ |
| | | GGSCPSHDTSYCGGQYKSYYYMDVWGKGTTVTVSS | ID NO: 1658) |
| | | (SEQ ID NO: 1464) | |
| 133 | AIP-173276 | EVQLVESGGALVKPGGSLRLSCAASGFTFSKAWMS | QSVLTQPPSASGTPGQRVTISCSGSSSNIGSSSVSWYQQ |
| | | WVRQAPGKGLEWVGRIKSVTDGETTDYAAPVKGRF | LPGTAPKLLIYKNNQRPSGVPDRFSGSKSGTSASLAISG |
| | | TISRDDSKSTLYLQMNSLKTEDTAVYYCTSSFCCR | LRSEDEADYYCSTWDDSLSIRVFGGGTKLTVL (SEQ |
| | | GGSCPSHDTSYCGGQYKSYYYMDVWGKGTTVTVSS | ID NO: 1659) |
| | | (SEQ ID NO: 1465) | |
| 134 | AIP-150485 | EVQLVESGGALVKPGGSLRLSCAASGFTFSKAWMS | QSVLTQPPSASGTPGQRVTISCSGSSSNIGSSSVSWYQQ |
| | | WVRQAPGKGLEWVGRIKSVTDGETTDYAAPVKGRF | LPGTAPKLLIYKNNQRPSGVPDRFSGSKSGTSASLAISG |
| | | TISRDDSKSTLYLQMNSLKTEDTAVYYCTSSFCCR | LRSEDEADYYCSTWDDSLSVKVFGGGTKLTVL (SEQ |
| | | GGSCPSHDTSYCGGQYKSYYYMDVWGKGTTVTVSS | ID NO: 1660) |
| | | (SEQ ID NO: 1466) | |
| 135 | AIP-135679 | EVQLVESGGALVKPGGSLRLSCAASGFTFSKAWMS | QSVLTQPPSASGTPGQRVTISCSGSSSNIGSSSVSWYQQ |
| | | WVRQAPGKGLEWVGRIKSVTDGETTDYAAPVKGRF | LPGTAPKLLIYKNNQRPSGVPDRFSGSKSGTSASLAISG |
| | | TISRDDSKSTLYLQMNSLKTEDTAVYYCTSSFCCR | LRSEDEADYYCSTWDDSLSVWVFGGGTKLTVL (SEQ |
| | | GGSCPSHDTSYCGGQYKSYYYMDVWGKGTTVTVSS | ID NO: 1661) |
| | | (SEQ ID NO: 1467) | |
| 136 | AIP-166847 | EVQLVESGGALVKPGGSLRLSCAASGFTFSKAWMS | QSVLTQPPSASGTPGQRVTISCSGSSSNIGSSSVSWYQQ |
| | | WVRQAPGKGLEWVGRIKSVTDGETTDYAAPVKGRF | LPGTAPKLLIYKNNQRPSGVPDRFSGSKSGTSASLAISG |
| | | TISRDDSKSTLYLQMNSLKTEDTAVYYCTSSFCCR | LRSEDEADYYCSTWDDSLSVHVFGGGTKLTVL (SEQ |
| | | GGSCPSHDTSYCGGQYKSYYYMDVWGKGTTVTVSS | ID NO: 1662) |
| | | (SEQ ID NO: 1468) | |
| 137 | AIP-124013 | EVQLVESGGALVKPGGSLRLSCAASGFTFSKAWMS | QSVLTQPPSASGTPGQRVTISCSGSSSNIGSSSVSWYQQ |
| | | WVRQAPGKGLEWVGRIKSVTDGETTDYAAPVKGRF | LPGTAPKLLIYKNNQRPSGVPDRFSGSKSGTSASLAISG |
| | | TISRDDSKSTLYLQMNSLKTEDTAVYYCTSSFCCR | LRSEDEADYYCSTWDDSLSVQVFGGGTKLTVL (SEQ |
| | | GGSCPSHDTSYCGGQYKSYYYMDVWGKGTTVTVSS | ID NO: 1663) |
| | | (SEQ ID NO: 1469) | |
| 138 | AIP-126285 | EVQLVESGGALVKPGGSLRLSCAASGFTFSKAWMS | QSVLTQPPSASGTPGQRVTISCSGSSSNIGSSSVSWYQQ |
| | | WVRQAPGKGLEWVGRIKSVTDGETTDYAAPVKGRF | LPGTAPKLLIYKNNQRPSGVPDRFSGSKSGTSASLAISG |
| | | TISRDDSKSTLYLQMNSLKTEDTAVYYCTSSFCCR | LRSEDEADYYCSTWDDSLSVRIFGGGTKLTVL (SEQ |
| | | GGSCPSHDTSYCGGQYKSYYYMDVWGKGTTVTVSS | ID NO: 1664) |
| | | (SEQ ID NO: 1470) | |
| 139 | AIP-190274 | EVQLVESGGALVKPGGSLRLSCAASGFTFSKAWMS | QSVLTQPPSASGTPGQRVTISCSGSSSDIGSSSVSWYQQ |
| | | WVRQAPGKGLEWVGRIKSVTDGETTDYAAPVKGRF | LPGTAPKLLIYKNNQRPSGVPDRFSGSKSGTSASLAISG |
| | | TISRDDSKSTLYLQMNSLKTEDTAVYYCTSSFCCR | LRSEDEADYYCSTWNDQLSVRVFGGGTKLTVL (SEQ |
| | | GGSCPSHDTSYCGGQYKSYYYMDVWGKGTTVTVSS | ID NO: 1665) |
| | | (SEQ ID NO: 1471) | |
| 140 | AIP-150277 | EVQLVESGGALVKPGGSLRLSCAASGFTFSKAWMS | QSVLTQPPSASGTPGQRVTISCHGSESDIGSHDVLWYQQ |
| | | WVRQAPGKGLEWVGRIKSVTDGETTDYAAPVKGRF | LPGTAPKLLIYKNNQRPSGVPDRFSGSKSGTSASLAISG |
| | | TISRDDSKSTLYLQMNSLKTEDTAVYYCTSSFCCR | LRSEDEADYYCSTWDDSLSVRVFGGGTKLTVL (SEQ |
| | | GGSCPSHDTSYCGGQYKSYYYMDVWGKGTTVTVSS | ID NO: 1666) |
| | | (SEQ ID NO: 1472) | |
| 141 | AIP-104364 | EVQLVESGGALVKPGGSLRLSCAASGFTFSKAWYS | QSVLTQPPSASGTPGQRVTISCSGSSSNIGSSSVSWYQQ |
| | | WVRQAPGKGLEWVGRIKSVTDGETTDYAAPVKGRF | LPGTAPKLLIYKNNQRPSGVPDRFSGSKSGTSASLAISG |
| | | TISRDDSKSTLYLQMNSLKTEDTAVYYCTSSFCCR | LRSEDEADYYCSTWDDSLSVRVFGGGTKLTVL (SEQ |
| | | GGSCPSHDTSYCGGQYKSYYYMDVWGKGTTVTVSS | ID NO: 1667) |
| | | (SEQ ID NO: 1473) | |
| 142 | AIP-180422 | EVQLVESGGGLVQPGGSLRLSCAASGFTFSKAWMS | QSVLTQPPSASGTPGQRVTISCSGSSSNIGSSSVSWYQQ |
| | | WVRQAPGKGLEWVGRIKSVTDGETTEYAASVKGRF | LPGTAPKLLIYKNNQRPSGVPDRFSGSKSGTSASLAISG |
| | | TISRDDSKNSLYLQMNSLKTEDTAVYYCTSSFCCR | LRSEDEADYYCSTWDDSLSVRVFGGGTKLTVL (SEQ |
| | | GGSCPSHDTSYCGGQYKSYYYMDVWGKGTTVTVSS | ID NO: 1668) |
| | | (SEQ ID NO: 1474) | |
| 143 | AIP-166722 | EVQLVESGGGLVQPGGSLRLSCAASGFTFSKAWMS | QSVLTQPPSVSGAPGQRVTISCSGSSSNIGSSSVSWYQQ |
| | | WVRQAPGKGLEWVGRIKSVTDGETTEYAASVKGRF | LPGTAPKLLIYKNNQRPSGVPDRFSGSKSGTSASLAITG |
| | | TISRDDSKNSLYLQMNSLKTEDTAVYYCTSSFCCR | LQAEDEADYYCSTWDDSLSVRVFGGGTKLTVL (SEQ |
| | | GGSCPSHDTSYCGGQYKSYYYMDVWGKGTTVTVSS | ID NO: 1669) |
| | | (SEQ ID NO: 1475) | |
| 144 | AIP-193490 | EVQLVESGGGLVQPGPSLRLSCTASGFTFSKAWMS | QSVLTQPPSVSGAPGQRVTISCSGSSSNIGSSSVSWYQQ |
| | | WVRQAPGKGLEWVGFIKSVTDGETTEYAASVKGRF | LPGTAPKLLIYKNNQRPSGVPDRFSGSKSGTSASLAITG |
| | | TISRDDSKSIAYLQMNSLKTEDTAVYYCTSSFCCR | LQAEDEADYYCSTWDDSLSVRVFGGGTKLTVL (SEQ |
| | | GGSCPSHDTSYCGGQYKSYYYMDVWGKGTTVTVSS | ID NO: 1670) |
| | | (SEQ ID NO: 1476) | |
| 145 | AIP-129967 | EVQLVESGGALVKPGGSLRLSCAASGFTFSKAWMS | QSVLTQPPSASGTPGQRVTISCSGSSSNIGSSSVSWYQQ |
| | | WVRQAPGKGLEWVGRIKSVTDGETTDYAAPVKGRF | LPGTAPKLLIYKNNQRPSGVPDRFSGSKSGTSASLAISG |
| | | TISRDDSKSTLYLQMNSLKTEDTAVYYCTSSYCCR | LRSEDEADYYCSTWDDSLSVRVFGGGTKLTVL (SEQ |
| | | SGSCPSSDTSYCNGQFKSYYYMDPWGKGTTVTVSS | ID NO: 1671) |
| | | (SEQ ID NO: 1477) | |
| 146 | AIP-126175 | EVQLVESGGALVKPGGSLRLSCAASGFTFSKAWMS | QSVLTQPPSASGTPGQRVTISCSGSSSNIGSSSVSWYQQ |
| | | WVRQAPGKGLEWVGRIKSVTDGETTDYAAPVKGRF | LPGTAPKLLIYKNNQRPSGVPDRFSGSKSGTSASLAISG |
| | | TISRDDSKSTLYLQMNSLKTEDTAVYYCTSSFCCR | LRSEDEADYYCSTWDDSLSVRVFGGGTKLTVL (SEQ |
| | | SGSCPSSDTSYCNGQYKSYYYVNIWGKGTTVTVSS | ID NO: 1672) |
| | | (SEQ ID NO: 1478) | |
| 147 | AIP-180905 | EVQLVESGGALVKPGGSLRLSCAASGFTFSKAWMS | QSVLTQPPSASGTPGQRVTISCSGSSSNIGSSSVSWYQQ |
| | | WVRQAPGKGLEWVGRIKSVTDGETTDYAAPVKGRF | LPGTAPKLLIYKNNQRPSGVPDRFSGSKSGTSASLAISG |
| | | TISRDDSKSTLYLQMNSLKTEDTAVYYCTTSFCCR | LRSEDEADYYCSTWDDSLSVRVFGGGTKLTVL (SEQ |
| | | GASCPSHDTSYCAGSYKSYYYVNIWGKGTTVTVS | ID NO: 1673) |
| | | (SEQ ID NO: 1479) | |
| 148 | AIP-141706 | EVQLVESGGALVKPGGSLRLSCAASGFTFSKAWMS | QSVLTQPPSASGTPGQRVTISCSGSSSNIGSSSVSWYQQ |
| | | WVRQAPGKGLEWVGRIKSVTDGETTDYAAPVKGRF | LPGTAPKLLIYKNNQRPSGVPDRFSGSKSGTSASLAISG |
| | | TISRDDSKSTLYLQMNSLKTEDTAVYYCTSAFCCR | LRSEDEADYYCSTWDDSLSVRVFGGGTKLTVL (SEQ |
| | | GKSCPSSDTSYCGGQYPSYYYVNIWGKGTTVTVSS | ID NO: 1674) |
| | | (SEQ ID NO: 1480) | |
| 149 | AIP-105092 | EVQLVESGGALVKPGGSLRLSCAASGFTFSKAWMS | QSVLTQPPSASGTPGQRVTISCSGSSSNIGSSSVSWYQQ |
| | | WVRQAPGKGLEWVGRIKSVTDGETTDYAAPVKGRF | LPGTAPKLLIYKNNQRPSGVPDRFSGSKSGTSASLAISG |
| | | TISRDDSKSTLYLQMNSLKTEDTAVYYCTSAFCCR | LRSEDEADYYCSTWDDSLSVRVFGGGTKLTVL (SEQ |
| | | GKSCPSHDTSFCGGQDKRYYYVNIWGKGTTVTVSS | ID NO: 1675) |
| | | (SEQ ID NO: 1481) | |
| 150 | AIP-152031 | EVQLVESGGALVKPGGSLRLSCAASGFTFSKAWMS | QSVLTQPPSASGTPGQRVTISCSGSSSNIGSSSVSWYQQ |
| | | WVRQAPGKGLEWVGRIKSVTDGETTDYAAPVKGRF | LPGTAPKLLIYKNNQRPSGVPDRFSGSKSGTSASLAISG |
| | | TISRDDSKSTLYLQMNSLKTEDTAVYYCTSAFCCR | LRSEDEADYYCSTWDDSLSVRVFGGGTKLTVL (SEQ |
| | | GGNCPSHETSYCNGQNKQYYYMDVWGKGTTVTVSS | ID NO: 1676) |
| | | (SEQ ID NO: 1482) | |
| 151 | AIP-163319 | EVQLVESGGALVKPGGSLRLSCAASGFTFSKAWMS | QSVLTQPPSASGTPGQRVTISCSGSSSNIGSSSVSWYQQ |
| | | WVRQAPGKGLEWVGRIKSVTDGETTDYAAPVKGRF | LPGTAPKLLIYKNNQRPSGVPDRFSGSKSGTSASLAISG |
| | | TISRDDSKSTLYLQMNSLKTEDTAVYYCTSSFCCR | LRSEDEADYYCSTWDDSLSVRVFGGGTKLTVL (SEQ |
| | | GNQCPSSDTSFCGGQDKRYYYMDPWGKGTTVTVSS | ID NO: 1677) |
| | | (SEQ ID NO: 1483) | |
| 152 | AIP-160621 | EVQLVESGGALVKPGGSLRLSCAASGFTFSKAWMS | QSVLTQPPSASGTPGQRVTISCSGSSSNIGSSSVSWYQQ |
| | | WVRQAPGKGLEWVGRIKSVTDGETTDYAAPVKGRF | LPGTAPKLLIYKNNQRPSGVPDRFSGSKSGTSASLAISG |
| | | TISRDDSKSTLYLQMNSLKTEDTAVYYCTTSFCCR | LRSEDEADYYCSTWDDSLSVRVFGGGTKLTVL (SEQ |
| | | GNQCPSHETSYCGGYYKSYFYMDVWGKGTTVTVSS | ID NO: 1678) |
| | | (SEQ ID NO: 1484) | |
| 153 | AIP-145212 | EVQLVESGGALVKPGGSLRLSCAASGFTFSKAWMS | QSVLTQPPSASGTPGQRVTISCSGSSSNIGSSSVSWYQQ |
| | | WVRQAPGKGLEWVGRIKSVTDGETTDYAAPVKGRF | LPGTAPKLLIYKNNQRPSGVPDRFSGSKSGTSASLAISG |
| | | TISRDDSKSTLYLQMNSLKTEDTAVYYCTSSYCCR | LRSEDEADYYCSTWDDSLSVRVFGGGTKLTVL (SEQ |
| | | GKQCPSHDTSYCAGQYADYYYMDVWGKGTTVTVSS | ID NO: 1679) |
| | | (SEQ ID NO: 1485) | |
| 154 | AIP-112328 | EVQLVESGGALVKPGGSLRLSCAASGFTFSKAWMS | QSVLTQPPSASGTPGQRVTISCSGSSSNIGSSSVSWYQQ |
| | | WVRQAPGKGLEWVGRIKSVTDGETTDYAAPVKGRF | LPGTAPKLLIYKNNQRPSGVPDRFSGSKSGTSASLAISG |
| | | TISRDDSKSTLYLQMNSLKTEDTAVYYCISSFCCR | LRSEDEADYYCSTWDDSLSVRVFGGGTKLTVL (SEQ |
| | | GKQCPSSDTSYCNGQYADYYYMDVWGKGTTVTVSS | ID NO: 1680) |
| | | (SEQ ID NO: 1486) | |
| 155 | AIP-193106 | EVQLVESGGALVKPGGSLRLSCAASGFTFSKAWMS | QSVLTQPPSASGTPGQRVTISCSGSSSNIGSSSVSWYQQ |
| | | WVRQAPGKGLEWVGRIKSVTDGETTDYAAPVKGRF | LPGTAPKLLIYKNNQRPSGVPDRFSGSKSGTSASLAISG |
| | | TISRDDSKSTLYLQMNSLKTEDTAVYYCTSSFCCR | LRSEDEADYYCSTWDDSLSVRVFGGGTKLTVL (SEQ |
| | | GKQCPSSDTSYCNGQYADYYYVNIWGKGTTVTVSS | ID NO: 1681) |
| | | (SEQ ID NO: 1487) | |
| 156 | AIP-125062 | EVQLVESGGALVKPGGSLRLSCAASGFTFSKAWMS | QSVLTQPPSASGTPGQRVTISCSGSSSNIGSSSVSWYQQ |
| | | WVRQAPGKGLEWVGRIKSVTDGETTDYAAPVKGRF | LPGTAPKLLIYKNNQRPSGVPDRFSGSKSGTSASLAISG |
| | | TISRDDSKSTLYLQMNSLKTEDTAVYYCISSFCCR | LRSEDEADYYCSTWDDSLSVRVFGGGTKLTVL (SEQ |
| | | GKQCPSSDTSFCGGQDKRYYFMDVWGKGTTVTVSS | ID NO: 1682) |
| | | (SEQ ID NO: 1488) | |
| 157 | AIP-124301 | EVQLVESGGALVKPGGSLRLSCAASGFTFSKAWMS | QSVLTQPPSASGTPGQRVTISCSGSSSNIGSSSVSWYQQ |
| | | WVRQAPGKGLEWVGRIKSVTDGETTDYAAPVKGRF | LPGTAPKLLIYKNNQRPSGVPDRFSGSKSGTSASLAISG |
| | | TISRDDSKSTLYLQMNSLKTEDTAVYYCTSAFCCR | LRSEDEADYYCSTWDDSLSVRVFGGGTKLTVL (SEQ |
| | | GKQCPSSDTSYCGGQYASFYYMDVWGKGTTVTVSS | ID NO: 1683) |
| | | (SEQ ID NO: 1489) | |
| 158 | AIP-124068 | EVQLVESGGALVKPGGSLRLSCAASGFTFSKAWMS | QSVLTQPPSASGTPGQRVTISCSGSSSNIGSSSVSWYQQ |
| | | WVRQAPGKGLEWVGRIKSVTDGETTDYAAPVKGRF | LPGTAPKLLIYKNNQRPSGVPDRFSGSKSGTSASLAISG |
| | | TISRDDSKSTLYLQMNSLKTEDTAVYYCTSSFCCQ | LRSEDEADYYCSTWDDSLSVRVFGGGTKLTVL (SEQ |
| | | GNNCPSSDTSYCGGYYKDYYYMDVWGKGTTVTVSS | ID NO: 1684) |
| | | (SEQ ID NO: 1490) | |
| 159 | AIP-144568 | EVQLVESGGALVKPGGSLRLSCAASGFTFSKAWMS | QSVLTQPPSASGTPGQRVTISCSGSSSNIGSSSVSWYQQ |
| | | WVRQAPGKGLEWVGRIKSVTDGETTDYAAPVKGRF | LPGTAPKLLIYKNNQRPSGVPDRFSGSKSGTSASLAISG |
| | | TISRDDSKSTLYLQMNSLKTEDTAVYYCTSSFCCQ | LRSEDEADYYCSTWDDSLSVRVFGGGTKLTVL (SEQ |
| | | GNNCPSHDTSYCGGQYKSYYYVNIWGKGTTVTVSS | ID NO: 1685) |
| | | (SEQ ID NO: 1491) | |
| 160 | AIP-139782 | EVQLVESGGALVKPGGSLRLSCAASGFTFSKAWMS | QSVLTQPPSASGTPGQRVTISCSGSSSNIGSSSVSWYQQ |
| | | WVRQAPGKGLEWVGRIKSVTDGETTDYAAPVKGRF | LPGTAPKLLIYKNNQRPSGVPDRFSGSKSGTSASLAISG |
| | | TISRDDSKSTLYLQMNSLKTEDTAVYYCTSSFCCQ | LRSEDEADYYCSTWDDSLSVRVFGGGTKLTVL (SEQ |
| | | GKNCPSHETSYCGGQYADYYYMDVWGKGTTVTVSS | ID NO: 1686) |
| | | (SEQ ID NO: 1492) | |
| 161 | AIP-171543 | EVQLVESGGALVKPGGSLRLSCAASGFTFSKAWMS | QSVLTQPPSASGTPGQRVTISCSGSSSNIGSSSVSWYQQ |
| | | WVRQAPGKGLEWVGRIKSVTDGETTDYAAPVKGRF | LPGTAPKLLIYKNNQRPSGVPDRFSGSKSGTSASLAISG |
| | | TISRDDSKSTLYLQMNSLKTEDTAVYYCTTSFCCH | LRSEDEADYYCSTWDDSLSVRVFGGGTKLTVL (SEQ |
| | | GNSCPSSDTSYCGGQNKQYYYMDVWGKGTTVTVSS | ID NO: 1687) |
| | | (SEQ ID NO: 1493) | |
| 162 | AIP-140148 | EVQLVESGGALVKPGGSLRLSCAASGFTFSKAWMS | QSVLTQPPSASGTPGQRVTISCSGSSSNIGSSSVSWYQQ |
| | | WVRQAPGKGLEWVGRIKSVTDGETTDYAAPVKGRF | LPGTAPKLLIYKNNQRPSGVPDRFSGSKSGTSASLAISG |
| | | TISRDDSKSTLYLQMNSLKTEDTAVYYCISSFCCH | LRSEDEADYYCSTWDDSLSVRVFGGGTKLTVL (SEQ |
| | | GNSCPSHDTSYCNGQNKQYYYMDVWGKGTTVTVSS | ID NO: 1688) |
| | | (SEQ ID NO: 1494) | |
| 163 | AIP-177193 | EVQLVESGGALVKPGGSLRLSCAASGFTFSKAWMS | QSVLTQPPSASGTPGQRVTISCSGSSSNIGSSSVSWYQQ |
| | | WVRQAPGKGLEWVGRIKSVTDGETTDYAAPVKGRF | LPGTAPKLLIYKNNQRPSGVPDRFSGSKSGTSASLAISG |
| | | TISRDDSKSTLYLQMNSLKTEDTAVYYCISSFCCH | LRSEDEADYYCSTWDDSLSVRVFGGGTKLTVL (SEQ |
| | | SNNCPSSDTSYCNGQYKQYYYMDVWGKGTTVTVSS | ID NO: 1689) |
| | | (SEQ ID NO: 1495) | |
| 164 | AIP-171348 | EVQLVESGGALVKPGGSLRLSCAASGFTFSKAWMS | QSVLTQPPSASGTPGQRVTISCSGSSSNIGSSSVSWYQQ |
| | | WVRQAPGKGLEWVGRIKSVTDGETTDYAAPVKGRF | LPGTAPKLLIYKNNQRPSGVPDRFSGSKSGTSASLAISG |
| | | TISRDDSKSTLYLQMNSLKTEDTAVYYCTSSFCCH | LRSEDEADYYCSTWDDSLSVRVFGGGTKLTVL (SEQ |
| | | SNNCPSHDTSYCGGQYKSYYYVNIWGKGTTVTVSS | ID NO: 1690) |
| | | (SEQ ID NO: 1496) | |
| 165 | AIP-193088 | EVQLVESGGALVKPGGSLRLSCAASGFTFSKAWMS | QSVLTQPPSASGTPGQRVTISCSGSSSNIGSSSVSWYQQ |
| | | WVRQAPGKGLEWVGRIKSVTDGETTDYAAPVKGRF | LPGTAPKLLIYKNNQRPSGVPDRFSGSKSGTSASLAISG |
| | | TISRDDSKSTLYLQMNSLKTEDTAVYYCISSFACH | LRSEDEADYYCSTWDDSLSVRVFGGGTKLTVL (SEQ |
| | | SNNCPSSDTSYVNGYYKQYYFMDVWGKGTTVTVSS | ID NO: 1691) |
| | | (SEQ ID NO: 1497) | |
| 166 | AIP-182087 | EVQLVESGGALVKPGGSLRLSCAASGFTFSKAWMS | QSVLTQPPSASGTPGQRVTISCSGSSSNIGSSSVSWYQQ |
| | | WVRQAPGKGLEWVGRIKSVTDGETTDYAAPVKGRF | LPGTAPKLLIYKNNQRPSGVPDRFSGSKSGTSASLAISG |
| | | TISRDDSKSTLYLQMNSLKTEDTAVYYCTSSFCCH | LRSEDEADYYCSTWDDSLSVRVFGGGTKLTVL (SEQ |
| | | SNNCPSSDTSYCNGQYKQYYYVNIWGKGTTVTVSS | ID NO: 1692) |
| | | (SEQ ID NO: 1498) | |
| 167 | AIP-151388 | EVQLVESGGALVKPGGSLRLSCAASGFTFSKAWMS | QSVLTQPPSASGTPGQRVTISCSGSSSNIGSSSVSWYQQ |
| | | WVRQAPGKGLEWVGRIKSVTDGETTDYAAPVKGRF | LPGTAPKLLIYKNNQRPSGVPDRFSGSKSGTSASLAISG |
| | | TISRDDSKSTLYLQMNSLKTEDTAVYYCTSSFCCL | LRSEDEADYYCSTWDDSLSVRVFGGGTKLTVL (SEQ |
| | | GKACLSHDTSYCGGQYASYYYVNIWGKGTTVTVSS | ID NO: 1693) |
| | | (SEQ ID NO: 1499) | |
| 168 | AIP-149787 | EVQLVESGGALVKPGGSLRLSCAASGFTFSKAWMS | QSVLTQPPSASGTPGQRVTISCSGSSSNIGSSSVSWYQQ |
| | | WVRQAPGKGLEWVGRIKSVTDGETTDYAAPVKGRF | LPGTAPKLLIYKNNQRPSGVPDRFSGSKSGTSASLAISG |
| | | TISRDDSKSTLYLQMNSLKTEDTAVYYCTSSFCCL | LRSEDEADYYCSTWDDSLSVRVFGGGTKLTVL (SEQ |
| | | GKACLSSDTSYCGGYYASYYFVNIWGKGTTVTVSS | ID NO: 1694) |
| | | (SEQ ID NO: 1500) | |
| 169 | AIP-126097 | EVQLVESGGALVKPGGSLRLSCAASGFTFSKAWMS | QSVLTQPPSASGTPGQRVTISCSGSSSNIGSSSVSWYQQ |
| | | WVRQAPGKGLEWVGRIKSVTDGETTDYAAPVKGRF | LPGTAPKLLIYKNNQRPSGVPDRFSGSKSGTSASLAISG |
| | | TISRDDSKSTLYLQMNSLKTEDTAVYYCTSSFACL | LRSEDEADYYCSTWDDSLSVRVFGGGTKLTVL (SEQ |
| | | GKACLSSDTSYVGGYYASYYFVNIWGKGTTVTVSS | ID NO: 1695) |
| | | (SEQ ID NO: 1501) | |
| 170 | AIP-107759 | EVQLVESGGALVKPGGSLRLSCAASGFTFSKAWMS | QSVLTQPPSASGTPGQRVTISCSGSSSNIGSSSVSWYQQ |
| | | WVRQAPGKGLEWVGRIKSVTDGETTDYAAPVKGRF | LPGTAPKLLIYKNNQRPSGVPDRFSGSKSGTSASLAISG |
| | | TISRDDSKSTLYLQMNSLKTEDTAVYYCTSSYCCL | LRSEDEADYYCSTWDDSLSVRVFGGGTKLTVL (SEQ |
| | | GNSCPSSDTSYCGGQFKSYYYMDPWGKGTTVTVSS | ID NO: 1696) |
| | | (SEQ ID NO: 1502) | |
| 171 | AIP-148484 | EVQLVESGGALVKPGGSLRLSCAASGFTFSKAWMS | QSVLTQPPSASGTPGQRVTISCSGSSSNIGSSSVSWYQQ |
| | | WVRQAPGKGLEWVGRIKSVTDGETTDYAAPVKGRF | LPGTAPKLLIYKNNQRPSGVPDRFSGSKSGTSASLAISG |
| | | TISRDDSKSTLYLQMNSLKTEDTAVYYCTSSYCCL | LRSEDEADYYCSTWDDSLSVRVFGGGTKLTVL (SEQ |
| | | GNSCPSSDTSFCGGQDKRYYYMDPWGKGTTVTVSS | ID NO: 1697) |
| | | (SEQ ID NO: 1503) | |
| 172 | AIP-186403 | EVQLVESGGALVKPGGSLRLSCAASGFTFSKAWMS | QSVLTQPPSASGTPGQRVTISCSGSSSNIGSSSVSWYQQ |
| | | WVRQAPGKGLEWVGRIKSVTDGETTDYAAPVKGRF | LPGTAPKLLIYKNNQRPSGVPDRFSGSKSGTSASLAISG |
| | | TISRDDSKSTLYLQMNSLKTEDTAVYYCISSFCCL | LRSEDEADYYCSTWDDSLSVRVFGGGTKLTVL (SEQ |
| | | GGNCPSSETSYCGNQYPSYYYMDVWGKGTTVTVSS | ID NO: 1698) |
| | | (SEQ ID NO: 1504) | |
| 173 | AIP-166629 | EVQLVESGGALVKPGGSLRLSCAASGFTFSKAWMS | QSVLTQPPSASGTPGQRVTISCSGSSSNIGSSSVSWYQQ |
| | | WVRQAPGKGLEWVGRIKSVTDGETTDYAAPVKGRF | LPGTAPKLLIYKNNQRPSGVPDRFSGSKSGTSASLAISG |
| | | TISRDDSKSTLYLQMNSLKTEDTAVYYCISSFCCL | LRSEDEADYYCSTWDDSLSVRVFGGGTKLTVL (SEQ |
| | | GGNCPSSETSYCGNYYPSYFYMDVWGKGTTVTVSS | ID NO: 1699) |
| | | (SEQ ID NO: 1505) | |
| 174 | AIP-165276 | EVQLVESGGALVKPGGSLRLSCAASGFTFSKAWMS | QSVLTQPPSASGTPGQRVTISCSGSSSNIGSSSVSWYQQ |
| | | WVRQAPGKGLEWVGRIKSVTDGETTDYAAPVKGRF | LPGTAPKLLIYKNNQRPSGVPDRFSGSKSGTSASLAISG |
| | | TISRDDSKSTLYLQMNSLKTEDTAVYYCTSSFCCL | LRSEDEADYYCSTWDDSLSVRVFGGGTKLTVL (SEQ |
| | | GGNCPSSETSYCGNQYPSYYYVNIWGKGTTVTVSS | ID NO: 1700) |
| | | (SEQ ID NO: 1506) | |
| 175 | AIP-188293 | EVQLVESGGALVKPGGSLRLSCAASGFTFSKAWMS | QSVLTQPPSASGTPGQRVTISCSGSSSNIGSSSVSWYQQ |
| | | WVRQAPGKGLEWVGRIKSVTDGETTDYAAPVKGRF | LPGTAPKLLIYKNNQRPSGVPDRFSGSKSGTSASLAISG |
| | | TISRDDSKSTLYLQMNSLKTEDTAVYYCTTSFACR | LRSEDEADYYCSTWDDSLSVRVFGGGTKLTVL (SEQ |
| | | GNQCPSHETSYVGGYYKSYFYMDVWGKGTTVTVSS | ID NO: 1701) |
| | | (SEQ ID NO: 1507) | |
| 176 | AIP-109364 | EVQLVESGGALVKPGGSLRLSCAASGFTFSKAWMS | QSVLTQPPSASGTPGQRVTISCSGSSSNIGSSSVSWYQQ |
| | | WVRQAPGKGLEWVGRIKSVTDGETTDYAAPVKGRF | LPGTAPKLLIYKNNQRPSGVPDRFSGSKSGTSASLAISG |
| | | TISRDDSKSTLYLQMNSLKTEDTAVYYCTTSFACR | LRSEDEADYYCSTWDDSLSVRVFGGGTKLTVL (SEQ |
| | | GNQCPSSETSYVGGYYKSYFFMDVWGKGTTVTVSS | ID NO: 1702) |
| | | (SEQ ID NO: 1508) | |
| 177 | AIP-191805 | EVQLVESGGALVKPGGSLRLSCAASGFTFSKAWMS | QSVLTQPPSASGTPGQRVTISCSGSSSNIGSSSVSWYQQ |
| | | WVRQAPGKGLEWVGRIKSVTDGETTDYAAPVKGRF | LPGTAPKLLIYKNNQRPSGVPDRFSGSKSGTSASLAISG |
| | | TISRDDSKSTLYLQMNSLKTEDTAVYYCISSFACR | LRSEDEADYYCSTWDDSLSVRVFGGGTKLTVL (SEQ |
| | | GKQCPSSDTSYVGGQFKSYYFMDVWGKGTTVTVSS | ID NO: 1703) |
| | | (SEQ ID NO: 1509) | |
| 178 | AIP-181592 | EVQLVESGGALVKPGGSLRLSCAASGFTFAKAWMS | QSVLTQPPSASGTPGQRVTISCSGSSTNIGSSSVYWYQQ |
| | | WVRQAPGKGLEWVGRIKAADDGKQTDYAAPVKGRF | LPGTAPKLLIYKNNYRPSGVPDRFSGSKSGTSASLAISG |
| | | TISRDDSKSTLYLQMNSLKTEDTAVYYCTSSFCCR | LRSEDEADYYCSTWDDSLSVRVFGGGTKLTVL (SEQ |
| | | GGSCLSHDTSYCGGQYKSYYWMDVWGKGTTVTVSS | ID NO: 1704) |
| | | (SEQ ID NO: 1510) | |
| 179 | AIP-155587 | EVQLVESGGALVKPGGSLRLSCAASGFTYAKAWMS | QSVLTQPPSASGTPGQRVTISCSGSSTNIGSSSVYWYQQ |
| | | WVRQAPGKGLEWVGRIKSVQDGEQTDYAAPVKGRF | LPGTAPKLLIYKNNYRASGVPDRFSGSKSGTSASLAISG |
| | | TISRDDSKSTLYLQMNSLKTEDTAVYYCTSSYCCL | LRSEDEADYYCSTWDDSLSVRVFGGGTKLTVL (SEQ |
| | | GGSCPSHDTSYCGGSYKSYYYMDVWGKGTTVTVSS | ID NO: 1705) |
| | | (SEQ ID NO: 1511) | |
| 180 | AIP-147945 | EVQLVESGGALVKPGGSLRLSCAASGFDASKAWFT | QSVLTQPPSASGTPGQRVTISCTGSSSNIGSSYVSWYQQ |
| | | WVRQAPGKGLEWVGRIKSVTDGETTDYAAPVKGRF | LPGTAPKLLIYKNSLRPQGVPDRFSGSKSGTSASLAISG |
| | | TISRDDSKSTLYLQMNSLKTEDTAVYYCTSSFCCR | LRSEDEADYYCSTWDDSLSVRVFGGGTKLTVL (SEQ |
| | | GGSCPSHDTSYCGGQNKQYYYMDVWGKGTTVTVSS | ID NO: 1706) |
| | | (SEQ ID NO: 1512) | |
| 181 | AIP-199264 | EVQLVESGGALVKPGGSLRLSCAASGFDASKAWFT | QSVLTQPPSASGTPGQRVTISCTGSSSNIGSSYVSWYQQ |
| | | WVRQAPGKGLEWVGRIKSVTDGETTDYAAPVKGRF | LPGTAPKLLIYKNSLRPQGVPDRFSGSKSGTSASLAISG |
| | | TISRDDSKSTLYLQMNSLKTEDTAVYYCTSSFACR | LRSEDEADYYCSTWDDSLSVRVFGGGTKLTVL (SEQ |
| | | GGSCPSHDTSYVGGQYKSYYYMDVWGKGTTVTVSS | ID NO: 1707) |
| | | (SEQ ID NO: 1513) | |
| 182 | AIP-123438 | EVQLVESGGALVKPGGSLRLSCAASGFDASKAWFT | QSVLTQPPSASGTPGQRVTISCTGSSSNIGSSYVSWYQQ |
| | | WVRQAPGKGLEWVGRIKSVTDGETTDYAAPVKGRF | LPGTAPKLLIYKNSLRPQGVPDRFSGSKSGTSASLAISG |
| | | TISRDDSKSTLYLQMNSLKTEDTAVYYCTSSYCCL | LRSEDEADYYCSTWDDSLSVRVFGGGTKLTVL (SEQ |
| | | GGSCPSHDTSYCGGSYKSYYYMDVWGKGTTVTVSS | ID NO: 1708) |
| | | (SEQ ID NO: 1514) | |
| 183 | AIP-151315 | EVQLVESGGALVKPGGSLRLSCAASGFDASKAWFT | QSVLTQPPSASGTPGQRVTISCTGSSSNIGSSYVSWYQQ |
| | | WVRQAPGKGLEWVGRIKSVTDGETTDYAAPVKGRF | LPGTAPKLLIYKNSLRPQGVPDRFSGSKSGTSASLAISG |
| | | TISRDDSKSTLYLQMNSLKTEDTAVYYCTSSFCCR | LRSEDEADYYCSTWDDSLSVRVFGGGTKLTVL (SEQ |
| | | GKSCPSHDTSYCGGQYASYYYMDVWGKGTTVTVSS | ID NO: 1709) |
| | | (SEQ ID NO: 1515) | |
| 184 | AIP-197785 | EVQLVESGGALVKPGGSLRLSCAASGFTFSKAWMS | QSVLTQPPSASGTPGQRVTISCSGSPSNIGSASTSWYQQ |
| | | WVRQAPGKGLEWVGRIKAADDGKQTDYAAPVKGRF | LPGTAPKLLIYKNNQRPSGVPDRFSGSKSGTSASLAISG |
| | | TISRDDSKSTLYLQMNSLKTEDTAVYYCTSSFCCR | LRSEDEADYYCSTWDDSLSVRVFGGGTKLTVL (SEQ |
| | | GGNCPSHETSYCGNQYKSYYYMDVWGKGTTVTVSS | ID NO: 1710) |
| | | (SEQ ID NO: 1516) | |
| 185 | AIP-115782 | EVQLVESGGALVKPGGSLRLSCAASGFTFSKAWMS | QSVLTQPPSASGTPGQRVTISCSGSPSNIGSASTSWYQQ |
| | | WVRQAPGKGLEWVGRIKAADDGKQTDYAAPVKGRF | LPGTAPKLLIYKNNQRPSGVPDRFSGSKSGTSASLAISG |
| | | TISRDDSKSTLYLQMNSLKTEDTAVYYCTSSFCCR | LRSEDEADYYCSTWDDSLSVRVFGGGTKLTVL (SEQ |
| | | SGSCPSSDTSYCNGQYKSYYYMDVWGKGTTVTVSS | ID NO: 1711) |
| | | (SEQ ID NO: 1517) | |
| 186 | AIP-138130 | EVQLVESGGALVKPGGSLRLSCAASGFTFSKAWMS | QSVLTQPPSASGTPGQRVTISCSGSPSNIGSASTSWYQQ |
| | | WVRQAPGKGLEWVGRIKAADDGKQTDYAAPVKGRF | LPGTAPKLLIYKNNQRPSGVPDRFSGSKSGTSASLAISG |
| | | TISRDDSKSTLYLQMNSLKTEDTAVYYCTSSFCCR | LRSEDEADYYCSTWDDSLSVRVFGGGTKLTVL (SEQ |
| | | GGSCPSHDTSYCGGQYKSYYYVNIWGKGTTVTVSS | ID NO: 1712) |
| | | (SEQ ID NO: 1518) | |
| 187 | AIP-170221 | EVQLVESGGALVKPGGSLRLSCAASGFTFSKAWMS | QSVLTQPPSASGTPGQRVTISCSGSPSNIGSASTSWYQQ |
| | | WVRQAPGKGLEWVGRIKAADDGKQTDYAAPVKGRF | LPGTAPKLLIYKNNQRPSGVPDRFSGSKSGTSASLAISG |
| | | TISRDDSKSTLYLQMNSLKTEDTAVYYCTTSFCCR | LRSEDEADYYCSTWDDSLSVRVFGGGTKLTVL (SEQ |
| | | GASCPSHDTSYCAGSYKSYYYMDVWGKGTTVTVSS | ID NO: 1713) |
| | | (SEQ ID NO: 1519) | |
| 188 | AIP-167482 | EVQLVESGGALVKPGGSLRLSCAASGFTFSKAWMS | QSVLTQPPSASGTPGQRVTISCSGSPSNIGSASTSWYQQ |
| | | WVRQAPGKGLEWVGRIKAADDGKQTDYAAPVKGRF | LPGTAPKLLIYKNNQRPSGVPDRFSGSKSGTSASLAISG |
| | | TISRDDSKSTLYLQMNSLKTEDTAVYYCISSFCCR | LRSEDEADYYCSTWDDSLSVRVFGGGTKLTVL (SEQ |
| | | GNSCPSSDTSYCNGQYKSYYYMDVWGKGTTVTVSS | ID NO: 1714) |
| | | (SEQ ID NO: 1520) | |
| 189 | AIP-189475 | EVQLVESGGALVKPGGSLRLSCAASGFTFSKAWMS | QSVLTQPPSASGTPGQRVTISCSGASSNIGHSSVYWYQQ |
| | | WVRQAPGKGLEWVGRIKAVTDGHTTDYAAPVKGRF | LPGTAPKLLIYRNNQQPLGVPDRFSGSKSGTSASLAISG |
| | | TISRDDSKSTLYLQMNSLKTEDTAVYYCTSSFCCR | LRSEDEADYYCSTWDDALSIRVFGGGTKLTVL (SEQ |
| | | GGSCPSHDTSYCGGQYKSYYYMDVWGKGTTVTVSS | ID NO: 1715) |
| | | (SEQ ID NO: 1521) | |
| 190 | AIP-102833 | EVQLVESGGALVKPGGSLRLSCAASGFVYSKAWMS | QSVLTQPPSASGTPGQRVTISCSGSSSNIGSSSVSWYQQ |
| | | WVRQAPGKGLEWVGRIKSVTDGQATDYAAPVKGRF | LPGTAPKLLIYKNTQRASGVPDRFSGSKSGTSASLAISG |
| | | TISRDDSKSTLYLQMNSLKTEDTAVYYCTSSFCCR | LRSEDEADYYCSTWDDALNVRVFGGGTKLTVL (SEQ |
| | | GGSCPSHDTSYCGGQYKSYYYMDVWGKGTTVTVSS | ID NO: 1716) |
| | | (SEQ ID NO: 1522) | |
| 191 | AIP-173396 | EVQLVESGGALVKPGGSLRLSCAASGFVFSKAWMS | QSVLTQPPSASGTPGQRVTISCSGSSSNIGSSSVSWYQQ |
| | | WVRQAPGKGLEWVGRIKSVTDGETTDYAAPVKGRF | LPGTAPKLLIYRNNQQPLGVPDRFSGSKSGTSASLAISG |
| | | TISRDDSKSTLYLQMNSLKTEDTAVYYCTSSFCCR | LRSEDEADYYCSSWDDSSLVRVFGGGTKLTVL (SEQ |
| | | GKSCPSHDTSYCGGQYASYYYMDVWGKGTTVTVSS | ID NO: 1717) |
| | | (SEQ ID NO: 1523) | |
| 192 | AIP-132355 | EVQLVESGGALVKPGGSLRLSCAASGFTFAKAWMS | QSVLTQPPSASGTPGQRVTISCSGSSSNIGSSSVTWYQQ |
| | | WVRQAPGKGLEWVGRIKSVTDAETTDYAAPVKGRF | LPGTAPKLLIYKNNARPSGVPDRFSGSKSGTSASLAISG |
| | | TISRDDSKSTLYLQMNSLKTEDTAVYYCTSSFCCR | LRSEDEADYYCATWDNSLSIRVFGGGTKLTVL (SEQ |
| | | SGSCPSSDTSYCNGQYKSYYWMDVWGKGTTVTVSS | ID NO: 1718) |
| | | (SEQ ID NO: 1524) | |
| 193 | AIP-118505 | EVQLVESGGALVKPGGSLRLSCAASGFTYAKAWFT | QSVLTQPPSASGTPGQRVTISCSGSKSNIGSSYVSWYQQ |
| | | WVRQAPGKGLEWVGRIKSTSDGGITDYAAPVKGRF | LPGTAPKLLIYKDNQRASGVPDRFSGSKSGTSASLAISG |
| | | TISRDDSKSTLYLQMNSLKTEDTAVYYCTSSFCCR | LRSEDEADYYCSTWDDALSVRVFGGGTKLTVL (SEQ |
| | | GGSCPSHDTSFCGGQYKSYYYMDVWGKGTTVTVSS | ID NO: 1719) |
| | | (SEQ ID NO: 1525) | |
| 194 | AIP-131972 | EVQLVESGGALVKPGGSLRLSCAASGFTFSKAWMS | QSVLTQPPSASGTPGQRVTISCSGSSSNIGSSSVYWYQQ |
| | | WVRQAPGKGLEWVGRIKSVSDGETTDYAAPVKGRF | LPGTAPKLLIYKNNQRPSGVPDRFSGSKSGTSASLAISG |
| | | TISRDDSKSTLYLQMNSLKTEDTAVYYCTSSFCCR | LRSEDEADYYCSTWDDSLSVRVFGGGTKLTVL (SEQ |
| | | GGSCPSRDTSYCGGQYKSYYFMDVWGKGTTVTVS | ID NO: 1720) |
| | | (SEQ ID NO: 1526) | |
| 195 | AIP-189526 | EVQLVESGGGLVKPGGSLRLSCAASGFTFSKAWMT | QSVLTQPPSASGTPGQRVTISCSGSSSNIGSSSVSWYQQ |
| | | WVRQAPGKGLEWVGRIKSTSDGGITDYAAPVKGRF | LPGTAPKLLIYKNNQRPSGVPDRFSGSKSGTSASLAISG |
| | | TISRDDSKNTLYLQMNSLKTEDTAVYYCTTSFCCR | LRSEDEADYYCSTWDDSLSVRVFGGGTKLTVL (SEQ |
| | | GGRCPSRDTSFCGGQYNSYYYMDVWGQGTTVTVSS | ID NO: 1726 |
| | | (SEQ ID NO: 1725) | |

TABLE 2B

| | AIP | | | | | | |
|---|---|---|---|---|---|---|---|
| | Number | HCDR1 | HCDR2 | HCDR3 | LCDR1 | LCDR2 | LCDR3 |
| 1 | AIP- | GFTFSKAWMT | RIKSVTEGET | TSSFCCRGGS | SGSSSNIGSS | KNNQRPS | STWDESLSVR |
| | 148327 | (SEQ ID | TDYAAPVKG | CPSHDTSFCG | SVS | (SEQ ID | V |
| | | NO: 169) | (SEQ ID | GODKRYYYMD | (SEQ ID | NO: 945) | (SEQ ID |
| | | | NO: 363) | V | NO: 751) | | NO: 1139) |
| | | | | (SEQ ID | | | |
| | | | | NO: 557) | | | |
| 2 | AIP- | GFTFSKAWMT | RIKSVTEGET | TSSFCCRGGS | SGSSSNIGSS | KNNORPS | STWDESLSVR |
| | 198092 | (SEQ ID | TDYAAPVKG | CPSHDTSFCG | SV | (SEQ ID | V |
| | | NO: 170) | (SEQ ID | GODKRYYYMD | (SEQ ID | NO: 946) | (SEQ ID |
| | | | NO: 364) | SV | NO: 752) | | NO: 1140) |
| | | | | (SEQ ID | | | |
| | | | | NO: 558) | | | |
| 3 | AIP- | GFTFSKAWMT | RIKSVTDGET | TSSFCCRGGS | SGSSSNIGSS | KNNQRPS | STWDESLSVR |
| | 184490 | (SEQ ID | TDYAAPVKG | CPSHDTSFCG | SV | (SEQ ID | V |
| | | NO: 171) | (SEQ ID | GSYKSYYYMD | (SEQ ID | NO: 947) | (SEQ ID |
| | | | NO: 365) | SV | NO: 753) | | NO: 1141) |
| | | | | (SEQ ID | | | |
| | | | | NO: 559) | | | |
| 4 | AIP- | GFTFSKAWMS | RIKSVTDGET | TSSFCCRGGS | SGSSSNIGSS | KNNORPS | STWDESLSVR |
| | 102396 | (SEQ ID | TDYAAPVKG | CPSHDTSYCG | SV | (SEQ ID | V |
| | | NO: 172) | (SEQ ID | GQYKSYYYMD | (SEQ ID | NO: 948) | (SEQ ID |
| | | | NO: 366) | SV | NO: 754) | | NO: 1142) |
| | | | | (SEQ ID | | | |
| | | | | NO: 560) | | | |
| 5 | AIP- | GYTFSKAWMS | RIKSVTDGET | TSSFCCRGGS | SGSSSNIGSS | KNNORPS | STWDDSLSVR |
| | 150055 | (SEQ ID | TDYAAPVKG | CPSHDTSYCG | SV | (SEQ ID | V |
| | | NO: 173) | (SEQ ID | GQYKSYYYMD | (SEQ ID | NO: 949) | (SEQ ID |
| | | | NO: 367) | SV | NO: 755) | | NO: 1143) |
| | | | | (SEQ ID | | | |
| | | | | NO: 561) | | | |
| 6 | AIP- | GFDFSKAWMS | RIKSVTDGET | TSSFCCRGGS | SGSSSNIGSS | KNNQRPS | STWDDSLSVR |
| | 167084 | (SEQ ID | TDYAAPVKG | CPSHDTSYCG | SVS | (SEQ ID | V |
| | | NO: 174) | (SEQ ID | GQYKSYYYMD | (SEQ ID | NO: 950) | (SEQ ID |
| | | | NO: 368) | V | NO: 756) | | NO: 1144) |
| | | | | (SEQ ID | | | |
| | | | | NO: 562) | | | |
| 7 | AIP- | GFVFSKAWMS | RIKSVTDGET | TSSFCCRGGS | SGSSSNIGSS | KNNQRPS | STWDDSLSVR |
| | 185304 | (SEQ ID | TDYAAPVKG | CPSHDTSYCG | SVS | (SEQ ID | V |
| | | NO: 175) | (SEQ ID | GQYKSYYYMD | (SEQ ID | NO: 951) | (SEQ ID |
| | | | NO: 369) | | NO: 757) | | NO: 1145) |
| 8 | AIP- | GFTYSKAWMS | RIKSVTDGET | TSSFCCRGGS | SGSSSNIGSS | KNNORPS | STWDDSLSVR |
| | 134770 | (SEQ ID | TDYAAPVKG | CPSHDTSYCG | SV | (SEQ ID | V |
| | | NO: 176) | (SEQ ID | GQYKSYYYMD | (SEQ ID | NO: 952) | (SEQ ID |
| | | | NO: 370) | SV | NO: 758) | | NO: 1146) |
| | | | | (SEQ ID | | | |
| | | | | NO: 564) | | | |
| 9 | AIP- | GFTFAKAWMS | RIKSVTDGET | TSSFCCRGGS | SGSSSNIGSS | KNNORPS | STWDDSLSVR |
| | 141887 | (SEQ ID | TDYAAPVKG | CPSHDTSYCG | SV | (SEQ ID | V |
| | | NO: 177) | (SEQ ID | GQYKSYYYMD | (SEQ ID | NO: 953) | (SEQ ID |
| | | | NO: 371) | SV | NO: 759) | | NO: 1147) |
| | | | | (SEQ ID | | | |
| | | | | NO: 565) | | | |
| 10 | AIP- | GFTFSNAWMS | RIKSVTDGET | TSSFCCRGGS | SGSSSNIGSS | KNNORPS | STWDDSLSVR |
| | 196203 | (SEQ ID | TDYAAPVKG | CPSHDTSYCG | SV | (SEQ ID | V |
| | | NO: 178) | (SEQ ID | GQYKSYYYMD | (SEQ ID | NO: 954) | (SEQ ID |
| | | | NO: 372) | SV | NO: 760) | | NO: 1148) |
| | | | | (SEQ ID | | | |
| | | | | NO: 566) | | | |
| 11 | AIP- | GFTFSRAWMS | RIKSVTDGET | TSSFCCRGGS | SGSSSNIGSS | KNNORPS | STWDDSLSVR |
| | 184151 | (SEQ ID | TDYAAPVKG | CPSHDTSYCG | SV | (SEQ ID | V |
| | | NO: 179) | (SEQ ID | GQYKSYYYMD | (SEQ ID | NO: 955) | (SEQ ID |
| | | | NO: 373) | SV | NO: 761) | | NO: 1149) |
| | | | | (SEQ ID | | | |
| | | | | NO: 567) | | | |
| 12 | AIP- | GFTFSMAWMS | RIKSVTDGET | TSSFCCRGGS | SGSSSNIGSS | KNNQRPS | STWDDSLSVR |
| | 128195 | (SEQ ID | TDYAAPVKG | CPSHDTSYCG | SV | (SEQ ID | V |
| | | NO: 180) | (SEQ ID | GQYKSYYYMD | (SEQ ID | NO: 956) | (SEQ ID |
| | | | NO: 374) | SV | NO: 762) | | NO: 1150) |
| | | | | (SEQ ID | | | |
| | | | | NO: 568) | | | |
| 13 | AIP- | GFTFSAAWMS | RIKSVTDGET | TSSFCCRGGS | SGSSSNIGSS | KNNORPS | STWDDSLSVR |
| | 116579 | (SEQ ID | TDYAAPVKG | CPSHDTSYCG | SV | (SEQ ID | V |
| | | NO: 181) | (SEQ ID | GQYKSYYYMD | (SEQ ID | NO: 957) | (SEQ ID |
| | | | NO: 375) | SV | NO: 763) | | NO: 1151) |
| | | | | (SEQ ID | | | |
| | | | | NO: 569) | | | |
| 14 | AIP- | GFTFSHAWMS | RIKSVTDGET | TSSFCCRGGS | SGSSSNIGSS | KNNQRPS | STWDDSLSVR |
| | 192329 | (SEQ ID | TDYAAPVKG | CPSHDTSYCG | SVS | (SEQ ID | V |
| | | NO: 182) | (SEQ ID | GQYKSYYYMD | (SEQ ID | NO: 958) | (SEQ ID |
| | | | NO: 376) | V | NO: 764) | | NO: 1152) |
| | | | | (SEQ ID | | | |
| | | | | NO: 570) | | | |
| 15 | AIP- | GFTFSHAWMS | RIKSVTDGET | TSSFCCRGGS | SGSSSNIGSS | KNNORPS | STWDDSLSVR |
| | 192329 | (SEQ ID | TDYAAPVKG | CPSHDTSYCG | SV | (SEQ ID | V |
| | | NO: 183) | (SEQ ID | GQYKSYYYMD | (SEQ ID | NO: 959) | (SEQ ID |
| | | | NO: 377) | SV | NO: 765) | | NO: 1153) |
| | | | | (SEQ ID | | | |
| | | | | NO: 571) | | | |
| 16 | AIP- | GFTFSKAYMS | RIKSVTDGET | TSSFCCRGGS | SGSSSNIGSS | KNNQRPS | STWDDSLSVR |
| | 197809 | (SEQ ID | TDYAAPVKG | CPSHDTSYCG | SV | (SEQ ID | V |
| | | NO: 184) | (SEQ ID | GQYKSYYYMD | (SEQ ID | NO: 960) | (SEQ ID |
| | | | NO: 378) | SV | NO: 766) | | NO: 1154) |
| | | | | (SEQ ID | | | |
| | | | | NO: 572) | | | |
| 17 | AIP- | GFTFSKAWFS | RIKSVTDGET | TSSFCCRGGS | SGSSSNIGSS | KNNQRPS | STWDDSLSVR |
| | 142489 | (SEQ ID | TDYAAPVKG | CPSHDTSYCG | SV | (SEQ ID | V |
| | | NO: 185) | (SEQ ID | GQYKSYYYMD | (SEQ ID | NO: 961) | (SEQ ID |
| | | | NO: 379) | SV | NO: 767) | | NO: 1155) |
| | | | | (SEQ ID | | | |
| | | | | NO: 573) | | | |
| 18 | AIP- | GFTFSKAWMS | RIQSVTDGET | TSSFCCRGGS | SGSSSNIGSS | KNNORPS | STWDDSLSVR |
| | 148102 | (SEQ ID | TDYAAPVKG | CPSHDTSYCG | SVS | (SEQ ID | V |
| | | NO: 186) | (SEQ ID | GQYKSYYYMD | (SEQ ID | NO: 962) | (SEQ ID |
| | | | NO: 380) | V | NO: 768) | | NO: 1156) |
| | | | | (SEQ ID | | | |
| | | | | NO: 574) | | | |
| 19 | AIP- | GFTFSKAWMS | RIKAVTDGET | TSSFCCRGGS | SGSSSNIGSS | KNNQRPS | STWDDSLSVR |
| | 167726 | (SEQ ID | TDYAAPVKG | CPSHDTSYCG | SV | (SEQ ID | V |
| | | NO: 187) | (SEQ ID | GQYKSYYYMD | (SEQ ID | NO: 963) | (SEQ ID |
| | | | NO: 381) | SV | NO: 769) | | NO: 1157) |
| | | | | (SEQ ID | | | |
| | | | | NO: 575) | | | |
| 20 | AIP- | GFTFSKAWMS | RIKSATDGET | TSSFCCRGGS | SGSSSNIGSS | KNNQRPS | STWDDSLSVR |
| | 199834 | (SEQ ID | TDYAAPVKG | CPSHDTSYCG | SV | (SEQ ID | V |
| | | NO: 188) | (SEQ ID | GQYKSYYYMD | (SEQ ID | NO: 964) | (SEQ ID |
| | | | NO: 382) | SV | NO: 770) | | NO: 1158) |
| | | | | (SEQ ID | | | |
| | | | | NO: 576) | | | |
| 21 | AIP- | GFTFSKAWMS | RIKSNTDGET | TSSFCCRGGS | SGSSSNIGSS | KNNQRPS | STWDDSLSVR |
| | 143179 | (SEQ ID | TDYAAPVKG | CPSHDTSYCG | SVS | (SEQ ID | V |
| | | NO: 189) | (SEQ ID | GQYKSYYYMD | (SEQ ID | NO: 965) | (SEQ ID |
| | | | NO: 383) | V | NO: 771) | | NO: 1159) |
| | | | | (SEQ ID | | | |
| | | | | NO: 577) | | | |
| 22 | AIP- | GFTFSKAWMS | RIKSVHDGET | TSSFCCRGGS | SGSSSNIGSS | KNNORPS | STWDDSLSVR |
| | 195587 | (SEQ ID | TDYAAPVKG | CPSHDTSYCG | SVS | (SEQ ID | V |
| | | NO: 190) | (SEQ ID | GQYKSYYYMD | (SEQ ID | NO: 966) | (SEQ ID |
| | | | NO: 384) | V | NO: 772) | | NO: 1160) |
| | | | | (SEQ ID | | | |
| | | | | NO: 578) | | | |
| 23 | AIP- | GFTFSKAWMS | RIKSVDDGET | TSSFCCRGGS | SGSSSNIGSS | KNNORPS | STWDDSLSVR |
| | 153462 | (SEQ ID | TDYAAPVKG | CPSHDTSYCG | SVS | (SEQ ID | V |
| | | NO: 191) | (SEQ ID | GQYKSYYYMD | (SEQ ID | NO: 967) | (SEQ ID |
| | | | NO: 385) | V | NO: 773) | | NO: 1161) |
| | | | | (SEQ ID | | | |
| | | | | NO: 579) | | | |
| 24 | AIP- | GFTFSKAWMS | RIKSVQDGET | TSSFCCRGGS | SGSSSNIGSS | KNNQRPS | STWDDSLSVR |
| | 115363 | (SEQ ID | TDYAAPVKG | CPSHDTSYCG | SV | (SEQ ID | V |
| | | NO: 192) | (SEQ ID | GQYKSYYYMD | (SEQ ID | NO: 968) | (SEQ ID |
| | | | NO: 386) | SV | NO: 774) | | NO: 1162) |
| | | | | (SEQ ID | | | |
| | | | | NO: 580) | | | |
| 25 | AIP- | GFTFSKAWMS | RIKSVINGET | TSSFCCRGGS | SGSSSNIGSS | KNNORPS | STWDDSLSVR |
| | 151090 | (SEQ ID | TDYAAPVKG | CPSHDTSYCG | SV | (SEQ ID | V |
| | | NO: 193) | (SEQ ID | GQYKSYYYMD | (SEQ ID | NO: 969) | (SEQ ID |
| | | | NO: 387) | SV | NO: 775) | | NO: 1163) |
| | | | | (SEQ ID | | | |
| | | | | NO: 581) | | | |
| 26 | AIP- | GFTFSKAWMS | RIKSVTDAET | TSSFCCRGGS | SGSSSNIGSS | KNNQRPS | STWDDSLSVR |
| | 168083 | (SEQ ID | TDYAAPVKG | CPSHDTSYCG | SVS | (SEQ ID | V |
| | | NO: 194) | (SEQ ID | GQYKSYYYMD | (SEQ ID | NO: 970) | (SEQ ID |
| | | | NO: 388) | V | NO: 776) | | NO: 1164) |
| | | | | (SEQ ID | | | |
| | | | | NO: 582) | | | |
| 27 | AIP- | GFTFSKAWMS | RIKSVTDGOT | TSSFCCRGGS | SGSSSNIGSS | KNNORPS | STWDDSLSVR |
| | 161082 | (SEQ ID | TDYAAPVKG | CPSHDTSYCG | SV | (SEQ ID | V |
| | | NO: 195) | (SEQ ID | GQYKSYYYMD | (SEQ ID | NO: 971) | (SEQ ID |
| | | | NO: 389) | SV | NO: 777) | | NO: 1165) |
| | | | | (SEQ ID | | | |
| | | | | NO: 583) | | | |
| 28 | AIP- | GFTFSKAWMS | RIKSVTDGHT | TSSFCCRGGS | SGSSSNIGSS | KNNQRPS | STWDDSLSVR |
| | 114196 | (SEQ ID | TDYAAPVKG | CPSHDTSYCG | SV | (SEQ ID | V |
| | | NO: 196) | (SEQ ID | GQYKSYYYMD | (SEQ ID | NO: 972) | (SEQ ID |
| | | | NO: 390) | SV | NO: 778) | | NO: 1166) |
| | | | | (SEQ ID | | | |
| | | | | NO: 584) | | | |
| 29 | AIP- | GFTFSKAWMS | RIKSVTDGEQ | TSSFCCRGGS | SGSSSNIGSS | KNNQRPS | STWDDSLSVR |
| | 189338 | (SEQ ID | TDYAAPVKG | CPSHDTSYCG | SVS | (SEQ ID | V |
| | | NO: 197) | (SEQ ID | GQYKSYYYMD | (SEQ ID | NO: 973) | (SEQ ID |
| | | | NO: 391) | V | NO: 779) | | NO: 1167) |
| | | | | (SEQ ID | | | |
| | | | | NO: 585) | | | |
| 30 | AIP- | GFTFSKAWMS | RIKSVTDGEA | TSSFCCRGGS | SGSSSNIGSS | KNNORPS | STWDDSLSVR |
| | 183190 | (SEQ ID | TDYAAPVKG | CPSHDTSYCG | SV | (SEQ ID | V |
| | | NO: 198) | (SEQ ID | GQYKSYYYMD | (SEQ ID | NO: 974) | (SEQ ID |
| | | | NO: 392) | SV | NO: 780) | | NO: 1168) |
| | | | | (SEQ ID | | | |
| | | | | NO: 586) | | | |
| 31 | AIP- | GFTFSKAWMS | RIKSVTDGET | TSSFCCRGGS | SGSSSNIGSS | KNNQRPS | STWDDSLSVR |
| | 110143 | (SEQ ID | TDYASPVKG | CPSHDTSYCG | SVS | (SEQ ID | V |
| | | NO: 199) | (SEQ ID | GQYKSYYYMD | (SEQ ID | NO: 975) | (SEQ ID |
| | | | NO: 393) | V | NO: 781) | | NO: 1169) |
| | | | | (SEQ ID | | | |
| | | | | NO: 587) | | | |
| 32 | AIP- | GFTFSKAWMS | RIKSVTDGET | TSSFCCRGGS | SGSSSNIGSS | KNNORPS | STWDDSLSVR |
| | 147176 | (SEQ ID | TDYAAPVQG | CPSHDTSYCG | SV | (SEQ ID | V |
| | | NO: 200) | (SEQ ID | GQYKSYYYMD | (SEQ ID | NO: 976) | (SEQ ID |
| | | | NO: 394) | SV | NO: 782) | | NO: 1170) |
| | | | | (SEQ ID | | | |
| | | | | NO: 588) | | | |
| 33 | AIP- | GFTFSKAWMS | RIKSVTDGET | ISSFCCRGGS | SGSSSNIGSS | KNNORPS | STWDDSLSVR |
| | 134312 | (SEQ ID | TDYAAPVKG | CPSHDTSYCG | SV | (SEQ ID | V |
| | | NO: 201) | (SEQ ID | GQYKSYYYMD | (SEQ ID | NO: 977) | (SEQ ID |
| | | | NO: 395) | SV | NO: 783) | | NO: 1171) |
| | | | | (SEQ ID | | | |
| | | | | NO: 589) | | | |
| 34 | AIP- | GFTFSKAWMS | RIKSVTDGET | TTSFCCRGGS | SGSSSNIGSS | KNNQRPS | STWDDSLSVR |
| | 128243 | (SEQ ID | TDYAAPVKG | CPSHDTSYCG | SV | (SEQ ID | V |
| | | NO: 202) | (SEQ ID | GQYKSYYYMD | (SEQ ID | NO: 978) | (SEQ ID |
| | | | NO: 396) | SV | NO: 784) | | NO: 1172) |
| | | | | (SEQ ID | | | |
| | | | | NO: 590) | | | |
| 35 | AIP- | GFTFSKAWMS | RIKSVTDGET | TSPFCCRGGS | SGSSSNIGSS | KNNORPS | STWDDSLSVR |
| | 156172 | (SEQ ID | TDYAAPVKG | CPSHDTSYCG | SV | (SEQ ID | V |
| | | NO: 203) | (SEQ ID | GQYKSYYYMD | (SEQ ID | NO: 979) | (SEQ ID |
| | | | NO: 397) | SV | NO: 785) | | NO: 1173) |
| | | | | (SEQ ID | | | |
| | | | | NO: 591) | | | |
| 36 | AIP- | GFTFSKAWMS | RIKSVTDGET | TSAFCCRGGS | SGSSSNIGSS | KNNQRPS | STWDDSLSVR |
| | 147389 | (SEQ ID | TDYAAPVKG | CPSHDTSYCG | SVS | (SEQ ID | V |
| | | NO: 204) | (SEQ ID | GQYKSYYYMD | (SEQ ID | NO: 980) | (SEQ ID |
| | | | NO: 398) | V | NO: 786) | | NO: 1174) |
| | | | | (SEQ ID | | | |
| | | | | NO: 592) | | | |
| 37 | AIP- | GFTFSKAWMS | RIKSVTDGET | TSSYCCRGGS | SGSSSNIGSS | KNNQRPS | STWDDSLSVR |
| | 124314 | (SEQ ID | TDYAAPVKG | CPSHDTSYCG | SV | (SEQ ID | V |
| | | NO: 205) | (SEQ ID | GQYKSYYYMD | (SEQ ID | NO: 981) | (SEQ ID |
| | | | NO: 399) | SV | NO: 787) | | NO: 1175) |
| | | | | (SEQ ID | | | |
| | | | | NO: 593) | | | |
| 38 | AIP- | GFTFSKAWMS | RIKSVTDGET | TSSFCCLGGS | SGSSSNIGSS | KNNQRPS | STWDDSLSVR |
| | 185291 | (SEQ ID | TDYAAPVKG | CPSHDTSYCG | SV | (SEQ ID | V |
| | | NO: 206) | (SEQ ID | GQYKSYYYMD | (SEQ ID | NO: 982) | (SEQ ID |
| | | | NO: 400) | SV | NO: 788) | | NO: 1176) |
| | | | | (SEQ ID | | | |
| | | | | NO: 594) | | | |
| 39 | AIP- | GFTFSKAWMS | RIKSVTDGET | TSSFCCHGGS | SGSSSNIGSS | KNNORPS | STWDDSLSVR |
| | 135247 | (SEQ ID | TDYAAPVKG | CPSHDTSYCG | SV | (SEQ ID | V |
| | | NO: 207) | (SEQ ID | GQYKSYYYMD | (SEQ ID | NO: 983) | (SEQ ID |
| | | | NO: 401) | SV | NO: 789) | | NO: 1177) |
| | | | | (SEQ ID | | | |
| | | | | NO: 595) | | | |
| 40 | AIP- | GFTFSKAWMS | RIKSVTDGET | TSSFCCOGGS | SGSSSNIGSS | KNNQRPS | STWDDSLSVR |
| | 113513 | (SEQ ID | TDYAAPVKG | CPSHDTSYCG | SV | (SEQ ID | V |
| | | NO: 208) | (SEQ ID | GQYKSYYYMD | (SEQ ID | NO: 984) | (SEQ ID |
| | | | NO: 402) | SV | NO: 790) | | NO: 1178) |
| | | | | (SEQ ID | | | |
| | | | | NO: 596) | | | |
| 41 | AIP- | GFTFSKAWMS | RIKSVTDGET | TSSFCCRSGS | SGSSSNIGSS | KNNQRPS | STWDDSLSVR |
| | 102299 | (SEQ ID | TDYAAPVKG | CPSHDTSYCG | SV | (SEQ ID | V |
| | | NO: 209) | (SEQ ID | GQYKSYYYMD | (SEQ ID | NO: 985) | (SEQ ID |
| | | | NO: 403) | SV | NO: 791) | | NO: 1179) |
| | | | | (SEQ ID | | | |
| | | | | NO: 597) | | | |
| 42 | AIP- | GFTFSKAWMS | RIKSVTDGET | TSSFCCRGAS | SGSSSNIGSS | KNNORPS | STWDDSLSVR |
| | 179097 | (SEQ ID | TDYAAPVKG | CPSHDTSYCG | SV | (SEQ ID | V |
| | | NO: 210) | (SEQ ID | GQYKSYYYMD | (SEQ ID | NO: 986) | (SEQ ID |
| | | | NO: 404) | SV | NO: 792) | | NO: 1180) |
| | | | | (SEQ ID | | | |
| | | | | NO: 598) | | | |
| 43 | AIP- | GFTFSKAWMS | RIKSVTDGET | TSSFCCRGKS | SGSSSNIGSS | KNNORPS | STWDDSLSVR |
| | 109343 | (SEQ ID | TDYAAPVKG | CPSHDTSYCG | SV | (SEQ ID | V |
| | | NO: 211) | (SEQ ID | GQYKSYYYMD | (SEQ ID | NO: 987) | (SEQ ID |
| | | | NO: 405) | SV | NO: 793) | | NO: 1181) |
| | | | | (SEQ ID | | | |
| | | | | NO: 599) | | | |
| 44 | AIP- | GFTFSKAWMS | RIKSVTDGET | TSSFCCRGNS | SGSSSNIGSS | KNNORPS | STWDDSLSVR |
| | 119622 | (SEQ ID | TDYAAPVKG | CPSHDTSYCG | SV | (SEQ ID | V |
| | | NO: 212) | (SEQ ID | GQYKSYYYMD | (SEQ ID | NO: 988) | (SEQ ID |
| | | | NO: 406) | SV | NO: 794) | | NO: 1182) |
| | | | | (SEQ ID | | | |
| | | | | NO: 600) | | | |
| 45 | AIP- | GFTFSKAWMS | RIKSVTDGET | TSSFCCRGGN | SGSSSNIGSS | KNNQRPS | STWDDSLSVR |
| | 191735 | (SEQ ID | TDYAAPVKG | CPSHDTSYCG | SV | (SEQ ID | V |
| | | NO: 213) | (SEQ ID | GQYKSYYYMD | (SEQ ID | NO: 989) | (SEQ ID |
| | | | NO: 407) | SV | NO: 795) | | NO: 1183) |
| | | | | (SEQ ID | | | |
| | | | | NO: 601) | | | |
| 46 | AIP- | GFTFSKAWMS | RIKSVTDGET | TSSFCCRGGQ | SGSSSNIGSS | KNNORPS | STWDDSLSVR |
| | 157078 | (SEQ ID | TDYAAPVKG | CPSHDTSYCG | SV | (SEQ ID | V |
| | | NO: 214) | (SEQ ID | GQYKSYYYMD | (SEQ ID | NO: 990) | (SEQ ID |
| | | | NO: 408) | SV | NO: 796) | | NO: 1184) |
| | | | | (SEQ ID | | | |
| | | | | NO: 602) | | | |
| 47 | AIP- | GFTFSKAWMS | RIKSVTDGET | TSSFCCRGGA | SGSSSNIGSS | KNNORPS | STWDDSLSVR |
| | 153475 | (SEQ ID | TDYAAPVKG | CPSHDTSYCG | SVS | (SEQ ID | V |
| | | NO: 215) | (SEQ ID | GQYKSYYYMD | (SEQ ID | NO: 991) | (SEQ ID |
| | | | NO: 409) | V | NO: 797) | | NO: 1185) |
| | | | | (SEQ ID | | | |
| | | | | NO: 603) | | | |
| 48 | AIP- | GFTFSKAWMS | RIKSVTDGET | TSSFCCRGGS | SGSSSNIGSS | KNNORPS | STWDDSLSVR |
| | 133650 | (SEQ ID | TDYAAPVKG | CASHDTSYCG | SV | (SEQ ID | V |
| | | NO: 216) | (SEQ ID | GQYKSYYYMD | (SEQ ID | NO: 992) | (SEQ ID |
| | | | NO: 410) | SV | NO: 798) | | NO: 1186) |
| | | | | (SEQ ID | | | |
| | | | | NO: 604) | | | |
| 49 | AIP- | GFTFSKAWMS | RIKSVTDGET | TSSFCCRGGS | SGSSSNIGSS | KNNQRPS | STWDDSLSVR |
| | 190915 | (SEQ ID | TDYAAPVKG | CLSHDTSYCG | SV | (SEQ ID | V |
| | | NO: 217) | (SEQ ID | GQYKSYYYMD | (SEQ ID | NO: 993) | (SEQ ID |
| | | | NO: 411) | SV | NO: 799) | | NO: 1187) |
| | | | | (SEQ ID | | | |
| | | | | NO: 605) | | | |
| 50 | AIP- | GFTFSKAWMS | RIKSVTDGET | TSSFCCRGGS | SGSSSNIGSS | KNNORPS | STWDDSLSVR |
| | 105241 | (SEQ ID | TDYAAPVKG | CPAHDTSYCG | SV | (SEQ ID | V |
| | | NO: 218) | (SEQ ID | GQYKSYYYMD | (SEQ ID | NO: 994) | (SEQ ID |
| | | | NO: 412) | SV | NO: 800) | | NO: 1188) |
| | | | | (SEQ ID | | | |
| | | | | NO: 606) | | | |
| 51 | AIP- | GFTFSKAWMS | RIKSVTDGET | TSSFCCRGGS | SGSSSNIGSS | KNNQRPS | STWDDSLSVR |
| | 167400 | (SEQ ID | TDYAAPVKG | CPNHDTSYCG | SV | (SEQ ID | V |
| | | NO: 219) | (SEQ ID | GQYKSYYYMD | (SEQ ID | NO: 995) | (SEQ ID |
| | | | NO: 413) | SV | NO: 801) | | NO: 1189) |
| | | | | (SEQ ID | | | |
| | | | | NO: 607) | | | |
| 52 | AIP- | GFTFSKAWMS | RIKSVTDGET | TSSFCCRGGS | SGSSSNIGSS | KNNORPS | STWDDSLSVR |
| | 109729 | (SEQ ID | TDYAAPVKG | CPSSDTSYCG | SVS | (SEQ ID | V |
| | | NO: 220) | (SEQ ID | GQYKSYYYMD | (SEQ ID | NO: 996) | (SEQ ID |
| | | | NO: 414) | V | NO: 802) | | NO: 1190) |
| | | | | (SEQ ID | | | |
| | | | | NO: 608) | | | |
| 53 | AIP- | GFTFSKAWMS | RIKSVTDGET | TSSFCCRGGS | SGSSSNIGSS | KNNORPS | STWDDSLSVR |
| | 151709 | (SEQ ID | TDYAAPVKG | CPSQDTSYCG | SV | (SEQ ID | V |
| | | NO: 221) | (SEQ ID | GQYKSYYYMD | (SEQ ID | NO: 997) | (SEQ ID |
| | | | NO: 415) | SV | NO: 803) | | NO: 1191) |
| | | | | (SEQ ID | | | |
| | | | | NO: 609) | | | |
| 54 | AIP- | GFTFSKAWMS | RIKSVTDGET | TSSFCCRGGS | SGSSSNIGSS | KNNORPS | STWDDSLSVR |
| | 137169 | (SEQ ID | TDYAAPVKG | CPSHETSYCG | SVS | (SEQ ID | V |
| | | NO: 222) | (SEQ ID | GQYKSYYYMD | (SEQ ID | NO: 998) | (SEQ ID |
| | | | NO: 416) | V | NO: 804) | | NO: 1192) |
| | | | | (SEQ ID | | | |
| | | | | NO: 610) | | | |
| 55 | AIP- | GFTFSKAWMS | RIKSVTDGET | TSSFCCRGGS | SGSSSNIGSS | KNNORPS | STWDDSLSVR |
| | 199616 | (SEQ ID | TDYAAPVKG | CPSHDQSYCG | SV | (SEQ ID | V |
| | | NO: 223) | (SEQ ID | GQYKSYYYMD | (SEQ ID | NO: 999) | (SEQ ID |
| | | | NO: 417) | SV | NO: 805) | | NO: 1193) |
| | | | | (SEQ ID | | | |
| | | | | NO: 611) | | | |
| 56 | AIP- | GFTFSKAWMS | RIKSVTDGET | TSSFCCRGGS | SGSSSNIGSS | KNNORPS | STWDDSLSVR |
| | 189296 | (SEQ ID | TDYAAPVKG | CPSHDTSMCG | SV | (SEQ ID | V |
| | | NO: 224) | (SEQ ID | GQYKSYYYMD | (SEQ ID | NO: 1000) | (SEQ ID |
| | | | NO: 418) | SV | NO: 806) | | NO: 1194) |
| | | | | (SEQ ID | | | |
| | | | | NO: 612) | | | |
| 57 | AIP- | GFTFSKAWMS | RIKSVTDGET | TSSFCCRGGS | SGSSSNIGSS | KNNQRPS | STWDDSLSVR |
| | 152283 | (SEQ ID | TDYAAPVKG | CPSHDTSYCA | SV | (SEQ ID | V |
| | | NO: 225) | (SEQ ID | GQYKSYYYMD | (SEQ ID | NO: 1001) | (SEQ ID |
| | | | NO: 419) | SV | NO: 807) | | NO: 1195) |
| | | | | (SEQ ID | | | |
| | | | | NO: 613) | | | |
| 58 | AIP- | GFTFSKAWMS | RIKSVTDGET | TSSFCCRGGS | SGSSSNIGSS | KNNORPS | STWDDSLSVR |
| | 136628 | (SEQ ID | TDYAAPVKG | CPSHDTSYCN | SV | (SEQ ID | V |
| | | NO: 226) | (SEQ ID | GQYKSYYYMD | (SEQ ID | NO: 1002) | (SEQ ID |
| | | | NO: 420) | SV | NO: 808) | | NO: 1196) |
| | | | | (SEQ ID | | | |
| | | | | NO: 614) | | | |
| 59 | AIP- | GFTFSKAWMS | RIKSVTDGET | TSSFCCRGGS | SGSSSNIGSS | KNNORPS | STWDDSLSVR |
| | 100340 | (SEQ ID | TDYAAPVKG | CPSHDTSYCG | SV | (SEQ ID | V |
| | | NO: 227) | (SEQ ID | NQYKSYYYMD | (SEQ ID | NO: 1003) | (SEQ ID |
| | | | NO: 421) | SV | NO: 809) | | NO: 1197) |
| | | | | (SEQ ID | | | |
| | | | | NO: 615) | | | |
| 60 | AIP- | GFTFSKAWMS | RIKSVTDGET | TSSFCCRGGS | SGSSSNIGSS | KNNORPS | STWDDSLSVR |
| | 166959 | (SEQ ID | TDYAAPVKG | CPSHDTSYCG | SV | (SEQ ID | V |
| | | NO: 228) | (SEQ ID | AQYKSYYYMD | (SEQ ID | NO: 1004) | (SEQ ID |
| | | | NO: 422) | SV | NO: 810) | | NO: 1198) |
| | | | | (SEQ ID | | | |
| | | | | NO: 616) | | | |
| 61 | AIP- | GFTFSKAWMS | RIKSVTDGET | TSSFCCRGGS | SGSSSNIGSS | KNNORPS | STWDDSLSVR |
| | 190362 | (SEQ ID | TDYAAPVKG | CPSHDTSYCG | SV | (SEQ ID | V |
| | | NO: 229) | (SEQ ID | GSYKSYYYMD | (SEQ ID | NO: 1005) | (SEQ ID |
| | | | NO: 423) | SV | NO: 811) | | NO: 1199) |
| | | | | (SEQ ID | | | |
| | | | | NO: 617) | | | |
| 62 | AIP- | GFTFSKAWMS | RIKSVTDGET | TSSFCCRGGS | SGSSSNIGSS | KNNORPS | STWDDSLSVR |
| | 101601 | (SEQ ID | TDYAAPVKG | CPSHDTSYCG | SV | (SEQ ID | V |
| | | NO: 230) | (SEQ ID | GYYKSYYYMD | (SEQ ID | NO: 1006) | (SEQ ID |
| | | | NO: 424) | SV | NO: 812) | | NO: 1200) |
| | | | | (SEQ ID | | | |
| | | | | NO: 618) | | | |
| 63 | AIP- | GFTFSKAWMS | RIKSVTDGET | TSSFCCRGGS | SGSSSNIGSS | KNNORPS | STWDDSLSVR |
| | 159023 | (SEQ ID | TDYJAAPVKG | CPSHDTSYCG | SV | (SEQ ID | V |
| | | NO: 231) | (SEQ ID | GOFKSYYYMD | (SEQ ID | NO: 1007) | (SEQ ID |
| | | | NO: 425) | SV | NO: 813) | | NO: 1201) |
| | | | | (SEQ ID | | | |
| | | | | NO: 619) | | | |
| 64 | AIP- | GFTFSKAWMS | RIKSVTDGET | TSSFCCRGGS | SGSSSNIGSS | KNNORPS | STWDDSLSVR |
| | 146871 | (SEQ ID | TDYAAPVKG | CPSHDTSYCG | SV | (SEQ ID | V |
| | | NO: 232) | (SEQ ID | GQYPSYYYMD | (SEQ ID | NO: 1008) | (SEQ ID |
| | | | NO: 426) | SV | NO: 814) | | NO: 1202) |
| | | | | (SEQ ID | | | |
| | | | | NO: 620) | | | |
| 65 | AIP- | GFTFSKAWMS | RIKSVTDGET | TSSFCCRGGS | SGSSSNIGSS | KNNORPS | STWDDSLSVR |
| | 170053 | (SEQ ID | TDYAAPVKG | CPSHDTSYCG | SV | (SEQ ID | V |
| | | NO: 233) | (SEQ ID | GOYASYYYMD | (SEQ ID | NO: 1009) | (SEQ ID |
| | | | NO: 427) | SV | NO: 815) | | NO: 1203) |
| | | | | (SEQ ID | | | |
| | | | | NO: 621) | | | |
| 66 | AIP- | GFTFSKAWMS | RIKSVTDGET | TSSFCCRGGS | SGSSSNIGSS | KNNORPS | STWDDSLSVR |
| | 199483 | (SEQ ID | TDYAAPVKG | CPSHDTSYCG | SVS | (SEQ ID | V |
| | | NO: 234) | (SEQ ID | GQYQSYYYMD | (SEQ ID | NO: 1010) | (SEQ ID |
| | | | NO: 428) | V | NO: 816) | | NO: 1204) |
| | | | | (SEQ ID | | | |
| | | | | NO: 622) | | | |
| 67 | AIP- | GFTFSKAWMS | RIKSVTDGET | TSSFCCRGGS | SGSSSNIGSS | KNNORPS | STWDDSLSVR |
| | 161048 | (SEQ ID | TDYAAPVKG | CPSHDTSYCG | SV | (SEQ ID | V |
| | | NO: 235) | (SEQ ID | GQYKDYYYMD | (SEQ ID | NO: 1011) | (SEQ ID |
| | | | NO: 429) | SV | NO: 817) | | NO: 1205) |
| | | | | (SEQ ID | | | |
| | | | | NO: 623) | | | |
| 68 | AIP- | GFTFSKAWMS | RIKSVTDGET | TSSFCCRGGS | SGSSSNIGSS | KNNORPS | STWDDSLSVR |
| | 162041 | (SEQ ID | TDYAAPVKG | CPSHDTSYCG | SV | (SEQ ID | V |
| | | NO: 236) | (SEQ ID | GQYKSFYYMD | (SEQ ID | NO: 1012) | (SEQ ID |
| | | | NO: 430) | SV | NO: 818) | | NO: 1206) |
| | | | | (SEQ ID | | | |
| | | | | NO: 624) | | | |
| 69 | AIP- | GFTFSKAWMS | RIKSVTDGET | TSSFCCRGGS | SGSSSNIGSS | KNNORPS | STWDDSLSVR |
| | 197886 | (SEQ ID | TDYJAAPVKG | CPSHDTSYCG | SV | (SEQ ID | V |
| | | NO: 237) | (SEQ ID | GQYKSYFYMD | (SEQ ID | NO: 1013) | (SEQ ID |
| | | | NO: 431) | SV | NO: 819) | | NO: 1207) |
| | | | | (SEQ ID | | | |
| | | | | NO: 625) | | | |
| 70 | AIP- | GFTFSKAWMS | RIKSVTDGET | TSSFCCRGGS | SGSSSNIGSS | KNNORPS | STWDDSLSVR |
| | 183133 | (SEQ ID | TDYAAPVKG | CPSHDTSYCG | SV | (SEQ ID | V |
| | | NO: 238) | (SEQ ID | GQYKSYYWMD | (SEQ ID | NO: 1014) | (SEQ ID |
| | | | NO: 432) | SV | NO: 820) | | NO: 1208) |
| | | | | (SEQ ID | | | |
| | | | | NO: 626) | | | |
| 71 | AIP- | GFTFSKAWMS | RIKSVTDGET | TSSFCCRGGS | SGSSSNIGSS | KNNQRPS | STWDDSLSVR |
| | 191470 | (SEQ ID | TDYAAPVKG | CPSHDTSYCG | SV | (SEQ ID | V |
| | | NO: 239) | (SEQ ID | GQYKSYYLMD | (SEQ ID | NO: 1015) | (SEQ ID |
| | | | NO: 433) | SV | NO: 821) | | NO: 1209) |
| | | | | (SEQ ID | | | |
| | | | | NO: 627) | | | |
| 72 | AIP- | GFTFSKAWMS | RIKSVTDGET | TSSFCCRGGS | SGSSSNIGSS | KNNORPS | STWDDSLSVR |
| | 151167 | (SEQ ID | TDYAAPVKG | CPSHDTSYCG | SV | (SEQ ID | V |
| | | NO: 240) | (SEQ ID | GQYKSYYYVD | (SEQ ID | NO: 1016) | (SEQ ID |
| | | | NO: 434) | SV | NO: 822) | | NO: 1210) |
| | | | | (SEQ ID | | | |
| | | | | NO: 628) | | | |
| 73 | AIP- | GFTFSKAWMS | RIKSVTDGET | TSSFCCRGGS | SGSSSNIGSS | KNNORPS | STWDDSLSVR |
| | 106633 | (SEQ ID | TDYAAPVKG | CPSHDTSYCG | SV | (SEQ ID | V |
| | | NO: 241) | (SEQ ID | GQYKSYYYFD | (SEQ ID | NO: 1017) | (SEQ ID |
| | | | NO: 435) | SV | NO: 823) | | NO: 1211) |
| | | | | (SEQ ID | | | |
| | | | | NO: 629) | | | |
| 74 | AIP- | GFTFSKAWMS | RIKSVTDGET | TSSFCCRGGS | SGSSSNIGSS | KNNORPS | STWDDSLSVR |
| | 102624 | (SEQ ID | TDYAAPVKG | CPSHDTSYCG | SVS | (SEQ ID | V |
| | | NO: 242) | (SEQ ID | GQYKSYYYMN | (SEQ ID | NO: 1018) | (SEQ ID |
| | | | NO: 436) | V | NO: 824) | | NO: 1212) |
| | | | | (SEQ ID | | | |
| | | | | NO: 630) | | | |
| 75 | AIP- | GFTFSKAWMS | RIKSVTDGET | TSSFCCRGGS | SGSSSNIGSS | KNNORPS | STWDDSLSVR |
| | 109484 | (SEQ ID | TDYAAPVKG | CPSHDTSYCG | SVS | (SEQ ID | V |
| | | NO: 243) | (SEQ ID | GQYKSYYYMD | (SEQ ID | NO: 1019) | (SEQ ID |
| | | | NO: 437) | I | NO: 825) | | NO: 1213) |
| | | | | (SEQ ID | | | |
| | | | | NO: 631) | | | |
| 76 | AIP- | GFTFSKAWMS | RIKSVTDGET | TSSFCCRGGS | SGSSSNIGSS | KNNQRPS | STWDDSLSVR |
| | 164754 | (SEQ ID | TDYAAPVKG | CPSHDTSYCG | SV | (SEQ ID | V |
| | | NO: 244) | (SEQ ID | GQYKSYYYMD | (SEQ ID | NO: 1020) | (SEQ ID |
| | | | NO: 438) | SP | NO: 826) | | NO: 1214) |
| | | | | (SEQ ID | | | |
| | | | | NO: 632) | | | |
| 77 | AIP- | GFTFSKAWMS | RIKSVTDGET | TSSFCCRGGS | SGSSSNIGSS | KNNORPS | STWDDSLSVR |
| | 169676 | (SEQ ID | TDYAAPVKG | CPSHDTSYCG | SV | (SEQ ID | V |
| | | NO: 245) | (SEQ ID | GQFKDYYYMD | (SEQ ID | NO: 1021) | (SEQ ID |
| | | | NO: 439) | SV | NO: 827) | | NO: 1215) |
| | | | | (SEQ ID | | | |
| | | | | NO: 633) | | | |
| 78 | AIP- | GFTFSKAWMS | RIKSVTDGET | TSSFCCRGGS | SGSSSNIGSS | KNNQRPS | STWDDSLSVR |
| | 177584 | (SEQ ID | TDYAAPVKG | CPSHDTSYCG | SVS | (SEQ ID | V |
| | | NO: 246) | (SEQ ID | GOSKQYYYMD | (SEQ ID | NO: 1022) | (SEQ ID |
| | | | NO: 440) | V | NO: 828) | | NO: 1216) |
| | | | | (SEQ ID | | | |
| | | | | NO: 634) | | | |
| 79 | AIP- | GFTFSKAWMS | RIKSVTDGET | TSSFCCRGGS | SGSSSNIGSS | KNNQRPS | STWDDSLSVR |
| | 174676 | (SEQ ID | TDYAAPVKG | CPSHDTSYCG | SV | (SEQ ID | V |
| | | NO: 247) | (SEQ ID | GQSKKYYYMD | (SEQ ID | NO: 1023) | (SEQ ID |
| | | | NO: 441) | SV | NO: 829) | | NO: 1217) |
| | | | | (SEQ ID | | | |
| | | | | NO: 635) | | | |
| 80 | AIP- | GFTFSKAWMS | RIKSVTDGET | TSSFSCRGGS | SGSSSNIGSS | KNNQRPS | STWDDSLSVR |
| | 120546 | (SEQ ID | TDYAAPVKG | CPSHDTSYSG | SV | (SEQ ID | V |
| | | NO: 248) | (SEQ ID | GQYKSYYYMD | (SEQ ID | NO: 1024) | (SEQ ID |
| | | | NO: 442) | SV | NO: 830) | | NO: 1218) |
| | | | | (SEQ ID | | | |
| | | | | NO: 636) | | | |
| 81 | AIP- | GFTFSKAWMS | RIKSVTDGET | TSSFACRGGS | SGSSSNIGSS | KNNORPS | STWDDSLSVR |
| | 186435 | (SEQ ID | TDYAAPVKG | CPSHDTSYVG | SVS | (SEQ ID | V |
| | | NO: 249) | (SEQ ID | GQYKSYYYMD | (SEQ ID | NO: 1025) | (SEQ ID |
| | | | NO: 443) | V | NO: 831) | | NO: 1219) |
| | | | | (SEQ ID | | | |
| | | | | NO: 637) | | | |
| 82 | AIP- | GFTFSKAWMS | RIKSVTDGET | TSSFCCRGNS | SGSSSNIGSS | KNNORPS | STWDDSLSVR |
| | 171074 | (SEQ ID | TDYAAPVKG | CPAHDTSYCG | SV | (SEQ ID | V |
| | | NO: 250) | (SEQ ID | GQYPSYYYMD | (SEQ ID | NO: 1026) | (SEQ ID |
| | | | NO: 444) | SV | NO: 832) | | NO: 1220) |
| | | | | (SEQ ID | | | |
| | | | | NO: 638) | | | |
| 83 | AIP- | GFTFSKAWMS | RIKSVTDGET | TSSYCCLGGS | SGSSSNIGSS | KNNORPS | STWDDSLSVR |
| | 163039 | (SEQ ID | TDYAAPVKG | CPSHDTSYCG | SVS | (SEQ ID | V |
| | | NO: 251) | (SEQ ID | GSYKSYYYMD | (SEQ ID | NO: 1027) | (SEQ ID |
| | | | NO: 445) | V | NO: 833) | | NO: 1221) |
| | | | | (SEQ ID | | | |
| | | | | NO: 639) | | | |
| 84 | AIP- | GFTFSKAWMS | RIKSVTDGET | TSSFCCRGKS | SGSSSNIGSS | KNNORPS | STWDDSLSVR |
| | 147652 | (SEQ ID | TDYAAPVKG | CPSHDNSYCG | SV | (SEQ ID | V |
| | | NO: 252) | (SEQ ID | GQYASYYYMD | (SEQ ID | NO: 1028) | (SEQ ID |
| | | | NO: 446) | SV | NO: 834) | | NO: 1222) |
| | | | | (SEQ ID | | | |
| | | | | NO: 640) | | | |
| 85 | AIP- | GFTFSKAWMS | RIKSVTDGET | TSSYCCRGGS | SGSSSNIGSS | KNNORPS | STWDDSLSVR |
| | 182061 | (SEQ ID | TDYAAPVKG | CPSHDTSYCG | SV | (SEQ ID | V |
| | | NO: 253) | (SEQ ID | GQFKSYYYMD | (SEQ ID | NO: 1029) | (SEQ ID |
| | | | NO: 447) | SP | NO: 835) | | NO: 1223) |
| | | | | (SEQ ID | | | |
| | | | | NO: 641) | | | |
| 86 | AIP- | GFTFSKAWMS | RIKSVTDGET | TSTFCCRGGS | SGSSSNIGSS | KNNQRPS | STWDDSLSVR |
| | 172643 | (SEQ ID | TDYAAPVKG | CLSHDTSYCG | SV | (SEQ ID | V |
| | | NO: 254) | (SEQ ID | GQYKSYYWMD | (SEQ ID | NO: 1030) | (SEQ ID |
| | | | NO: 448) | SV | NO: 836) | | NO: 1224) |
| | | | | (SEQ ID | | | |
| | | | | NO: 642) | | | |
| 87 | AIP- | GFDFSKAWMS | RIKSVTDGOT | TSSFCCRGGS | TGSSSNIGSS | KNNQRPS | STWDDSLSVR |
| | 171912 | (SEQ ID | TDYAAPVKG | CPSHDTSYCG | SVS | (SEQ ID | V |
| | | NO: 255) | (SEQ ID | GQYKSYYYMD | (SEQ ID | NO: 1031) | (SEQ ID |
| | | | NO: 449) | V | NO: 837) | | NO: 1225) |
| | | | | (SEQ ID | | | |
| | | | | NO: 643) | | | |
| 88 | AIP- | GFTFAKAWMS | RIKSVTDGET | TSSFCCRGGS | SGSSTNIGSS | KNNYRPS | STWDDSLSVR |
| | 167833 | (SEQ ID | TDYJAAPVKG | CPSHDTSYCG | SVS | (SEQ ID | V |
| | | NO: 256) | (SEQ ID | GQYKSYYYMD | (SEQ ID | NO: 1032) | (SEQ ID |
| | | | NO: 450) | V | NO: 838) | | NO: 1226) |
| | | | | (SEQ ID | | | |
| | | | | NO: 644) | | | |
| 89 | AIP- | GFDASKAWFT | RIKSVTDGET | TSSFCCRGGS | SGSSSNIGSS | KNNORPS | STWDDSLSVR |
| | 145518 | (SEQ ID | TDYAAPVKG | CPSHDTSYCG | SVS | (SEQ ID | V |
| | | NO: 257) | (SEQ ID | GQYKSYYYMD | (SEQ ID | NO: 1033) | (SEQ ID |
| | | | NO: 451) | V | NO: 839) | | NO: 1227) |
| | | | | (SEQ ID | | | |
| | | | | NO: 645) | | | |
| 90 | AIP- | GFTFSKAWMS | RIKSVTDGET | TSSFCCRGGS | TGSSSNIGSS | KNNORPS | STWDDSLSVR |
| | 143155 | (SEQ ID | TDYAAPVKG | CPSHDTSYCG | SV | (SEQ ID | V |
| | | NO: 258) | (SEQ ID | GQYKSYYYMD | (SEQ ID | NO: 1034) | (SEQ ID |
| | | | NO: 452) | SV | NO: 840) | | NO: 1228) |
| | | | | (SEQ ID | | | |
| | | | | NO: 646) | | | |
| 91 | AIP- | GFTFSKAWMS | RIKSVTDGET | TSSFCCRGGS | SGASSNIGSS | KNNORPS | STWDDSLSVR |
| | 119664 | (SEQ ID | TDYAAPVKG | CPSHDTSYCG | SVS | (SEQ ID | V |
| | | NO: 259) | (SEQ ID | GQYKSYYYMD | (SEQ ID | NO: 1035) | (SEQ ID |
| | | | NO: 453) | V | NO: 841) | | NO: 1229) |
| | | | | (SEQ ID | | | |
| | | | | NO: 647) | | | |
| 92 | AIP- | GFTFSKAWMS | RIKSVTDGET | TSSFCCRGGS | SGSPSNIGSS | KNNQRPS | STWDDSLSVR |
| | 190526 | (SEQ ID | TDYAAPVKG | CPSHDTSYCG | SV | (SEQ ID | V |
| | | NO: 260) | (SEQ ID | GQYKSYYYMD | (SEQ ID | NO: 1036) | (SEQ ID |
| | | | NO: 454) | SV | NO: 842) | | NO: 1230) |
| | | | | (SEQ ID | | | |
| | | | | NO: 648) | | | |
| 93 | AIP- | GFTFSKAWMS | RIKSVTDGET | TSSFCCRGGS | SGSKSNIGSS | KNNORPS | STWDDSLSVR |
| | 114403 | (SEQ ID | TDYAAPVKG | CPSHDTSYCG | SV | (SEQ ID | V |
| | | NO: 261) | (SEQ ID | GQYKSYYYMD | (SEQ ID | NO: 1037) | (SEQ ID |
| | | | NO: 455) | SV | NO: 843) | | NO: 1231) |
| | | | | (SEQ ID | | | |
| | | | | NO: 649) | | | |
| 94 | AIP- | GFTFSKAWMS | RIKSVTDGET | TSSFCCRGGS | SGSSTNIGSS | KNNORPS | STWDDSLSVR |
| | 156760 | (SEQ ID | TDYAAPVKG | CPSHDTSYCG | SV | (SEQ ID | V |
| | | NO: 262) | (SEQ ID | GQYKSYYYMD | (SEQ ID | NO: 1038) | (SEQ ID |
| | | | NO: 456) | SV | NO: 844) | | NO: 1232) |
| | | | | (SEQ ID | | | |
| | | | | NO: 650) | | | |
| 95 | AIP- | GFTFSKAWMS | RIKSVTDGET | TSSFCCRGGS | SGSSSDIGSS | KNNORPS | STWDDSLSVR |
| | 103803 | (SEQ ID | TDYAAPVKG | CPSHDTSYCG | SV | (SEQ ID | V |
| | | NO: 263) | (SEQ ID | GQYKSYYYMD | (SEQ ID | NO: 1039) | (SEQ ID |
| | | | NO: 457) | SV | NO: 845) | | NO: 1233) |
| | | | | (SEQ ID | | | |
| | | | | NO: 651) | | | |
| 96 | AIP- | GFTFSKAWMS | RIKSVTDGET | TSSFCCRGGS | SGSSSNIQSS | KNNORPS | STWDDSLSVR |
| | 182722 | (SEQ ID | TDYAAPVKG | CPSHDTSYCG | SV | (SEQ ID | V |
| | | NO: 264) | (SEQ ID | GQYKSYYYMD | (SEQ ID | NO: 1040) | (SEQ ID |
| | | | NO: 458) | SV | NO: 846) | | NO: 1234) |
| | | | | (SEQ ID | | | |
| | | | | NO: 652) | | | |
| 97 | AIP- | GFTFSKAWMS | RIKSVTDGET | TSSFCCRGGS | SGSSSNIGHS | KNNORPS | STWDDSLSVR |
| | 195588 | (SEQ ID | TDYAAPVKG | CPSHDTSYCG | SV | (SEQ ID | V |
| | | NO: 265) | (SEQ ID | GQYKSYYYMD | (SEQ ID | NO: 1041) | (SEQ ID |
| | | | NO: 459) | SV | NO: 847) | | NO: 1235) |
| | | | | (SEQ ID | | | |
| | | | | NO: 653) | | | |
| 98 | AIP- | GFTFSKAWMS | RIKSVTDGET | TSSFCCRGGS | SGSSSNIGST | KNNQRPS | STWDDSLSVR |
| | 145722 | (SEQ ID | TDYAAPVKG | CPSHDTSYCG | SV | (SEQ ID | V |
| | | NO: 266) | (SEQ ID | GQYKSYYYMD | (SEQ ID | NO: 1042) | (SEQ ID |
| | | | NO: 460) | SV | NO: 848) | | NO: 1236) |
| | | | | (SEQ ID | | | |
| | | | | NO: 654) | | | |
| 99 | AIP- | GFTFSKAWMS | RIKSVTDGET | TSSFCCRGGS | SGSSSNIGSN | KNNORPS | STWDDSLSVR |
| | 178251 | (SEQ ID | TDYAAPVKG | CPSHDTSYCG | SV | (SEQ ID | V |
| | | NO: 267) | (SEQ ID | GQYKSYYYMD | (SEQ ID | NO: 1043) | (SEQ ID |
| | | | NO: 461) | SV | NO: 849) | | NO: 1237) |
| | | | | (SEQ ID | | | |
| | | | | NO: 655) | | | |
| 100 | AIP- | GFTFSKAWMS | RIKSVTDGET | TSSFCCRGGS | SGSSSNIGSA | KNNORPS | STWDDSLSVR |
| | 116142 | (SEQ ID | TDYAAPVKG | CPSHDTSYCG | SV | (SEQ ID | V |
| | | NO: 268) | (SEQ ID | GQYKSYYYMD | (SEQ ID | NO: 1044) | (SEQ ID |
| | | | NO: 462) | SV | NO: 850) | | NO: 1238) |
| | | | | (SEQ ID | | | |
| | | | | NO: 656) | | | |
| 101 | AIP- | GFTFSKAWMS | RIKSVTDGET | TSSFCCRGGS | SGSSSNIGSS | KNNORPS | STWDDSLSVR |
| | 183350 | (SEQ ID | TDYAAPVKG | CPSHDTSYCG | TV | (SEQ ID | V |
| | | NO: 269) | (SEQ ID | GQYKSYYYMD | (SEQ ID | NO: 1045) | (SEQ ID |
| | | | NO: 463) | SV | NO: 851) | | NO: 1239) |
| | | | | (SEQ ID | | | |
| | | | | NO: 657) | | | |
| 102 | AIP- | GFTFSKAWMS | RIKSVTDGET | TSSFCCRGGS | SGSSSNIGSS | KNNQRPS | STWDDSLSVR |
| | 127108 | (SEQ ID | TDYAAPVKG | CPSHDTSYCG | AV | (SEQ ID | V |
| | | NO: 270) | (SEQ ID | GQYKSYYYMD | (SEQ ID | NO: 1046) | (SEQ ID |
| | | | NO: 464) | SV | NO: 852) | | NO: 1240) |
| | | | | (SEQ ID | | | |
| | | | | NO: 658) | | | |
| 103 | AIP- | GFTFSKAWMS | RIKSVTDGET | TSSFCCRGGS | SGSSSNIGSS | KNNORPS | STWDDSLSVR |
| | 128147 | (SEQ ID | TDYAAPVKG | CPSHDTSYCG | YV | (SEQ ID | V |
| | | NO: 271) | (SEQ ID | GQYKSYYYMD | (SEQ ID | NO: 1047) | (SEQ ID |
| | | | NO: 465) | SV | NO: 853) | | NO: 1241) |
| | | | | (SEQ ID | | | |
| | | | | NO: 659) | | | |
| 104 | AIP- | GFTFSKAWMS | RIKSVTDGET | TSSFCCRGGS | SGSSSNIGSS | KNNORPS | STWDDSLSVR |
| | 109510 | (SEQ ID | TDYAAPVKG | CPSHDTSYCG | ST | (SEQ ID | V |
| | | NO: 272) | (SEQ ID | GQYKSYYYMD | (SEQ ID | NO: 1048) | (SEQ ID |
| | | | NO: 466) | SV | NO: 854) | | NO: 1242) |
| | | | | (SEQ ID | | | |
| | | | | NO: 660) | | | |
| 105 | AIP- | GFTFSKAWMS | RIKSVTDGET | TSSFCCRGGS | SGSSSNIGSS | KNNORPS | STWDDSLSVR |
| | 104086 | (SEQ ID | TDYAAPVKG | CPSHDTSYCG | SV | (SEQ ID | V |
| | | NO: 273) | (SEQ ID | GQYKSYYYMD | (SEQ ID | NO: 1049) | (SEQ ID |
| | | | NO: 467) | AV | NO: 855) | | NO: 1243) |
| | | | | (SEQ ID | | | |
| | | | | NO: 661) | | | |
| 106 | AIP- | GFTFSKAWMS | RIKSVTDGET | TSSFCCRGGS | SGSSSNIGSS | KNNQRPS | STWDDSLSVR |
| | 143132 | (SEQ ID | TDYAAPVKG | CPSHDTSYCG | SV | (SEQ ID | V |
| | | NO: 274) | (SEQ ID | GQYKSYYYMD | (SEQ ID | NO: 1050) | (SEQ ID |
| | | | NO: 468) | TV | NO: 856) | | NO: 1244) |
| | | | | (SEQ ID | | | |
| | | | | NO: 662) | | | |
| 107 | AIP- | GFTFSKAWMS | RIKSVTDGET | TSSFCCRGGS | SGSSSNIGSS | RNNQRPS | STWDDSLSVR |
| | 169636 | (SEQ ID | TDYAAPVKG | CPSHDTSYCG | SV | (SEQ ID | V |
| | | NO: 275) | (SEQ ID | GQYKSYYYMD | (SEQ ID | NO: 1051) | (SEQ ID |
| | | | NO: 469) | SV | NO: 857) | | NO: 1245) |
| | | | | (SEQ ID | | | |
| | | | | NO: 663) | | | |
| 108 | AIP- | GFTFSKAWMS | RIKSVTDGET | TSSFCCRGGS | SGSSSNIGSS | NNNORPS | STWDDSLSVR |
| | 152243 | (SEQ ID | TDYAAPVKG | CPSHDTSYCG | SVS | (SEQ ID | V |
| | | NO: 276) | (SEQ ID | GQYKSYYYMD | (SEQ ID | NO: 1052) | (SEQ ID |
| | | | NO: 470) | V | NO: 858) | | NO: 1246) |
| | | | | (SEQ ID | | | |
| | | | | NO: 664) | | | |
| 109 | AIP- | GFTFSKAWMS | RIKSVTDGET | TSSFCCRGGS | SGSSSNIGSS | MNNQRPS | STWDDSLSVR |
| | 138776 | (SEQ ID | TDYAAPVKG | CPSHDTSYCG | SV | (SEQ ID | V |
| | | NO: 277) | (SEQ ID | GQYKSYYYMD | (SEQ ID | NO: 1053) | (SEQ ID |
| | | | NO: 471) | SV | NO: 859) | | NO: 1247) |
| | | | | (SEQ ID | | | |
| | | | | NO: 665) | | | |
| 110 | AIP- | GFTFSKAWMS | RIKSVTDGET | TSSFCCRGGS | SGSSSNIGSS | ANNQRPS | STWDDSLSVR |
| | 103817 | (SEQ ID | TDYAAPVKG | CPSHDTSYCG | SVS | (SEQ ID | V |
| | | NO: 278) | (SEQ ID | GQYKSYYYMD | (SEQ ID | NO: 1054) | (SEQ ID |
| | | | NO: 472) | V | NO: 860) | | NO: 1248) |
| | | | | (SEQ ID | | | |
| | | | | NO: 666) | | | |
| 111 | AIP- | GFTFSKAWMS | RIKSVTDGET | TSSFCCRGGS | SGSSSNIGSS | HINNORPS | STWDDSLSVR |
| | 130491 | (SEQ ID | TDYAAPVKG | CPSHDTSYCG | SVS | (SEQ ID | V |
| | | NO: 279) | (SEQ ID | GQYKSYYYMD | (SEQ ID | NO: 1055) | (SEQ ID |
| | | | NO: 473) | V | NO: 861) | | NO: 1249) |
| | | | | (SEQ ID | | | |
| | | | | NO: 667) | | | |
| 112 | AIP- | GFTFSKAWMS | RIKSVTDGET | TSSFCCRGGS | SGSSSNIGSS | KDNORPS | STWDDSLSVR |
| | 188155 | (SEQ ID | TDYAAPVKG | CPSHDTSYCG | SV | (SEQ ID | V |
| | | NO: 280) | (SEQ ID | GQYKSYYYMD | (SEQ ID | NO: 1056) | (SEQ ID |
| | | | NO: 474) | SV | NO: 862) | | NO: 1250) |
| | | | | (SEQ ID | | | |
| | | | | NO: 668) | | | |
| 113 | AIP- | GFTFSKAWMS | RIKSVTDGET | TSSFCCRGGS | SGSSSNIGSS | KNTORPS | STWDDSLSVR |
| | 167246 | (SEQ ID | TDYAAPVKG | CPSHDTSYCG | SV | (SEQ ID | V |
| | | NO: 281) | (SEQ ID | GQYKSYYYMD | (SEQ ID | NO: 1057) | (SEQ ID |
| | | | NO: 475) | SV | NO: 863) | | NO: 1251) |
| | | | | (SEQ ID | | | |
| | | | | NO: 669) | | | |
| 114 | AIP- | GFTFSKAWMS | RIKSVTDGET | TSSFCCRGGS | SGSSSNIGSS | KNNYRPS | STWDDSLSVR |
| | 106139 | (SEQ ID | TDYAAPVKG | CPSHDTSYCG | SV | (SEQ ID | V |
| | | NO: 282) | (SEQ ID | GQYKSYYYMD | (SEQ ID | NO: 1058) | (SEQ ID |
| | | | NO: 476) | SV | NO: 864) | | NO: 1252) |
| | | | | (SEQ ID | | | |
| | | | | NO: 670) | | | |
| 115 | AIP- | GFTFSKAWMS | RIKSVTDGET | TSSFCCRGGS | SGSSSNIGSS | KNNARPS | STWDDSLSVR |
| | 198351 | (SEQ ID | TDYAAPVKG | CPSHDTSYCG | SV | (SEQ ID | V |
| | | NO: 283) | (SEQ ID | GQYKSYYYMD | (SEQ ID | NO: 1059) | (SEQ ID |
| | | | NO: 477) | SV | NO: 865) | | NO: 1253) |
| | | | | (SEQ ID | | | |
| | | | | NO: 671) | | | |
| 116 | AIP- | GFTFSKAWMS | RIKSVTDGET | TSSFCCRGGS | SGSSSNIGSS | KNNLRPS | STWDDSLSVR |
| | 159326 | (SEQ ID | TDYJAAPVKG | CPSHDTSYCG | SVS | (SEQ ID | V |
| | | NO: 284) | (SEQ ID | GQYKSYYYMD | (SEQ ID | NO: 1060) | (SEQ ID |
| | | | NO: 478) | V | NO: 866) | | NO: 1254) |
| | | | | (SEQ ID | | | |
| | | | | NO: 672) | | | |
| 117 | AIP- | GFTFSKAWMS | RIKSVTDGET | TSSFCCRGGS | SGSSSNIGSS | KNNQQPS | STWDDSLSVR |
| | 192275 | (SEQ ID | TDYJAAPVKG | CPSHDTSYCG | SV | (SEQ ID | V |
| | | NO: 285) | (SEQ ID | GQYKSYYYMD | (SEQ ID | NO: 1061) | (SEQ ID |
| | | | NO: 479) | SV | NO: 867) | | NO: 1255) |
| | | | | (SEQ ID | | | |
| | | | | NO: 673) | | | |
| 118 | AIP- | GFTFSKAWMS | RIKSVTDGET | TSSFCCRGGS | SGSSSNIGSS | KNNORAS | STWDDSLSVR |
| | 190761 | (SEQ ID | TDYAAPVKG | CPSHDTSYCG | SV | (SEQ ID | V |
| | | NO: 286) | (SEQ ID | GQYKSYYYMD | (SEQ ID | NO: 1062) | (SEQ ID |
| | | | NO: 480) | SV | NO: 868) | | NO: 1256) |
| | | | | (SEQ ID | | | |
| | | | | NO: 674) | | | |
| 119 | AIP- | GFTFSKAWMS | RIKSVTDGET | TSSFCCRGGS | SGSSSNIGSS | KNNQRPY | STWDDSLSVR |
| | 166832 | (SEQ ID | TDYAAPVKG | CPSHDTSYCG | SV | (SEQ ID | V |
| | | NO: 287) | (SEQ ID | GQYKSYYYMD | (SEQ ID | NO: 1063) | (SEQ ID |
| | | | NO: 481) | SV | NO: 869) | | NO: 1257) |
| | | | | (SEQ ID | | | |
| | | | | NO: 675) | | | |
| 120 | AIP- | GFTFSKAWMS | RIKSVTDGET | TSSFCCRGGS | SGSSSNIGSS | KNNORPL | STWDDSLSVR |
| | 148062 | (SEQ ID | TDYAAPVKG | CPSHDTSYCG | SV | (SEQ ID | V |
| | | NO: 288) | (SEQ ID | GQYKSYYYMD | (SEQ ID | NO: 1064) | (SEQ ID |
| | | | NO: 482) | SV | NO: 870) | | NO: 1258) |
| | | | | (SEQ ID | | | |
| | | | | NO: 676) | | | |
| 121 | AIP- | GFTFSKAWMS | RIKSVTDGET | TSSFCCRGGS | SGSSSNIGSS | KNNQRPS | ATWDDSLSVR |
| | 129145 | (SEQ ID | TDYAAPVKG | CPSHDTSYCG | SVS | (SEQ ID | V |
| | | NO: 289) | (SEQ ID | GQYKSYYYMD | (SEQ ID | NO: 1065) | (SEQ ID |
| | | | NO: 483) | V | NO: 871) | | NO: 1259) |
| | | | | (SEQ ID | | | |
| | | | | NO: 677) | | | |
| 122 | AIP- | GFTFSKAWMS | RIKSVTDGET | TSSFCCRGGS | SGSSSNIGSS | KNNORPS | SSWDDSLSVR |
| | 111240 | (SEQ ID | TDYAAPVKG | CPSHDTSYCG | SVS | (SEQ ID | V |
| | | NO: 290) | (SEQ ID | GQYKSYYYMD | (SEQ ID | NO: 1066) | (SEQ ID |
| | | | NO: 484) | V | NO: 872) | | NO: 1260) |
| | | | | (SEQ ID | | | |
| | | | | NO: 678) | | | |
| 123 | AIP- | GFTFSKAWMS | RIKSVTDGET | TSSFCCRGGS | SGSSSNIGSS | KNNORPS | STFDDSLSVR |
| | 190749 | (SEQ ID | TDYAAPVKG | CPSHDTSYCG | SV | (SEQ ID | V |
| | | NO: 291) | (SEQ ID | GQYKSYYYMD | (SEQ ID | NO: 1067) | (SEQ ID |
| | | | NO: 485) | SV | NO: 873) | | NO: 1261) |
| | | | | (SEQ ID | | | |
| | | | | NO: 679) | | | |
| 124 | AIP- | GFTFSKAWMS | RIKSVTDGET | TSSFCCRGGS | SGSSSNIGSS | KNNQRPS | STWDNSLSVR |
| | 153888 | (SEQ ID | TDYAAPVKG | CPSHDTSYCG | SVS | (SEQ ID | V |
| | | NO: 292) | (SEQ ID | GQYKSYYYMD | (SEQ ID | NO: 1068) | (SEQ ID |
| | | | NO: 486) | V | NO: 874) | | NO: 1262) |
| | | | | (SEQ ID | | | |
| | | | | NO: 680) | | | |
| 125 | AIP- | GFTFSKAWMS | RIKSVTDGET | TSSFCCRGGS | SGSSSNIGSS | KNNORPS | STWDDQLSVR |
| | 130915 | (SEQ ID | TDYAAPVKG | CPSHDTSYCG | SVS | (SEQ ID | V |
| | | NO: 293) | (SEQ ID | GQYKSYYYMD | (SEQ ID | NO: 1069) | (SEQ ID |
| | | | NO: 487) | V | NO: 875) | | NO: 1263) |
| | | | | (SEQ ID | | | |
| | | | | NO: 681) | | | |
| 126 | AIP- | GFTFSKAWMS | RIKSVTDGET | TSSFCCRGGS | SGSSSNIGSS | KNNQRPS | STWDDALSVR |
| | 109048 | (SEQ ID | TDYAAPVKG | CPSHDTSYCG | SVS | (SEQ ID | V |
| | | NO: 294) | (SEQ ID | GQYKSYYYMD | (SEQ ID | NO: 1070) | (SEQ ID |
| | | | NO: 488) | V | NO: 876) | | NO: 1264) |
| | | | | (SEQ ID | | | |
| | | | | NO: 682) | | | |
| 127 | AIP- | GFTFSKAWMS | RIKSVTDGET | TSSFCCRGGS | SGSSSNIGSS | KNNORPS | STWDDDLSVR |
| | 170569 | (SEQ ID | TDYAAPVKG | CPSHDTSYCG | SV | (SEQ ID | V |
| | | NO: 295) | (SEQ ID | GQYKSYYYMD | (SEQ ID | NO: 1071) | (SEQ ID |
| | | | NO: 489) | SV | NO: 877) | | NO: 1265) |
| | | | | (SEQ ID | | | |
| | | | | NO: 683) | | | |
| 128 | AIP- | GFTFSKAWMS | RIKSVTDGET | TSSFCCRGGS | SGSSSNIGSS | KNNORPS | STWDDSNSVR |
| | 154873 | (SEQ ID | TDYAAPVKG | CPSHDTSYCG | SV | (SEQ ID | V |
| | | NO: 296) | (SEQ ID | GQYKSYYYMD | (SEQ ID | NO: 1072) | (SEQ ID |
| | | | NO: 490) | SV | NO: 878) | | NO: 1266) |
| | | | | (SEQ ID | | | |
| | | | | NO: 684) | | | |
| 129 | AIP- | GFTFSKAWMS | RIKSVTDGET | TSSFCCRGGS | SGSSSNIGSS | KNNORPS | STWDDSSSVR |
| | 159037 | (SEQ ID | TDYAAPVKG | CPSHDTSYCG | SV | (SEQ ID | V |
| | | NO: 297) | (SEQ ID | GQYKSYYYMD | (SEQ ID | NO: 1073) | (SEQ ID |
| | | | NO: 491) | SV | NO: 879) | | NO: 1267) |
| | | | | (SEQ ID | | | |
| | | | | NO: 685) | | | |
| 130 | AIP- | GFTFSKAWMS | RIKSVTDGET | TSSFCCRGGS | SGSSSNIGSS | KNNORPS | STWDDSLNVR |
| | 186826 | (SEQ ID | TDYAAPVKG | CPSHDTSYCG | SVS | (SEQ ID | V |
| | | NO: 298) | (SEQ ID | GQYKSYYYMD | (SEQ ID | NO: 1074) | (SEQ ID |
| | | | NO: 492) | V | NO: 880) | | NO: 1268) |
| | | | | (SEQ ID | | | |
| | | | | NO: 686) | | | |
| 131 | AIP- | GFTFSKAWMS | RIKSVTDGET | TSSFCCRGGS | SGSSSNIGSS | KNNORPS | STWDDSLLVR |
| | 156514 | (SEQ ID | TDYJAAPVKG | CPSHDTSYCG | SV | (SEQ ID | V |
| | | NO: 299) | (SEQ ID | GQYKSYYYMD | (SEQ ID | NO: 1075) | (SEQ ID |
| | | | NO: 493) | SV | NO: 881) | | NO: 1269) |
| | | | | (SEQ ID | | | |
| | | | | NO: 687) | | | |
| 132 | AIP- | GFTFSKAWMS | RIKSVTDGET | TSSFCCRGGS | SGSSSNIGSS | KNNORPS | STWDDSLTVR |
| | 157122 | (SEQ ID | TDYAAPVKG | CPSHDTSYCG | SV | (SEQ ID | V |
| | | NO: 300) | (SEQ ID | GQYKSYYYMD | (SEQ ID | NO: 1076) | (SEQ ID |
| | | | NO: 494) | SV | NO: 882) | | NO: 1270) |
| | | | | (SEQ ID | | | |
| | | | | NO: 688) | | | |
| 133 | AIP- | GFTFSKAWMS | RIKSVTDGET | TSSFCCRGGS | SGSSSNIGSS | KNNORPS | STWDDSLSIR |
| | 173276 | (SEQ ID | TDYAAPVKG | CPSHDTSYCG | SV | (SEQ ID | V |
| | | NO: 301) | (SEQ ID | GQYKSYYYMD | (SEQ ID | NO: 1077) | (SEQ ID |
| | | | NO: 495) | SV | NO: 883) | | NO: 1271) |
| | | | | (SEQ ID | | | |
| | | | | NO: 689) | | | |
| 134 | AIP- | GFTFSKAWMS | RIKSVTDGET | TSSFCCRGGS | SGSSSNIGSS | KNNQRPS | STWDDSLSVK |
| | 150485 | (SEQ ID | TDYAAPVKG | CPSHDTSYCG | SV | (SEQ ID | V |
| | | NO: 302) | (SEQ ID | GQYKSYYYMD | (SEQ ID | NO: 1078) | (SEQ ID |
| | | | NO: 496) | SV | NO: 884) | | NO: 1272) |
| | | | | (SEQ ID | | | |
| | | | | NO: 690) | | | |
| 135 | AIP- | GFTFSKAWMS | RIKSVTDGET | TSSFCCRGGS | SGSSSNIGSS | KNNORPS | STWDDSLSVW |
| | 135679 | (SEQ ID | TDYAAPVKG | CPSHDTSYCG | SVS | (SEQ ID | V |
| | | NO: 303) | (SEQ ID | GQYKSYYYMD | (SEQ ID | NO: 1079) | (SEQ ID |
| | | | NO: 497) | V | NO: 885) | | NO: 1273) |
| | | | | (SEQ ID | | | |
| | | | | NO: 691) | | | |
| 136 | AIP- | GFTFSKAWMS | RIKSVTDGET | TSSFCCRGGS | SGSSSNIGSS | KNNQRPS | STWDDSLSVH |
| | 166847 | (SEQ ID | TDYAAPVKG | CPSHDTSYCG | SV | (SEQ ID | V |
| | | NO: 304) | (SEQ ID | GQYKSYYYMD | (SEQ ID | NO: 1080) | (SEQ ID |
| | | | NO: 498) | SV | NO: 886) | | NO: 1274) |
| | | | | (SEQ ID | | | |
| | | | | NO: 692) | | | |
| 137 | AIP- | GFTFSKAWMS | RIKSVTDGET | TSSFCCRGGS | SGSSSNIGSS | KNNORPS | STWDDSLSVQ |
| | 124013 | (SEQ ID | TDYAAPVKG | CPSHDTSYCG | SV | (SEQ ID | V |
| | | NO: 305) | (SEQ ID | GQYKSYYYMD | (SEQ ID | NO: 1081) | (SEQ ID |
| | | | NO: 499) | SV | NO: 887) | | NO: 1275) |
| | | | | (SEQ ID | | | |
| | | | | NO: 693) | | | |
| 138 | AIP- | GFTFSKAWMS | RIKSVTDGET | TSSFCCRGGS | SGSSSNIGSS | KNNORPS | STWDDSLSVR |
| | 126285 | (SEQ ID | TDYAAPVKG | CPSHDTSYCG | SVS | (SEQ ID | I |
| | | NO: 306) | (SEQ ID | GQYKSYYYMD | (SEQ ID | NO: 1082) | (SEQ ID |
| | | | NO: 500) | V | NO: 888) | | NO: 1276) |
| | | | | (SEQ ID | | | |
| | | | | NO: 694) | | | |
| 139 | AIP- | GFTFSKAWMS | RIKSVTDGET | TSSFCCRGGS | SGSSSDIGSS | KNNORPS | STWNDQLSVR |
| | 190274 | (SEQ ID | TDYAAPVKG | CPSHDTSYCG | SVS | (SEQ ID | V |
| | | NO: 307) | (SEQ ID | GQYKSYYYMD | (SEQ ID | NO: 1083) | (SEQ ID |
| | | | NO: 501) | V | NO: 889) | | NO: 1277) |
| | | | | (SEQ ID | | | |
| | | | | NO: 695) | | | |
| 140 | AIP- | GFTFSKAWMS | RIKSVTDGET | TSSFCCRGGS | HGSESDIGSH | KNNQRPS | STWDDSLSVR |
| | 150277 | (SEQ ID | TDYAAPVKG | CPSHDTSYCG | DV | (SEQ ID | V |
| | | NO: 308) | (SEQ ID | GQYKSYYYMD | (SEQ ID | NO: 1084) | (SEQ ID |
| | | | NO: 502) | LV | NO: 890) | | NO: 1278) |
| | | | | (SEQ ID | | | |
| | | | | NO: 696) | | | |
| 141 | AIP- | GFTFSKAWYS | RIKSVTDGET | TSSFCCRGGS | SGSSSNIGSS | KNNORPS | STWDDSLSVR |
| | 104364 | (SEQ ID | TDYAAPVKG | CPSHDTSYCG | SVS | (SEQ ID | V |
| | | NO: 309) | (SEQ ID | GQYKSYYYMD | (SEQ ID | NO: 1085) | (SEQ ID |
| | | | NO: 503) | V | NO: 891) | | NO: 1279) |
| | | | | (SEQ ID | | | |
| | | | | NO: 697) | | | |
| 142 | AIP- | GFTFSKAWMS | RIKSVTDGET | TSSFCCRGGS | SGSSSNIGSS | KNNQRPS | STWDDSLSVR |
| | 180422 | (SEQ ID | TEYAASVKG | CPSHDTSYCG | SV | (SEQ ID | V |
| | | NO: 310) | (SEQ ID | GQYKSYYYMD | (SEQ ID | NO: 1086) | (SEQ ID |
| | | | NO: 504) | SV | NO: 892) | | NO: 1280) |
| | | | | (SEQ ID | | | |
| | | | | NO: 698) | | | |
| 143 | AIP- | GFTFSKAWMS | RIKSVTDGET | TSSFCCRGGS | SGSSSNIGSS | KNNORPS | STWDDSLSVR |
| | 166722 | (SEQ ID | TEYAASVKG | CPSHDTSYCG | SV | (SEQ ID | V |
| | | NO: 311) | (SEQ ID | GQYKSYYYMD | (SEQ ID | NO: 1087) | (SEQ ID |
| | | | NO: 505) | SV | NO: 893) | | NO: 1281) |
| | | | | (SEQ ID | | | |
| | | | | NO: 699) | | | |
| 144 | AIP- | GFTFSKAWMS | FIKSVTDGET | TSSFCCRGGS | SGSSSNIGSS | KNNORPS | STWDDSLSVR |
| | 193490 | (SEQ ID | TEYAASVKG | CPSHDTSYCG | SV | (SEQ ID | V |
| | | NO: 312) | (SEQ ID | GQYKSYYYMD | (SEQ ID | NO: 1088) | (SEQ ID |
| | | | NO: 506) | SV | NO: 894) | | NO: 1282) |
| | | | | (SEQ ID | | | |
| | | | | NO: 700) | | | |
| 145 | AIP- | GFTFSKAWMS | RIKSVTDGET | TSSYCCRSGS | SGSSSNIGSS | KNNORPS | STWDDSLSVR |
| | 129967 | (SEQ ID | TDYAAPVKG | CPSSDTSYCN | SV | (SEQ ID | V |
| | | NO: 313) | (SEQ ID | GQFKSYYYMD | (SEQ ID | NO: 1089) | (SEQ ID |
| | | | NO: 507) | SP | NO: 895) | | NO: 1283) |
| | | | | (SEQ ID | | | |
| | | | | NO: 701) | | | |
| 146 | AIP- | GFTFSKAWMS | RIKSVTDGET | TSSFCCRSGS | SGSSSNIGSS | KNNORPS | STWDDSLSVR |
| | 126175 | (SEQ ID | TDYAAPVKG | CPSSDTSYCN | SV | (SEQ ID | V |
| | | NO: 314) | (SEQ ID | GQYKSYYYVN | (SEQ ID | NO: 1090) | (SEQ ID |
| | | | NO: 508) | SI | NO: 896) | | NO: 1284) |
| | | | | (SEQ ID | | | |
| | | | | NO: 702) | | | |
| 147 | AIP- | GFTFSKAWMS | RIKSVTDGET | TTSFCCRGAS | SGSSSNIGSS | KNNORPS | STWDDSLSVR |
| | 180905 | (SEQ ID | TDYAAPVKG | CPSHDTSYCA | SV | (SEQ ID | V |
| | | NO: 315) | (SEQ ID | GSYKSYYYVN | (SEQ ID | NO: 1091) | (SEQ ID |
| | | | NO: 509) | SI | NO: 897) | | NO: 1285) |
| | | | | (SEQ ID | | | |
| | | | | NO: 703) | | | |
| 148 | AIP- | GFTFSKAWMS | RIKSVTDGET | TSAFCCRGKS | SGSSSNIGSS | KNNORPS | STWDDSLSVR |
| | 141706 | (SEQ ID | TDYAAPVKG | CPSSDTSYCG | SV | (SEQ ID | V |
| | | NO: 316) | (SEQ ID | GQYPSYYYVN | (SEQ ID | NO: 1092) | (SEQ ID |
| | | | NO: 510) | SI | NO: 898) | | NO: 1286) |
| | | | | (SEQ ID | | | |
| | | | | NO: 704) | | | |
| 149 | AIP- | GFTFSKAWMS | RIKSVTDGET | TSAFCCRGKS | SGSSSNIGSS | KNNORPS | STWDDSLSVR |
| | 105092 | (SEQ ID | TDYAAPVKG | CPSHDTSFCG | SV | (SEQ ID | V |
| | | NO: 317) | (SEQ ID | GODKRYYYVN | (SEQ ID | NO: 1093) | (SEQ ID |
| | | | NO: 511) | SI | NO: 899) | | NO: 1287) |
| | | | | (SEQ ID | | | |
| | | | | NO: 705) | | | |
| 150 | AIP- | GFTFSKAWMS | RIKSVTDGET | TSAFCCRGGN | SGSSSNIGSS | KNNORPS | STWDDSLSVR |
| | 152031 | (SEQ ID | TDYAAPVKG | CPSHETSYCN | SVS | (SEQ ID | V |
| | | NO: 318) | (SEQ ID | GONKQYYYMD | (SEQ ID | NO: 1094) | (SEQ ID |
| | | | NO: 512) | V | NO: 900) | | NO: 1288) |
| | | | | (SEQ ID | | | |
| | | | | NO: 706) | | | |
| 151 | AIP- | GFTFSKAWMS | RIKSVTDGET | TSSFCCRGNQ | SGSSSNIGSS | KNNORPS | STWDDSLSVR |
| | 163319 | (SEQ ID | TDYAAPVKG | CPSSDTSFCG | SV | (SEQ ID | V |
| | | NO: 319) | (SEQ ID | GODKRYYYMD | (SEQ ID | NO: 1095) | (SEQ ID |
| | | | NO: 513) | SP | NO: 901) | | NO: 1289) |
| | | | | (SEQ ID | | | |
| | | | | NO: 707) | | | |
| 152 | AIP- | GFTFSKAWMS | RIKSVTDGET | TTSFCCRGNQ | SGSSSNIGSS | KNNQRPS | STWDDSLSVR |
| | 160621 | (SEQ ID | TDYAAPVKG | CPSHETSYCG | SV | (SEQ ID | V |
| | | NO: 320) | (SEQ ID | GYYKSYFYMD | (SEQ ID | NO: 1096) | (SEQ ID |
| | | | NO: 514) | SV | NO: 902) | | NO: 1290) |
| | | | | (SEQ ID | | | |
| | | | | NO: 708) | | | |
| 153 | AIP- | GFTFSKAWMS | RIKSVTDGET | TSSYCCRGKQ | SGSSSNIGSS | KNNORPS | STWDDSLSVR |
| | 145212 | (SEQ ID | TDYAAPVKG | CPSHDTSYCA | SV | (SEQ ID | V |
| | | NO: 321) | (SEQ ID | GQYADYYYMD | (SEQ ID | NO: 1097) | (SEQ ID |
| | | | NO: 515) | SV | NO: 903) | | NO: 1291) |
| | | | | (SEQ ID | | | |
| | | | | NO: 709) | | | |
| 154 | AIP- | GFTFSKAWMS | RIKSVTDGET | ISSFCCRGKQ | SGSSSNIGSS | KNNORPS | STWDDSLSVR |
| | 112328 | (SEQ ID | TDYAAPVKG | CPSSDTSYCN | SV | (SEQ ID | V |
| | | NO: 322) | (SEQ ID | GQYADYYYMD | (SEQ ID | NO: 1098) | (SEQ ID |
| | | | NO: 516) | SV | NO: 904) | | NO: 1292) |
| | | | | (SEQ ID | | | |
| | | | | NO: 710) | | | |
| 155 | AIP- | GFTFSKAWMS | RIKSVTDGET | TSSFCCRGKQ | SGSSSNIGSS | KNNORPS | STWDDSLSVR |
| | 193106 | (SEQ ID | TDYAAPVKG | CPSSDTSYCN | SV | (SEQ ID | V |
| | | NO: 323) | (SEQ ID | GQYADYYYVN | (SEQ ID | NO: 1099) | (SEQ ID |
| | | | NO: 517) | SI | NO: 905) | | NO: 1293) |
| | | | | (SEQ ID | | | |
| | | | | NO: 711) | | | |
| 156 | AIP- | GFTFSKAWMS | RIKSVTDGET | ISSFCCRGKQ | SGSSSNIGSS | KNNQRPS | STWDDSLSVR |
| | 125062 | (SEQ ID | TDYAAPVKG | CPSSDTSFCG | SV | (SEQ ID | V |
| | | NO: 324) | (SEQ ID | GODKRYYFMD | (SEQ ID | NO: 1100) | (SEQ ID |
| | | | NO: 518) | SV | NO: 906) | | NO: 1294) |
| | | | | (SEQ ID | | | |
| | | | | NO: 712) | | | |
| 157 | AIP- | GFTFSKAWMS | RIKSVTDGET | TSAFCCRGKQ | SGSSSNIGSS | KNNORPS | STWDDSLSVR |
| | 124301 | (SEQ ID | TDYAAPVKG | CPSSDTSYCG | SV | (SEQ ID | V |
| | | NO: 325) | (SEQ ID | GQYASFYYMD | (SEQ ID | NO: 1101) | (SEQ ID |
| | | | NO: 519) | SV | NO: 907) | | NO: 1295) |
| | | | | (SEQ ID | | | |
| | | | | NO: 713) | | | |
| 158 | AIP- | GFTFSKAWMS | RIKSVTDGET | TSSFCCOGNN | SGSSSNIGSS | KNNORPS | STWDDSLSVR |
| | 124068 | (SEQ ID | TDYAAPVKG | CPSSDTSYCG | SV | (SEQ ID | V |
| | | NO: 326) | (SEQ ID | GYYKDYYYMD | (SEQ ID | NO: 1102) | (SEQ ID |
| | | | NO: 520) | SV | NO: 908) | | NO: 1296) |
| | | | | (SEQ ID | | | |
| | | | | NO: 714) | | | |
| 159 | AIP- | GFTFSKAWMS | RIKSVTDGET | TSSFCCOGNN | SGSSSNIGSS | KNNQRPS | STWDDSLSVR |
| | 144568 | (SEQ ID | TDYAAPVKG | CPSHDTSYCG | SV | (SEQ ID | V |
| | | NO: 327) | (SEQ ID | GQYKSYYYVN | (SEQ ID | NO: 1103) | (SEQ ID |
| | | | NO: 521) | SI | NO: 909) | | NO: 1297) |
| | | | | (SEQ ID | | | |
| | | | | NO: 715) | | | |
| 160 | AIP- | GFTFSKAWMS | RIKSVTDGET | TSSFCCOGKN | SGSSSNIGSS | KNNORPS | STWDDSLSVR |
| | 139782 | (SEQ ID | TDYAAPVKG | CPSHETSYCG | SVS | (SEQ ID | V |
| | | NO: 328) | (SEQ ID | GQYADYYYMD | (SEQ ID | NO: 1104) | (SEQ ID |
| | | | NO: 522) | V | NO: 910) | | NO: 1298) |
| | | | | (SEQ ID | | | |
| | | | | NO: 716) | | | |
| 161 | AIP- | GFTFSKAWMS | RIKSVTDGET | TTSFCCHGNS | SGSSSNIGSS | KNNQRPS | STWDDSLSVR |
| | 171543 | (SEQ ID | TDYAAPVKG | CPSSDTSYCG | SVS | (SEQ ID | V |
| | | NO: 329) | (SEQ ID | GONKQYYYMD | (SEQ ID | NO: 1105) | (SEQ ID |
| | | | NO: 523) | V | NO: 911) | | NO: 1299) |
| | | | | (SEQ ID | | | |
| | | | | NO: 717) | | | |
| 162 | AIP- | GFTFSKAWMS | RIKSVTDGET | ISSFCCHGNS | SGSSSNIGSS | KNNQRPS | STWDDSLSVR |
| | 140148 | (SEQ ID | TDYAAPVKG | CPSHDTSYCN | SVS | (SEQ ID | V |
| | | NO: 330) | (SEQ ID | GQNKQYYYMD | (SEQ ID | NO: 1106) | (SEQ ID |
| | | | NO: 524) | V | NO: 912) | | NO: 1300) |
| | | | | (SEQ ID | | | |
| | | | | NO: 718) | | | |
| 163 | AIP- | GFTFSKAWMS | RIKSVTDGET | ISSFCCHSNN | SGSSSNIGSS | KNNQRPS | STWDDSLSVR |
| | 177193 | (SEQ ID | TDYAAPVKG | CPSSDTSYCN | SVS | (SEQ ID | V |
| | | NO: 331) | (SEQ ID | GQYKQYYYMD | (SEQ ID | NO: 1107) | (SEQ ID |
| | | | NO: 525) | V | NO: 913) | | NO: 1301) |
| | | | | (SEQ ID | | | |
| | | | | NO: 719) | | | |
| 164 | AIP- | GFTFSKAWMS | RIKSVTDGET | TSSFCCHSNN | SGSSSNIGSS | KNNORPS | STWDDSLSVR |
| | 171348 | (SEQ ID | TDYAAPVKG | CPSHDTSYCG | SV | (SEQ ID | V |
| | | NO: 332) | (SEQ ID | GQYKSYYYVN | (SEQ ID | NO: 1108) | (SEQ ID |
| | | | NO: 526) | SI | NO: 914) | | NO: 1302) |
| | | | | (SEQ ID | | | |
| | | | | NO: 720) | | | |
| 165 | AIP- | GFTFSKAWMS | RIKSVTDGET | ISSFACHSNN | SGSSSNIGSS | KNNORPS | STWDDSLSVR |
| | 193088 | (SEQ ID | TDYAAPVKG | CPSSDTSYVN | SV | (SEQ ID | V |
| | | NO: 333) | (SEQ ID | GYYKQYYFMD | (SEQ ID | NO: 1109) | (SEQ ID |
| | | | NO: 527) | SV | NO: 915) | | NO: 1303) |
| | | | | (SEQ ID | | | |
| | | | | NO: 721) | | | |
| 166 | AIP- | GFTFSKAWMS | RIKSVTDGET | TSSFCCHSNN | SGSSSNIGSS | KNNORPS | STWDDSLSVR |
| | 182087 | (SEQ ID | TDYAAPVKG | CPSSDTSYCN | SV | (SEQ ID | V |
| | | NO: 334) | (SEQ ID | GQYKQYYYVN | (SEQ ID | NO: 1110) | (SEQ ID |
| | | | NO: 528) | SI | NO: 916) | | NO: 1304) |
| | | | | (SEQ ID | | | |
| | | | | NO: 722) | | | |
| 167 | AIP- | GFTFSKAWMS | RIKSVTDGET | TSSFCCLGKA | SGSSSNIGSS | KNNORPS | STWDDSLSVR |
| | 151388 | (SEQ ID | TDYAAPVKG | CLSHDTSYCG | SVS | (SEQ ID | V |
| | | NO: 335) | (SEQ ID | GQYASYYYVN | (SEQ ID | NO: 1111) | (SEQ ID |
| | | | NO: 529) | I | NO: 917) | | NO: 1305) |
| | | | | (SEQ ID | | | |
| | | | | NO: 723) | | | |
| 168 | AIP- | GFTFSKAWMS | RIKSVTDGET | TSSFCCLGKA | SGSSSNIGSS | KNNORPS | STWDDSLSVR |
| | 149787 | (SEQ ID | TDYAAPVKG | CLSSDTSYCG | SV | (SEQ ID | V |
| | | NO: 336) | (SEQ ID | GYYASYYFVN | (SEQ ID | NO: 1112) | (SEQ ID |
| | | | NO: 530) | SI | NO: 918) | | NO: 1306) |
| | | | | (SEQ ID | | | |
| | | | | NO: 724) | | | |
| 169 | AIP- | GFTFSKAWMS | RIKSVTDGET | TSSFACLGKA | SGSSSNIGSS | KNNORPS | STWDDSLSVR |
| | 126097 | (SEQ ID | TDYAAPVKG | CLSSDTSYVG | SV | (SEQ ID | V |
| | | NO: 337) | (SEQ ID | GYYASYYFVN | (SEQ ID | NO: 1113) | (SEQ ID |
| | | | NO: 531) | SI | NO: 919) | | NO: 1307) |
| | | | | (SEQ ID | | | |
| | | | | NO: 725) | | | |
| 170 | AIP- | GFTFSKAWMS | RIKSVTDGET | TSSYCCLGNS | SGSSSNIGSS | KNNORPS | STWDDSLSVR |
| | 107759 | (SEQ ID | TDYAAPVKG | CPSSDTSYCG | SV | (SEQ ID | V |
| | | NO: 338) | (SEQ ID | GOFKSYYYMD | (SEQ ID | NO: 1114) | (SEQ ID |
| | | | NO: 532) | SP | NO: 920) | | NO: 1308) |
| | | | | (SEQ ID | | | |
| | | | | NO: 726) | | | |
| 171 | AIP- | GFTFSKAWMS | RIKSVTDGET | TSSYCCLGNS | SGSSSNIGSS | KNNORPS | STWDDSLSVR |
| | 148484 | (SEQ ID | TDYAAPVKG | CPSSDTSFCG | SVS | (SEQ ID | V |
| | | NO: 339) | (SEQ ID | GODKRYYYMD | (SEQ ID | NO: 1115) | (SEQ ID |
| | | | NO: 533) | P | NO: 921) | | NO: 1309) |
| | | | | (SEQ ID | | | |
| | | | | NO: 727) | | | |
| 172 | AIP- | GFTFSKAWMS | RIKSVTDGET | ISSFCCLGGN | SGSSSNIGSS | KNNQRPS | STWDDSLSVR |
| | 186403 | (SEQ ID | TDYAAPVKG | CPSSETSYCG | SV | (SEQ ID | V |
| | | NO: 340) | (SEQ ID | NQYPSYYYMD | (SEQ ID | NO: 1116) | (SEQ ID |
| | | | NO: 534) | SV | NO: 922) | | NO: 1310) |
| | | | | (SEQ ID | | | |
| | | | | NO: 728) | | | |
| 173 | AIP- | GFTFSKAWMS | RIKSVTDGET | ISSFCCLGGN | SGSSSNIGSS | KNNQRPS | STWDDSLSVR |
| | 166629 | (SEQ ID | TDYAAPVKG | CPSSETSYCG | SV | (SEQ ID | V |
| | | NO: 341) | (SEQ ID | NYYPSYFYMD | (SEQ ID | NO: 1117) | (SEQ ID |
| | | | NO: 535) | SV | NO: 923) | | NO: 1311) |
| | | | | (SEQ ID | | | |
| | | | | NO: 729) | | | |
| 174 | AIP- | GFTFSKAWMS | RIKSVTDGET | TSSFCCLGGN | SGSSSNIGSS | KNNORPS | STWDDSLSVR |
| | 165276 | (SEQ ID | TDYAAPVKG | CPSSETSYCG | SV | (SEQ ID | V |
| | | NO: 342) | (SEQ ID | NQYPSYYYVN | (SEQ ID | NO: 1118) | (SEQ ID |
| | | | NO: 536) | SI | NO: 924) | | NO: 1312) |
| | | | | (SEQ ID | | | |
| | | | | NO: 730) | | | |
| 175 | AIP- | GFTFSKAWMS | RIKSVTDGET | TTSFACRGNQ | SGSSSNIGSS | KNNORPS | STWDDSLSVR |
| | 188293 | (SEQ ID | TDYAAPVKG | CPSHETSYVG | SV | (SEQ ID | V |
| | | NO: 343) | (SEQ ID | GYYKSYFYMD | (SEQ ID | NO: 1119) | (SEQ ID |
| | | | NO: 537) | SV | NO: 925) | | NO: 1313) |
| | | | | (SEQ ID | | | |
| | | | | NO: 731) | | | |
| 176 | AIP- | GFTFSKAWMS | RIKSVTDGET | TTSFACRGNO | SGSSSNIGSS | KNNORPS | STWDDSLSVR |
| | 109364 | (SEQ ID | TDYAAPVKG | CPSSETSYVG | SV | (SEQ ID | V |
| | | NO: 344) | (SEQ ID | GYYKSYFFMD | (SEQ ID | NO: 1120) | (SEQ ID |
| | | | NO: 538) | SV | NO: 926) | | NO: 1314) |
| | | | | (SEQ ID | | | |
| | | | | NO: 732) | | | |
| 177 | AIP- | GFTFSKAWMS | RIKSVTDGET | ISSFACRGKQ | SGSSSNIGSS | KNNQRPS | STWDDSLSVR |
| | 191805 | (SEQ ID | TDYAAPVKG | CPSSDTSYVG | SV | (SEQ ID | V |
| | | NO: 345) | (SEQ ID | GQFKSYYFMD | (SEQ ID | NO: 1121) | (SEQ ID |
| | | | NO: 539) | SV | NO: 927) | | NO: 1315) |
| | | | | (SEQ ID | | | |
| | | | | NO: 733) | | | |
| 178 | AIP- | GFTFAKAWMS | RIKAADDGKQ | TSSFCCRGGS | SGSSTNIGSS | KNNYRPS | STWDDSLSVR |
| | 181592 | (SEQ ID | TDYAAPVKG | CLSHDTSYCG | SVY | (SEQ ID | V |
| | | NO: 346) | (SEQ ID | GQYKSYYWMD | (SEQ ID | NO: 1122) | (SEQ ID |
| | | | NO: 540) | V | NO: 928) | | NO: 1316) |
| | | | | (SEQ ID | | | |
| | | | | NO: 734) | | | |
| 179 | AIP- | GFTYAKAWMS | RIKSVQDGEQ | TSSYCCLGGS | SGSSTNIGSS | KNNYRAS | STWDDSLSVR |
| | 155587 | (SEQ ID | TDYAAPVKG | CPSHDTSYCG | SVY | (SEQ ID | V |
| | | NO: 347) | (SEQ ID | GSYKSYYYMD | (SEQ ID | NO: 1123) | (SEQ ID |
| | | | NO: 541) | V | NO: 929) | | NO: 1317) |
| | | | | (SEQ ID | | | |
| | | | | NO: 735) | | | |
| 180 | AIP- | GFDASKAWFT | RIKSVTDGET | TSSFCCRGGS | TGSSSNIGSS | KNSLRPQ | STWDDSLSVR |
| | 147945 | (SEQ ID | TDYAAPVKG | CPSHDTSYCG | YVS | (SEQ ID | V |
| | | NO: 348) | (SEQ ID | GONKQYYYMD | (SEQ ID | NO: 1124) | (SEQ ID |
| | | | NO: 542) | V | NO: 930) | | NO: 1318) |
| | | | | (SEQ ID | | | |
| | | | | NO: 736) | | | |
| 181 | AIP- | GFDASKAWFT | RIKSVTDGET | TSSFACRGGS | TGSSSNIGSS | KNSLRPQ | STWDDSLSVR |
| | 199264 | (SEQ ID | TDYAAPVKG | CPSHDTSYVG | YVS | (SEQ ID | V |
| | | NO: 349) | (SEQ ID | GQYKSYYYMD | (SEQ ID | NO: 1125) | (SEQ ID |
| | | | NO: 543) | V | NO: 931) | | NO: 1319) |
| | | | | (SEQ ID | | | |
| | | | | NO: 737) | | | |
| 182 | AIP- | GFDASKAWFT | RIKSVTDGET | TSSYCCLGGS | TGSSSNIGSS | KNSLRPQ | STWDDSLSVR |
| | 123438 | (SEQ ID | TDYAAPVKG | CPSHDTSYCG | YV | (SEQ ID | V |
| | | NO: 350) | (SEQ ID | GSYKSYYYMD | (SEQ ID | NO: 1126) | (SEQ ID |
| | | | NO: 544) | SV | NO: 932) | | NO: 1320) |
| | | | | (SEQ ID | | | |
| | | | | NO: 738) | | | |
| 183 | AIP- | GFDASKAWFT | RIKSVTDGET | TSSFCCRGKS | TGSSSNIGSS | KNSLRPQ | STWDDSLSVR |
| | 151315 | (SEQ ID | TDYAAPVKG | CPSHDTSYCG | YV | (SEQ ID | V |
| | | NO: 351) | (SEQ ID | GOYASYYYMD | (SEQ ID | NO: 1127) | (SEQ ID |
| | | | NO: 545) | SV | NO: 933) | | NO: 1321) |
| | | | | (SEQ ID | | | |
| | | | | NO: 739) | | | |
| 184 | AIP- | GFTFSKAWMS | RIKAADDGKQ | TSSFCCRGGN | SGSPSNIGSA | KNNORPS | STWDDSLSVR |
| | 197785 | (SEQ ID | TDYAAPVKG | CPSHETSYCG | ST | (SEQ ID | V |
| | | NO: 352) | (SEQ ID | NQYKSYYYMD | (SEQ ID | NO: 1128) | (SEQ ID |
| | | | NO: 546) | SV | NO: 934) | | NO: 1322) |
| | | | | (SEQ ID | | | |
| | | | | NO: 740) | | | |
| 185 | AIP- | GFTFSKAWMS | RIKAADDGKQ | TSSFCCRSGS | SGSPSNIGSA | KNNQRPS | STWDDSLSVR |
| | 115782 | (SEQ ID | TDYAAPVKG | CPSSDTSYCN | STS | (SEQ ID | V |
| | | NO: 353) | (SEQ ID | GQYKSYYYMD | (SEQ ID | NO: 1129) | (SEQ ID |
| | | | NO: 547) | V | NO: 935) | | NO: 1323) |
| | | | | (SEQ ID | | | |
| | | | | NO: 741) | | | |
| 186 | AIP- | GFTFSKAWMS | RIKAADDGKQ | TSSFCCRGGS | SGSPSNIGSA | KNNORPS | STWDDSLSVR |
| | 138130 | (SEQ ID | TDYAAPVKG | CPSHDTSYCG | ST | (SEQ ID | V |
| | | NO: 354) | (SEQ ID | GQYKSYYYVN | (SEQ ID | NO: 1130) | (SEQ ID |
| | | | NO: 548) | SI | NO: 936) | | NO: 1324) |
| | | | | (SEQ ID | | | |
| | | | | NO: 742) | | | |
| 187 | AIP- | GFTFSKAWMS | RIKAADDGKQ | TTSFCCRGAS | SGSPSNIGSA | KNNORPS | STWDDSLSVR |
| | 170221 | (SEQ ID | TDYAAPVKG | CPSHDTSYCA | STS | (SEQ ID | V |
| | | NO: 355) | (SEQ ID | GSYKSYYYMD | (SEQ ID | NO: 1131) | (SEQ ID |
| | | | NO: 549) | V | NO: 937) | | NO: 1325) |
| | | | | (SEQ ID | | | |
| | | | | NO: 743) | | | |
| 188 | AIP- | GFTFSKAWMS | RIKAADDGKO | ISSFCCRGNS | SGSPSNIGSA | KNNORPS | STWDDSLSVR |
| | 167482 | (SEQ ID | TDYAAPVKG | CPSSDTSYCN | ST | (SEQ ID | V |
| | | NO: 356) | (SEQ ID | GQYKSYYYMD | (SEQ ID | NO: 1132) | (SEQ ID |
| | | | NO: 550) | SV | NO: 938) | | NO: 1326) |
| | | | | (SEQ ID | | | |
| | | | | NO: 744) | | | |
| 189 | AIP- | GFTFSKAWMS | RIKAVTDGHT | TSSFCCRGGS | SGASSNIGHS | RNNQQPL | STWDDALSIR |
| | 189475 | (SEQ ID | TDYAAPVKG | CPSHDTSYCG | SVY | (SEQ ID | V |
| | | NO: 357) | (SEQ ID | GQYKSYYYMD | (SEQ ID | NO: 1133) | (SEQ ID |
| | | | NO: 551) | V | NO: 939) | | NO: 1327) |
| | | | | (SEQ ID | | | |
| | | | | NO: 745) | | | |
| 190 | AIP- | GFVYSKAWMS | RIKSVTDGQA | TSSFCCRGGS | SGSSSNIGSS | KNTORAS | STWDDALNVR |
| | 102833 | (SEQ ID | TDYJAAPVKG | CPSHDTSYCG | SV | (SEQ ID | V |
| | | NO: 358) | (SEQ ID | GQYKSYYYMD | (SEQ ID | NO: 1134) | (SEQ ID |
| | | | NO: 552) | SV | NO: 940) | | NO: 1328) |
| | | | | (SEQ ID | | | |
| | | | | NO: 746) | | | |
| 191 | AIP- | GFVFSKAWMS | RIKSVTDGET | TSSFCCRGKS | SGSSSNIGSS | RNNQQPL | SSWDDSSLVR |
| | 173396 | (SEQ ID | TDYAAPVKG | CPSHDTSYCG | SV | (SEQ ID | V |
| | | NO: 359) | (SEQ ID | GQYASYYYMD | (SEQ ID | NO: 1135) | (SEQ ID |
| | | | NO: 553) | SV | NO: 941) | | NO: 1329) |
| | | | | (SEQ ID | | | |
| | | | | NO: 747) | | | |
| 192 | AIP- | GFTFAKAWMS | RIKSVTDAET | TSSFCCRSGS | SGSSSNIGSS | KNNARPS | ATWDNSLSIR |
| | 132355 | (SEQ ID | TDYAAPVKG | CPSSDTSYCN | SV | (SEQ ID | V |
| | | NO: 360) | (SEQ ID | GQYKSYYWMD | (SEQ ID | NO: 1136) | (SEQ ID |
| | | | NO: 554) | TV | NO: 942) | | NO: 1330) |
| | | | | (SEQ ID | | | |
| | | | | NO: 748) | | | |
| 193 | AIP- | GFTYAKAWFT | RIKSTSDGGI | TSSFCCRGGS | SGSKSNIGSS | KDNORAS | STWDDALSVR |
| | 118505 | (SEQ ID | TDYAAPVKG | CPSHDTSFCG | YV | (SEQ ID | V |
| | | NO: 361) | (SEQ ID | GQYKSYYYMD | (SEQ ID | NO: 1137) | (SEQ ID |
| | | | NO: 555) | SV | NO: 943) | | NO: 1331) |
| | | | | (SEQ ID | | | |
| | | | | NO: 749) | | | |
| 194 | AIP- | GFTFSKAWMS | RIKSVSDGET | TSSFCCRGGS | SGSSSNIGSS | KNNORPS | STWDDSLSVR |
| | 131972 | (SEQ ID | TDYAAPVKG | CPSRDTSYCG | SV | (SEQ ID | V |
| | | NO: 362) | (SEQ ID | GQYKSYYFMD | (SEQ ID | NO: 1138) | (SEQ ID |
| | | | NO: 556) | YV | NO: 944) | | NO: 1332) |
| | | | | (SEQ ID | | | |
| | | | | NO: 750) | | | |
| 195 | AIP- | GFTFSKAWMT | RIKSTSDGGI | TTSFCCRGGR | SGSSSNIGSS | KNNORPS | STWDDSLSVR |
| | 189526 | (SEQ ID | TDYAAPVKG | CPSRDTSFCG | SVS | (SEQ ID | V |
| | | NO: 171) | (SEQ ID | GQYNSYYYMD | (SEQ ID | NO: 1138) | (SEQ ID |
| | | | NO: 555) | V | NO: 941) | | NO: 1332) |
| | | | | (SEQ ID | | | |
| | | | | NO: 1727) | | | |

TABLE 3A

| | AIP | | |
|---|---|---|---|
| | Number | V_{H} Sequence | V_{L} Sequence |
| 1 | AIP- | EVQLVESGGALVKPGGSLRLSCAASGFTFSKAWMS | QSVLTQPPSASGTPGQRVTISCSGSSSNIGSSSVSWYQQLPG |
| | 131762 | WVRQAPGKGLEWVGRIKSVTDGETTDYAAPVKGRF | TAPKLLIYKNNQRPSGVPDRFSGSKSGTSASLAISGLRSEDE |
| | | TISRDDSKSTLYLQMNSLKTEDTAVYYCTSSFCGG | ADYYCSTWDDSLSVRVFGGGTKLTVL (SEQ ID NO: |
| | | PSHDTSYCGGQYKSYYYMDVWGKGTTVTVSS | 123) |
| | | (SEQ ID NO: 1747) | |
| 2 | AIP- | EVQLVESGGALVKPGGSLRLSCAASGFTFSKAWMS | QSVLTQPPSASGTPGQRVTISCSGSSSNIGSSSVSWYQQLPG |
| | 194564 | WVRQAPGKGLEWVGRIKSVTDGETTDYAAPVKGRF | TAPKLLIYKNNQRPSGVPDRFSGSKSGTSASLAISGLRSEDE |
| | | TISRDDSKSTLYLQMNSLKTEDTAVYYCTSSFCCR | ADYYCSTWDDSLSVRVFGGGTKLTVL (SEQ ID NO: |
| | | SGGSCPSHDTSYCGGQYKSYYYMDVWGKGTTVTVS | 123) |
| | | S (SEQ ID NO: 1748) | |
| 3 | AIP- | EVQLVESGGALVKPGGSLRLSCAASGFTFSKAWMS | QSVLTQPPSASGTPGQRVTISCSGSSSNIGSSSVSWYQQLPG |
| | 169752 | WVRQAPGKGLEWVGRIKSVTDGETTDYAAPVKGRF | TAPKLLIYKNNQRPSGVPDRFSGSKSGTSASLAISGLRSEDE |
| | | TISRDDSKSTLYLQMNSLKTEDTAVYYCTSSFCCR | ADYYCSTWDDSLSVRVFGGGTKLTVL (SEQ ID NO: |
| | | GGGSCPSHDTSYCGGQYKSYYYMDVWGKGTTVTVS | 123) |
| | | S (SEQ ID NO: 1749) | |
| 4 | AIP- | EVQLVESGGALVKPGGSLRLSCAASGFTFSKAWMS | QSVLTQPPSASGTPGORVTISCSGSSSNIGSSSVSWYQQLPG |
| | 185680 | WVRQAPGKGLEWVGRIKSVTDGETTDYAAPVKGRF | TAPKLLIYKNNQRPSGVPDRFSGSKSGTSASLAISGLRSEDE |
| | | TISRDDSKSTLYLOMNSLKTEDTAVYYCTSSFCCR | ADYYCSTWDDSLSVRVFGGGTKLTVL (SEQ ID NO: |
| | | GGGGSCPSHDTSYCGGQYKSYYYMDVWGKGTTVTV | 123) |
| | | SS (SEQ ID NO: 1750) | |
| 5 | AIP- | EVQLVESGGALVKPGGSLRLSCAASGFTFSKAWMS | QSVLTQPPSASGTPGQRVTISCSGSSSNIGSSSVSWYQQLPG |
| | 145328 | WVRQAPGKGLEWVGRIKSVTDGETTDYAAPVKGRF | TAPKLLIYKNNQRPSGVPDRFSGSKSGTSASLAISGLRSEDE |
| | | TISRDDSKSTLYLQMNSLKTEDTAVYYCTSSFCCR | ADYYCSTWDDSLSVRVFGGGTKLTVL (SEQ ID NO: |
| | | GGSGGSCPSHDTSYCGGQYKSYYYMDVWGKGTTVT | 123) |
| | | VSS (SEQ ID NO: 1751) | |
| 6 | AIP- | EVQLVESGGALVKPGGSLRLSCAASGFTFSKAWMS | QSVLTQPPSASGTPGQRVTISCSGSSSNIGSSSVSWYQQLPG |
| | 182606 | WVRQAPGKGLEWVGRIKSVTDGETTDYAAPVKGRF | TAPKLLIYKNNQRPSGVPDRFSGSKSGTSASLAISGLRSEDE |
| | | TISRDDSKSTLYLQMNSLKTEDTAVYYCTSSFSCR | ADYYCSTWDDSLSVRVFGGGTKLTVL (SEQ ID NO: |
| | | GGSCPSHDTSYGGQYKSYYYMDVWGKGTTVTVSS | 123) |
| | | (SEQ ID NO: 1752) | |
| 7 | AIP- | EVQLVESGGALVKPGGSLRLSCAASGFTFSKAWMS | QSVLTQPPSASGTPGQRVTISCSGSSSNIGSSSVSWYQQLPG |
| | 107388 | WVRQAPGKGLEWVGRIKSVTDGETTDYAAPVKGRF | TAPKLLIYKNNQRPSGVPDRFSGSKSGTSASLAISGLRSEDE |
| | | TISRDDSKSTLYLQMNSLKTEDTAVYYCTSSFCCR | ADYYCSTWDDSLSVRVFGGGTKLTVL (SEQ ID NO: |
| | | GGSCPSHDTSYCGGGQYKSYYYMDVWGKGTTVTVS | 123) |
| | | S (SEQ ID NO: 1753) | |
| 8 | AIP- | EVQLVESGGALVKPGGSLRLSCAASGFTFSKAWMS | QSVLTQPPSASGTPGQRVTISCSGSSSNIGSSSVSWYQQLPG |
| | 191692 | WVRQAPGKGLEWVGRIKSVTDGETTDYAAPVKGRF | TAPKLLIYKNNQRPSGVPDRFSGSKSGTSASLAISGLRSEDE |
| | | TISRDDSKSTLYLQMNSLKTEDTAVYYCTSSFCCR | ADYYCSTWDDSLSVRVFGGGTKLTVL (SEQ ID NO: |
| | | GGSCPSHDTSYCGGGSGYKSYYYMDVWGKGTTVTV | 123) |
| | | SS (SEQ ID NO: 1754) | |
| 9 | AIP- | EVQLVESGGALVKPGGSLRLSCAASGFTFSKAWMS | QSVLTQPPSASGTPGQRVTISCSGSSSNIGSSSVSWYQQLPG |
| | 163723 | WVRQAPGKGLEWVGRIKSVTDGETTDYAAPVKGRF | TAPKLLIYKNNQRPSGVPDRFSGSKSGTSASLAISGLRSEDE |
| | | TISRDDSKSTLYLQMNSLKTEDTAVYYCTSSFCCR | ADYYCSTWDDSLSVRVFGGGTKLTVL (SEQ ID NO: |
| | | GGSCPSHDTSYCGGOKSYYYMDVWGKGTTVTVSS | 123) |
| | | (SEQ ID NO: 1755) | |
| 10 | AIP- | EVQLVESGGALVKPGGSLRLSCAASGFTFSKAWMS | QSVLTQPPSASGTPGQRVTISCSGSSSNIGSSSVSWYQQLPG |
| | 167295 | WVRQAPGKGLEWVGRIKSVTDGETTDYAAPVKGRF | TAPKLLIYKNNQRPSGVPDRFSGSKSGTSASLAISGLRSEDE |
| | | TISRDDSKSTLYLQMNSLKTEDTAVYYCTSSFCCR | ADYYCSTWDDSLSVRVFGGGTKLTVL (SEQ ID NO: |
| | | GGSCPSHDTSYCGGQYSYYYMDVWGKGTTVTVSS | 123) |
| | | (SEQ ID NO: 1756) | |
| 11 | AIP- | EVQLVESGGALVKPGGSLRLSCAASGFTFSKAWMS | QSVLTQPPSASGTPGQRVTISCSGSSSNIGSSSVSWYQQLPG |
| | 125646 | WVRQAPGKGLEWVGRIKSVTDGETTDYAAPVKGRF | TAPKLLIYKNNQRPSGVPDRFSGSKSGTSASLAISGLRSEDE |
| | | TISRDDSKSTLYLOMNSLKTEDTAVYYCTSSFCCR | ADYYCSTWDDSLSVRVFGGGTKLTVL (SEQ ID NO: |
| | | GGSCPSHDTSYCGQYKSYYYMDVWGKGTTVTVSS | 123) |
| | | (SEQ ID NO: 1746) | |
| | AIP- | EVQLVESGGALVKPGGSLRLSCAASGFTFSKAWMS | QSVLTQPPSASGTPGQRVTISCSGSSSNIGSSSVSWYQQLPG |
| | 114111 | WVRQAPGKGLEWVGRIKSVTDGETTDYAAPVKGRF | TAPKLLIYKNNQRPSGVPDRFSGSKSGTSASLAISGLRSEDE |
| | | TISRDDSKSTLYLQMNSLKTEDTAVYYCTSSFCCR | ADYYCSTWDDSLSVRVFGGGTKLTVL (SEQ ID NO: |
| | | GGSCPSHDTSYCGGQYKSSYYYMDVWGKGTTVTVS | 123) |
| | | S (SEQ ID NO: 1757) | |

TABLE 3B

| AIP | | | | | | |
|---|---|---|---|---|---|---|
| Number | HCDR1 | HCDR2 | HCDR3 | LCDR1 | LCDR2 | LCDR3 |
| AIP- | GFTFSKAWMS | RIKSVTDGETTDYA | TSSFCGGPSHDTSYC | SGSSSNIGSSSV | KNNQRPS | STWDDSLSVRV |
| 131762 | (SEQ ID NO: | APVKG (SEQ ID | GGQYKSYYYMDV | S (SEQ ID | (SEQ ID NO: | (SEQ ID NO: |
| | 1) | NO: 9) | (SEQ ID NO: | NO: 48) | 59) | 68) |
| | | | 1734) | | | |
| AIP- | GFTFSKAWMS | RIKSVTDGETTDYA | TSSFCCRSGGSCPSH | SGSSSNIGSSSV | KNNQRPS | STWDDSLSVRV |
| 194564 | (SEQ ID NO: | APVKG (SEQ ID | DTSYCGGQYKSYYYM | S (SEQ ID | (SEQ ID NO: | (SEQ ID NO: |
| | 1) | NO: 9) | DV (SEQ ID NO: | NO: 48) | 59) | 68) |
| | | | 1735) | | | |
| AIP- | GFTFSKAWMS | RIKSVTDGETTDYA | TSSFCCRGGGSCPSH | SGSSSNIGSSSV | KNNQRPS | STWDDSLSVRV |
| 169752 | (SEQ ID NO: | APVKG (SEQ ID | DTSYCGGQYKSYYYM | S (SEQ ID | (SEQ ID NO: | (SEQ ID NO: |
| | 1) | NO: 9) | DV (SEQ ID NO: | NO: 48) | 59) | 68) |
| | | | 1730) | | | |
| AIP- | GFTFSKAWMS | RIKSVTDGETTDYA | TSSFCCRGGGGSCPS | SGSSSNIGSSSV | KNNQRPS | STWDDSLSVRV |
| 185680 | (SEQ ID NO: | APVKG (SEQ ID | HDTSYCGGQYKSYYY | S (SEQ ID | (SEQ ID NO: | (SEQ ID NO: |
| | 1) | NO: 9) | MDV (SEQ ID NO: | NO: 48) | 59) | 68) |
| | | | 1737) | | | |
| AIP- | GFTFSKAWMS | RIKSVTDGETTDYA | TSSFCCRGGSGGSCP | SGSSSNIGSSSV | KNNQRPS | STWDDSLSVRV |
| 145328 | (SEQ ID NO: | APVKG (SEQ ID | SHDTSYCGGQYKSYY | S (SEQ ID | (SEQ ID NO: | (SEQ ID NO: |
| | 1) | NO: 9) | YMDV (SEQ ID | NO: 48) | 59) | 68) |
| | | | NO: 1738) | | | |
| AIP- | GFTFSKAWMS | RIKSVTDGETTDYA | TSSFSCRGGSCPSHD | SGSSSNIGSSSV | KNNQRPS | STWDDSLSVRV |
| 182606 | (SEQ ID NO: | APVKG (SEQ ID | TSYGGQYKSYYYMDV | S (SEQ ID | (SEQ ID NO: | (SEQ ID NO: |
| | 1) | NO: 9) | (SEQ ID NO: | NO: 48) | 59) | 68) |
| | | | 1739) | | | |
| AIP- | GFTFSKAWMS | RIKSVTDGETTDYA | TSSFCCRGGSCPSHD | SGSSSNIGSSSV | KNNQRPS | STWDDSLSVRV |
| 107388 | (SEQ ID NO: | APVKG (SEQ ID | TSYCGGGQYKSYYYM | S (SEQ ID | (SEQ ID NO: | (SEQ ID NO: |
| | 1) | NO: 9) | DV (SEQ ID NO: | NO: 48) | 59) | 68) |
| | | | 1740) | | | |
| AIP- | GFTFSKAWMS | RIKSVTDGETTDYA | TSSFCCRGGSCPSHD | SGSSSNIGSSSV | KNNQRPS | STWDDSLSVRV |
| 191692 | (SEQ ID NO: | APVKG (SEQ ID | TSYCGGGSGYKSYYY | S (SEQ ID | (SEQ ID NO: | (SEQ ID NO: |
| | 1) | NO: 9) | MDV (SEQ ID NO: | NO: 48) | 59) | 68 |
| | | | 1741) | | | |
| AIP- | GFTFSKAWMS | RIKSVTDGETTDYA | TSSFCCRGGSCPSHD | SGSSSNIGSSSV | KNNQRPS | STWDDSLSVRV |
| 163723 | (SEQ ID NO: | APVKG (SEQ ID | TSYCGGQKSYYYMDV | S (SEQ ID | (SEQ ID NO: | (SEQ ID NO: |
| | 1) | NO: 9) | (SEQ ID NO: | NO: 48) | 59) | 68) |
| | | | 1742) | | | |
| AIP- | GFTFSKAWMS | RIKSVTDGETTDYA | TSSFCCRGGSCPSHD | SGSSSNIGSSSV | KNNQRPS | STWDDSLSVRV |
| 167295 | (SEQ ID NO: | APVKG (SEQ ID | TSYCGGQYSYYYMDV | S (SEQ ID | (SEQ ID NO: | (SEQ ID NO: |
| | 1) | NO: 9) | (SEQ ID NO: | NO: 48) | 59) | 68 |
| | | | 1743) | | | |
| AIP- | GFTFSKAWMS | RIKSVTDGETTDYA | TSSFCCRGGSCPSHD | SGSSSNIGSSSV | KNNQRPS | STWDDSLSVRV |
| 125646 | (SEQ ID NO: | APVKG (SEQ ID | TSYCGQYKSYYYMDV | S (SEQ ID | (SEQ ID NO: | (SEQ ID NO: |
| | 1) | NO: 9) | (SEQ ID NO: | NO: 48) | 59) | 68) |
| | | | 1744) | | | |
| AIP- | GFTFSKAWMS | RIKSVTDGETTDYA | TSSFCCRGGSCPSHD | SGSSSNIGSSSV | KNNQRPS | STWDDSLSVRV |
| 114111 | (SEQ ID NO: | APVKG (SEQ ID | TSYCGGQYKSSYYYM | S (SEQ ID | (SEQ ID NO: | (SEQ ID NO: |
| | 1) | NO: 9) | DV (SEQ ID NO: | NO: 48) | 59) | 68) |
| | | | 1745) | | | |

### Exemplar Expressed Scaffold Sequences

TABLE 4A

| | AIP | | |
|---|---|---|---|
| | Number | V_{H} Sequence | V_{L} Sequence |
| 1 | AIP- | EVQLVESGGALVKPGGSLRLSCAASGFTFSKAWMT | QSVLTQAPSASETPGQRVIISCSGSSSNIGSSSVSWYQQLPG |
| | 192482 | WVRQAPGKGLEWVGRIKSVTDGETTDYAAPVKGRF | TAPKLLIYKNNQRPSGVPDRFSGSKSGTSASLAISGLRSEDE |
| | | TISRDDSKSVLYLQMSSLKTEDTAVYFCTSSFCCR | ADYYCSTWDDSLSVRVFGGGTKLTVL (SEQ ID NO: |
| | | GGSCPSHDTSFCGGODKRYYYMDVWGKGTTVTVSS | 141) |
| | | (SEQ ID NO: 95) | |
| 2 | AIP- | EVQLVESGGGLVKPGGSLRLSCAGSGFTFNKAWMS | QQSVLTQPPSASGTPGQTVTISCSGSSSHIESNYVEWYQQLP |
| | 186044 | WVRQAPGKGLEWVGRIKSTSDGGITDYAAPVKGRF | ETAPKLLIHRNDQRPSGVPDRFSGSKSGTSASLAISGLRSED |
| | | TISRDDSKDTLYLQMNSLKTEDAAVYYCTTSFCCR | DADYYCAAWDDSLSVWVFGGGTKLTVL (SEQ ID NO: |
| | | GGRCPSRDTSFCGGQYNSYYYMDVWGRGTTVTVSS | 1759) |
| | | (SEQ ID NO: 1758) | |
| 3 | AIP- | EVQLVESGGALVKPGGSLRLSCAASGFTFSKAWMT | QSVLTQPPSASGTPGQRVTISCSGSSSNIGSSSVSWYQQLPG |
| | 122563 | WVRQAPGKGLEWVGRIKSVTDGETTDYAAPVKGRF | TAPKLLIYKNNQRPSGVPDRFSGSKSGTSASLAISGLRSEDE |
| | | TISRDDSKSTLYLQMNSLKTEDTAVYYCTSSFCCR | ADYYCSTWDDSLSVRVFGGGTKLTVL (SEQ ID NO: |
| | | GGSCPSHDTSFCGGQDKRYYYMDVWGKGTTVTVSS | 123) |
| | | (SEQ ID NO: 100) | |
| 4 | AIP- | EVQLVESGGALVKPGGSLRLSCAASGFTFSKAWMS | QSVLTQPPSASGTPGQRVTISCSGSSSNIGSSSVSWYQQLPG |
| | 160470 | WVRQAPGKGLEWVGRIKSVTDGETTDYAAPVKGRF | TAPKLLIYKNNQRPSGVPDRFSGSKSGTSASLAISGLRSEDE |
| | | TISRDDSKSTLYLQMNSLKTEDTAVYYCTSSFCCR | ADYYCSTWDDSLSVRVFGGGTKLTVL (SEQ ID NO: |
| | | GGSCPSHDTSYCGGQYKSYYYMDVWGKGTTVTVSS | 123) |
| | | (SEQ ID NO: 94) | |
| 5 | AIP - | EVQLVESGGALVKPGGSLRLSCAASGFDASKAWFT | QSVLTQPPSASGTPGQRVTISCSGSSSNIGSSSVSWYQQLPG |
| | 145518 | WVRQAPGKGLEWVGRIKSVTDGETTDYAAPVKGRF | TAPKLLIYKNNQRPSGVPDRFSGSKSGTSASLAISGLRSEDE |
| | | TISRDDSKSTLYLQMNSLKTEDTAVYYCTSSFCCR | ADYYCSTWDDSLSVRVFGGGTKLTVL (SEQ ID NO: |
| | | GGSCPSHDTSYCGGQYKSYYYMDVWGKGTTVTVSS | 123) |
| | | (SEQ ID NO: 1421) | |
| 6 | AIP - | EVQLVESGGALVKPGGSLRLSCAASGFTFSKAWMS | QSVLTQPPSASGTPGQRVTISCSGSSSNIGSSSVSWYQQLPG |
| | 167533 | WVRQAPGKGLEWVGRIKAADDGKQTDYAAPVKGRF | TAPKLLIYKNNQRPSGVPDRFSGSKSGTSASLAISGLRSEDE |
| | | TISRDDSKSTLYLQMNSLKTEDTAVYYCTSSFCCR | ADYYCSTWDDSLSVRVFGGGTKLTVL (SEQ ID NO: |
| | | GGSCPSHDTSYCGGQYKSYYYMDVWGKGTTVTVSS | 123) |
| | | (SEQ ID NO: 120) | |
| 7 | AIP- | EVQLVESGGALVKPGGSLRLSCAASGFTFSKAWMS | QSVLTQPPSASGTPGQRVTISCSGSSSNIGSSSVSWYQQLPG |
| | 112580 | WVRQAPGKGLEWVGRIKSVTDGETTDYAAPVKGRF | TAPKLLIYKNNQRPSGVPDRFSGSKSGTSASLAISGLRSEDE |
| | | TISRDDSKSTLYLOMNSLKTEDTAVYYCISSFCCR | ADYYCSTWDDSLSVRVFGGGTKLTVL (SEQ ID NO: |
| | | GNSCPSSDTSYCNGQYKSYYYMDVWGKGTTVTVSS | 123) |
| | | (SEQ ID NO: 121) | |
| 8 | AIP - | EVQLVESGGALVKPGGSLRLSCAASGFTFSKAWMS | QSVLTQPPSASGTPGQRVTISCHGSESDIGSHDVLWYQQLPG |
| | 150277 | WVRQAPGKGLEWVGRIKSVTDGETTDYAAPVKGRF | TAPKLLIYKNNQRPSGVPDRFSGSKSGTSASLAISGLRSEDE |
| | | TISRDDSKSTLYLQMNSLKTEDTAVYYCTSSFCCR | ADYYCSTWDDSLSVRVFGGGTKLTVL (SEQ ID NO: |
| | | GGSCPSHDTSYCGGQYKSYYYMDVWGKGTTVTVSS | 1666) |
| | | (SEQ ID NO: 94) | |
| 9 | AIP- | EVQLVESGGALVKPGGSLRLSCAASGFTFSKAWMS | QSVLTQPPSASGTPGQRVTISCSGSSSNIGSSSVSWYQQLPG |
| | 136060 | WVRQAPGKGLEWVGRIKSVTDGETTDYAAPVKGRF | TAPKLLIYKNSLRPQGVPDRFSGSKSGTSASLAISGLRSEDE |
| | | TISRDDSKSTLYLQMNSLKTEDTAVYYCTSSFCCR | ADYYCSTWDDSLSVRVFGGGTKLTVL (SEQ ID NO: |
| | | GGSCPSHDTSYCGGQYKSYYYMDVWGKGTTVTVSS | 168) |
| | | (SEQ ID NO: 94) | |
| 10 | 154708 | EVQLVESGGALVKPGGSLRLSCAASGFTFSKAWMS | QSVLTQPPSASGTPGQRVTISCSGSSSNIGSSSVSWYQQLPG |
| | AIP- | WVRQAPGKGLEWVGRIKSVTDGETTDYAAPVKGRF | TAPKLLIYKNNQRPSGVPDRFSGSKSGTSASLAISGLRSEDE |
| | | TISRDDSKSTLYLQMNSLKTEDTAVYYCTSSFCCR | ADYYCATIDESMKIKVFGGGTKLTVL (SEQ ID NO: |
| | | GGSCPSHDTSYCGGQYKSYYYMDVWGKGTTVTVSS | 1760) |
| | | (SEQ ID NO: 94) | |

TABLE 4B

| AIP | | | | | | |
|---|---|---|---|---|---|---|
| Number | HCDR1 | HCDR2 | HCDR3 | LCDR1 | LCDR2 | LCDR3 |
| AIP- | GFTFSKAWMT | RIKSVTDGETTDYA | TSSFCCRGGSCPSHD | SGSSSNIGSSSV | KNNQRPS | STWDDSLSVRV |
| 192482 | (SEQ ID NO: | APVKG (SEQ ID | TSFCGGODKRYYYMD | S (SEQ ID | (SEQ ID NO: | (SEQ ID NO: |
| | 8) | NO: 9) | V (SEQ ID NO: | NO: 48) | 59) | 68) |
| | | | 34) | | | |
| AIP - | GFTFNKAWMS | RIKSTSDGGITDYA | TTSFCCRGGRCPSRD | SGSSSHIESNYV | RNDQRPS | AAWDDSLSVWV |
| 186044 | (SEQ ID NO: | APVKG (SEQ ID | TSFCGGQYNSYYYMD | E (SEQ ID | (SEQ ID NO: | (SEQ ID NO: |
| | 1761) | NO: 16) | V (SEQ ID NO: | NO: 1762) | 1763) | 1764) |
| | | | 38 | | | |
| AIP- | GFTFSKAWMT | RIKSVTDGETTDYA | TSSFCCRGGSCPSHD | SGSSSNIGSSSV | KNNQRPS | STWDDSLSVRV |
| 122563 | (SEQ ID NO: | APVKG (SEQ ID | TSFCGGQDKRYYYMD | S (SEQ ID | (SEQ ID NO: | (SEQ ID NO: |
| | 8) | NO: 9) | V (SEQ ID NO: | NO: 48) | 59) | 68) |
| | | | 34) | | | |
| AIP - | GFTFSKAWMS | RIKSVTDGETTDYA | TSSFCCRGGSCPSHD | SGSSSNIGSSSV | KNNQRPS | STWDDSLSVRV |
| 160470 | (SEQ ID NO: | APVKG (SEQ ID | TSYCGGQYKSYYYMD | S (SEQ ID | (SEQ ID NO: | (SEQ ID NO: |
| | 1) | NO: 9) | V (SEQ ID NO: | NO: 48) | 59) | 68) |
| | | | 21) | | | |
| AIP - | GFDASKAWFT | RIKSVTDGETTDYA | TSSFCCRGGSCPSHD | SGSSSNIGSSSV | KNNQRPS | STWDDSLSVRV |
| 145518 | (SEQ ID NO: | APVKG (SEQ ID | TSYCGGQYKSYYYMD | S (SEQ ID | (SEQ ID NO: | (SEQ ID NO: |
| | 257) | NO: 9) | V (SEQ ID NO: | NO: 48) | 59) | 68) |
| | | | 21) | | | |
| AIP- | GFTFSKAWMS | RIKAADDGKQTDYA | TSSFCCRGGSCPSHD | SGSSSNIGSSSV | KNNQRPS | STWDDSLSVRV |
| 167533 | (SEQ ID NO: | APVKG (SEQ ID | TSYCGGQYKSYYYMD | S (SEQ ID | (SEQ ID NO: | (SEQ ID NO: |
| | 1) | NO: 19) | V (SEQ ID NO: | NO: 48) | 59) | 68) |
| | | | 21) | | | |
| AIP - | GFTFSKAWMS | RIKSVTDGETTDYA | ISSFCCRGNSCPSSD | SGSSSNIGSSSV | KNNQRPS | STWDDSLSVRV |
| 112580 | (SEQ ID NO: | APVKG (SEQ ID | TSYCNGQYKSYYYMD | S (SEQ ID | (SEQ ID NO: | (SEQ ID NO: |
| | 1) | NO: 9) | V (SEQ ID NO: | NO: 48) | 59) | 68) |
| | | | 27) | | | |
| AIP- | GFTFSKAWMS | RIKSVTDGETTDYA | TSSFCCRGGSCPSHD | HGSESDIGSHDV | KNNQRPS | STWDDSLSVRV |
| 150277 | (SEQ ID NO: | APVKG (SEQ ID | TSYCGGQYKSYYYMD | L (SEQ ID | (SEQ ID NO: | (SEQ ID NO: |
| | 1) | NO: 9) | V (SEQ ID NO: | NO: 890) | 59) | 68) |
| | | | 21) | | | |
| AIP - | GFTFSKAWMS | RIKSVTDGETTDYA | TSSFCCRGGSCPSHD | SGSSSNIGSSSV | KNSLRPQ | STWDDSLSVRV |
| 136060 | (SEQ ID NO: | APVKG (SEQ ID | TSYCGGQYKSYYYMD | S (SEQ ID | (SEQ ID NO: | (SEQ ID NO: |
| | 1) | NO: 9) | V (SEQ ID NO: | NO: 48) | 67) | 68) |
| | | | 21) | | | |
| AIP- | GFTFSKAWMS | RIKSVTDGETTDYA | TSSFCCRGGSCPSHD | SGSSSNIGSSSV | KNNQRPS | ATIDESMKIKV |
| 154708 | (SEQ ID NO: | APVKG (SEQ ID | TSYCGGQYKSYYYMD | S (SEQ ID | (SEQ ID NO: | (SEQ ID NO: |
| | 1) | NO: 9) | V (SEQ ID NO: | NO: 48) | 59) | 1765) |
| | | | 21) | | | |

1. An antibody that binds an RNA-protein complex, wherein the antibody comprises a heavy chain variable region comprising a HCDR1, a HCDR2, and a HCDR3, and a light chain variable region comprising a LCDR1, a LCDR2, and a LCDR3,
wherein the antibody comprises a pocket that is 13-20 A deep,
and the HCDR3 comprises
i. at least 25 amino acids
ii. a first intra-HCDR3 disulfide bond, and
iii. optionally, a second intra-HCDR3 disulfide bond.

2. The antibody of claim 1, wherein the HCDR3 comprises:
amino acid sequence X¹CCX²CX³CX⁴, wherein X¹=4 amino acid residues, X²=4-7 amino acid residues, X³=6-8 amino acid residues, and X⁴=10⁻¹⁴ amino acid residues,
amino acid sequence X¹CGGX²CX³ wherein X¹=4 amino acid residues, X²=7 amino acid residues, and X³=12 amino acid residues (SEQ ID NO: 1732);
amino acid sequence of X¹SCX²CX³ wherein X¹=4 amino acid residues, X²=4 amino acid residues, and X³=19 amino acid residues:
amino acid sequence X¹CCX²CX³CX⁴, wherein X¹=4 amino acid residues, X²=4 amino acid residues, X³=7 amino acid residues, and X⁴=12 amino acid residues (SEQ ID NO: 1731) or
(I/T)(S/T)X₃(F/Y)CCX₇(G/S)X₉X₁₀CX₁₂(N/S)X₁₄(D/E)TS(F/Y)CX₂₀(G/N)X₂₂X₂₃ X₂₄,X₂₅ (F/Y)YX₂₈X₂₉(D/N)X₃₁, wherein X₃ is A, P, or S; X₇ is H, L, Q, or R: X₉ is A, G, K, or N; X₁₀ is A, N, Q, R, or S; X₁₂ is A, L, or P; X₁₄ is H, Q, R, or S; X₂₀ is A, G, or N: X₂₂ is Q, S, or Y; X₂₃ is D, F, N, or Y; X₂₄ is A, K, N, P, or Q; X₂₅ is D, Q, R, or S: X₂₈ is F, L, W, or Y; X₂₉ is F, M, or V; and X₃₁ is I, P, or V (SEQ ID NO: 1729); or
(I/T)(S/T)X₃(F/Y)X₅CX₇(G/S)X₉X₁₀CX₁₂X₁₃X₁₄(D/E)X₁₆SX₁₈X₁₉X₂₀X₂₁X₂₂X₂₃X₂₄X₂₅(F/Y)(F/Y)X₂₈X₂₉(D/N)X₃₁, wherein X₃ is A, P, S, or T; X₅ is A, C, or S: X₇ is H, L, Q, or R; X₉ is A, G, K, or N; X₁₀ is A, N, Q, R, or S; X₁₂ is A, L, or P: X₁₃ is A, N, or S: X₁₄ is H, Q, R, or S: X₁₆ is N, Q, or T: X₁₈ is F, M, or Y; X₁₉ is C, S, or V; X₂₀ is A, G, or N; X₂₁ is A, G, or N; X₂₂ is Q, S, or Y: X₂₃ is D, F, N, S, or Y; X₂₄ is A, K, N, P, Q, or S; X₂₅ is D, K, Q, R, or S;
X₂₈ is F, L, W, or Y: X₂₉ is F, M, or V; and X₃₁ is I, P, or V.

3-7. (canceled)

8. The antibody of claim 1, wherein the HCDR3 comprises the amino acid sequence of TSSFCCRGGSCPSHDTSYCGGQYKSYYYMDV (SEQ ID NO: 21) comprises at least 1, 2, 3, 4, 5, 6, 7, 8, 9, or 10 amino acid substitutions at positions that maintain the first disulfide bond and, optionally the second disfulfide bond.

9. The antibody of claim 1, wherein the pocket comprises amino acid residues 36, 37, 38, 39, 40, 52, 55, 58, 60, 61, 105, 106, 107, 108, 109, 111.5, 111.7, 111.8, 111.9, and 114 in the V_{H} per IMGT numbering and amino acid residues 37, 38, 40, 42, 55, 56, 107, and 116 in the V_{L} per IMGT numbering.

10. The antibody of claim 9, wherein the pocket further comprises:
one or more amino acid residues selected from the group consisting of 57, 111.6, 112.9, 112.7 and 116 in the V_{H} per IMGT numbering; or all of amino acid residues 57, 111.6, 112.9, 112.7 and 116 in the V_{H} per IMGT numbering; and/or
one or more amino acid residues selected from the group consisting of 39, 52, 57, 80, 105, and 106 in the V_{L} per IMGT numbering: or all of amino acid residues 39, 52, 57, 80, 105, and 106 in the V_{L} per IMGT numbering;

11.-13. (canceled)

14. The antibody of claim 9, wherein the pocket comprises
at least 1, 2, 3, 4, 5, 6, 7, 8, 9, 10, 11, 12, 13, 14, 15, 16, 17, 18, 19 or 20 of the following residues in the V_{H}: A, C, H, K, M, N or R at position 36; A or V at position 37, F, H, V, W, or Y at position 38; F, M or Y at position 39; S, T, or Y at position 40, W at position 52; F or R at position 55, A or S at position 58; D, H, Q, S, or T at position 60; D, E, N, or S at position 61; I, T, or V at position 105; F, S or T at position 106; A, P, S, or T at position 107; F or Y at position 108; A, C, or S at position 109; A, C, L or P at position 111.5; H, Q, R, or S at position 111.7; D or E at position 111.8; C, N, Q, or T at position 111.9; or C, F, L, W, or Y at position 114; and/or
at least 1, 2, 3, 4, 5, 6, 7, or 8 of the following residues in the V_{L}: A, H, N, S, or T at position 37; A, D, F, S, T, or Y at position 38; A, D, E, L, S, T, or Y at position 40; Y at position 42; H or Y at position 55; A, H, K, M, N, or R at position 56; F, I or W at position 107; or H, K, Q, R, V, or W at position 116.

15. The antibody of claim 14, further comprising
at least 1, 2, 3, 4, or 5 of the following residues in the V_{H} per IMGT numbering: K or Q at position 57; A, C, D, N, or S at position 111.6; S at position 112.9; A, C, S, or V at position 112.7; or D or N at position 116; and/or
at least 1, 2, 3, 4, 5, or 6 following residues in the V_{L} per IMGT numbering: T or V at position 39; L at position 52; D or N at position 57; K at position 80; A or S at position 105; or A, I, S, or T at position 106; or
the pocket comprises:
at least 1, 2, 3, 4, 5, 6, 7, 8, 9, 10, 11, 12, 13, 14, 15, 16, 17, 18, 19 or 20 of the following residues in the V_{H}: K at position 36: A at position 37: W at position 38: F or M at position 39: S or T at position 40: W at position 52: R at position 55: A or S at position 58; D, S, or T at position 60: D at position 61: I or T at position 105: S or T at position 106: S at position 107: F at position 108: C at position 109: P at position 111.5: H, R, or S at position 111.7: D at position 111.8: T at position 111.9: or Y at position 114; and/or
at least 1, 2, 3, 4, 5, 6, 7, or 8 of the following residues in the V_{L}: H, N, or S at position 37: D, S or Y at position 38: E, L, or S at position 40: Y at position 42: H or Y at position 55: K or R at position 56: I or W at position 107: or K, R, or W at position 116.

16. (canceled)

17. The antibody of claim 15, further comprising
at least 1, 2, 3, 4, or 5 of the following residues in the V_{H} per IMGT numbering: K at position 57; S at position 111.6; S at position 112.9; C at position 112.7; or D at position 116; and/or
at least 1, 2, 3, 4, 5, or 6 following residues in the V_{L} per IMGT numbering: V at position 39; L at position 52; N at position 57; K at position 80; A or S at position 105; or A or T at position 106.

18. A method of generating an antibody that binds to an RNA-protein complex comprising mutating a scaffold antibody that binds to a first RNA-protein complex to generate an antibody that binds to a second RNA-protein complex and has an antigen binding specificity that differs from the antigen binding specificity of the scaffold antibody, wherein the scaffold antibody comprises a heavy chain variable region comprising a HCDR1, a HCDR2, and a HCDR3, and a light chain variable region comprising a LCDR1, a LCDR2, and a LCDR3, and comprises a pocket that is 13-20 Å deep, and the HCDR3 comprises
i. at least 25 amino acids
ii. a first intra-HCDR3 disulfide bond, and
iii. optionally, a second intra-HCDR3 disulfide bond.

19. The method of claim 18, wherein the HCDR3 comprises:
the amino acid sequence of X¹CCX²CX³CX⁴, wherein X¹=4 amino acid residues, X₂₌₄-7 amino acid residues, X³=6-8 amino acid residues, and X⁴=10-14 amino acid residues (SEQ ID NO: 1728); or
the amino acid sequence of (I/T)(S/T)X₃(F/Y)CCX₇(G/S)X₉X₁₀CX₁₂(N/S)X₁₄(D/E)TS(F/Y)CX₂₀(G/N)X₂₂X₂₃X₂₄,X₂₅ (F/Y)YX₂₈X₂₉(D/N)X₃₁, wherein X₃ is A, P, or S; X₇ is H, L, Q, or R; X₉ is A, G, K, or N: X₁₀ is A, N, Q, R, or S; X₁₂ is A, L, or P: X₁₄ is H, Q, R, or S; X₂₀ is A, G, or N; X₂₂ is Q, S, or Y: X₂₃ is D, F, N, or Y; X₂₄ is A, K, N, P, or Q; X₂₅ is D, Q, R, or S; X₂₈ is F, L, W, or Y: X₂₉ is F, M, or V; and X₃₁ is I, P, or V (SEQ ID NO: 1729); or
the amino acid sequence of X¹CCX²CX³ C X⁴, wherein X¹=4 amino acid residues, X²=4 amino acid residues, X³=7 amino acid residues, and X⁴=12 amino acid residues (SEQ ID NO: 1731): or
the amino acid sequence of X¹CGGX²CX³ wherein X¹=4 amino acid residues, X²=7 amino acid residues, and X³=12 amino acid residues (SEQ ID NO: 1732); or
the amino acid sequence of (I/T)(S/T)X₃(F/Y)X₅CX₇(G/S)X₉X₁₀CX₁₂X₁₃X₁₄(D/E)X₁₆SX₁₈X₁₉X₂₀X₂₁X₂₂X₂₃X₂₄X₂₅(F/Y)(F/Y)X₂₈X₂₉(D/N)X₃₁, wherein X₃ is A, P, S, or T; X₅ is A, C, or S; X₇ is H, L, Q, or R; X₉ is A, G, K, or N; X₁₀ is A, N, Q, R, or S: X₁₂ is A, L, or P: X₁₃ is A, N, or S: X₁₄ is H, Q, R, or S: X₁₆ is N, Q, or T; X₁₈ is F, M, or Y: X₁₉ is C, S, or V: X₂₀ is A, G, or N: X₂₁ is A, G, or N: X₂₂ is Q, S, or Y: X₂₃ is D, F, N, S, or Y: X₂₄ is A, K, N, P, Q, or S; X₂₅ is D, K, Q, R, or S: X₂₈ is F, L, W, or Y: X₂₉ is F, M, or V; and X₃₁ is I, P, or V: or
the amino acid sequence of X¹SCX²CX³ wherein X¹=4 amino acid residues, X²=4 amino acid residues, and X³=19 amino acid residues: or
the amino acid sequence of TSSFCCRGGSCPSHDTSYCGGQYKSYYYMDV (SEQ ID NO: 21) comprises at least 1, 2, 3, 4, 5, 6, 7, 8, 9, or 10 amino acid substitutions at positions that maintain the first disulfide bond and, optionally the second disfulfide bond.

20.-25. (canceled)

26. The method of claim 18, wherein the pocket comprises amino acid residues 36, 37, 38, 39, 40, 52, 55, 58, 60, 61, 105, 106, 107, 108, 109, 111.5, 111.7, 111.8, 111.9, and 114 in the V_{H} per IMGT numbering and amino acid residues 37, 38, 40, 42, 55, 56, 107, and 116 in the V_{L} per IMGT numbering, optionally wherein
the pocket further comprises one or more amino acid residues selected from the group consisting of 57, 111.6, 112.9, 112.7 and 116 in the V_{H} per IMGT numbering, or all of the amino acid residues 57, 111.6, 112.9, 112.7 and 116 in the V_{H} per IMGT numbering; and/or
the pocket further comprises one or more amino acid residues selected from the group consisting of 39, 52, 57, 80, 105, and 106 in the V_{L} per IMGT numbering: or all of the amino acid residues 9, 52, 57, 80, 105, and 106 in the V_{L} per IMGT numbering.

27.-30. (canceled)

31. The method of claim 26, wherein the pocket comprises
at least 1, 2, 3, 4, 5, 6, 7, 8, 9, 10, 11, 12, 13, 14, 15, 16, 17, 18, 19 or 20 of the following residues in the V_{H}: A, C, H, K, M, N or R at position 36; A or V at position 37, F, H, V, W, or Y at position 38; F, M or Y at position 39; S, T, or Y at position 40, W at position 52; F or R at position 55, A or S at position 58; D, H, Q, S, or T at position 60; D, E, N, or S at position 61; I, T, or V at position 105; F, S or T at position 106; A, P, S, or T at position 107; F or Y at position 108; A, C, or S at position 109; A, C, L or P at position 111.5; H, Q, R, or S at position 111.7; D or E at position 111.8; C, N, Q, or T at position 111.9; or C, F, L, W, or Y at position 114; and/or
at least 1, 2, 3, 4, 5, 6, 7, or 8 of the following residues in the V_{L}: A, H, N, S, or T at position 37; A, D, F, S, T, or Y at position 38; A, D, E, L, S, T, or Y at position 40; Y at position 42; H or Y at position 55; A, H, K, M, N, or R at position 56; F, I or W at position 107;
or H, K, Q, R, V, or W at position 116.

32. The method of claim 31, further comprising
(a) at least 1, 2, 3, 4, or 5 of the following residues in the V_{H} per IMGT numbering: K or Q at position 57; A, C, D, N, or S at position 111.6; S at position 112.9; A, C, S, or V at position 112.7; or D or N at position 116; and/or
at least 1, 2, 3, 4, 5, or 6 following residues in the V_{L} per IMGT numbering: T or V at position 39; L at position 52; D or N at position 57; K at position 80; A or S at position 105; or A, I, S, or T at position 106; or
(b) at least 1, 2, 3, 4, 5, 6, 7, 8, 9, 10, 11, 12, 13, 14, 15, 16, 17, 18, 19 or 20 of the following residues in the V_{H}: K at position 36: A at position 37: W at position 38: F or M at position 39: S or T at position 40: W at position 52: R at position 55: A or S at position 58; D, S, or T at position 60: D at position 61: I or T at position 105: S or T at position 106: S at position 107: F at position 108: C at position 109: P at position 111.5: H, R, or S at position 111.7: D at position 111.8: T at position 111.9: or Y at position 114; and/or
at least 1, 2, 3, 4, 5, 6, 7, or 8 of the following residues in the V_{L}: H, N, or S at position 37: D, S or Y at position 38: E, L, or S at position 40: Y at position 42: H or Y at position 55: K or R at position 56: I or W at position 107: or K, R, or W at position 116.

33. (canceled)

34. The method of claim 33, wherein (b) further comprises
at least 1, 2, 3, 4, or 5 of the following residues in the V_{H} per IMGT numbering: K at position 57; S at position 111.6; S at position 112.9; C at position 112.7; or D at position 116;
and/or at least 1, 2, 3, 4, 5, or 6 following residues in the V_{L} per IMGT numbering: V at position 39; L at position 52; N at position 57; K at position 80; A or S at position 105; or A or T at position 106.

35. The method of claim 18, wherein the scaffold antibody comprises an HCDR3 as set forth in Table 1B, Table 2B, or Table 3B: or comprises the six CDRS of an antibody as set forth in Table 1B, Table 2B, or Table 3B.

36. (canceled)

37. The method of claim 18, wherein the method comprises generating a plurality of mutated scaffold antibodies;
screening the mutated scaffold antibodies for binding to the second RNA-protein complex; and
selecting a mutated scaffold antibody that binds to the second RNA-protein complex.

38. A library comprising a plurality of mutated scaffold antibodies produced by the method of claim 18.

39. An antibody that binds to tumor tissue, wherein the antibody comprises:
(a) a heavy chain variable region comprising HCDR1, HCDR2, and HCDR3 from Table 3B; and
a light chain variable region comprising LCDR1, LCDR2, and LCDR3 from Table 3B; or
(b) a heavy chain variable region (V_{H}) and a light chain variable region (V_{L}) from Table 3 Å; or
(c) a heavy chain variable region comprising:
an HCDR1 comprising the sequence GFTFSKAWMS (SEQ ID NO: 1), or a variant HCDR1 in which 1, 2, 3, 4, or 5 amino acids are substituted relative to the sequence;
an HCDR2 comprising the sequence RIKSVTDGETTDYAAPVKG (SEQ ID NO: 9), or a variant HCDR2 in which 1, 2, 3, 4, or 5 amino acids are substituted relative to the sequence; and
an HCDR3 comprising the sequence of an HCDR3 set forth in Table 3B, or a variant HCDR3 in which 1, 2, 3, 4, or 5 amino acids are substituted relative to the sequence; and
a light chain variable region comprising:
an LCDR1 comprising the sequence SGSSSNIGSSSVS (SEQ ID NO: 48), or a variant LCDR1 in which 1, 2, 3, 4, or 5 amino acids are substituted relative to the sequence;
an LCDR2 comprising the sequence KNNQRPS (SEQ ID NO: 59), or variant LCDR2 in which 1, 2, or 3 amino acids are substituted relative to the sequence; and
an LCDR3 comprising the sequence STWDDSLSVRV (SEQ ID NO: 68), or a variant LCDR3 in which 1, 2, 3, 4, or 5 amino acids are substituted relative to the sequence.

40. (canceled)

41. The antibody of claim 39, wherein the antibody comprises a CDR3 sequence: TSSFCGGPSHDTSYCGGQYKSYYYMDV (SEQ ID NO: 1734),
TSSFCCRSGGSCPSHDTSYCGGQYKSYYYMDV (SEQ ID NO: 1735),
TSSFCCRGGGSCPSHDTSYCGGQYKSYYYMDV (SEQ ID NO: 1730),
TSSFCCRGGGGSCPSHDTSYCGGQYKSYYYMDV (SEQ ID NO: 1737),
TSSFCCRGGSGGSCPSHDTSYCGGQYKSYYYMDV (SEQ ID NO: 1738),
TSSFSCRGGSCPSHDTSYGGQYKSYYYMDV (SEQ ID NO: 1739),
TSSFCCRGGSCPSHDTSYCGGGQYKSYYYMDV (SEQ ID NO: 1740),
TSSFCCRGGSCPSHDTSYCGGGSGYKSYYYMDV (SEQ ID NO: 1741),
TSSFCCRGGSCPSHDTSYCGGQKSYYYMDV (SEQ ID NO: 1742),
TSSFCCRGGSCPSHDTSYCGGQYSYYYMDV (SEQ ID NO: 1743),
TSSFCCRGGSCPSHDTSYCGQYKSYYYMDV (SEQ ID NO: 1744), or
TSSFCCRGGSCPSHDTSYCGGQYKSSYYYMDV (SEQ ID NO: 1745); or a variant thereof having 1, 2, or 3 amino acid substitutions.

42. The antibody of claim 41, wherein
the HCDR1 comprises the sequence GFTFSKAWMS (SEQ ID NO: 1),
the HCDR2 comprises the sequence RIKSVTDGETTDYAAPVKG (SEQ ID NO: 9),
the HCDR3 comprises the sequence
TSSFCCRGGSCPSHDTSYCGQYKSYYYMDV (SEQ ID NO: 1744) or
TSSFCCRGGSCPSHDTSYCGGQYKSSYYYMDV (SEQ ID NO: 1745),
the LCDR1 comprises the sequence SGSSSNIGSSSVS (SEQ ID NO: 48),
the LCDR2 comprises the sequence KNNQRPS (SEQ ID NO: 59), and
the LCDR3 comprises the sequence STWDDSLSVRV (SEQ ID NO: 68).

43.-44. (canceled)
